(19) United States
(12) Patent Application Publication
(10) Pub. No.: US 2021/0284728 A1
(43) Pub. Date: Sep. 16, 2021

(54) DUAL BINDING MOIETY

(71) Applicant: HARPOON THERAPEUTICS, INC., South San Francisco, CA (US)

(72) Inventors: Shuoyen Jack LIN, San Bruno, CA (US); Richard J. AUSTIN, San Francisco, CA (US); Bryan D. LEMON, Mountain View, CA (US); Holger WESCHE, San Francisco, CA (US)

(21) Appl. No.: 17/055,100

(22) PCT Filed: May 14, 2019
(86) PCT No.: PCT/US2019/032302

§ 371 Date: Nov. 12, 2020

Related U.S. Application Data
(60) Provisional application No. 62/756,480, filed on Nov. 6, 2018.
(60) Provisional application No. 62/671,351, filed on May 14, 2018.

(51) Int. Cl. C07K 16/28; C07K 16/46; C07K 16/18; C07K 16/24

(52) U.S. Cl. CPC C07K 16/2809; C07K 16/468; C07K 16/18; A61K 2039/505; C07K 16/246; C07K 2319/35; C07K 16/2863

(57) ABSTRACT

Disclosed herein is a dual binding moiety that comprises non-CDR loops for masking the binding of a domain, such as a target antigen binding domain to its target, by steric occlusion and specific masking, and CDRs for binding bulk serum proteins. Pharmaceutical compositions comprising the dual binding moiety disclosed herein and methods of using such compositions are further provided.

### CROSS-REFERENCE

This application claims the benefit of U.S. Provisional Application Nos. 62/671,351 filed May 14, 2018 and 62/756,480 filed Nov. 6, 2018, each of which is incorporated by reference herein in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if set forth in their entireties.

### BACKGROUND OF THE INVENTION

There is a need to extend the half-life of a therapeutic, diagnostic, or imaging molecule in circulation and also improve its ability to reach its target within an intended location (e.g., a tumor cell) without non-specific binding.

### SUMMARY OF THE INVENTION

An embodiment of this disclosure provides a dual binding moiety comprising a non-CDR loop and a cleavable linker, wherein the moiety is capable of an interaction with a domain by specific binding or by steric occlusion, wherein upon the interaction between the moiety and the domain the moiety is capable of masking the domain from binding its target, and wherein upon cleavage of the linker the moiety is capable of unmasking the domain. In some embodiments, the moiety is further capable of an interaction with a half-life extending protein. In some embodiments, the moiety comprises a binding site specific for the domain. In some embodiments, the non-CDR loop provides the binding site specific for the domain. In some embodiments, the non-CDR loop comprises AB, C″D, EF, and CC′ loops and wherein the binding site specific for the domain is within the AB, C″D, EF, or CC′ loop. In some embodiments, the binding site specific for the domain is within the CC′ loop. In some embodiments, the binding site specific for the domain is introduced within the CC′ loop by mutagenesis. In some embodiments, the non-CDR loop comprises AB, C″D, EF, and CC′ loops and wherein a CD3 epitope is grafted into at least one of the AB, C″D, EF, and CC′ loops. In some embodiments, the CD3 binding epitope grafted into at least one of the AB, C″D, EF, and CC′ loops enables the moiety to mask binding of a CD3 binding domain to its target. In some embodiments, the moiety is capable of competitively binding to the CD3 binding domain in order to mask binding of the CD3 binding domain to its target. In some embodiments, the moiety is a natural peptide, a synthetic peptide, an engineered scaffold, or an engineered bulk serum protein. In some embodiments, the engineered scaffold comprises an sdAb, an scFv, an Fab, a VHH, a IgNAR, a VH, a VL, a fibronectin type III domain, an immunoglobulin-like scaffold, a bacterial albumin-binding domain, an adnectin, a monobody, an affibody, an affilin, an affimer, an affitin, an alphabody, an anticalin, an avimer, a centyrin, a DARPin, a cystine knot peptide, a lipocalin, a three-helix bundle scaffold, a protein G-related albumin-binding module, a DNA or RNA aptamer scaffold, or any combinations thereof. In some embodiments, the non-CDR-loop is from a variable domain, a constant domain, a C1-set domain, a C2-set domain, an I-domain, or any combinations thereof. In some embodiments, the dual binding moiety comprises albumin. In some embodiments, the moiety further comprises a complementarity determining region (CDR). In some embodiments, the moiety comprises a binding site specific for a bulk serum protein. In some embodiments, the bulk serum protein is albumin, transferrin, IgG1, IgG2, IgG4, IgG3, IgA monomer, Factor XIII, Fibrinogen, IgE, or pentameric IgM. In some embodiments, the moiety comprises a binding site specific for an immunoglobulin light chain. In some embodiments, the immunoglobulin light chain is an Igκ free light chain. In some embodiments, the CDR provides the binding site specific for the bulk serum protein or the immunoglobulin light chain. In some embodiments, the cleavable linker comprises a cleavage site. In some embodiments, the cleavage site is a protease cleavage site. In some embodiments, the domain is a target antigen binding domain, wherein the moiety is capable of masking the target antigen binding domain from binding to a tumor antigen. In some embodiments, the tumor antigen comprises EpCAM, EGFR, HER-3, c-Met, FoIR, PSMA, CD38, BCMA, and CEA. 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the domain is a target antigen binding domain, wherein the moiety is capable of masking the target antigen binding domain from binding to an immune checkpoint protein. In some embodiments, the immune checkpoint protein is CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA. In some embodiments, the domain is a target antigen binding domain, wherein the moiety is capable of masking the target antigen binding domain from binding an immune cell. In some embodiments, the domain is a target antigen binding domain, wherein the moiety is capable of masking the target antigen binding domain from binding a T-cell. In some embodiments, the protease cleavage site is recognized by a serine protease, a cysteine protease, an aspartate protease, a threonine protease, a glutamic acid protease, a metalloproteinase, a gelatinase, or a asparagine peptide lyase. In some embodiments, the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a refill, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP7, a MMP8, a MMP9, a MMP10, a MMP11, a MMP12, a MMP13, a MMP14, an ADAMS, an ADAM10, an ADAM12, an urokinase plasminogen activator (uPA), an enterokinase, a prostate-specific target (PSA, hK3), an interleukin-1β converting enzyme, a thrombin, a FAP (FAP-α), a dipeptidyl peptidase, a type II transmembrane serine protease (TTSP), a neutrophil elastase, a cathepsin G, a proteinase 3, a neutrophil serine protease 4, a mast cell chymase, and a mast cell tryptase.

One embodiment provides a dual binding moiety comprising a non-CDR loop and a cleavable linker, wherein the moiety is capable of masking a target antigen binding domain from binding to its target. In some embodiments, the moiety is further capable of binding a half-life extending protein. In some embodiments, the moiety comprises a binding site specific for the target antigen binding domain. In some embodiments, the non-CDR loop provides the binding site specific for the target antigen binding domain. In some embodiments, the non-CDR loop comprises AB, C″D, EF, and CC′ loops and wherein the binding site specific for the target antigen binding domain is within the AB, C″D, EF, or CC′ loop. In some embodiments, the binding site specific for the target antigen binding domain is within the CC′ loop. In some embodiments, the binding site specific for the target antigen binding domain is introduced within the CC′ loop by mutagenesis. In some embodiments, the non-CDR loop comprises AB, C″D, EF, and CC′ loops and wherein a CD3 epitope is grafted into at least one of the AB, C″D, EF, and CC′ loops. In some embodiments, the CD3 binding epitope grafted into at least one of the AB, C″D, EF, and CC′ loops enables the moiety to mask binding of a CD3 binding domain to its target. In some embodiments, the moiety is capable of competitively binding to the CD3 binding domain in order to mask binding of the CD3 binding domain to its target. In some embodiments, the moiety is a natural peptide, a synthetic peptide, an engineered scaffold, or an engineered bulk serum protein. In some embodiments, the engineered scaffold comprises an sdAb, an scFv, an Fab, a VHH, a IgNAR, a VH, a VL, a fibronectin type III domain, an immunoglobulin-like scaffold, a bacterial albumin-binding domain, an adnectin, a monobody, an affibody, an affilin, an affimer, an affitin, an alphabody, an anticalin, an avimer, a centyrin, a DARPin, a cystine knot peptide, a lipocalin, a three-helix bundle scaffold, a protein G-related albumin-binding module, a DNA or RNA aptamer scaffold, or any combinations thereof. In some embodiments, the non-CDR-loop is from a variable domain, a constant domain, a C1-set domain, a C2-set domain, an I-domain, or any combinations thereof. In some embodiments, the moiety comprises albumin. In some embodiments, the moiety further comprises a complementarity determining region (CDR). In some embodiments, the moiety comprises a binding site specific for a bulk serum protein. In some embodiments, the bulk serum protein is albumin, transferrin, IgG1, IgG2, IgG4, IgG3, IgA monomer, Factor XIII, Fibrinogen, IgE, or pentameric IgM. In some embodiments, the moiety comprises a binding site specific for an immunoglobulin light chain. In some embodiments, the immunoglobulin light chain is an Igκ free light chain. In some embodiments, the CDR provides the binding site specific for the bulk serum protein or the immunoglobulin light chain. In some embodiments, the cleavable linker comprises a cleavage site In some embodiments, the cleavage site is a protease cleavage site. In some embodiments, the tumor antigen comprises EpCAM, EGFR, HER-2, HER-3, c-Met, FoIR, PSMA, CD38, BCMA, and CEA. 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the tumor antigen comprises at least one of: EpCAM (exemplary protein sequences comprise UniProtkB ID No. P16422, B5MCA4), EGFR (exemplary protein sequence comprises UniProtkB ID No. P00533), HER-2 (exemplary protein sequence comprises UniProtkB ID No. P04626), HER-3 (exemplary protein sequence comprises UniProtkB ID No. P21860), c-Met (exemplary protein sequence comprises UniProtkB ID No. P08581), FoIR (exemplary protein sequence comprises UniProtkB ID No. P15238), PSMA (exemplary protein sequence comprises UniProtkB ID No. Q04609), CD38 (exemplary protein sequence comprises UniProtkB ID No. P28907), BCMA (exemplary protein sequence comprises UniProtkB ID No. Q02223), and CEA (exemplary protein sequence comprises UniProtkB ID No. P06731, 5T4 (exemplary protein sequence comprises UniProtkB ID No. Q13641), AFP (exemplary protein sequence comprises UniProtkB ID No. P02771), B7-H3 (exemplary protein sequence comprises UniProtkB ID No. Q5ZPR3), CDH-6 (exemplary protein sequence comprises UniProtkB ID No. P97326), CAIX (exemplary protein sequence comprises UniProtkB ID No. Q16790), CD117 (exemplary protein sequence comprises UniProtkB ID No. P10721), CD123 (exemplary protein sequence comprises UniProtkB ID No. P26951), CD138 (exemplary protein sequence comprises UniProtkB ID No. P18827), CD166 (exemplary protein sequence comprises UniProtkB ID No. Q13740), CD19 (exemplary protein sequence comprises UniProtkB ID No. P15931), CD20 (exemplary protein sequence comprises UniProtkB ID No. P11836), CD205 (exemplary protein sequence comprises UniProtkB ID No. 060449), CD22 (exemplary protein sequence comprises UniProtkB ID No. P20273), CD30 (exemplary protein sequence comprises UniProtkB ID No. P28908), CD33 (exemplary protein sequence comprises UniProtkB ID No. P20138), CD352 (exemplary protein sequence comprises UniProtkB ID No. Q96DU3), CD37 (exemplary protein sequence comprises UniProtkB ID No. P11049), CD44 (exemplary protein sequence comprises UniProtkB ID No. P16070), CD52 (exemplary protein sequence comprises UniProtkB ID No. P31358), CD56 (exemplary protein sequence comprises UniProtkB ID No. P13591), CD70 (exemplary protein sequence comprises UniProtkB ID No. P32970), CD71 (exemplary protein sequence comprises UniProtkB ID No. P02786), CD74 (exemplary protein sequence comprises UniProtkB ID No. P04233), CD79b (exemplary protein sequence comprises UniProtkB ID No. P40259), DLL3 (exemplary protein sequence comprises UniProtkB ID No. Q9NYJ7), EphA2 (exemplary protein sequence comprises UniProtkB ID No. P29317), FAP (exemplary protein sequence comprises UniProtkB ID No. Q12884), FGFR2 (exemplary protein sequence comprises UniProtkB ID No. P21802), FGFR3 (exemplary protein sequence comprises UniProtkB ID No. P22607), GPC3 (exemplary protein sequence comprises UniProtkB ID No. P51654), gpA33 (exemplary protein sequence comprises UniProtkB ID No. Q99795), FLT-3 (exemplary protein sequence comprises UniProtkB ID No. P36888), gpNMB (exemplary protein sequence comprises UniProtkB ID No. Q14956), HPV-16 E6 (exemplary protein sequence comprises UniProtkB ID No. P03126), HPV-16 E7 (exemplary protein sequence comprises UniProtkB ID No. P03129), ITGA2 (exemplary protein sequence comprises UniProtkB ID No. P17301), ITGA3 (exemplary protein sequence comprises UniProtkB ID No. P26006), SLC39A6 (exemplary protein sequence comprises UniProtkB ID No. Q13433), MAGE (exemplary protein sequence comprises UniProtkB ID No. Q9HC15), mesothelin (exemplary protein sequence comprises UniProtkB ID No. Q13421), Muc1 (exemplary protein sequence comprises UniProtkB ID No. P15941), Muc16 (exemplary protein sequence comprises UniProtkB ID No. Q8WX17), NaPi2b (exemplary protein sequence comprises UniProtkB ID No. 095436), Nectin-4 (exemplary protein sequence comprises UniProtkB ID No. Q96918), CDH-3 (exemplary protein sequence comprises UniProtkB ID No. Q8WX17), CDH-17 (exemplary protein sequence comprises UniProtkB ID No. E5RJT3), EPHB2 (exemplary protein sequence comprises UniProtkB ID No. P29323), ITGAV (exemplary protein sequence comprises UniProtkB ID No. P06756), ITGB6 (exemplary protein sequence comprises UniProtkB ID No. P18564), NY-ESO-1 (exemplary protein sequence comprises UniProtkB ID No. P78358), PRLR (exemplary protein sequence comprises UniProtkB ID No. P16471), PSCA (exemplary protein sequence comprises UniProtkB ID No. 043653), PTK7 (exemplary protein sequence comprises UniProtkB ID No. Q13308), ROR1 (exemplary protein sequence comprises UniProtkB ID No. Q01973), SLC44A4 (exemplary protein sequence comprises UniProtkB ID No. Q53GD3), SLITRK5 (exemplary protein sequence comprises UniProtkB ID No. Q81W52), SLITRK6 (exemplary protein sequence comprises UniProtkB ID No. Q9HY7), STEAP1 (exemplary protein sequence comprises UniProtkB ID No. Q9UHE8), TIM1 (exemplary protein sequence comprises UniProtkB ID No. Q96D42), Trop2 (exemplary protein sequence comprises UniProtkB ID No. P09758), or WT1 (exemplary protein sequence comprises UniProtkB ID No. P19544), or any combinations thereof. In some embodiments, the moiety is capable of masking the target antigen binding domain from binding to a tumor antigen. In some embodiments, the moiety is capable of masking the target antigen binding domain from binding to an immune checkpoint protein. In some embodiments, the immune checkpoint protein is at least one of: CD27 (exemplary protein sequence comprises UniProtkB ID No. P26842), CD137 (exemplary protein sequence comprises UniProtkB ID No. Q07011), 2B4 (exemplary protein sequence comprises UniProtkB ID No. Q9bZW8), TIGIT (exemplary protein sequence comprises UniProtkB ID No. Q495A1), CD155 (exemplary protein sequence comprises UniProtkB ID No. P15151), ICOS (exemplary protein sequence comprises UniProtkB ID No. Q9Y6W8), HVEM (exemplary protein sequence comprises UniProtkB ID No. 043557), CD40L (exemplary protein sequence comprises UniProtkB ID No. P29965), LIGHT (exemplary protein sequence comprises UniProtkB ID No. 043557), OX40 (exemplary protein sequence comprises UniProtkB ID No.), DNAM-1 (exemplary protein sequence comprises UniProtkB ID No. Q15762), PD-L1 (exemplary protein sequence comprises UniProtkB ID No. Q9ZQ7), PD1 (exemplary protein sequence comprises UniProtkB ID No. Q15116), PD-L2 (exemplary protein sequence comprises UniProtkB ID No. Q9BQ51), CTLA-4 (exemplary protein sequence comprises UniProtkB ID No. P16410), CD8 (exemplary protein sequence comprises UniProtkB ID No. P10966, P01732), CD40 (exemplary protein sequence comprises UniProtkB ID No. P25942), CEACAM1 (exemplary protein sequence comprises UniProtkB ID No. P13688), CD48 (exemplary protein sequence comprises UniProtkB ID No. P09326), CD70 (exemplary protein sequence comprises UniProtkB ID No. P32970), AA2AR (exemplary protein sequence comprises UniProtkB ID No. P29274), CD39 (exemplary protein sequence comprises UniProtkB ID No. P49961), CD73 (exemplary protein sequence comprises UniProtkB ID No. P21589), B7-H3 (exemplary protein sequence comprises UniProtkB ID No. Q5ZPR3), B7-H4 (exemplary protein sequence comprises UniProtkB ID No. Q7Z7D3), BTLA (exemplary protein sequence comprises UniProtkB ID No. Q76A9), IDO1 (exemplary protein sequence comprises UniProtkB ID No. P14902), IDO2 (exemplary protein sequence comprises UniProtkB ID No. Q6ZQW0), TDO (exemplary protein sequence comprises UniProtkB ID No. P48755), KIR (exemplary protein sequence comprises UniProtkB ID No. Q99706), LAG-3 (exemplary protein sequence comprises UniProtkB ID No. P18627), TIM-3 (also known as HAVCR2, exemplary protein sequence comprises UniProtkB ID No. Q8TDQ0), or VISTA (exemplary protein sequence comprises UniProtkB ID No. Q9D659) In some embodiments, the moiety is capable of masking the target antigen binding domain from binding an immune cell. In some embodiments, the moiety is capable of masking the target antigen binding domain from binding a T-cell. In some embodiments, the protease cleavage site is recognized by a serine protease, a cysteine protease, an aspartate protease, a threonine protease, a glutamic acid protease, a metalloproteinase, a gelatinase, or a asparagine peptide lyase. In some embodiments, the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP7, a MMP8, a MMP9, a MMP10, a MMP11, a MMP12, a MMP13, a MMP14, an ADAM9, an ADAM10, an ADAM12, an urokinase plasminogen activator (uPA), an enterokinase, a prostate-specific target (PSA, hK3), an interleukin-1β converting enzyme, a thrombin, a FAP (FAP-11), a dipeptidyl peptidase, a type II transmembrane serine protease (TTSP), a neutrophil elastase, a cathepsin G, a proteinase 3, a neutrophil serine protease 4, a mast cell chymase, and a mast cell tryptase. In some embodiments, the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP7, a MMP8, a MMP9, a MMP10, a MMP11, a MMP12, a MMP13, a MMP14, an ADAMS, an ADAM10, an ADAM12, an urokinase plasminogen activator (uPA), an enterokinase, a prostate-specific target (PSA, hK3), an interleukin-1β converting enzyme, a thrombin, a FAP (FAP-α), a dipeptidyl peptidase, a type II transmembrane serine protease (TTSP), a neutrophil elastase, a cathepsin G, a proteinase 3, a neutrophil serine protease 4, a mast cell chymase, and a mast cell tryptase.

In some embodiments, the moiety is capable of an interaction with the target antigen binding domain by specific binding or by steric occlusion, wherein the interaction between the moiety and the target antigen binding domain enables the masking of the target antigen binding domain from binding to its target. In some embodiments, upon cleavage of the linker the moiety is capable of unmasking the target antigen binding domain whereby the target antigen binding domain is capable of binding to its target. In some embodiments, the dual binding moiety comprises two or more polypeptides linked by a non-cleavable linker. In some embodiments, the CDRs and the non-CDR loops are in separate polypeptides. In some embodiments, the CDRs and the non-CDR loops are in a first polypeptide and wherein a second polypeptide comprises residue for extending the non-CDR loops.

In some embodiments, the dual binding moiety comprises an Ig, an Ig-like scaffold, a beta-sandwich scaffold, or a non beta-sandwich scaffold. In some embodiments, the dual binding moiety comprises a non-CDR loops that comprises a CC′ loop of at least one of: a camelid VHH domain, a human VH domain, a humanized VH domain, or a single domain antibody. In some embodiments, the moiety comprises a binding site specific for a CD3ε domain, and wherein the binding site for the CD3ε domain comprises at least one of the following motifs: QDGNE, QDGNEE, DGNE, and DGNEE.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which.

FIG. 1 illustrates a variable domain of an exemplary immunoglobulin domain, comprising complementarity determining regions (CDR1, CDR2, and CDR3), and non-CDR loops connecting the beta strand (AB, CC′, C″ D, EF, and DE).

FIGS. 2A-2B provide exemplary arrangements of various domains of a conditionally active binding protein of this disclosure. FIG. 2A: Version 1. FIG. 2B: Version 2.

FIG. 3 shows an exemplary conditionally active target binding protein of this disclosure.

FIGS. 4A-4B show activation and possible mode of action of trispecific molecules (ProTriTAC). FIG. 4A shows ProTriTAC molecules in circulation, in tumor environment, and in circulation. FIG. 4B shows exemplary sequence for the protease cleavable site in a linker tethered to an anti-albumin dual binding moiety and an SDS-PAGE gel showing the ProTriTAC in its activatable (prodrug) and activated (active drug) states.

FIG. 5 shows a schematic flowchart for manufacturing a ProTriTAC molecule and an SDS-PAGE gel showing three purified ProTriTAC molecules.

FIGS. 6A-6B show analytical size exclusion chromatograms on a ProTriTAC molecule exposed to different stress conditions in graph form in FIG. 6A with the corresponding data in FIG. 6B.

FIG. 7 shows protease-dependent, anti-tumor activity of exemplary ProTriTAC molecules in HCT116 Colorectal Tumor Xenograft Model in NSG Mice.

FIGS. 8A-8D show various designs of exemplary ProTriTAC and control molecules. FIG. 8A illustrates an exemplary Control #1. FIG. 8B illustrates an exemplary Control #2.

FIG. 8C illustrates an exemplary ProTriTAC molecule. FIG. 8D illustrates an exemplary activated ProTriTAC molecule.

FIG. 9 shows pharmacokinetic profiles for exemplary ProTriTAC and control molecules.

FIG. 10 shows conversion of an exemplary prodrug ProTriTAC molecule into an active drug with the empirical half-lives.

FIG. 11 shows plasma clearance of an exemplary ProTriTAC molecule and its converted active drug format.

FIG. 12 shows CD3 binding potential of an exemplary ProTriTAC molecule, its converted active drug format, and a control non-cleavable ProTriTAC molecule.

FIG. 13 shows human primary T cell binding potential of an exemplary ProTriTAC molecule, its converted active drug format, and a control non-cleavable ProTriTAC molecule.

FIG. 14 shows T cell killing potential of an exemplary ProTriTAC molecule, its converted active drug format, and a control non-cleavable ProTriTAC molecule.

FIG. 15 shows the schematic structure of an exemplary trispecific molecule containing a dual binding moiety as described herein (also referred to herein as ProTriTAC or activatable ProTriTAC).

FIG. 16A-16E shows exemplary schematic structures for Prodrug molecules combining functional masking and half-life extension. FIG. 16A shows a ProDrug molecule comprising an anti-albumin moiety which includes a masking moiety, and a cleavable linker connecting the anti-albumin moiety and the drug. FIG. 16B shows a ProDrug molecule comprising an anti-albumin moiety comprising two peptide motifs linked by linker, one of which includes a masking moiety, and a cleavable linker connecting the albumin binding moiety to a drug. FIG. 16C shows a ProDrug molecule comprising a modified albumin (containing a masking moiety) linked to a drug by a cleavable linker. FIG. 16D shows a ProDrug molecule comprising a modified albumin (containing a masking moiety and a protease cleavage site) linked to a drug. FIG. 16E shows an activated ProDrug. In each schematic structure (FIGS. 16A-16D) the drug molecule is functionally masked by the anti-albumin moiety or the modified albumin from binding its target or from being activated at an undesired site or from binding at non-target sites and thereby creating a drug sink.

FIGS. 17A-17E show exemplary schematic structures for ProTriTAC molecules combining functional masking and half-life extension. FIG. 17A shows a ProTriTAC molecule comprising an anti-albumin moiety which includes a masking moiety, and a cleavable linker connecting the anti-albumin moiety and a T cell engager molecule. FIG. 17B shows a ProTriTAC molecule comprising an anti-albumin moiety comprising two peptide motifs linked by linker, one of which includes a masking moiety, and a cleavable linker connecting the albumin binding moiety to a T cell engager molecule. FIG. 17C shows a ProTriTAC molecule comprising a modified albumin (containing a masking moiety) linked to a T cell engager by a cleavable linker. FIG. 17D shows a ProTriTAC molecule comprising a modified albumin (containing a masking moiety and a protease cleavage site) linked to a T cell engager. FIG. 17E shows an activated ProTriTAC. In each schematic structure (FIGS. 17A-17D) a target binding interface within the ProTriTAC molecule is functionally masked by the anti-albumin moiety or the modified albumin from binding its target or from being activated at an undesired site or from binding at non-target sites and thereby creating a sink.

FIG. 18 shows anti-tumor activity of exemplary ProTriTAC molecules and TriTAC molecules of this disclosure.

FIG. 19 shows pharmacokinetic profile of exemplary ProTriTAC molecules and TriTAC molecules of this disclosure.

FIGS. 20A-20F show admix xenograft individual tumor volumes following administering exemplary ProTriTAC molecules or TriTAC molecules of this disclosure. FIG. 20A illustrates results from a GFP TriTAC. FIG. 20B. illustrates results from EGFR ProTriTAC (NCLV). FIG. 20C illustrates results from EGFR ProTriTAC (L001). FIG. 20D illustrates results from EGFR ProTriTAC (L041). FIG. 20E illustrates results from EGFR ProTriTAC (L040). FIG. 20F illustrates results from EGFR ProTriTAC (L045).

FIGS. 21A-21C shows cytokine levels (IFN-gamma (FIG. 21A), IL-6 (FIG. 21B), and IL-10 (FIG. 21C)) following administering exemplary ProTriTAC molecules or TriTAC molecules of this disclosure.

FIGS. 22A-22E show body weight percent change in mice, following administering exemplary ProTriTAC molecules and TriTAC molecules of this disclosure. FIG. 22A illustrates results of 30 μg/kg; FIG. 22B illustrates results of 100 μg/kg; FIG. 22C illustrates results of 300 μg/kg; FIG. 22D illustrates results of 1000 μg/kg; and FIG. 22E provides fold protection at the various concentrations.

FIGS. 23A-23C shows body weight percent change in mice, following administering varying concentrations of exemplary ProTriTAC molecules of this disclosure, containing non-cleavable or cleavable linkers. FIG. 23A illustrates results of 300 μg/kg; FIG. 23B illustrates results of 1000 μg/kg; FIG. 23C provides fold protection at the various concentrations.

FIGS. 24A-24C show serum concentrations of aspartate aminotransferase (AST), in mice, following administering varying concentrations of a ProTriTAC molecule containing a non-cleavable linker (ProTriTAC (NCLV)) (FIG. 24C), a TriTAC molecule (FIG. 24A), or a ProtriTAC molecule (FIG. 24B) containing a cleavable linker.

FIGS. 25A-25C show serum concentrations of alanine aminotransferase (ALT), in mice, following administering varying concentrations of a ProTriTAC molecule containing a non-cleavable linker (ProTriTAC (NCLV)) (FIG. 25C), a TriTAC molecule (FIG. 25A), or a ProtriTAC molecule (FIG. 25B) containing a cleavable linker.

FIGS. 26A-26B show serum concentrations of ALT (right panel; FIG. 26B) or AST (left pane; FIG. 26A), in cynomolgus monkeys, following administering varying concentrations of an EGFR ProTriTAC molecule, or an EGFR ProTriTAC (NCLV) molecule.

FIGS. 27A-27D show tumor volume in mice following administration of a GFP TriTAC molecule, an EGFR TriTAC molecule, or an EGFR ProTriTAC molecule, in varying concentrations. FIG. 27A shows GFP TriTAC (at 300 μg/kg) and an EGFR TriTAC (at 10 μg/kg). FIG. 27B shows the EGFR TriTAC (at 30 μg/kg and at 100 μg/kg). FIG. 27C shows the EGFR TriTAC (at 300 μg/kg) and an EGFR ProTriTAC (at 30 μg/kg and 100 μg/kg). FIG. 27D shows the EGFR ProTriTAC (at 300 μg/kg and 1000 μg/kg)

FIG. 28 shows serum concentrations of ALT and AST, in mice, following administration of varying concentrations of a GFP TriTAC, an EGFR TriTAC, and an EGFR ProTriTAC molecule.

FIG. 29 shows grafting of a CD3ε epitope into the CC′ loop of a dual binding moiety of this disclosure. HuCD3e: SEQ ID NO: 971; CC10: SEQ ID NO: 260; CC12: SEQ ID NO: 259; and CC16: SEQ ID NO: 261.

FIG. 30 shows separation of a dual binding moiety of this disclosure, from a ProTriTAC molecule that contained the dual binding moiety, upon tumor associated protease activation by matriptase.

FIG. 31 shows CD3 binding of ProTriTAC molecules, with or without activation, containing an exemplary dual binding moiety of this disclosure.

FIG. 32 shows cell killing potential of a ProTriTAC molecule, with or without activation, containing an exemplary dual binding moiety of this disclosure.

FIG. 33 shows the soft library mutagenesis approach carried out to explore the non-CDR loops within an exemplary dual binding moiety of this disclosure.

FIGS. 34A-34B illustrate the results of the soft library mutagenesis approach carried out to explore the non-CDR loops within an exemplary dual binding moiety of this disclosure in a naiive library (FIG. 34A) and after panning against HSA (human serum albumin or also referred to herein as albumin; FIG. 34B).

FIG. 35 illustrates that a dual binding moiety of this disclosure is able to expand the therapeutic window of a molecule containing it (e.g., a ProTriTAC molecule or a ProCAR) by both steric masking and specific masking.

FIG. 36 illustrates an exemplary arrangement of a target antigen binding domain (aTarget 1), cleavable linker, and a dual binding moiety (aTarget 2) of the present disclosure.

FIG. 37 illustrates an example of a conditionally active receptor as described herein.

FIGS. 38A-38E show examples of receptor constructs which were generated as disclosed herein used in Examples 26-30. Each construct comprises an intracellular domain comprising a CD8 hinge/transmembrane domain (SEQ ID NO: 823), a 4-1BB intracellular domain (SEQ ID NO: 824), and a CD3 zeta intracellular domain (SEQ ID NO: 825). Construct C1081 (SEQ ID NO: 804; FIG. 38A) does not comprise a target antigen binding domain or a dual binding moiety. Construct C1824 (SEQ ID NO: 805; FIG. 38B) comprises the target antigen binding domain EH4 (an anti-EGFR binding domain) (SEQ ID NO: 821) but does not comprise a dual binding moiety. Construct 1826 (SEQ ID NO: 806; FIG. 38C) comprises target antigen binding domain EH4 and the dual binding moiety 10G with an unmodified non-CDR loop (SEQ ID NO: 817) along with a non-cleavable linker (SEQ ID NO: 826). Construct 1950 (SEQ ID NO: 807; FIG. 38D) comprises target antigen binding domain EH4 and dual binding moiety 10G with a modified non-CDR loop (SEQ ID NO: 818) along with a non-cleavable linker (SEQ ID NO: 826). Construct 2031 (SEQ ID NO: 808; FIG. 38E) comprises target antigen binding domain EH4 and dual binding moiety 10G with a modified non-CDR loop (SEQ ID NO: 818) along with protease cleavable linker 1 (SEQ ID NO: 827).

FIG. 39 shows results from experiments described in Example 26.

FIG. 40 shows results from experiments described in Example 27.

FIGS. 41A-C show results from experiments described in Example 28 for low (FIG. 41A), medium (FIG. 41B), and high (FIG. 41C) CAR expression based on anti-FLAG staining.

FIG. 42 shows results from experiments described in Example 29.

FIG. 43 shows results from experiments in Example 30.

FIGS. 44A-4411 show examples of receptor constructs which were generated as disclosed herein. Construct C1081 (SEQ ID NO: 804; FIG. 44A) does not comprise a target antigen binding domain or a dual binding moiety. Construct 1827 (SEQ ID NO: 809; FIG. 44B) comprises the target antigen binding domain EH104 (SEQ ID NO: 822) but does not comprise a dual binding moiety. Construct 1829 (SEQ ID NO: 810; FIG. 44C) comprises target antigen binding domain EH104 (an anti-EGFR binding domain) and the dual binding moiety 10G with an unmodified non-CDR loop (SEQ ID NO: 817) along with a non-cleavable linker (SEQ ID NO: 826). Construct 1952 (SEQ ID NO: 811; FIG. 44D) comprises target antigen binding domain EH104 and dual binding moiety 10G with a modified non-CDR loop (SEQ ID NO: 819) along with a non-cleavable linker (SEQ ID NO: 826). Construct 1954 (SEQ ID NO: 812; FIG. 44E) comprises target antigen binding domain EH104 and dual binding moiety 10G with a modified non-CDR loop (SEQ ID NO: 59) along with a non-cleavable linker (SEQ ID NO: 820). Construct 2033 (SEQ ID NO: 813; FIG. 44F) comprises target antigen binding domain EH104 and dual binding moiety 10G with a modified non-CDR loop (SEQ ID NO: 819) along with protease cleavable linker 1 (SEQ ID NO: 827). Construct 1953 (SEQ ID NO: 814; FIG. 44G) comprises target antigen binding domain EH104 and dual binding moiety 10G with a modified non-CDR loop (SEQ ID NO: 820) along with protease cleavable linker 2 (SEQ ID NO: 828). Construct 2035 (SEQ ID NO: 815; FIG. 44H) comprises target antigen binding domain EH104 and dual binding moiety 10G with a modified non-CDR loop (SEQ ID NO: 820) along with protease cleavable linker 1 (SEQ ID NO: 827).

FIG. 45 shows results from experiments described in Example 31.

FIG. 46 shows results from experiments described in Example 32.

FIGS. 47A-C show results from experiments described in Example 33 for low (FIG. 47A), medium (FIG. 47B), and high (FIG. 47C) CAR expression based on anti-FLAG staining.

FIGS. 48A-48C show results from experiments described in Example 34 for low (FIG. 48A), medium (FIG. 48B), and high (FIG. 49C) CAR expression based on anti-FLAG staining.

FIG. 49 shows results from experiments described in Example 35.

FIG. 50 shows results from experiments described in Example 36.

FIG. 51 shows results from experiments described in Example 37.

FIGS. 52A-E show exemplary ProCAR constructs. FIG. 52A illustrates construct C2446 which includes an anti-human serine albumin sdAb, a protease cleavage site 3, an anti-human EGFR sdAb, a FLAG epitope, a CD8 hinge/transmembrane domain, a 4-1BB intracellular domain, and a CD3 zeta intracellular domain (SEQ ID NO: 832). FIG. 52B illustrates construct C2510 which includes an anti-human serine albumin sdAb, a protease cleavage site, an anti-human EGFR sdAb, a FLAG epitope, a dimerization-deficient CD8a hinge/transmembrane domain, a 4-1BB intracellular domain, and a CD3 zeta intracellular domain (SEQ ID NO: 834). FIG. 52C illustrates construct C2513 which includes an anti-human serine albumin sdAb, a protease cleavage site 3, an anti-human EGFR sdAb, a FLAG epitope, a dimerization-deficient CD8a hinge/transmembrane domain, a 4-1BB intracellular domain, and a CD3 zeta intracellular domain (SEQ ID NO: 835). FIG. 52D illustrates construct C2514 which includes an anti-human serine albumin sdAb, an anti-human EGFR sdAb, a FLAG epitope, a dimerization-deficient CD8a hinge/transmembrane domain, a 4-1BB intracellular domain, and a CD3 zeta intracellular domain (SEQ ID NO: 836). FIG. 52E illustrates construct C2448, which includes an anti-human EGFR sdAb, a FLAG epitope, a dimerization-deficient CD8a hinge/transmembrane domain, a 4-1BB intracellular domain, and a CD3 zeta intracellular domain (SEQ ID NO: 833).

FIGS. 53A-53B show that the level of protease site-dependent cell killing activity of the ProCARs constructs. The level of protease site-dependent cell killing activity of the ProCARs constructs was reduced by eliminating the dimerization activity of the CD8a transmembrane domain (FIG. 53B) compared to wild-type (FIG. 53A).

FIG. 54 shows that the dimerization deficient EGFR CAR ad similar cell killing activity compared to wild-type.

FIGS. 55A-55G show exemplary ProCAR constructs. FIG. 55A illustrates construct C2483 which includes an anti-human EpCAM sdAb, a FLAG epitope, a CD8 hinge/transmembrane domain, a 4-1BB intracellular domain, and a CD3 zeta intracellular domain (SEQ ID NO: 838). FIG. 55B illustrates construct C2790 which includes an anti-human serum albumin sdAb, an anti-human EpCAM sdAb, a FLAG epitope, a CD8 hinge/transmembrane domain, a 4-1BB intracellular domain, and a CD3 zeta intracellular domain (SEQ ID NO: 839). FIG. 55C illustrates construct C3269 which includes an anti-human serum albumin sdAb that contains a mask in the CC′ loop, an anti-human EpCAM sdAb, a FLAG epitope, a CD8 hinge/transmembrane domain, a 4-1BB intracellular domain, and a CD3 zeta intracellular domain (SEQ ID NO: 840). FIG. 55D illustrates construct C3272 which includes an anti-human serum albumin sdAb that contains a mask in the CC′ loop, an anti-human EpCAM sdAb, a FLAG epitope, a CD8 hinge/transmembrane domain, a 4-1BB intracellular domain, and a CD3 zeta intracellular domain (SEQ ID NO: 841). FIG. 55E illustrates construct C3329 which includes an anti-human serum albumin sdAb, a protease cleavage site 3, an anti-human EpCAM sdAb, a FLAG epitope, a CD8 hinge/transmembrane domain, a 4-1BB intracellular domain, and a CD3 zeta intracellular domain (SEQ ID NO: 843).

FIG. 55F illustrates construct C3328 which includes an anti-human serum albumin sdAb that contains a mask in the CC′ loop, a protease cleavage site 3, an anti-human EpCAM sdAb, a FLAG epitope, a CD8 hinge/transmembrane domain, a 4-1BB intracellular domain, and a CD3 zeta intracellular domain (SEQ ID NO: 842). FIG. 55G illustrates construct C2780 which includes an anti-GFP sdAb, a FLAG epitope, a CD8 hinge/transmembrane domain, a 4-1BB intracellular domain, and a CD3 zeta intracellular domain (SEQ ID NO: 837).

FIG. 56 demonstrates steric blocking of anti-EpCAM sdAb H90 ProCAR-T cell killing activity by HSA when assayed at ratios 10:1, 5:1, 2.5:1, and 1.25:1 CAR-T:Target cells.

FIGS. 57A-57C provide histograms of EpCAM-Fc/Alexa Fluor 647 staining of CAR-T cells from FIG. 55 that have been grouped into low (FIG. 57A), medium (FIG. 57B), or high (FIG. 57C) CAR expression based on anti-FLAG staining that demonstrate the efficacy of EpCAM mask 1 in blocking ProCAR EpCAM-binding activity.

FIGS. 58A-58C provide histograms of EpCAM-Fc/Alexa Fluor 647 staining of CAR-T cells from FIG. 55 that have been grouped into low (FIG. 58A), medium (FIG. 58B), or high (FIG. 58C) CAR expression based on anti-FLAG staining that demonstrate the efficacy of EpCAM mask 2 in blocking ProCAR EpCAM-binding activity.

FIG. 59 demonstrates masking of the anti-EpCAM sdAb H90 of constructs of FIG. 55 at ratios 10:1, 5:1, 2.5:1, and 1.25:1 CAR-T:Target cells.

FIG. 60 demonstrates protease site-dependent activation of EpCAM ProCAR mask 2 cell killing activity.

FIGS. 61A-61C illustrate protease activation of EpCAM Mask 1 ProCAR antigen binding activity at low (FIG. 61A), medium (FIG. 61B), or high (FIG. 61C) CAR expression based on anti-FLAG staining.

FIGS. 62A-62C illustrate protease activation of EpCAM Mask 2 ProCAR antigen binding activity at low (FIG. 62A), medium (FIG. 62B), or high (FIG. 62C) CAR expression based on anti-FLAG.

FIGS. 63A-63C show body weight percent change in mice, following administering exemplary EpCAM ProTriTAC molecules (EpCAM ProTriTAC containing linker L040 in FIG. 63B; EpCAM ProTriTAC containing a non-cleavable linker in FIG. 63C) and EpCAM TriTAC molecules (FIG. 63A) of this disclosure.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

A “single chain Fv” or “scFv”, as used herein, refers to a binding protein in which the variable domains of the heavy chain and of the light chain of a traditional two chain antibody are joined to form one chain. Typically, a linker peptide is inserted between the two chains to allow for proper folding and creation of an active binding site.

A “cleavage site for a protease,” or “protease cleavage site”, as meant herein, is an amino acid sequence that can be cleaved by a protease, such as, for example, a matrix metalloproteinase or a furin. Examples of such sites include Gly-Pro-Leu-Gly-Ile-Ala-Gly-Gln or Ala-Val-Arg-Trp-Leu-Leu-Thr-Ala, which can be cleaved by metalloproteinases, and Arg-Arg-Arg-Arg-Arg-Arg, which is cleaved by a furin. In therapeutic applications, the protease cleavage site can be cleaved by a protease that is produced by target cells, for example cancer cells or infected cells, or pathogens.

A “ProTriTAC molecule,” or a “protrispecific molecule,” as used herein, refers to a molecule comprising a dual binding moiety comprising non-CDR loops and a cleavable linker, as described herein; a first domain that is capable of binding a target; and a second domain that is capable of binding a target and further requires that the molecule is in a configuration (also referred to herein as in the “masked state”) where the first or the second domain is masked from binding its target by the dual binding moiety, by specific binding between the dual binding moiety and the first or second domain through the non-CDR loops, by steric occlusion, or a combination of both. In some cases, the first or the second domain is a scFv specific for CD3. Upon cleavage of the cleavable linker, the ProTriTAC molecule is separated from the dual binding moiety. It is possible for the dual binding moiety of the ProTriTAC molecule to be further bound to a bulk serum protein, such as albumin, to form a “ProTetraTAC” molecule. The ProTriTAC molecule, in some example, comprises a masked state and an active state.

“TriTAC,” as used herein refers to a trispecific binding protein that is not conditionally activated.

As used herein, “elimination half-time” is used in its ordinary sense, as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t₁₁₂ the time required for 50% completion of the process. The units of these two constants are time⁻¹ and time, respectively. A first-order rate constant and the half-time of the reaction are simply related (k×t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e. after drug absorption and distribution are complete). The half-time for drug elimination can be accurately determined from such a graph.

As used herein, “non-CDR loops” within immunoglobulin (Ig) molecules are regions of a polypeptide other than the complementarity determining regions (CDRs) of an antibody. These regions may be derived from an antibody or an antibody fragment. These regions may also be synthetically or artificially derived, such as through mutagenesis or polypeptide synthesis.

In an Ig, Ig-like, or beta-sandwich scaffold that has 9 beta-strands (e.g., a VH, a VL, a camelid VHH, a sdAb), the non-CDR loops can refer to the AB, CC′, C″D, EF loops or loops connecting beta-strands proximal to the C-terminus. In an Ig, Ig-like, or beta-sandwich scaffold that has 7 beta-strands (e.g., a CH, a CL, an adnectin, a Fn-III), the non-CDR loops can refer to the AB, CD, and EF loops or loops connecting beta-strands proximal the C-terminus. In other Ig-like or beta-sandwich scaffolds, the non-CDR loops are the loops connecting beta-strands proximal to the C-terminus or topologically equivalent residues using the framework established in the Halaby 1999 publication (Prot Eng Des Sel 12:563-571).

In a non-beta-sandwich scaffold (e.g., a DARPin, an affimer, an affibody), the “non-CDR loops” refer to an area that is (1) amenable for sequence randomization to allow engineered specificities to a second antigen, and (2) distal to the primary specificity determining region(s) typically used on the scaffold to allow simultaneous engagement of the scaffold to both antigens without steric interference. For this purpose, the primary specificity determining region(s) can be defined using the framework established in the Skrlec 2015 publication (Trends in Biotechnol, 33:408-418). An excerpt of the framework is listed below.

| Scaffold | Primary specificity determining region(s) |
|---|---|
| Affibody | 13 residues in two helices |
| Affimer | 12-36 residues |
| Anticalin | Four loops (up to 24 aa) |
| Avimer | 11 residues |
| Centyrin | 13 residues |
| DARPin | 7 residues in each n-repeat, or |
| | 8 residues in each n-repeat |
| Fynomer | 6 residues in the RT- and n-Src-loop |
| Kunitz domain | 1-2 loops |

“Target antigen binding domain”, as used herein, refers to a region which targets a specific antigen. A target antigen binding domain comprises, for example an sdAb, an scFv, a variable heavy chain antibody (VHH), a variable heavy (VH) or a variable light domain (VL), a full length antibody, or any other peptide that has a binding affinity towards a specific antigen.

The term “non-immunoglobulin binding molecules,” as used herein, include but is not limited to examples such as a growth factor, a hormone, a signaling protein, an inflammatory mediator, ligand, a receptor, or a fragment thereof, a native hormone or a variant thereof being able to bind to its natural receptor; a nucleic acid or polynucleotide sequence being able to bind to complementary sequence or a soluble cell surface or intracellular nucleic acid/polynucleotide binding proteins, a carbohydrate binding moiety being able to bind to other carbohydrate binding moieties, cell surface or intracellular proteins, a low molecular weight compound (drug) that binds to a soluble or cell surface or intracellular target protein. The non-immunoglobulin binding molecules, in some cases, include coagulation factors, plasma proteins, fusion proteins, and imaging agents. The non-immunoglobulin binding molecules do not include a cytokine.

A “cytokine,” as meant herein, refers to intercellular signaling molecules, and active fragments and portions thereof, which are involved in the regulation of mammalian somatic cells. A number of families of cytokines, for example, interleukins, interferons, and transforming growth factors are included.

### Dual Binding Moiety Comprising Cleavable Linker

An embodiment of the disclosure provides a dual binding moiety that is capable of masking a domain from binding to its target. In some cases, the masking is through a building between the dual binding moiety and the domain via a masking moiety (also referred to herein as a “masking sequence”) present within the dual binding moiety. In some cases, the dual binding moiety is capable of interacting with a domain and the interaction between the dual binding moiety and the domain is a specific binding, such as a specific intermolecular interaction, or via steric occlusion. In some embodiments, through its interaction with the domain, the dual binding moiety masks the domain from binding to its target. In some examples, the domain is a target antigen binding domain. Provided herein, in one embodiment, is a dual binding moiety that is capable of masking a target antigen binding domain from binding its target. The dual binding moiety, in some cases, is further capable of binding to a bulk-serum protein, such as a half-life extending protein, e.g., albumin. Thus, in some embodiments, the dual binding moiety is able to mask the domain from binding its target as well as is able to extend the half-life of the domain through its binding to a bulk serum protein. The dual binding moiety, in certain instances, comprises a cleavable linker attached to it. The cleavable linker, for example, comprises a protease cleavage site or a pH dependent cleavage site. The cleavable linker, in certain instances, is cleaved only in a protease rich environment, such as a tumor micro-environment. The dual binding moiety, in certain instances, is able to keep a domain, such as a target antigen binding domain, in an inert state, by masking the domain from binding its target until the dual binding moiety is in a cellular environment suitable for cleavage of the linker comprising a cleavage site, such as a protease cleavage site. In some cases, the cellular environment suitable for cleavage of the linker is a protease rich environment, such as a tumor microenvironment.

In some embodiments, the dual binding moiety is capable of interacting with another molecule, such as a non-immunoglobulin molecule, such as therapeutic agent, a small protein, an imaging agent, or a diagnostic agent, whereby the other molecule is kept in an inert state as long as the dual binding moiety is in interaction with it. In certain examples, the other molecule is masked by the dual binding moiety from interacting with its targets or binding partners, wherein the masking is through the interaction between the dual binding moiety and the other molecule by specific binding or by steric occlusion.

The dual binding moiety, in some embodiments, acts as a safety-switch for the domain or other molecule which is masked by the moiety. Such a safety switch provides several advantages, some examples including (i) expanding the therapeutic window of the domain, such as a target antigen binding domain, (ii) reducing target-mediated drug disposition by maintaining the domain, such as a target antigen binding domain, or a therapeutic agent, in an inert state when it is in systemic circulation, (iii) reducing the concentration of undesirable activated proteins in systemic circulation, thereby minimizing the spread of chemistry, manufacturing, and controls related impurities, e.g., pre-activated drug product, endogenous viruses, host-cell proteins, DNA, leachables, anti-foam, antibiotics, toxins, solvents, heavy metals; (iv) reducing the concentration of undesirable activated proteins in systemic circulation, thereby minimizing the spread of product related impurities, aggregates, breakdown products, product variants due to: oxidation, deamidation, denaturation, loss of C-term Lys in MAbs; (v) preventing aberrant activation of the target antigen binding domain in circulation; (vi) reducing the toxicities associated with the leakage of activated species from diseased tissue or other pathophysiological conditions, e.g., tumors, autoimmune diseases, inflammations, viral infections, tissue remodeling events (such as myocardial infarction, skin wound healing), or external injury (such as X-ray, CT scan, UV exposure); and (vii) reducing non-specific binding of the domain, such as a target antigen binding domain. Furthermore, after breaking of the safety switch, the domain, such as a target antigen binding domain is separated from the safety switch which provided extended half-life, and thus is readily cleared from circulation.

In addition, the dual binding moiety as described herein, in some cases, is used to generate a “biobetter” version of a biologic. Generally, preparing a biobetter form of a molecule, e.g., an antibody or an antigen binding fragment thereof, involves taking the originator molecule and making specific alterations in it to improve its parameters and thereby make it a more efficacious, less frequently dosed, better targeted, and/or a better tolerated drug. Thus, in some embodiments, the dual binding moiety, which is capable of masking a domain, such as a target antigen binding domain from binding its target, extending the half-life of the domain by binding to a half-life extending protein, such as albumin, and unmasking the domain by separating from it upon cleavage of the cleavable linker, for example, in a tumor microenvironment, gives the target antigen binding domain a significantly longer serum half-life and reduces the likelihood of its undesirable activation in circulation, thereby producing a “biobetter” version of it.

The dual binding moiety described herein comprises at least one non-CDR loop. In some embodiments, a non-CDR loop provides a binding site for binding of the moiety to a target antigen binding domain. In some embodiments, the dual binding moiety masks the target antigen binding domain from binding to its target, e.g., via steric occlusion, via specific intermolecular interactions, or a combination of steric occlusion and specific intermolecular interactions. Specific intermolecular interactions include, in some cases, Van der Waal interactions, electrostatic interactions, hydrogen bonds (H-bonds). In some cases, masking via an interaction that is a combination of steric occlusion and specific intermolecular interactions, creates an additive or a synergistic effect. T cell engagers transiently tether T cells to tumor cells and mediate T cell-directed tumor killing. T cell engagers, such as blinatumomab (BLINCYTO®), have demonstrated clinical activity in several hematological malignancies. Adoption of T cell engagers in solid tumors is limited by the scarcity of tumor antigens with sufficient differential expression between tumor and normal tissue. T cell engagers that are preferentially active in the tumor microenvironment may enable the safe targeting of more solid tumor antigens. In some cases, the dual binding moieties disclosed herein, are part of T cell engagers that are preferentially active in the tumor microenvironment.

In some embodiments, the dual binding moiety combines both steric masking (for example, via binding to a bulky serum albumin) and specific masking (for example, via non-CDR loops binding to the CDRs of an anti-CD3 scFv domain). In some cases, modifying non-CDR loops within the dual binding moiety does not affect albumin binding. The protease cleavable linker, in some cases, enables activation of a prodrug molecule comprising the dual binding moiety, in a single proteolytic event, thereby allowing more efficient conversion of the prodrug molecule in tumor microenvironment. Further, tumor-associated proteolytic activation, in some cases, reveals active T cell engager (such as a ProTriTAC molecule comprising a dual binding moiety as described herein, a CD3 binding domain, and an albumin binding domain) with minimal off-tumor activity after activation. The present disclosure, in some embodiments, provides a half-life extended T cell engager format (ProTriTAC) comprising a dual binding moiety as described herein, which in some cases represents a new and improved approach to engineer conditionally active T cell engagers.

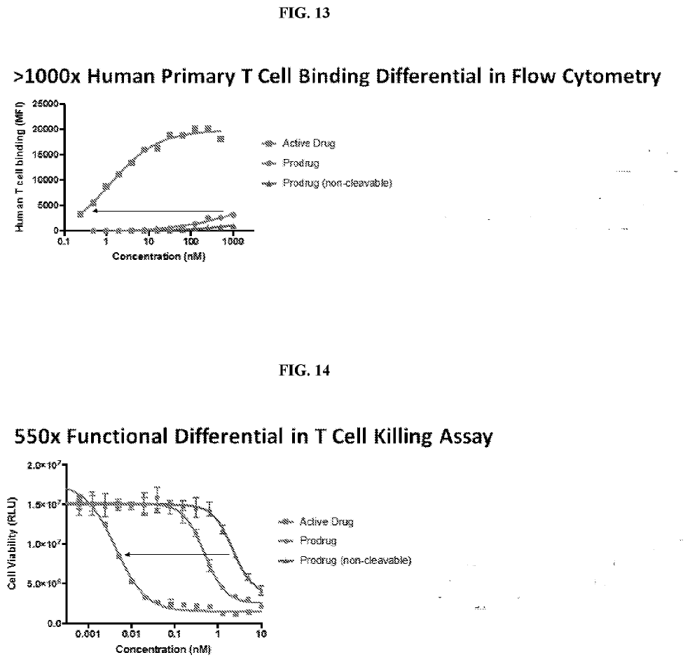

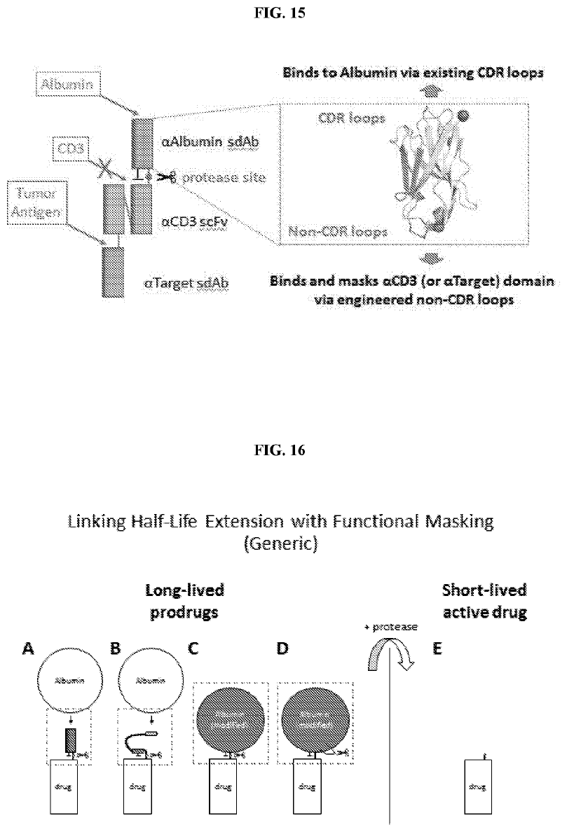

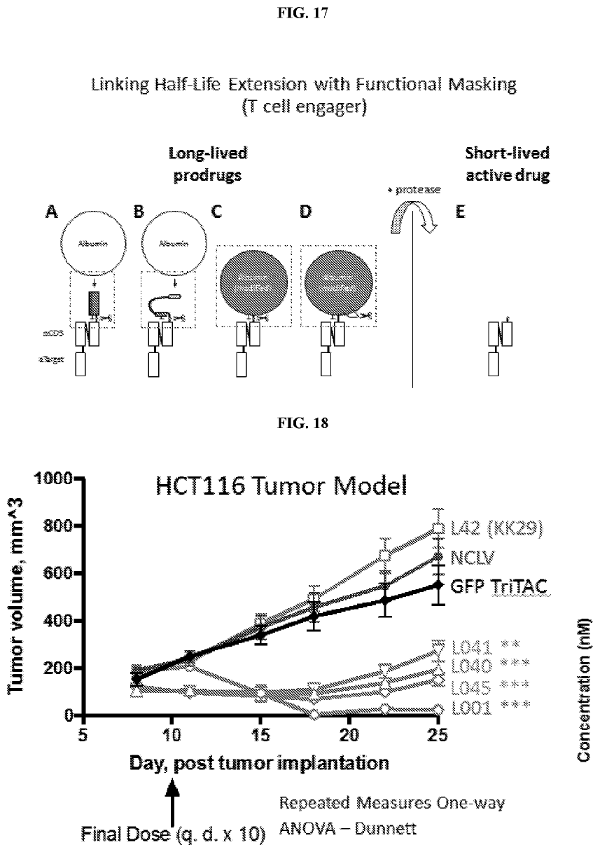

FIG. 2B provides a schematic for an exemplary ProTriTAC molecule comprising a dual binding moiety as described herein (the αalbumin sdAb) and a gel showing the ProTriTAC before and after activation by cleaving of the protease cleavable linker, and FIG. 2A shows a possible mode of action of the same. FIG. 13 shows the schematic structure of an exemplary trispecific molecule (also referred to herein as ProTriTAC or activatable ProTriTAC) containing a dual binding moiety as described herein. The exemplary trispecific molecule contains an anti-albumin domain comprising tethered to a cleavable linker and a masking domain; an anti-CD3 binding domain; and an anti-target domain (such as, a domain specific for a tumor antigen). In some cases, non-CDR loops in the anti-albumin domain is capable of binding and masking the anti-target domain. In some cases, non-CDR loops in the anti-albumin domain is capable of binding and masking the anti-CD3 domain. The anti-albumin domain, in some embodiments, comprises a CDR loop specific for binding albumin.

In some embodiments, the dual binding moiety is part of a cleavable conditionally active chimeric antigen receptor (CAR), a T-cell receptor fusion protein, a T-cell receptor. A T-cell receptor generally comprises multiple subunits, including alpha, beta, delta, gamma, epsilon, and zeta subunits. The conditionally active T-cell receptor of the present disclosure comprises a dual binding moiety. In some embodiments, the dual binding moiety is attached to a T-cell receptor subunit including, but not limited to, the alpha subunit, beta subunit, or a combination thereof. As used herein, a “T-cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T-cell. A CAR generally comprises multiple domains, including a target antigen binding domain, a transmembrane domain, and an intracellular signaling domain. The conditionally active CAR of the present disclosure comprises multiple domains, including a dual binding moiety, a target antigen binding domain, a transmembrane domain, and an intracellular signaling domain. In some embodiments, the intracellular signaling domain is a signaling domain of a protein including, but not limited to, ZAP70, CD3 zeta, and 4-1BB.

FIG. 36 shows a schematic representation of a portion of an example cleavable conditionally active receptor of the present disclosure. The conditionally active receptor comprises a target antigen binding domain (aTarget1), a cleavable linker, and a dual binding moiety (aTarget2). The target antigen binding domain has specificity for a first target, while the dual binding moiety has specificity for a second target. The dual binding moiety also has a modified non-CDR loop, which inhibits binding of the target antigen binding domain to its target. Once cleaved at the cleavable linker, the dual binding moiety can be released, enabling binding of the target antigen binding domain.

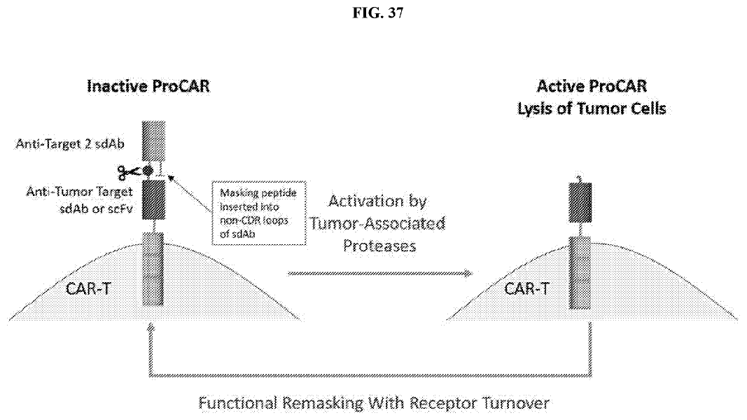

FIG. 37 shows a schematic representation of the activation of an example conditionally active receptor of the present disclosure. Inactive receptor (Inactive ProCAR) comprises a target antigen binding domain (Anti-Tumor Target sdAb or scFv) connected to a dual binding moiety (Anti-Target 2 sdAb) via a linker comprising a protease cleavage site. The dual binding moiety comprises a masking peptide, inserted into one or more non-CDR loops, such that the dual binding moiety binds to and inhibits the target antigen binding domain. In some embodiments, the dual binding moiety has specificity for a given target, as described further elsewhere herein. The receptor also comprises a transmembrane domain and an intracellular signaling domain. The receptor is provided in a T-cell (CAR-T). Upon exposure to a tumor environment, the protease cleavage site is cleaved by tumor-associated proteases, thereby activating the receptor, generating the active receptor which does not comprise the dual binding moiety. The receptor now comprises an active antigen-binding domain. As the receptor is internalized by the cell, new receptors are generated which comprise the dual binding moiety and are inactive.

The conditionally active receptor, the conditionally active T-cell receptor fusion protein, in some cases, comprise at least one of: a transmembrane domain, and a hinge domain, a stimulatory domain, a costimulatory domain, or any combinations thereof.

In some embodiments, the transmembrane domain is interposed between the target antigen binding domain and the intracellular domain. In some embodiments, the transmembrane domain is interposed between the target antigen binding domain and the costimulatory domain. Any transmembrane (TM) domain that provides for insertion of a polypeptide into the cell membrane of a eukaryotic (e.g., mammalian) cell is suitable for use. As one non-limiting example, the TM sequence IYIWAPLAGTCGVLLLSLVITLYC (SEQ ID NO: 971) can be used. Additional non-limiting examples of suitable TM sequences include: a) CD8 beta derived: GLLVAGVLVLLVSLGVAIHLCC (SEQ ID NO: 9721); b) CD4 derived: ALIVLGGVAGLLLFIGLGIFFCVRC (SEQ ID NO: 973); c) CD3 zeta derived: LCYLLDGILFIYGVILTALFLRV (SEQ ID NO: 974); d) CD28 derived: WVLVVVGGVLACYSLLVTVAFIIFWV (SEQ ID NO: 975); e) CD134 (OX40) derived: AAILGLGLVLGLLGPLAILLALYLL (SEQ ID NO: 976); and f) CD7 derived: ALPAALAVISFLLGLGLGVACVLA (SEQ ID NO: 977). In some embodiments, the transmembrane domain comprises a transmembrane domain of a protein including, but not limited to, a TCR alpha chain, a TCR beta chain, a TCR zeta chain, a CD3 epsilon TCR subunit, a CD3 gamma TCR subunit, a CD3 delta TCR subunit, CD45, CD4, CDS, CD8, CD9, CD16, CD22, CD33, CD28, CD37, CD64, CD80, CD86, CD134, CD137, CD154, functional fragments thereof, or amino acid sequences thereof having at least one, two, or three modifications but not more than 20, 10, or 5 modifications thereto.

In some embodiments, the T-cell receptor intracellular domain comprises a stimulatory domain. The stimulatory domain may be from T-cell receptor subunit, including but not limited to the beta subunit, alpha subunit, delta subunit, gamma subunit, epsilon subunit, or a combination thereof. In some embodiments, the stimulatory domain comprises an immunoreceptor tyrosine-based activation motif (ITAM) or portion thereof including, but not limited to, CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b 1 chain, Fc gamma receptor 2b2 chain, Fc gamma receptor 3a chain, Fc gamma receptor 3b chain, Fc beta receptor 1 chain, TYROBP (DAP12), CDS, CD16a, CD16b, CD22, CD23, CD32, CD64, CD79a, CD79b, CD89, CD278, CD66d, functional fragments thereof, and amino acid sequences thereof having at least one, two, or three modifications but not more than 20, 10, or 5 modifications thereto.

In some embodiments, the conditionally active TFP further comprises a costimulatory domain. In some embodiments, the costimulatory domain is a functional signaling domain of a protein including, but not limited to, OX40, CD2, CD27, CD28, CDS, ICAM-1, LFA-1 (CD11a/CD18), ICOS (CD278), and 4-1BB (CD137), and amino acid sequences thereof having at least one, two, or three modifications but not more than 20, 10, or 5 modifications thereto.

In some cases, the conditionally active chimeric antigen receptors, T-cell receptor fusion proteins, and T-cell receptors of the present disclosure comprise a hinge region (also referred to herein as a “spacer”), where the hinge region is interposed between the target antigen binding domain and the transmembrane domain. In some cases, the hinge region is an immunoglobulin heavy chain hinge region. In some cases, the hinge region is a hinge region polypeptide derived from a receptor (e.g., a CD8-derived hinge region).

The hinge region can have a length of from about 4 amino acids to about 50 amino acids (aa), e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

Suitable spacers can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary spacers include glycine polymers (G)n, glycine-serine polymers (including, for example, (GS)n, (GSGGS)n and (GGGS)n, where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary spacers comprise amino acid sequences including, but not limited to, GGSG, GGSGG, GSGSG, GSGGG, GGGSG, GSSSG, and the like.

Immunoglobulin hinge region amino acid sequences are known in the art; see, e.g., Tan et al. (1990) Proc. Natl. Acad. Sci. USA 87: 162; and Huck et al. (1986) Nucl. Acids Res. 14: 1779. As non-limiting examples, an immunoglobulin hinge region can include one of the following amino acid sequences: DKTHT; CPPC; CPEPKSCDTPPPCPR (SEQ ID NO: 978); (see, e.g., Glaser et al. (2005) J. Biol. Chem. 280:41494); ELKTPLGDTTHT (SEQ ID NO: 979); KSCDKTHTCP (SEQ ID NO: 980); KCCVDCP (SEQ ID NO: 981); KYGPPCP (SEQ ID NO: 982); EPKSCDKTHTCPPCP (SEQ ID NO: 983); human IgG1 hinge); ERKCCVECPPCP (SEQ ID NO: 984); human IgG2 hinge); ELKTPLGDTTHTCPRCP (SEQ ID NO: 985); human IgG3 hinge); SPNMVPHAHHAQ (SEQ ID NO: 986); human IgG4 hinge); and the like.

In some embodiments, the hinge region comprises an amino acid sequence of a human IgG1, IgG2, IgG3, or IgG4, hinge region. The hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO: 986); see, e.g., Yan et al. (2012) J. Biol. Chem. 287. 5891.

In some embodiments, the hinge region comprises an amino acid sequence derived from human CD8; e.g., the hinge region comprises the amino acid sequence: TTTPAPRPPTPAPTIASQPLSLRPEACRPAAGGAVHTRGLDFACD (SEQ ID NO: 987), or a variant thereof.

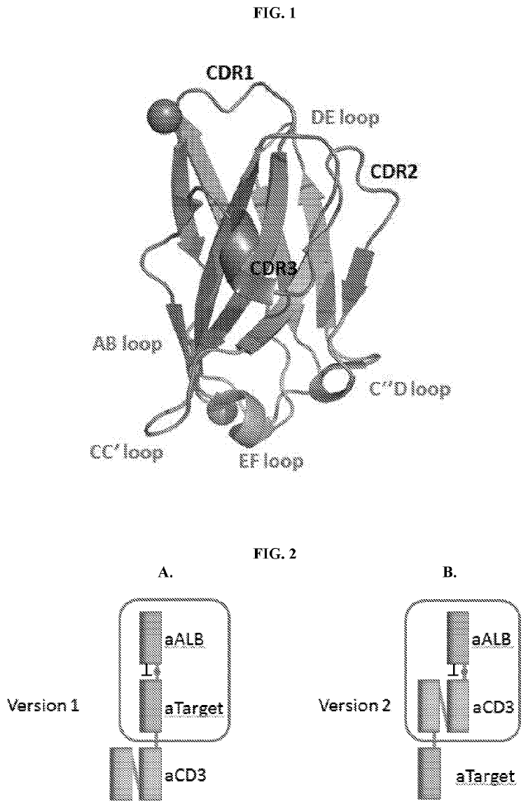

In some embodiments, the dual binding moiety further comprises complementarity determining regions (CDRs). In some instances, the dual binding moiety is a domain derived from an immunoglobulin binding molecule (Ig molecule). The Ig molecule, in some instances, is of any class or subclass, such as IgG1, IgG2, IgG3, IgG4, IgA, IgE, IgM. A polypeptide chain of an Ig molecule folds into a series of parallel beta strands linked by loops. In the variable region, three of the loops constitute the “complementarity determining regions” (CDRs) which determine the antigen binding specificity of the molecule. An IgG molecule comprises at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs) with are hypervariable in sequence and/or involved in antigen recognition and/or usually form structurally defined loops, interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments of this disclosure, at least some or all of the amino acid sequences of FR1, FR2, FR3, and FR4 are part of the “non-CDR loop” of the dual binding moiety described herein. As shown in FIG. 1, a variable domain of an immunoglobulin binding molecule has several beta strands that are arranged in two sheets. The variable domains of both light and heavy immunoglobulin chains contain three hypervariable loops, or complementarity-determining regions (CDRs). The three CDRs of a V domain (CDR1, CDR2, CDR3) cluster at one end of the beta barrel. The CDRs are the loops that connect beta strands B-C, C-C″, and F-G of the immunoglobulin fold, whereas the bottom loops that connect beta strands AB, CC′, C″D and EF of the immunoglobulin fold, and the top loop that connects the D-E strands of the immunoglobulin fold are the non-CDR loops. In some embodiments of this disclosure, at least some amino acid residues of a constant domain, CH1, CH2, or CH3, are part of the “non-CDR loop” of the binding moieties described herein. Non-CDR loops comprise, in some embodiments, one or more of AB, CD, EF, and DE loops of a C1-set domain of an Ig or an Ig-like molecule; AB, CC′, EF, FG, BC, and EC′ loops of a C2-set domain of an Ig or an Ig-like molecule; DE, BD, GF, A(A1A2)B, and EF loops of I(Intermediate)-set domain of an Ig or Ig-like molecule.

Within the variable domain, the CDRs are believed to be responsible for antigen recognition and binding, while the FR residues are considered a scaffold for the CDRs. However, in certain cases, some of the FR residues play an important role in antigen recognition and binding. Framework region residues that affect Ag binding are divided into two categories. The first are FR residues that contact the antigen, thus are part of the binding-site, and some of these residues are close in sequence to the CDRs. Other residues are those that are far from the CDRs in sequence, but are in close proximity to it in the 3-D structure of the molecule, e.g., a loop in heavy chain.

In some embodiments, the non-CDR loop within the dual binding moiety is modified to generate an antigen binding site specific for a target antigen binding domain. This site is also referred to herein as the masking sequence or the masking moiety. It is contemplated that various techniques can be used for modifying the non-CDR loop, e.g., site-directed mutagenesis, random mutagenesis, insertion of at least one amino acid that is foreign to the non-CDR loop amino acid sequence, amino acid substitution. An antigen peptide is inserted into a non-CDR loop, in some examples. In some examples, an antigenic peptide is substituted for the non-CDR loop. In some embodiments, one or more non-CDR loop is extended, for example by adding glycine residues, in addition to other modifications, such as, for generating a binding site, grafting an epitope, or combination thereof. The extension of one or more non-CDR loop within the dual binding moiety, in some instances, allows the dual binding moiety to sterically occlude a domain, such as a target antigen binding domain, from binding its target. The modification, to generate an antigen binding site, is in some cases in only one non-CDR loop. In other instances, more than one non-CDR loop are modified. For instance, the modification is in any one of the non-CDR loops shown in FIG. 1, i.e., AB, CC′, C″D, EF, and DE. In some cases, the modification is in the DE loop. In some cases, the modification is in the CC′ loop. In other cases the modifications are in all four of AB, CC′, C″ D, EF loops. In certain examples, the dual binding moiety is capable of an interaction with a domain, such as a target antigen binding domain via one or more of the AB, CC′, C″ D, and EF loops and is capable of binding to a bulk-serum protein, such as albumin, via one or more of the BC, C-C″, and FG loops. In certain examples, the dual binding moiety is capable of binding to a domain, such as a target antigen binding domain via specific intermolecular interactions involving at least one of the AB, CC′, C″ D, and EF loops and is capable of binding to a bulk-serum protein, such as albumin, via one or more of the BC, C′C″, and FG loop. In certain examples, the dual binding moiety is capable of binding to a domain, such as a target antigen binding domain via specific intermolecular interactions involving at least one of the AB, CC′, C″ D, and EF loops, as well as via steric occlusion, and is further capable of binding to a bulk-serum protein, such as albumin, via its BC, C′C″, and FG loop. In certain examples, the dual binding moiety is capable of binding to a bulk serum protein, such as albumin, via one or more of the AB, CC′, C″ D, and EF loops and is capable of binding to the target antigen binding domain via one or more of the BC, C′C″, and FG loops. In certain examples, the dual binding moiety is capable of binding to a bulk serum protein, such as albumin, via specific intermolecular interactions involving one or more of the AB, CC′, C″ D, and EF loops and is capable of binding to the target antigen binding domain via specific intermolecular interactions involving one or more of the BC, C′C″, and FG loops. In certain examples, the dual binding moiety is capable of binding to a bulk serum protein, such as albumin, via specific intermolecular interactions involving one or more of the AB, CC′, C″ D, and EF loops and is capable of binding to the target antigen binding domain via specific intermolecular interactions involving one or more of the BC, C′C″, and FG loops, as well as via steric occlusion.

In some embodiments, an epitope is grafted into the non-CDR loop within the dual binding moiety. For instance, in some cases, a human CD3ε sequence is grafted into the non-CDR loop, such as into the CC′ loop. The dual binding moiety, in such embodiment, is capable of binding to a half-life extending protein via the CDRs and an anti-CD3 domain via the non-CDR loops, e.g., the CC′ loop. The presence of the CD3 epitope in the non-CDR loop allows competitive binding between the CD3 epitope within the dual binding moiety and an anti-CD3 domain, in some embodiments, thereby masking the anti-CD3 domain from binding to its target. The anti-CD3 domain, in some cases, is part of a scFv.

TABLE 1
Exemplary sequences for the masking moiety are provided in SEQ ID Nos. 50,
259-301, and 795.

| MaskID | Sequence | Alt. Names | SEQ ID No. |
|---|---|---|---|
| MASK001 | APGKG | WT | SEQ ID No. 795 |
| MASK002 | GGQDGNEEMGGG | CC12 (masking moiety | SEQ ID No. 259 |
| | | comprising a CD3 | |
| | | sequence grafted) | |
| MASK004 | GGQDGNEEGG | CC10 (masking moiety | SEQ ID No. 260 |
| | | comprising a CD3 | |
| | | sequence grafted) | |
| MASK006 | GGGGQDGNEEMGGGGG | CC16 (masking moiety | SEQ ID No. 261 |
| | | comprising a CD3 | |
| | | sequence grafted) | |
| MASK007 | APFGSEM | | SEQ ID No. 262 |
| MASK008 | AWNGPYE | | SEQ ID No. 263 |
| MASK009 | AQDNGDTKTG | | SEQ ID No. 264 |
| MASK010 | AEAKETQG | | SEQ ID No. 265 |
| MASK011 | ATRREQVEG | | SEQ ID No. 266 |
| MASK012 | AQAPSQP | | SEQ ID No. 267 |
| MASK013 | ATRSRTRNDG | | SEQ ID No. 268 |
| MASK014 | ADVDAPDGLG | | SEQ ID No. 269 |
| MASK015 | AADISDPGG | | SEQ ID No. 270 |
| MASK016 | ALSVDPSG | | SEQ ID No. 271 |
| MASK017 | ARLSVDPG | | SEQ ID No. 272 |
| MASK018 | AVEAADRG | | SEQ ID No. 273 |
| MASK020 | GGPDGNEEMGGG | CC12-Q1P | SEQ ID No. 274 |
| MASK021 | GGFDGNEEMGGG | CC12-Q1F | SEQ ID No. 275 |
| MASK022 | GGGDGNEEMGGG | CC12-Q1G | SEQ ID No. 276 |
| MASK023 | GGEMDGEGQNGG | CC12-scramble | SEQ ID No. 277 |
| MASK024 | GGGGGPDGNEEPGG | | SEQ ID No. 278 |
| MASK025 | GGGGSLDGNEEPGG | | SEQ ID No. 279 |
| MASK026 | GGGGALDGNEEPGG | | SEQ ID No. 280 |
| MASK027 | GGGGGLDGNEEPGG | | SEQ ID No. 281 |
| MASK028 | GGGALDGNEEPGG | | SEQ ID No. 282 |
| MASK029 | GGGGGPDGNEEPGGG | | SEQ ID No. 283 |
| MASK030 | GGSGALDGNEEPGG | | SEQ ID No. 284 |
| MASK031 | GGSGSLDGNEEPGG | | SEQ ID No. 285 |
| MASK038 | GGSGGPDGNEEPGG | | SEQ ID No. 286 |
| MASK039 | GGVRDGPDGNEEPGG | | SEQ ID No. 287 |
| MASK040 | GGSGGPDGNEEPGGGG | | SEQ ID No. 288 |
| MASK041 | GGGRGPDGNEEPGG | | SEQ ID No. 289 |
| MASK042 | GGSGGLDGNEEPGG | | SEQ ID No. 290 |
| MASK043 | GGGVGPDGNEEPGG | | SEQ ID No. 291 |
| MASK044 | GGGEGPDGNEEPGG | | SEQ ID No. 292 |
| MASK046 | GGGVALDGNEEPGG | | SEQ ID No. 293 |
| MASK047 | GGGRALDGNEEPGG | | SEQ ID No. 294 |
| MASK048 | GGYAGLDGNEEPGG | | SEQ ID No. 295 |
| MASK049 | GGAGGPDGNEEPGG | | SEQ ID No. 296 |
| MASK051 | GGRGGPDGNEEPGG | | SEQ ID No. 297 |
| MASK052 | GGGGPDGNEEPGGGG | | SEQ ID No. 298 |
| MASK053 | GGGEALDGNEEPGG | | SEQ ID No. 299 |
| MASK054 | GGDASLDGNEEPGG | | SEQ ID No. 300 |
| MASK055 | GGRDAPDGNEEGG | | SEQ ID No. 301 |

TABLE 2
Exemplary sequences for the dual binding moieties
comprising a cleavable linker are provided in SEQ
ID Nos. 796-800.
Sequence

SEQ ID No. 796
EVQLVESGGGLVQPGNSLRLSCAASGFTFSKFGMSWVRQGGGGGLDGNEE
PGGLEWVSSISGSGRDTLYADSVKGRFTISRDNAKTTLYLQMNSLRPEDT
AVYYCTIGGSLSVSSQGTLVTVSSGGGGKPLGLQARVVGGGGT
SEQ ID No. 797
EVQLVESGGGLVQPGNSLRLSCAASGFTFSKFGMSWVRQGGGGGLDGNEE
PGGLEWVSSISGSGRDTLYADSVKGRFTISRDNAKTTLYLQMNSLRPEDT
AVYYCTIGGSLSVSSQGTLVTVSSGGGGPQASTGRSGGGGGGT
SEQ ID No. 798
EVQLVESGGGLVQPGNSLRLSCAASGFTFSKFGMSWVRQGGGGGLDGNEE
PGGLEWVSSISGSGRDTLYADSVKGRFTISRDNAKTTLYLQMNSLRPEDT
AVYYCTIGGSLSVSSQGTLVTVSSGGGGPQGSTGRAAGGGGGT
SEQ ID No. 799
EVQLVESGGGLVQPGNSLRLSCAASGFTFSKFGMSWVRQGGGGGLDGNEE
PGGLEWVSSISGSGRDTLYADSVKGRFTISRDNAKTTLYLQMNSLRPEDT
AVYYCTIGGSLSVSSQGTLVTVSSGGGGPPASSGRAGGGGGT
SEQ ID No. 800
EVQLVESGGGLVQPGNSLRLSCAASGFTFSKFGMSWVRQGGGGGLDGNEE
PGGLEWVSSISGSGRDTLYADSVKGRFTISRDNAKTTLYLCIMNSLRPED
TAVYYCTIGGSLSVSSQGTLVTVSSGGGGPIPVQGRAHGGGGT

The dual binding moiety, in certain embodiments, is any kind of polypeptide. In some embodiments, the dual binding moiety is a natural peptide, a synthetic peptide, or a fibronectin scaffold. In some embodiments, the dual binding moiety is an engineered bulk serum protein. In some embodiments, the dual binding moiety comprises one or more polypeptides. In some cases, the dual binding moiety comprises two or more polypeptides that are linked together by a non-cleavable linker. In certain examples, the non-CDR loop is in one of the two polypeptides and the CDR loop is on the other.

The bulk serum protein comprises, for example, albumin, fibrinogen, or a globulin. In some embodiments, the dual binding moiety is an engineered scaffold. The engineered scaffold comprises, for example, sdAb, a scFv, a Fab, a VHH, a fibronectin type III domain, immunoglobulin-like scaffold, Darin, cystine knot peptide, lipocalin, three-helix bundle scaffold, protein G-related albumin-binding module, or a DNA or RNA aptamer scaffold.

In some embodiments, the dual binding moiety is capable of interaction with at least one target antigen. In some embodiments, the non-CDR loops provide a binding site for the at least one target antigen binding domain, such that the moiety is capable of masking the target antigen binding domain from binding to its target. Target antigens, in some cases, are expressed on the surface of a diseased cell or tissue, for example a tumor or a cancer cell. Target antigens include but are not limited to EpCAM (exemplary protein sequence comprises UniProtkB ID No. P16422), EGFR (exemplary protein sequence comprises UniProtkB ID No. P00533), HER-2 (exemplary protein sequence comprises UniProtkB ID No. P04626), HER-3 (exemplary protein sequence comprises UniProtkB ID No. P21860), c-Met (exemplary protein sequence comprises UniProtkB ID No. P08581), FoIR (exemplary protein sequence comprises UniProtkB ID No. P15238), PSMA (exemplary protein sequence comprises UniProtkB ID No. Q04609), CD38 (exemplary protein sequence comprises UniProtkB ID No. P28907), BCMA (exemplary protein sequence comprises UniProtkB ID No. Q02223), and CEA (exemplary protein sequence comprises UniProtkB ID No. P06731, 5T4 (exemplary protein sequence comprises UniProtkB ID No. Q13641), AFP (exemplary protein sequence comprises UniProtkB ID No. P02771), B7-H3 (exemplary protein sequence comprises UniProtkB ID No. Q5ZPR3), CDH-6 (exemplary protein sequence comprises UniProtkB ID No. P97326), CAIX (exemplary protein sequence comprises UniProtkB ID No. Q16790), CD117 (exemplary protein sequence comprises UniProtkB ID No. P10721), CD123 (exemplary protein sequence comprises UniProtkB ID No. P26951), CD138 (exemplary protein sequence comprises UniProtkB ID No. P18827), CD166 (exemplary protein sequence comprises UniProtkB ID No. Q13740), CD19 (exemplary protein sequence comprises UniProtkB ID No. P15931), CD20 (exemplary protein sequence comprises UniProtkB ID No. P11836), CD205 (exemplary protein sequence comprises UniProtkB ID No. 060449), CD22 (exemplary protein sequence comprises UniProtkB ID No. P20273), CD30 (exemplary protein sequence comprises UniProtkB ID No. P28908), CD33 (exemplary protein sequence comprises UniProtkB ID No. P20138), CD352 (exemplary protein sequence comprises UniProtkB ID No. Q96DU3), CD37 (exemplary protein sequence comprises UniProtkB ID No. P11049), CD44 (exemplary protein sequence comprises UniProtkB ID No. P16070), CD52 (exemplary protein sequence comprises UniProtkB ID No. P31358), CD56 (exemplary protein sequence comprises UniProtkB ID No. P13591), CD70 (exemplary protein sequence comprises UniProtkB ID No. P32970), CD71 (exemplary protein sequence comprises UniProtkB ID No. P02786), CD74 (exemplary protein sequence comprises UniProtkB ID No. P04233), CD79b (exemplary protein sequence comprises UniProtkB ID No. P40259), DLL3 (exemplary protein sequence comprises UniProtkB ID No. Q9NYJ7), EphA2 (exemplary protein sequence comprises UniProtkB ID No. P29317), FAP (exemplary protein sequence comprises UniProtkB ID No. Q12884), FGFR2 (exemplary protein sequence comprises UniProtkB ID No. P21802), FGFR3 (exemplary protein sequence comprises UniProtkB ID No. P22607), GPC3 (exemplary protein sequence comprises UniProtkB ID No. P51654), gpA33 (exemplary protein sequence comprises UniProtkB ID No. Q99795), FLT-3 (exemplary protein sequence comprises UniProtkB ID No. P36888), gpNMB (exemplary protein sequence comprises UniProtkB ID No. Q14956), HPV-16 E6 (exemplary protein sequence comprises UniProtkB ID No. P03126), HPV-16 E7 (exemplary protein sequence comprises UniProtkB ID No. P03129), ITGA2 (exemplary protein sequence comprises UniProtkB ID No. P17301), ITGA3 (exemplary protein sequence comprises UniProtkB ID No. P26006), SLC39A6 (exemplary protein sequence comprises UniProtkB ID No. Q13433), MAGE (exemplary protein sequence comprises UniProtkB ID No. Q9HC15), mesothelin (exemplary protein sequence comprises UniProtkB ID No. Q13421), Muc1 (exemplary protein sequence comprises UniProtkB ID No. P15941), Muc16 (exemplary protein sequence comprises UniProtkB ID No. Q8WX17), NaPi2b (exemplary protein sequence comprises UniProtkB ID No. 095436), Nectin-4 (exemplary protein sequence comprises UniProtkB ID No. Q96918), CDH-3 (exemplary protein sequence comprises UniProtkB ID No. Q8WX17), CDH-17 (exemplary protein sequence comprises UniProtkB ID No. E5RJT3), EPHB2 (exemplary protein sequence comprises UniProtkB ID No. P29323), ITGAV (exemplary protein sequence comprises UniProtkB ID No. P06756), ITGB6 (exemplary protein sequence comprises UniProtkB ID No. P18564), NY-ESO-1 (exemplary protein sequence comprises UniProtkB ID No. P78358), PRLR (exemplary protein sequence comprises UniProtkB ID No. P16471), PSCA (exemplary protein sequence comprises UniProtkB ID No. 043653), PTK7 (exemplary protein sequence comprises UniProtkB ID No. Q13308), ROR1 (exemplary protein sequence comprises UniProtkB ID No. Q01973), SLC44A4 (exemplary protein sequence comprises UniProtkB ID No. Q53GD3), SLITRK5 (exemplary protein sequence comprises UniProtkB ID No. Q81W52), SLITRK6 (exemplary protein sequence comprises UniProtkB ID No. Q9HY7), STEAP1 (exemplary protein sequence comprises UniProtkB ID No. Q9UHE8), TIM1 (exemplary protein sequence comprises UniProtkB ID No. Q96D42), Trop2 (exemplary protein sequence comprises UniProtkB ID No. P09758), or WT1 (exemplary protein sequence comprises UniProtkB ID No. P19544), or any combinations thereof. In some embodiments, the target antigen is an immune checkpoint protein. Examples of immune checkpoint proteins include but are not limited to CD27 (exemplary protein sequence comprises UniProtkB ID No. P26842), CD137 (exemplary protein sequence comprises UniProtkB ID No. Q07011), 2B4 (exemplary protein sequence comprises UniProtkB ID No. Q9bZW8), TIGIT (exemplary protein sequence comprises UniProtkB ID No. Q495A1), CD155 (exemplary protein sequence comprises UniProtkB ID No. P15151), ICOS (exemplary protein sequence comprises UniProtkB ID No. Q9Y6W8), HVEM (exemplary protein sequence comprises UniProtkB ID No. 043557), CD40L (exemplary protein sequence comprises UniProtkB ID No. P29965), LIGHT (exemplary protein sequence comprises UniProtkB ID No. 043557), OX40 (exemplary protein sequence comprises UniProtkB ID No.), DNAM-1 (exemplary protein sequence comprises UniProtkB ID No. Q15762), PD-L1 (exemplary protein sequence comprises UniProtkB ID No. Q9ZQ7), PD1 (exemplary protein sequence comprises UniProtkB ID No. Q15116), PD-L2 (exemplary protein sequence comprises UniProtkB ID No. Q9BQ51), CTLA-4 (exemplary protein sequence comprises UniProtkB ID No. P16410), CD8 (exemplary protein sequence comprises UniProtkB ID No. P10966, P01732), CD40 (exemplary protein sequence comprises UniProtkB ID No. P25942), CEACAM1 (exemplary protein sequence comprises UniProtkB ID No. P13688), CD48 (exemplary protein sequence comprises UniProtkB ID No. P09326), CD70 (exemplary protein sequence comprises UniProtkB ID No. P32970), AA2AR (exemplary protein sequence comprises UniProtkB ID No. P29274), CD39 (exemplary protein sequence comprises UniProtkB ID No. P49961), CD73 (exemplary protein sequence comprises UniProtkB ID No. P21589), B7-H3 (exemplary protein sequence comprises UniProtkB ID No. Q5ZPR3), B7-H4 (exemplary protein sequence comprises UniProtkB ID No. Q7Z7D3), BTLA (exemplary protein sequence comprises UniProtkB ID No. Q76A9), IDO1 (exemplary protein sequence comprises UniProtkB ID No. P14902), IDO2 (exemplary protein sequence comprises UniProtkB ID No. Q6ZQW0), TDO (exemplary protein sequence comprises UniProtkB ID No. P48755), KIR (exemplary protein sequence comprises UniProtkB ID No. Q99706), LAG-3 (exemplary protein sequence comprises UniProtkB ID No. P18627), TIM-3 (exemplary protein sequence comprises UniProtkB ID No. Q8TDQ0), or VISTA (exemplary protein sequence comprises UniProtkB ID No. Q9D659). In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, inflammed or fibrotic tissue cell.

In some embodiments, the dual binding moiety comprises a binding site for a bulk serum protein. In some embodiments, the CDRs provide the binding site for the bulk serum protein. The bulk serum protein is, in some examples, globulin, albumin, transferrin, IgG1, IgG2, IgG4, IgG3, IgA monomer, Factor XIII, Fibrinogen, IgE, or pentameric IgM. In some embodiments, the dual binding moiety comprises a binding site for an immunoglobulin light chain. In some embodiments, the CDRs provide a binding site for the immunoglobulin light chain. The immunoglobulin light chain may be, for example, an Igκ free light chain or an Igλ free light chain.

In one embodiment, the dual binding moiety comprises any type of binding domain, including but not limited to, domains from a monoclonal antibody, a polyclonal antibody, a recombinant antibody, a human antibody, a humanized antibody. In some embodiments, the dual binding moiety is a single chain variable fragment (scFv), a soluble TCR fragment, a single-domain antibody such as a heavy chain variable domain (VH), a light chain variable domain (VL) and a variable domain (VHH) of camelid derived nanobody. In other embodiments, the dual binding moiety is a non-Ig binding domain, i.e., antibody mimetic, such as anticalins, affilins, affibody molecules, affimers, affitins, alphabodies, avimers, DARPins, fynomers, kunitz domain peptides, and monobodies.

Further examples of target antigen binding domains that the dual binding moiety is capable of interacting with include, but are not limited to, a T cell engager, a bispecific T cell engager, a dual-affinity re-targeting antibody, a variable heavy domain (VH), a variable light domain (VL), a soluble TCR fragment comprising a Valpha and Vbeta domain, an scFv comprising a VH and a VL domain, a single domain antibody (sdAb), or a variable domain of camelid derived nanobody (VHH), a non-Ig binding domain, i.e., antibody mimetic, such as anticalins, affilins, affibody molecules, affimers, affitins, alphabodies, avimers, DARPins, fynomers, kunitz domain peptides, and monobodies, a ligand or peptide. In some embodiments, the first or the second target antigen binding domain is a VHH domain. In some embodiments, the dual binding moiety is capable of binding a target antigen binding domain, wherein the target antigen binding domain is an sdAb. In some instances, the dual binding moiety is capable of binding aCD3 binding domain.

It is contemplated herein that the dual binding moiety described herein comprises at least one cleavable linker. In one aspect, the cleavable linker comprises a polypeptide having a sequence recognized and cleaved in a sequence-specific manner. The cleavage, in certain examples, is enzymatic, based on pH sensitivity of the cleavable linker, or by chemical degradation. A protease cleavable linker, in some cases, is recognized in a sequence-specific manner by a matrix metalloprotease (MMP), for example a MMP9. In some cases, the protease cleavable linker is recognized by a MMP9 comprises a polypeptide having an amino acid sequence PR(S/T)(L/I)(S/T). In some cases, the protease cleavable linker is recognized by a MMP9 and comprises a polypeptide having an amino acid sequence LEATA. In some cases, the protease cleavable linker is recognized in a sequence-specific manner by MMP11 In some cases, the protease cleavable linker recognized by MMP11 comprises a polypeptide having an amino acid sequence GGAANLVRGG (SEQ IN NO: 3). In some cases, the protease cleavable linker is recognized by a protease disclosed in Table 3. In some cases, the protease cleavable linker is recognized by a protease disclosed in Table 3 comprises a polypeptide having an amino acid sequence selected from a sequence disclosed in Table 3 (SEQ ID NOS: 1-42, 53, and 58-62).

Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mir1-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP7, MMP8, MMP9, MMP13, MMP11, MMP14, urokinase plasminogen activator (uPA), enterokinase, prostate-specific antigen (PSA, hK3), interleukin-1β converting enzyme, thrombin, FAP (FAP-α), dipeptidyl peptidase, type II transmembrane serine proteases (TTSP), neutrophil serine protease, cathepsin G, proteinase 3, neutrophil serine protease 4, mast cell chymase, mast cell tryptases

TABLE 3
Exemplary Proteases and Protease Recognition
Sequences

| | | SEQ |
|---|---|---|
| | Cleavage Domain | ID |
| Protease | Sequence | NO: |
| MMP7 | KRALGLPG | 1 |
| MMP7 | (DE)₈RPLALWRS(DR)₈ | 2 |
| MMP9 | PR(S/T)(L/I)(S/T) | 3 |
| MMP9 | LEATA | 4 |
| MMP11 | GGAANLVRGG | 5 |
| MMP14 | SGRIGFLRTA | 6 |
| MMP | PLGLAG | 7 |
| MMP | PLGLAX | 8 |
| MMP | PLGC(me)AG | 9 |
| MMP | ESPAYYTA | 10 |
| MMP | RLQLKL | 11 |
| MMP | RLQLKAC | 12 |
| MMP2, MMP9, MMP14 | EP(Cit)G(Hof)YL | 13 |
| Urokinase plasminogen | SGRSA | 14 |
| activator (uPA) | | |
| Urokinase plasminogen | DAFK | 15 |
| activator (uPA) | | |
| Urokinase plasminogen | GGGRR | 16 |
| activator (uPA) | | |
| Lysosomal Enzyme | GFLG | 17 |
| Lysosomal Enzyme | ALAL | 18 |
| Lysosomal Enzyme | FK | 19 |
| Cathepsin B | NLL | 20 |
| Cathepsin D | PIC(Et)FF | 21 |
| Cathepsin K | GGPRGLPG | 22 |
| Prostate Specific Antigen | HSSKLQ | 23 |
| Prostate Specific Antigen | HSSKLQL | 24 |
| Prostate Specific Antigen | HSSKLQEDA | 25 |
| Herpes Simplex Virus | LVLASSSFGY | 26 |
| Protease | | |
| HIVProtease | GVSQNYPIVG | 27 |
| CMVProtease | GVVQASCRLA | 28 |
| Thrombin | F(Pip)RS | 29 |
| Thrombin | DPRSFL | 30 |
| Thrombin | PPRSFL | 31 |
| Caspase-3 | DEVD | 32 |
| Caspase-3 | DEVDP | 33 |
| Caspase-3 | KGSGDVEG | 34 |
| Interleukin 1β converting | GWEHDG | 35 |
| enzyme | | |
| Enterokinase | EDDDDKA | 36 |
| FAP | KQEQNPGST | 37 |
| Kallikrein 2 | GKAFRR | 38 |
| Plasmin | DAFK | 39 |
| Plasmin | DVLK | 40 |
| Plasmin | DAFK | 41 |
| TOP | ALLLALL | 42 |
| MMP9 + matriptase | KPLGLQARVV | 58 |
| MMP9 + matriptase + uPA | PQASTGRSGG | 59 |
| MMP9 + matriptase + uPA | PQGSTGRAAG | 60 |
| Matriptase + uPA | PPASSGRAGG | 61 |
| MMP9 + matriptase | PIPVQGRAH | 62 |
| MMP9 + matriptase + uPA | PQGSTARSAG | 988 |

Proteases are known to be secreted by some diseased cells and tissues, for example tumor or cancer cells, creating a microenvironment that is rich in proteases or a protease-rich microenvironment. In some case, the blood of a subject is rich in proteases. In some cases, cells surrounding the tumor secrete proteases into the tumor microenvironment. Cells surrounding the tumor secreting proteases include but are not limited to the tumor stromal cells, myofibroblasts, blood cells, mast cells, B cells, NK cells, regulatory T cells, macrophages, cytotoxic T lymphocytes, dendritic cells, mesenchymal stem cells, polymorphonuclear cells, and other cells. In some cases, proteases are present in the blood of a subject, for example proteases that target amino acid sequences found in microbial peptides. This feature allows for targeted therapeutics such as antigen binding proteins to have additional specificity because T cells will not be bound by the antigen binding protein except in the protease rich microenvironment of the targeted cells or tissue.

In some embodiments of this disclosure the dual binding moieties described herein comprise at least one non-cleavable linker. The non-cleavable linker comprises, in some examples, a sequence as set forth in SEQ ID No. 51, SEQ ID No. 302, SEQ ID No. 303, SEQ ID No. 304, or SEQ ID No. 305.

TABLE 4
Exemplary non-cleavable linker sequences

| Non-cleavable | | |
|---|---|---|
| Linker ID | Sequence | SEQ ID No. |
| L002 | SGGGGSGGVV | SEQ ID No. 302 |
| L016 | SGGGGSGGGGSGGGGS | SEQ ID No. 303 |
| L017 | SGGGGSGGGGSGGGGGS | SEQ ID No. 304 |
| L046 | SGGGGSGGGS | SEQ ID No. 305 |

The dual binding moiety comprising the cleavable linker thus masks the binding of a first or a second target antigen binding domain to their respective targets. In some embodiments, the dual binding moiety is bound to a first target antigen binding domain, which is further bound to a second target antigen binding domain, in the following order: dual binding moiety (M): cleavable linker (L): first target antigen binding domain (T1): second antigen binding domain (T2), to form a ProTriTAC molecule. In other examples, the domains are organized in any one of the following orders: M:L:T2:T1; T2:T1:L:M, T1:T2:L:M. The dual binding moiety is, in some cases, further bound to a half-life extending protein, such as albumin or any other of its targets as described below. In some instances, the dual binding moiety is albumin or comprises a binding site for albumin. In some instances the dual binding moiety comprises a binding site for IgE. In some embodiments, the dual binding moiety comprises a binding site for Igκ free light chain. In some embodiments, the dual binding moiety comprises an albumin binding domain (anti-Alb), the first target antigen binding domain (T1) comprises a CD3 binding domain (e.g., an anti-CD3 scFv), and a ProTriTAC molecule has the following orientation: anti-Alb: anti-CD3: T2. In some embodiments, the dual binding moiety comprises an albumin binding domain (anti-Alb), the second target antigen binding domain (T2) comprises a CD3 binding domain (e.g., an anti-CD3 scFv), and a ProTriTAC molecule has the following orientation: anti-Alb:T1: anti-CD3. The T1 domain, in certain examples, is a tumor antigen binding domain, such as, but not limited to, an anti-EGFR domain, an anti-MSLN domain, an anti-BCMA domain, an anti-EpCAM domain, an anti-PSMA domain, or an anti-DLL3 domain.

ProTriTAC molecules (also referred to herein as protrispecific molecules) comprising a dual binding moiety as described herein are, in some embodiments, T cell engager prodrugs designed to be conditionally active in a tumor microenvironment. In some cases, this enables targeting of a wider selection of tumor antigens (e.g., solid tumor antigens). The ProTriTAC molecules, in some examples, combine the desirable attributes of several prodrug approaches, including, but not limited to: combination of steric and specific masking, wherein the steric masking is, in some cases, is through albumin that is recognized by an anti-albumin domain in a ProTriTAC molecule, and the specific masking, in some cases, is through specific intermolecular interactions between an anti-albumin domain (in some examples) and a target antigen binding domain of the ProTriTAC molecule (such as, an anti-CD3 scFv domain, in some examples); additional safety imparted by half-life differential of prodrug versus an active drug, derived by activation of the conditionally activated ProTriTAC molecule; ability to plug-and-play with different tumor target binders.

### Binding Moiety Variants

As used herein, the term “binding moiety variants” refers to variants and derivatives of the dual binding moiety described herein, comprising non-CDR loops that are capable of binding to domain, such as a target antigen binding domain and CDRs that are capable of binding to a half-life extending protein, such as albumin. In certain embodiments, amino acid sequence variants of the dual binding moiety described herein are contemplated. For example, in certain embodiments amino acid sequence variants of the dual binding moiety described herein are contemplated to improve the binding affinity alone or along with other biological properties of the antibodies. Exemplary method for preparing such amino acid variants include, but are not limited to, introducing appropriate modifications into the nucleotide sequence encoding the antibody, or by peptide synthesis. Such modifications include, for example, deletions from, and/or insertions into and/or substitutions of residues within the amino acid sequences of the dual binding moiety.

Any combination of deletion, insertion, and substitution are made, in various embodiments, to the dual binding moiety, to arrive at the final construct, provided that the final construct possesses a desired characteristic, e.g., capability of binding a target antigen binding domain and a half-life extending protein. In certain embodiments, binding protein variants having one or more amino acid substitutions are provided. Sites of interest for substitution mutagenesis include the CDRs, the non-CDR loops, and the framework regions. Amino acid substitutions are introduced, in some cases, into the non-CDR loops of a dual binding moiety and the products screened for a desired activity, e.g., retained/improved antigen binding, decreased immunogenicity, or improved antibody-dependent cell mediated cytotoxicity (ADCC) or complement dependent cytotoxicity (CDC). Both conservative and non-conservative amino acid substitutions are contemplated for preparing the dual binding moiety variants. Amino acid substitutions may be conservative or semi-conservative. For example, the amino acids glycine, alanine, valine, leucine and isoleucine can often be substituted for one another (amino acids having aliphatic side chains). Of these possible substitutions, typically glycine and alanine are used to substitute for one another since they have relatively short side chains and valine, leucine and isoleucine are used to substitute for one another since they have larger aliphatic side chains which are hydrophobic. Other amino acids which may often be substituted for one another include but are not limited to: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur-containing side chains). In some embodiments, the conditionally active target-binding proteins are isolated by screening combinatorial libraries, for example, by generating phage display libraries and screening such libraries for antibodies possessing the desired binding characteristics towards a target antigen, such as a tumor antigen expressed on a cell surface.

In another example of a substitution to create a variant dual binding moiety, one or more hypervariable region residues of a parent molecule, such as a domain of an antibody are substituted. In general, variants are then selected based on improvements in desired properties compared to a parent antibody, for example, increased affinity, reduced affinity, reduced immunogenicity, increased pH dependence of binding. For example, an affinity matured variant dual binding moiety is generated, in some cases, e.g., using phage display-based affinity maturation techniques.

In some cases, substitutions are made in hypervariable regions (HVR) of an immunoglobulin binding molecule to generate variants and then selected based on binding affinity to a target antigen binding domain, to a half-life extending domain, or both, i.e., by affinity maturation. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. Substitutions can be in one, two, three, four, or more sites within a parent antibody sequence.

In some embodiments, a dual binding moiety, as described herein is “humanized”, or “camelized,” i.e., by replacing one or more amino acid residues in the amino acid sequence of said naturally occurring immunoglobulin binding molecule (and in particular in the framework sequences) by one or more of the amino acid residues that occur at the corresponding position(s) in an equivalent binding moiety from a conventional 4-chain antibody from a human being, or a camelid species.

### Affinity Maturation

In designing dual binding moieties for therapeutic applications, it is desirable to create proteins that, for example, modulate a functional activity of a target, and/or improved binding proteins such as binding proteins with higher specificity and/or affinity and/or and binding proteins that are more bioavailable, or stable or soluble in particular cellular or tissue environments.

The dual binding moieties described in the present disclosure exhibit improved the binding affinities towards domain target antigen binding domain. In some embodiments, the dual binding moiety of the present disclosure is affinity matured to increase its binding affinity to a target antigen binding domain, using any known technique for affinity-maturation (e.g., mutagenesis, chain shuffling, CDR amino acid substitution). Amino acid substitutions may be conservative or semi-conservative. For example, the amino acids glycine, alanine, valine, leucine and isoleucine can often be substituted for one another (amino acids having aliphatic side chains). Of these possible substitutions, typically glycine and alanine are used to substitute for one another since they have relatively short side chains and valine, leucine and isoleucine are used to substitute for one another since they have larger aliphatic side chains which are hydrophobic. Other amino acids which may often be substituted for one another include but are not limited to: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur-containing side chains). In some embodiments, the dual binding moiety is isolated by screening combinatorial libraries, for example, by generating phage display libraries and screening such libraries for binding moieties possessing the desired binding characteristics towards a target antigen binding domain, such as a domain that binds a tumor antigen expressed on a cell surface.

### Dual Binding Moiety Modifications

The dual binding moieties described herein encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence to block an immunogenic domain and/or for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in the dual binding moieties described herein, including the peptide backbone, the amino acid side-chains, and the amino or carboxyl termini. Certain common peptide modifications that are useful for modification of the conditionally active binding proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

In some embodiments, the dual binding moieties of the disclosure are capable of being conjugated with drugs to form antibody-drug conjugates (ADCs). In general, ADCs are used in oncology applications, where the use of antibody-drug conjugates for the local delivery of cytotoxic or cytostatic agents allows for the targeted delivery of the drug moiety to tumors, which can allow higher efficacy, lower toxicity, etc.

### Polynucleotides Encoding the Dual Binding Moiety

Also provided, in some embodiments, are polynucleotide molecules encoding the dual binding moiety as described herein. In some embodiments, the polynucleotide molecules are provided as a DNA construct. In other embodiments, the polynucleotide molecules are provided as a messenger RNA transcript.

The polynucleotide molecules are constructed by methods such as by combining the genes encoding the dual binding moiety comprising a single domain or in some cases comprising two or more polypeptides separated by peptide linkers or, in other embodiments, two or more polypeptides directly linked by a peptide bond, into a single genetic construct operably linked to a suitable promoter, and optionally a suitable transcription terminator, and expressing it in bacteria or other appropriate expression system such as, for example CHO cells. Depending on the vector system and host utilized, any number of suitable transcription and translation elements, including constitutive and conditionally active promoters, may be used. The promoter is selected such that it drives the expression of the polynucleotide in the respective host cell.

In some embodiments, the polynucleotide is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described conditionally active binding protein. Examples of expression vectors for expression in E. coli are pSKK (Le Gall et al., J Immunol Methods. (2004) 285(1):111-27) or pcDNA5 (Invitrogen) for expression in mammalian cells.

Thus, the dual binding moiety or the conditionally active binding proteins comprising the dual binding moiety as described herein, in some embodiments, are produced by introducing a vector encoding the moiety or the protein as described above into a host cell and culturing said host cell under conditions whereby the moiety or the protein domains are expressed.

### Pharmaceutical Compositions

Also provided, in some embodiments, are pharmaceutical compositions comprising a therapeutically effective amount a dual binding moiety of the present disclosure, and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The dual binding moieties described herein are contemplated for use as a medicament. Administration is effected by different ways, e.g., by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

### Methods of Treatment

Also provided herein, in some embodiments, are methods and uses for stimulating the immune system of an individual in need thereof comprising administration of a dual binding moiety as described herein. In some instances, administration induces and/or sustains cytotoxicity towards a cell expressing a target antigen. In some instances, the cell expressing a target antigen is a cancer or tumor cell, a virally infected cell, a bacterially infected cell, an autoreactive T or B cell, damaged red blood cells, arterial plaques, or fibrotic tissue. In some embodiments, the target antigen is an immune checkpoint protein.

Also provided herein are methods and uses for a treatment of a disease, disorder or condition associated with a target antigen comprising administering to an individual in need thereof a dual binding protein as described herein, which is capable of binding a half-life extending protein and further capable of interacting with a domain, such as a target antigen binding domain and masking the same from binding its target. Diseases, disorders or conditions associated with a target antigen include, but are not limited to, viral infection, bacterial infection, auto-immune disease, transplant rejection, atherosclerosis, or fibrosis. In other embodiments, the disease, disorder or condition associated with a target antigen is a proliferative disease, a tumorous disease, an inflammatory disease, an immunological disorder, an auto-immune disease, an infectious disease, a viral disease, an allergic reaction, a parasitic reaction, a graft-versus-host disease or a host-versus-graft disease. In one embodiment, the disease, disorder or condition associated with a target antigen is cancer. In one instance, the cancer is a hematological cancer. In another instance, the cancer is a melanoma. In a further instance, the cancer is non-small cell lung cancer. In yet further instance, the cancer is breast cancer.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments of the methods described herein, the dual binding protein as described herein, which is capable of binding a half-life extending protein and further capable of interacting with a domain, such as a target antigen binding domain and masking the same from binding its target is administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (γ-rays, X-rays, and/or the directed delivery of radioisotopes, microwaves, UV radiation and the like), gene therapies (e.g., antisense, retroviral therapy and the like) and other immunotherapies. In some embodiments, the conditionally active binding protein described herein is administered in combination with anti-diarrheal agents, anti-emetic agents, analgesics, opioids and/or non-steroidal anti-inflammatory agents. In some embodiments, the dual binding protein as described herein, which is capable of binding a half-life extending protein and further capable of interacting with a domain, such as a target antigen binding domain and masking the same from binding its target is administered before, during, or after surgery.

### EXAMPLES

The examples below further illustrate the described embodiments without limiting the scope of the disclosure.

### Example 1: Construction of an Exemplary Dual Binding Moiety which Binds to Albumin and a Target Antigen Binding Domain Whose Target is EGFR

The sequence of an engineered protein scaffold comprising CDR loops capable of binding albumin and non-CDR loops is obtained. Overlapping PCR is used to introduce random mutations in the non-CDR loop regions, thereby generating a library. The resultant sequences are cloned into a phage display vector, thereby generating a phage display library. Escherichia coli cells are transformed with the library and used to construct a phage display library. ELISA is performed using an immobilized target antigen binding domain with specificity for EGFR. A clone with high specificity for EGFR is selected. Affinity maturation is performed by re-randomizing residues in the non-CDR loop regions as before.

Sequence alignment of non-CDR loop regions of the resultant proteins is performed to determine sequence conservation between proteins with high affinity for the EGFR binding target antigen binding domain. Site directed mutagenesis of one or more amino acids within these regions of sequence conservation is performed to generate additional proteins. Binding of the resultant proteins to an immobilized target antigen binding domain whose target is EGFR is measured in an ELISA. A protein with the highest affinity for the target antigen binding domain is selected.

The sequence of this dual binding moiety is cloned into a vector comprising a sequence for a cleavable linker, and sequences for a second target antigen binding domain that binds to a second target antigen, e.g., CD3. The resultant vector is expressed in a heterologous expression system to obtain a conditionally active target binding protein that comprises a dual binding moiety comprising a cleavable linker and non-CDR loops which provide a binding site specific for the target antigen binding domain whose target is EGFR, and CDR loops which are specific for albumin.

### Example 2: An Exemplary Dual Binding Moiety which Comprises an Extended Non-CDR Loop into which a Human CD3ε Epitope is Grafted

The sequence of a dual binding moiety comprising non-CDR loops (AB, EF, C″D, and CC′) is obtained. A portion of the human CD3ε sequence is grafted into the CC′ loop of the non-CDR loops within the dual binding moiety, along with glycine residues to further extend the CC′ loop. The dual binding moiety comprising extended non-CDR loops comprising the CD3ε sequences, as described above, are cloned into a vector further comprising coding sequences for a protease cleavable linker, a scFv containing a CD3 binding domain, and an EGFR binding domain, to express a conditionally activated molecule. The multidomain molecule is subsequently exposed to a tumor associated protease, matriptase, to assay activation of the molecule upon cleavage of the protease cleavable linker, which separates the dual binding moiety comprising the cleavable linker from the rest of the molecule, i.e., the scFv containing the CD3 binding domain and the EGFR binding domain (aEGFR). Activation of the multidomain molecule, containing the extended CC′ non-CDR loop, CD3 binding scFv in a VH-VL or a VL-VH format, upon treatment with matriptase, is observed. A multidomain molecule containing a wild-type CC′ loop in the dual binding moiety is used as a control for the protease activation assay. In addition, a control multidomain molecule that is not in the “pro” form, i.e., a molecule that includes the same domains as the ProTriTAC molecule, except that it has a half-life extension domain, such as albumin, instead of a dual binding moiety, is also treated with matriptase and used as a control. Results indicate that the multidomain molecules are activated upon cleavage, to generate a free albumin binding domain whereas the albumin domain did not separate from the control multidomain molecule. Thus, the multidomain molecules containing a dual binding moiety of this disclosure is able to readily dissociate from the half-life extending domain upon cleavage in a tumor microenvironment, unlike the control multidomain molecules, and thereby are amenable to rapid clearance from the systemic circulation upon activation.

Further studies are carried out to assay the binding of the dual binding moiety containing the human CD3ε to CD3. It was observed that the activated forms of multidomain molecule that contained the dual binding moiety containing the human CD3ε are about 20 times potent in binding CD3 than their activated forms which do not contain the dual binding moiety.

Cell killing potential of a multidomain molecule that contains a dual binding moiety as described herein are also assayed in a study where a cancer cell line is treated with the multidomain molecule or its activated form.

### Example 3: A Conditionally Active Binding Protein of this Disclosure Exhibits Reduced Specificity Toward Cell Line which Overexpresses EGFR but is Protease Deficient

Cells overexpressing EGFR and exhibiting low expression of a matrix metalloprotease are separately incubated with an exemplary conditionally active binding protein according to the present disclosure and a non-conditionally active control binding protein. Cells expressing normal levels of EGFR and proteases are also incubated with a conditionally active binding protein according to the present disclosure and a non-conditionally active control binding protein. Both proteins comprise a target antigen binding domain with specificity toward PSMA.

Results indicate that in the absence of protease secretion, the conditionally active binding protein of the present disclosure interacts with the protease expressing cells but does not interact with the EGFR expressed on the surface of the protease deficient cells. In contrast, the non-conditionally active control binding protein lacks the ability to selectively bind the protease expressing cells over the protease deficient ones. Thus, the exemplary conditionally active binding protein receptor of the present disclosure is advantageous, for example, in terms of reducing off-tumor toxicity.

### Example 4: Treatment with an Exemplary Conditionally Active Binding Protein of the Present Disclosure Inhibits In Vivo Tumor Growth

Murine tumor line CT26 is implanted subcutaneously in BALB/c mice and on day 7 post-implantation the average size of the tumor is measured. Test mice are treated with an exemplary conditionally active binding protein which has a target antigen binding domain specific for CTLA4 and another target antigen binding domain specific for CD3, wherein either the CTLA4 or the CD3 specific domain is bound to a dual binding moiety via its non-CDR loops, the dual binding moiety comprises a cleavable linker, and is bound to albumin. Control mice are treated with binding protein that contains CD3/CTLA4 specific domains but do not contain the dual binding moiety or the cleavable linker, and are not conditionally active. Results show that treatment with the exemplary conditionally active binding protein of the present disclosure inhibits tumor more efficiently than the comparator binding protein which does not contain the moiety with non-CDR loops.

### Example 5: Exemplary Conditionally Active Binding Protein Exhibits Reduced Specificity Toward Cell Line which Overexpresses Antigen but is Protease Deficient

Cells overexpressing CTLA-4 and exhibiting low expression of a matrix metalloprotease are separately incubated with an exemplary CTLA4 specific conditionally active binding protein of this disclosure, containing a dual binding moiety that binds to a CTLA4 binding domain via its non-CDR loops and albumin via its CDRs; or a control CTLA-4 binding antibody which does not contain the dual binding moiety which binds to a CTLA4 binding domain via its non-CDR loops and to albumin via its CDRs. Cells expressing normal levels of antigens and proteases are also incubated with the exemplary CTLA4 specific conditionally active binding protein, or the control CTLA4 binding antibody.

Results indicate that in the absence of protease secretion, the conditionally active binding protein of the present disclosure binds the protease expressing cells but does not bind the protease-deficient antigen expressing cells. In contrast, the control antibody lacks the ability to selectively bind the protease expressing cells over the protease deficient ones. Thus, the exemplary conditionally active binding protein of the present disclosure is advantageous, for example, in terms of reducing off-tumor toxicity.

### Example 6: Purification, Pharmacokinetic Analysis, and Potency Assays for Exemplary ProTriTAC Molecules Containing Exemplary Dual Binding Moieties of this Disclosure

A) Expression, Purification and Stability of Exemplary ProTriTAC Molecules

Protein Production

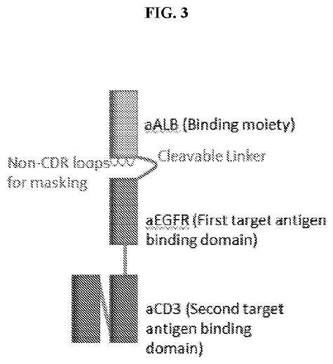

Sequences of exemplary ProTriTAC (also referred to as trispecific) molecules were cloned into mammalian expression vector pcDNA 3.4 (Invitrogen) preceded by a leader sequence and followed by a 6× Histidine Tag. Expi293F cells (Life Technologies A14527) were maintained in suspension in Optimum Growth Flasks (Thomson) between 0.2 to 8×1e6 cells/ml in Expi 293 media. Purified plasmid DNA was transfected into Expi293 cells in accordance with Expi293 Expression System Kit (Life Technologies, A14635) protocols, and maintained for 4-6 days post transfection. Alternatively, sequences of trispecific molecules were cloned into mammalian expression vector pDEF38 (CMC ICOS) transfected into CHO-DG44 dhfr-cells, stable pools generated, and cultured in production media for up to 12 days prior to purification. The amount of the exemplary trispecific proteins in conditioned media was quantitated using an Octet RED 96 instrument with Protein A tips (ForteBio/Pall) using a control trispecific protein for a standard curve. Conditioned media from either host cell was filtered and partially purified by affinity and desalting chromatography. Trispecific proteins were subsequently polished by ion exchange and upon fraction pooling formulated in a neutral buffer containing excipients. Final purity was assessed by SDS-PAGE and analytical SEC using an Acquity BEH SEC 200 1.7u 4.6×150 mm column (Waters Corporation) resolved in an aqueous/organic mobile phase with excipients at neutral pH on a 1290 LC system and peaks integrated with Chemstation CDS software (Agilent). Trispecific proteins purified from CHO host cells were analyzed by running an SDS-PAGE, as shown in FIG. 3.

Stability Assessment

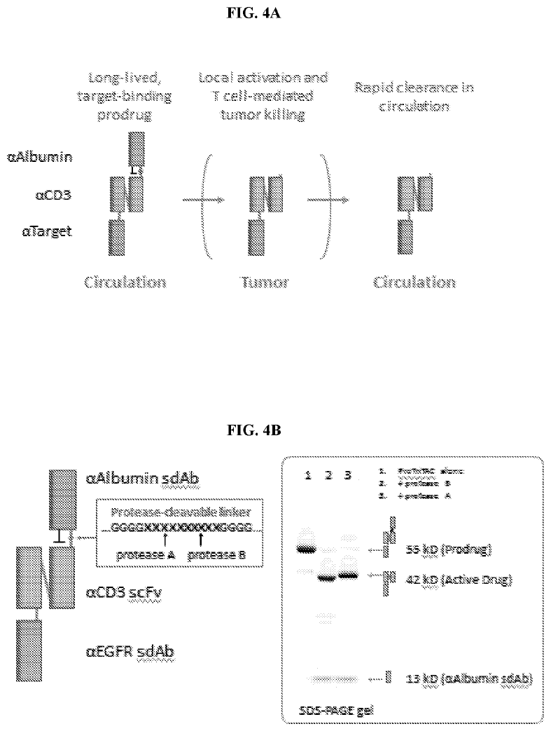

Purified trispecific molecules in two formulations were sub-aliquoted into sterile tubes and stressed by five freeze-thaw cycles each comprising greater than 1 hour at −80° C. and room temperature or by incubation at 37° C. for 1 week. Stressed samples were evaluated for concentration and turbidity by UV spectrometry using UV transparent 96 well plates (Corning 3635) with a SpectraMax M2 and SoftMaxPro Software (Molecular Devices), SDS-PAGE, and analytical SEC and compared to the same analysis of control non-stressed samples. An overlay of chromatograms from analytical SEC of control and stressed samples for a single exemplary trispecific ProTriTAC molecule purified from 293 host cells is depicted in FIG. 4.

B) ProTriTAC Exhibits Potent, Protease-Dependent, Anti-Tumor Activity in a Rodent Tumor Xenograft Model

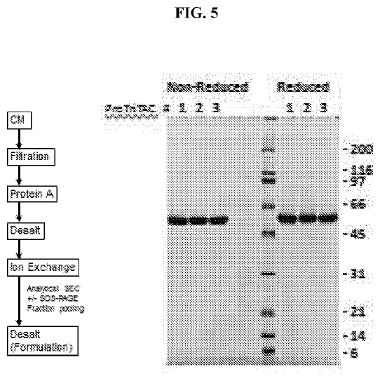

An exemplary ProTriTAC molecule (SEQ ID NO: 46) containing an EGFR binding domain as the target binding domain, a CD3 binding domain, and an albumin binding domain comprising a masking moiety (SEQ ID NO: 50) and a cleavable linker (SEQ ID NO: 53), was evaluated for anti-tumor activity in vivo in an HCT116 subcutaneous xenograft tumor admixed with expanded human T cells in immunocompromised NCG mice. A non-cleavable EGFR targeting ProTriTAC (SEQ ID NO: 47) molecule and a GFP targeting ProTriTAC molecule (SEQ ID NO: 49) were also used in the study. Specifically, 5×10⁶ HCT116 cells were admixed with 2.5×10⁶ expanded T cells per mouse on day 0. Dosing of the test molecules (EGFR targeting ProTriTAC, non-cleavable EGFR targeting Pro-TriTAC, and GFP targeting ProTriTAC) were performed starting on the following day with a q.d.×10 (single daily dose for 10 days) schedule via intraperitoneal injection, at a dose of 0.03 mg/kg. Tumor volumes were determined using caliper measurements and calculated using the formula V=(length×width×width)/2, at the indicated times. Results shown in FIG. 5 indicate that following final dose of test molecules, on day 10, tumor growth was arrested in mice administered the activatable EGFR targeting ProTriTAC molecule. Whereas, administering the GFP targeting ProTriTAC molecule was not effective in inhibiting tumor growth and the administration of the EGFR targeting non-cleavable ProTriTAC molecule was not as potent as the activatable ProTriTAC, in arresting tumor growth.

C) Demonstration of Functional Masking and Stability of ProTriTAC In Vivo in a Three-Week Cynomolgus Monkey Pharmacokinetic Study

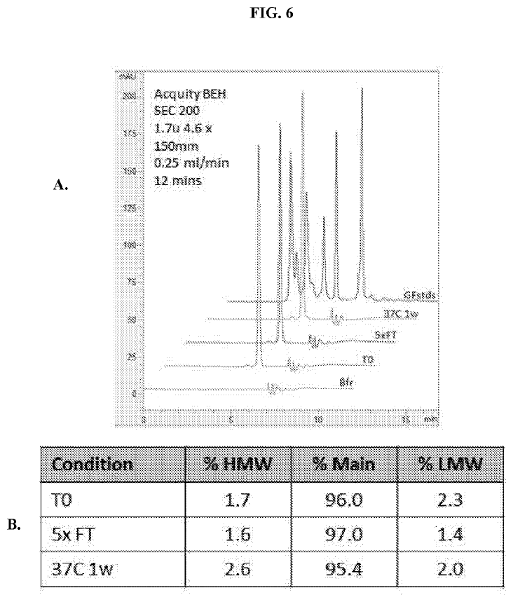

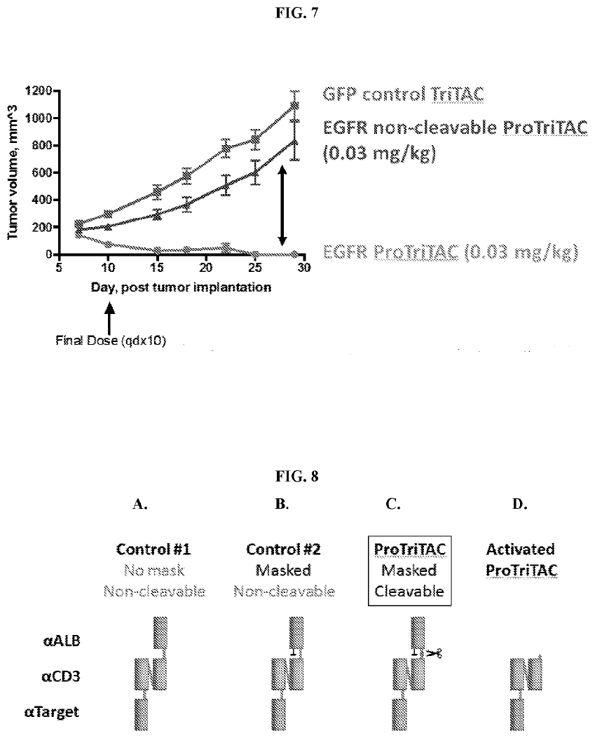

Single doses of an exemplary PSMA targeting ProTriTAC (SEQ ID NO: 43) containing a PSMA binding domain as the target binding domain, a CD3 binding domain, and an albumin binding domain comprising a masking moiety (SEQ ID NO: 50) and a cleavable linker (SEQ ID NO: 53), non-cleavable PSMA targeting ProTriTAC (SEQ ID NO: 44); non-masked/non-cleavable PSMA targeting TriTAC (SEQ ID NO: 52); and active drug mimicking protease-activated PSMA targeting ProTriTAC (SEQ ID NO: 45) were dosed into cynomolgus monkeys at 0.1 mg/kg via intravenous injection. Plasma samples were collected at the time points indicated in FIG. 7 The designs of the above described test molecules are shown in FIG. 6. Concentrations of the various test molecules, as described above, were determined using ligand binding assays with biotinylated recombinant human PSMA (R&D systems) and sulfo-tagged anti-CD3 idiotype antibody cloned 11D3 in a MSD assay (Meso Scale Diagnostic, LLC). Pharmacokinetic parameters were estimated using Phoenix WinNonlin pharmacokinetic software using a non-compartmental approach consistent with the intravenous bolus route of administration.

To calculate the rate of in vivo conversion of the test molecules (i.e., conversion of PSMA targeting ProTriTAC, non-cleavable PSMA targeting ProTriTAC, non-masked/non-cleavable PSMA targeting ProTriTAC, and active drug mimicking protease-activated PSMA targeting ProTriTAC) the concentration of active drug in circulation was estimated by solving the following system of differential equations where P is the concentration of prodrug, A is the concentration of active drug, kₐ is the rate of prodrug activation in circulation, k_{c,P} is the clearance rate of the prodrug, and k_{c,A} is the clearance rate of the active drug.

$$\frac{d P}{dt} = - {k}_{c , P} P$$

$$\frac{d A}{dt} = {k}_{a} P - {k}_{c , A} A$$

The clearance rates of the prodrug, active drug, and a non-cleavable prodrug control (k_{c,NCLV}) were determined empirically in cynomolgus monkeys. To estimate the rate of prodrug activation in circulation, it was assumed that the difference between the clearance rate of cleavable prodrug and the non-cleavable prodrug arose solely from non-specific activation in circulation. Therefore, the rate of prodrug conversion to active drug in circulation was estimated by subtracting the clearance rate of the cleavable prodrug from the non-cleavable prodrug.

kₐ=k_{c,NCLV}−k_{c,P}

The initial concentration of prodrug in circulation was determined empirically and the initial concentration of active drug was assumed to be zero. Further calculations showed that the ProTriTAC comprising the protease cleavable linker was sufficiently stable in circulation, with 50% non-tumor mediated conversion every 194 hours and the t_{1/2} of the molecule was determined, empirically, to be around 211 hours. This indicated that ProTriTAC molecules are sufficiently stable and protected against off-tumor effects. In contrast, the t_{1/2} of the active drug fragment mimicking the activated ProTriTAC molecule was determined, empirically, to be 0.97 hours. Thus, active drug was rapidly cleared from circulation. Results are shown in FIG. 8.

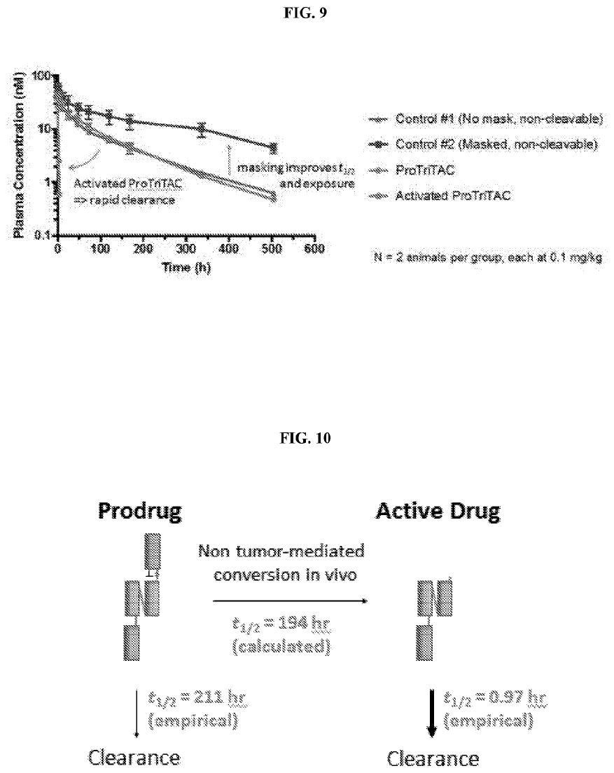

FIG. 9 shows that because the active drug mimicking the activated ProTriTAC molecule was rapidly cleared from circulation and that the ProTriTAC and the control non-masked non-cleavable ProTriTAC had longer longer half-lives, there is a significant >200-fold differential between the ProTriTAC and the activated ProTriTAC circulating exposure. It was also observed that the masked but non-cleavable ProTriTAC control molecule was present in circulation for a longer time than the non-masked non-cleavable ProTriTAC control, thereby indicating that masking plays a role in increased circulation half-life (t_{1/2}) and limited peripheral T cell binding. The combination of the functional masking that renders ProTriTAC inert outside the tumor environment and the half-life differential that renders any aberrantly activated ProTriTAC in circulation to be rapidly cleared in vivo works together to ensure on-target, off-tumor activity of ProTriTAC is minimized. Supporting pharmacokinetic parameters are shown in Table 4.

TABLE 4
Pharmacokinetics of ProTriTAC, Activated
ProTriTAC and control molecules

| | Terminal t_{1/2} | Cₘₐₓ | AUC, 0-last | Clearance |
|---|---|---|---|---|
| Test Article | (hr) | (nM) | (hr*nM) | (mL/hr/kg) |
| Control #1 (No mask, | 118 | 48.2 | 2490 | 0.735 |
| non-cleavable) | | | | |
| Control #2 (Masked, | 211 | 58.0 | 7000 | 0.238 |
| non-cleavable) | | | | |
| ProTriTAC | 101 | 42.7 | 2670 | 0.686 |
| Activated ProTriTAC | 0.969 | 66.6 | 41.5 | 58.5 |

D) Protease Activation of ProTriTAC Molecule Leads to Significantly Enhanced Activity In Vitro

The aim of this study was to assess the relative potency of protease activatable ProTriTAC molecules, non-cleavable ProTriTAC molecules and recombinant active drug fragment mimicking the protease-activated ProTriTAC molecule, in CD3 binding and T cell mediated cell killing. The active drug fragment mimicking the protease activated ProTriTAC molecule contained the CD3 binding domain and the target antigen binding domain but lacked the albumin binding domain. Whereas the protease activatable ProTriTAC molecule contained the albumin binding domain comprising a masking domain and a protease cleavable site, the CD3 binding domain, and the target antigen binding domain. The non-cleavable ProTriTAC molecule lacked the protease cleavable site but was otherwise identical to the protease activatable ProTriTAC molecule.

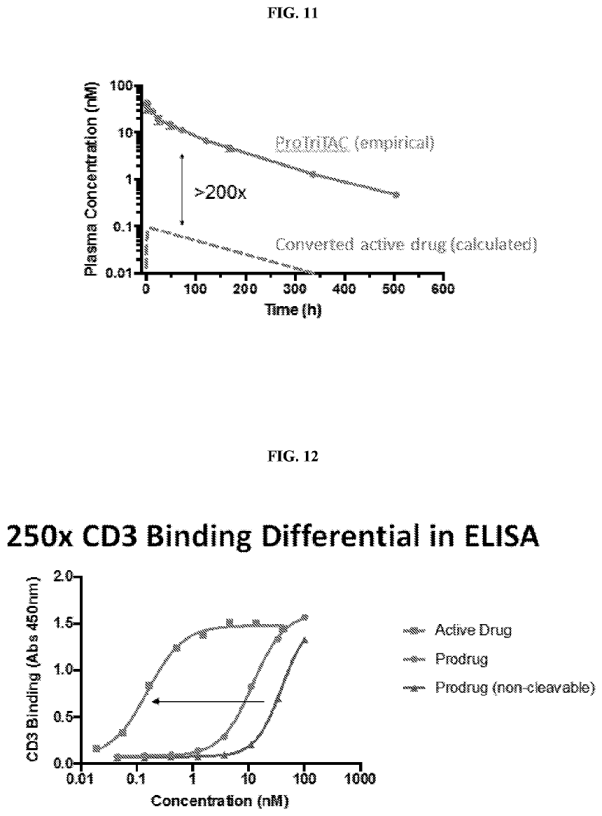

Purified ProTriTAC (labeled as prodrug in FIGS. 10-12), non-cleavable ProTriTAC (labeled as prodrug (non-cleavable) in FIGS. 10-12), and recombinant active drug fragment mimicking the protease-activated ProTriTAC (labeled as active drug in FIGS. 10-12) were tested for binding to recombinant human CD3 in an ELISA assay (FIG. 10), binding to purified human primary T cells in a flow cytometry assay (FIG. 11), and functional potency in a T cell-dependent cellular cytotoxicity assay (FIG. 12).

For ELISA, soluble test molecules (i.e., active drug, prodrug, and prodrug (non-cleavable) at the indicated concentrations were incubated, in multi-well plates, with immobilized recombinant human CD3a (R&D Systems) for 1 hour at room temperature in PBS supplemented with 15 mg/mL human serum albumin. Plates were blocked using SuperBlock (Thermo Fisher), washed using PBS with 0.05% Tween-20, and detected using a non-competitive anti-CD3 idiotype monoclonal antibody 11D3 followed by peroxidase-labeled secondary antibody and TMB-ELISA substrate solution (Thermo Fisher). Results shown in FIG. 10 demonstrate that the active drug fragment mimicking the protease-activated ProTriTAC molecule was about 250 times more potent in binding CD3 as compared to prodrug non-cleavable. The EC₅₀ values are provided in Table 5. The masking ratio is the ratio between the prodrug EC₅₀ over the active drug EC₅₀: the higher the number, the higher the fold-shift between prodrug and active drug and thus greater functional masking.

TABLE 5
CD3 binding potential

| | EC₅₀ (nM) | Masking Ratio |
|---|---|---|
| Active Drug | 0.16 | — |
| Prodrug | 11.91 | 74 |
| Prodrug (non-cleavable) | 39.44 | 247 |

For binding to human primary T cells, determined by flow cytometry, soluble test molecules (i.e., active drug, prodrug, and prodrug (non-cleavable)) at the indicated concentrations (shown in FIG. 11) were incubated, in multi-well plates, with purified human primary T cells for 1 h at 4° C. in the presence of PBS with 2% fetal bovine serum and 15 mg/ml human serum albumin. Plates were washed with PBS with 2% fetal bovine serum, detected using AlexaFluor 647-labeled non-competitive anti-CD3 idiotype monoclonal antibody 11D3, and data was analyzed using FlowJo 10 (FlowJo, LLC). Results shown in FIG. 11 demonstrate that the active drug fragment mimicking the protease-activated ProTriTAC molecule was greater than 1000 times more potent in binding human primary T cells as compared to prodrug non-cleavable. The EC₅₀ values are provided in Table 6.

TABLE 6
Human primary T cell binding potential

| | EC₅₀ (nM) | Masking Ratio |
|---|---|---|
| Active Drug | 1.19 | — |
| Prodrug | >1000 | n/a |
| Prodrug (non-cleavable) | >1000 | n/a |

For functional potency in a T cell-dependent cellular cytotoxicity assays, soluble test molecules (i.e., active drug, prodrug, and prodrug (non-cleavable)) at the indicated concentrations, shown in FIG. 12, were incubated, in multi-well plates, with purified resting human T cells (effector cell) and HCT116 cancer cell (target cell) at 10:1 effector: target cell ratio for 48 h at 37° C. The HCT116 target cell line had been stably transfected with a luciferase reporter gene to allow specific T cell-mediated cell killing measurement by ONE-Glo (Promega). Results shown in FIG. 12 demonstrate that the active drug fragment mimicking the protease-activated ProTriTAC molecule was about 500 times more potent in T cell mediated killing of cancer cells, as compared to prodrug non-cleavable. The EC₅₀ values are provided in Table 7.

TABLE 7
T cell mediated cell killing potential

| | EC₅₀ (nM) | Masking Ratio |
|---|---|---|
| Active Drug | 0.004 | — |
| Prodrug | 0.485 | 121 |
| Prodrug (non-cleavable) | 2.197 | 549 |

### Example 7: Anti-Tumor Activity of Exemplary ProTriTAC Molecules Containing Various Exemplary Linkers, in an Admixed Mouse Tumor Model

The aim of this study was to explore the anti-tumor activity of ProTriTAC molecules containing different linkers. NSG female mice, 7 weeks old, were used for this study. At the commencement of the study, on day 0, the NSG female mice were injected with 2.5×10⁶ expanded human T cells, and 5×10⁶ HCT116 (human colorectal carcinoma) tumor cells. The following day, on day 1, the mice were divided into groups and each group was treated with at least one of the ProTriTAC molecules listed in Table 8 (SEQ ID Nos. 786-790), or with a control GFP TriTAC molecule (SEQ ID No. 792), or with a ProTriTAC molecule that contains a non-cleavable linker (NCLV) (SEQ ID No. 791).

The ProTriTAC molecules and the ProTriTAC NCLV molecule used in the following examples were targeted to EGFR and had the following orientation of the individual domains: (anti-albumin binding domain (sdAb): anti-CD3 domain (scFV): anti-EGFR domain (sdAb)). The only differences between the ProTriTAC molecules listed in Table 7 were in the linker sequences. The ProTriTAC molecules, ProTriTAC NCLV molecule, or the GFP TriTAC molecule (the GFP TriTAC molecule had the following orientation of individual domains: anti-GFP sdAb: anti-Alb sdAb: anti-CD3 scFv) were administered daily for a period of 10 days (i.e., final dose was administered on day 10 following injection of tumor cells and expanded cells to the animals) and tumor volumes were measured at regular intervals, beginning a few days prior to the administration of the final dose at day 10.

TABLE 8
Exemplary ProTriTAC molecules and linkers

| ProTriTAC | Linker in the | | Cleavability/Recognition of |
|---|---|---|---|
| molecule | ProTriTAC | | the linker sequence by |
| sequence | molecule | Linker Sequence | enzymes |
| SEQ ID No. 786 | L001 | KPLGLQARVV | MMP9 + matriptase |
| SEQ ID No. 787 | L040 | PQASTGRSGG | MMP9 + matriptase + uPA |
| SEQ ID No. 788 | L041 | PQGSTGRAAG | MMP9 + matriptase + uPA |
| SEQ ID No. 789 | L042 | PPASSGRAGG | matriptase + uPA |
| SEQ ID No. 790 | L045 | PIPVQGRAH | MMP9 + matriptase |
| | L043 (SEQ ID | PQGSTARSAG | MMP9 + matriptase + uPA |
| | No. 988) | | |

As shown in FIG. 18, the ProTriTAC molecules containing linker sequences L001, L045, L040, and L041 demonstrated more potent anti-tumor activity as compared to the GFP TriTAC control of the ProTriTAC NCLV molecule. The statistical significance of the data was determined by repeated measures one-way ANOVA-Dunnett post-test. The mean tumor volume of each group of mice were compared to the mean tumor volume of the mice group that received the NCLV molecule.

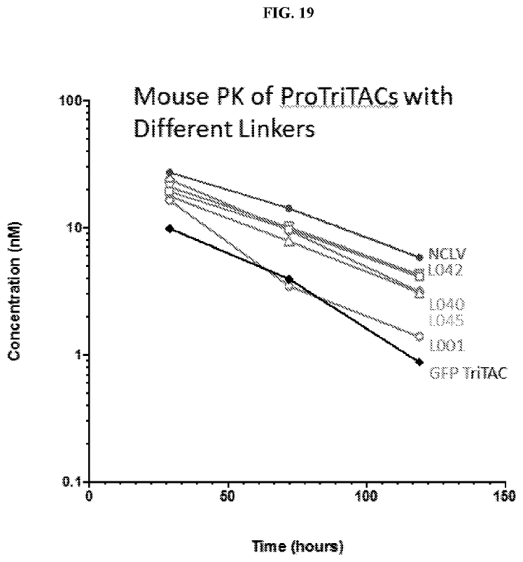

The pharmacokinetics following administration of the various molecules, as described above, were also assessed and the data is shown in FIG. 19. The control GFP TriTAC molecule was rapidly cleared from circulation, following administration, whereas the NCLV molecule remained in circulation for the longest time. The pharmacokinetic clearance profile of the test ProTriTAC molecules were in between the GFP TriTAC and NCLV, except for the ProTriTAC containing the linker sequence L001, which was cleared almost as rapidly as the control GFP TriTAC.

### Example 8: Individual Tumor Volumes of Admixed Xenograft Tumors, Following Treatment with Exemplary ProTriTAC Molecules Containing Various Exemplary Linkers

The ProTriTAC molecules listed in Table 7, the control GFP TriTAC molecule, and the ProTriTAC NCLV molecule were evaluated in an admixed xenograft model, in order to determine the efficacy of the ProTriTAC molecules containing different linkers, in vivo. As described in previous example (Example 7), the xenograft tumor model was generated by injecting 7 week old NSG mice with 2.5×10⁶ expanded human T cells, and 5×10⁶ HCT116 (human colorectal carcinoma) tumor cells. The mice were divided into groups and each group was treated with at least one of the ProTriTAC molecules listed in Table 7, with the control GFP TriTAC molecule, or with the ProTriTAC NCLV molecule. Tumor volumes were measured at regular intervals, starting from day 10 post injection of tumor cells and expanded T cells.

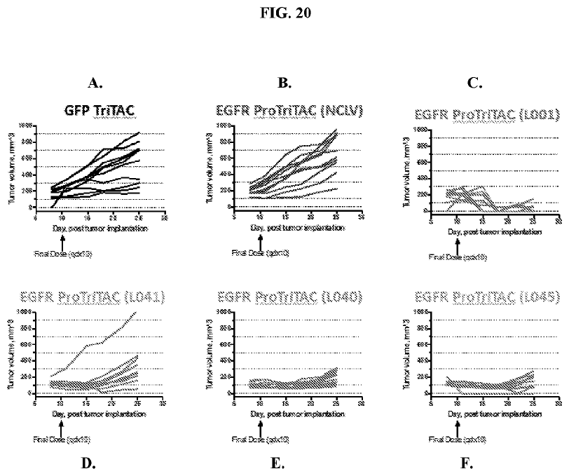

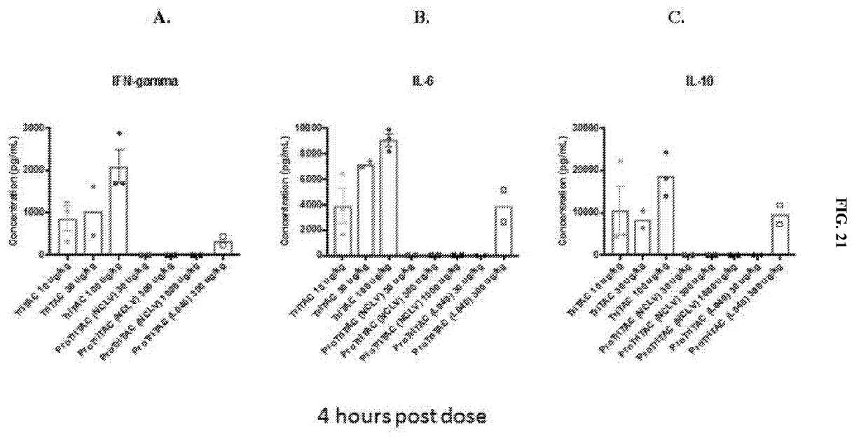

It was observed that in animals treated with the exemplary ProTriTAC molecules containing linker L040 there was a statistically significant delay in tumor growth as compared to the mice group which was treated with the control GFP TriTAC molecule, or the mice group that was treated with the ProTriTAC NCLV molecule. Similar observation was also made for the ProTriTAC molecules containing linker sequences L001, L041, and L045. The data is shown in FIG. 20.

It is also possible to carry out a similar study with xenograft models using other cell lines, such as A549 (non-small cell lung carcinoma) cells, DU-145 (prostate) cells, MCF-7 (breast) cells, Colo 205 (colon) cells, 3T3/]GF-IR (mouse fibroblast) cells, NCI H441 cells, HEP G2 (hepatoma) cells, MDA MB 231 (breast) cells, HT-29 (colon) cells, MDA-MB-435s (breast) cells, U266 cells, SH-SYSY cells, Sk-Mel-2 cells, NCI-H929, RPM18226, and A431 cells.

### Example 9: Demonstration of Reduced Cytokine Levels in Cynomolgus Monkeys, Correlated with Masking of TriTAC Molecules

In this study, cynomolgus monkeys were treated with three different concentrations (30 μg/kg; 300 μg/kg; and 1000 μg/kg) of an exemplary EGFR targeting ProTriTAC molecule containing a non-cleavable linker (ProTriTAC (NCLV), or with three different concentrations (10 μg/kg; 30 μg/kg; and 100 μg/kg) of an exemplary EGFR targeting TriTAC molecule (SEQ ID No. 793).

As shown in FIG. 21, after 4 hours following administering the ProTriTAC (NCLV) molecule, IFN-gamma IL-6 and IL-10 levels were significantly lower in comparison with administering the EGFR targeting TriTAC molecule.

### Example 10: Demonstration of Improved Tolerability in Mouse, Conferred by an Exemplary EGFR Targeting ProTriTAC Molecule

In this study, the tolerability of an exemplary EGFR targeting ProTriTAC molecule was assessed. Seven weeks old NSG female tumor free mice were intraperitoneally injected with 2×10⁷ expanded human T cells at the commencement of the study, i.e., at day 0. On day 2, treatment was started by dividing the mice into various groups and administering to them varying concentrations of the exemplary EGFR targeting ProTriTAC molecule, containing the linker sequence L001, an EGFR targeting TriTAC molecule, and an EGFR targeting ProTriTAC molecule containing a non-cleavable linker (ProTriTAC (NCLV). The molecules were administered once daily for 10 days, at the following dosages: 30 μg/kg, 100 μg/kg, 300 μg/kg Starting from day 2, body weight of the animals was recorded daily.

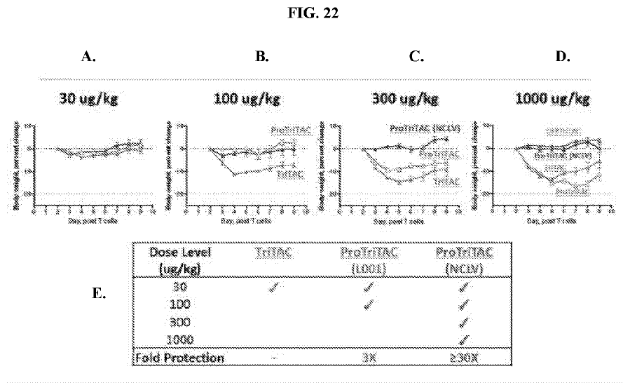

As shown in FIG. 22, the EGFR targeting ProTriTAC molecule containing a non-cleavable linker (ProTriTAC (NCLV)) and a GFP TriTAC (used as a negative control) were very well tolerated in mice even at the highest dose of 1000 μg/kg. The EGFR targeted ProTriTAC molecule containing the linker sequence of L001 was well tolerated at the dosage of 100 μg/kg, whereas the EGFR targeted TriTAC was well tolerated at 30 μg/kg. It was thus observed that the ProTriTAC containing the L001 linker sequence conferred about 3 fold increase in tolerability and the ProTriTAC (NCLV) conferred about a 30 fold increase in tolerability, in mouse. The mouse maximum tolerated dose for the ProTriTAC (NCLV) and TriTAC molecules was consistent with what was observed in cynomolgus monkeys.

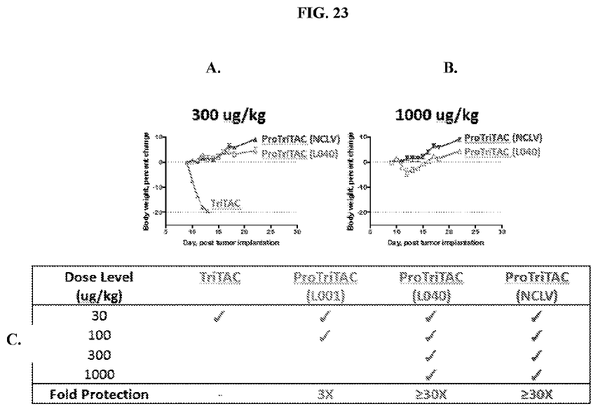

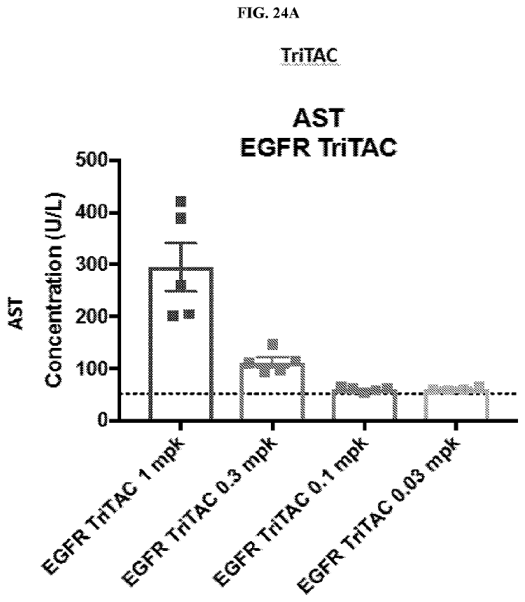

To further explore the role of the linker in tolerability of the EGFR targeting ProTriTAC molecule in mouse, the linker sequence was changed from L001 to that of L040. In this experiment, seven weeks old NSG female tumor free mice were subcutaneously injected with 5×10⁶HCT116 tumor cells, at the commencement of the study, i.e., at day 0. At day 7 following the tumor cell injection, when the tumor volumes were about 180-200 mm³ (e.g., 183 mm³), the mice were injected intraperitoneally with 2×10⁷ expanded human T cells. Treatment was started on day 9, by dividing the mice into various groups and each group was administered an EGFR targeting TriTAC molecule, an EGFR targeting ProTriTAC molecule with linker sequence L040 (ProTriTAC(L040), and a ProTriTAC molecule containing a non-cleavable linker (ProTriTAC(NCLV). The molecules were administered once daily for 10 days, at the following dosages: 300 μg/kg and 1000 μg/kg. Starting from day 2, body weight of the animals were recorded daily. The results shown in FIG. 23 provide that the EGFR targeting ProTriTAC molecule with the linker sequence L040 conferred better tolerability than when the linker sequence L001 was used. The ProTriTAC (L040) was well-tolerated at 300 μg/kg and at 1000 μg/kg, with body weight percentage change comparable to that with the ProTriTAC (NCLV) molecule. It was thus observed that compared to the TriTAC molecule, the ProTriTAC containing the L040 linker sequence conferred about a 30 fold increase in tolerability, in mouse, similar to the 30 fold increase in tolerability observed with the ProTriTAC (NCLV) molecule.

### Example 11: Clinical Pathology Studies in Mice and Cynomolgus Monkeys

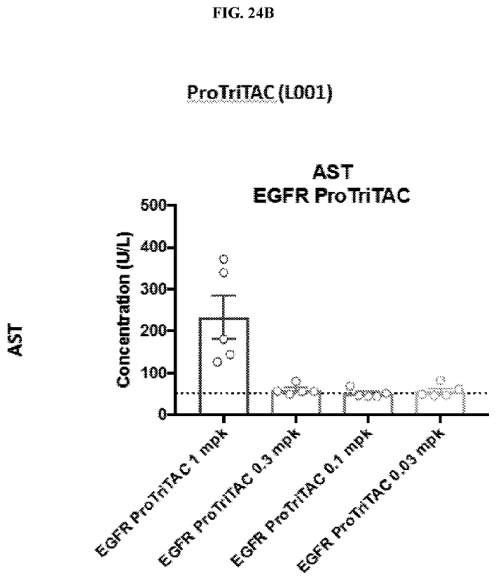

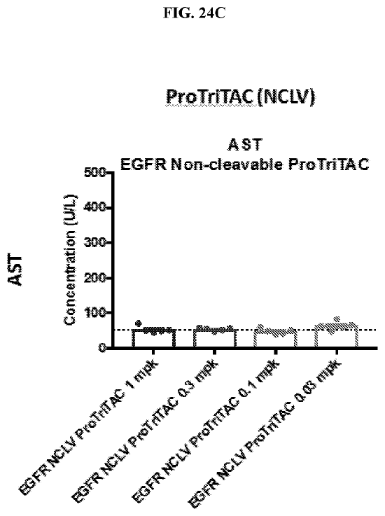

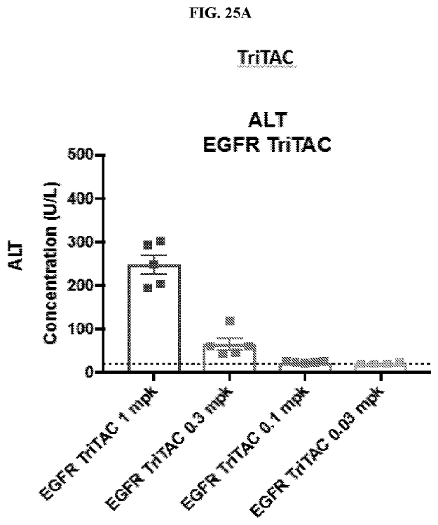

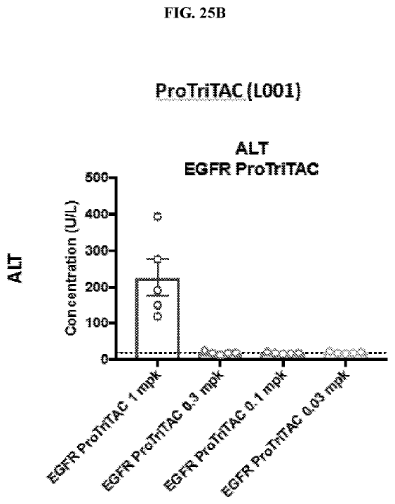

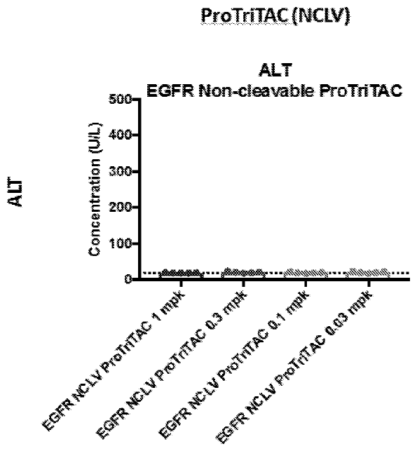

In this study, mice were treated with various concentrations of an EFGR targeting TriTAC molecule, an EGFR targeting ProTriTAC molecule containing the linker sequence L001 (ProTriTAC (L001), and an EGFR targeting ProTriTAC molecule containing a non-cleavable linker (ProTriTAC(NCLV)). Tolerability was assessed by measuring serum concentration of ALT (alanine aminotransferase) and AST (aspartate aminotransferase). Results are shown in FIGS. 24A, 24B, and 24C and FIGS. 25A, 25B, and 25C. It was observed that serum concentrations of AST and ALT were not elevated following administering the ProTriTAC (L001) at dosages up to 0.3 mg/kg, and that the serum concentrations of AST and ALT were not elevated following administering the ProTriTAC(NCLV) at dosages up to 1 mg/kg. In contrast, serum concentration of AST and ALT were not elevated following administering the TriTAC molecule at a dosage of 0.3 mg/kg.

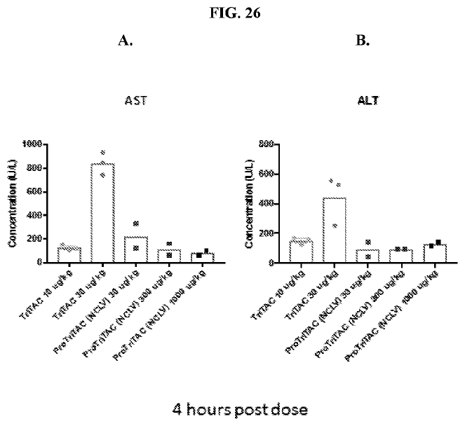

In another study, cynomolgus monkeys were treated with various concentrations of an EFGR targeting TriTAC molecule, and an EGFR targeting ProTriTAC molecule containing a non-cleavable linker (ProTriTAC(NCLV)). Tolerability was assessed by measuring serum concentration of ALT (alanine aminotransferase) and AST (aspartate aminotransferase). Results are shown in FIG. 26. It was observed that serum concentrations of AST and ALT were not elevated following administering the ProTriTAC (NCLV) at dosages up to 1000 μg/kg. In contrast, serum concentration of AST and ALT were not elevated following administering the TriTAC molecule at a dosage of 10 μg/kg.

### Example 12: Demonstration of Therapeutic Window Expansion with an Exemplary ProTriTAC Molecule of this Disclosure in a Tumor-Bearing Mouse Model

The aim of this study was to evaluate the expansion of therapeutic window by measuring anti-tumor activity and observable on-target toxicity in the same tumor-bearing mice. NSG female mice, 7 weeks old, were used for this study.

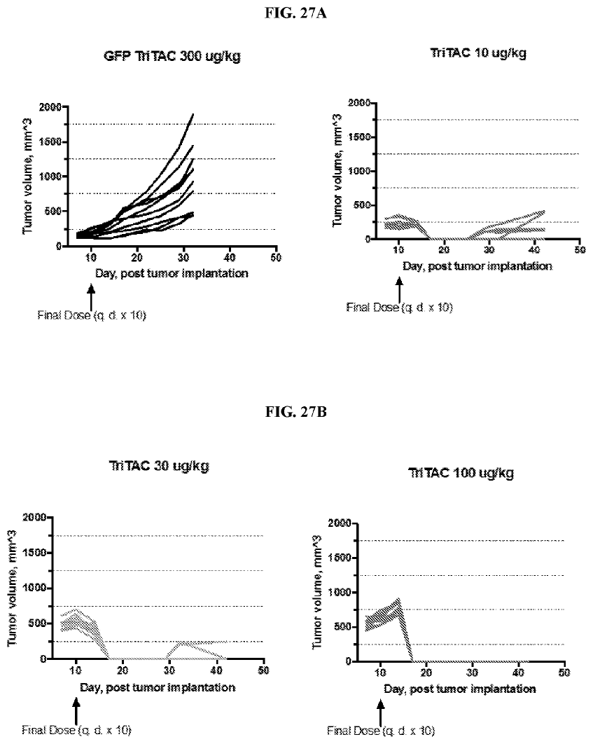

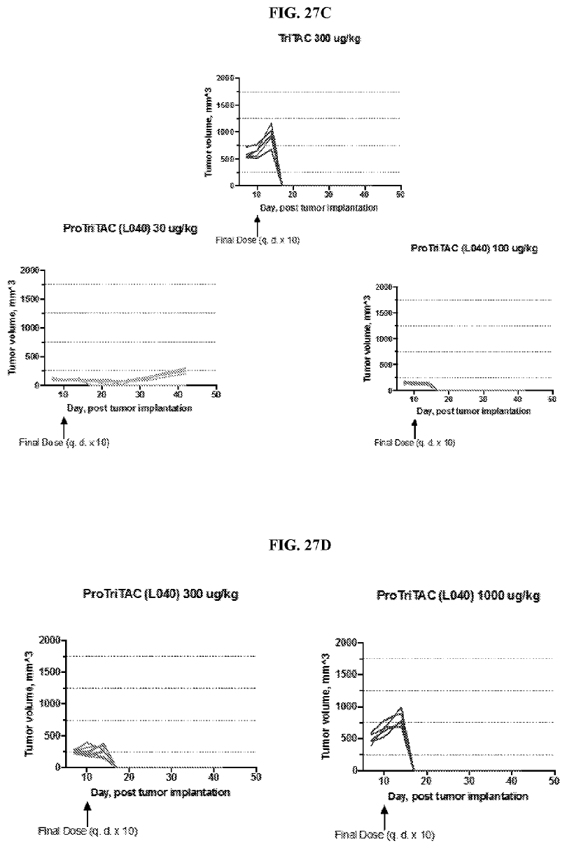

At the commencement of the study, on day 0, the NSG female mice were injected with 2.5×10⁶ expanded human T cells, and 5×10⁶ HCT116 (human colorectal carcinoma) tumor cells. The following day, on day 1, the mice were divided into groups and each group was treated with either GFP TriTAC molecule (SEQ ID No. 792), EGFR TriTAC molecule (SEQ ID No. 793), or an EGFR targeting ProTriTAC molecule containing linker L040, (SEQ ID No. 787) at the indicated dose levels in FIGS. 27A-297D (for GFP TriTAC the dosage was 300 μg/kg; for EGFR TriTAC dosages were 10 μg/kg, 30 μg/kg, 100 μg/kg, and 300 μg/kg; for EGFR ProTriTAC dosages were 30 μg/kg, 100 μg/kg, 300 μg/kg, and 1000 μg/kg) and administered daily for a period of 10 days (i.e., final dose was administered on day 10 following injection of tumor cells and expanded cells to the animals) and tumor volumes were measured at regular intervals, beginning a few days prior to the administration of the final dose at day 10. Results shown in FIGS. 27A-27D.

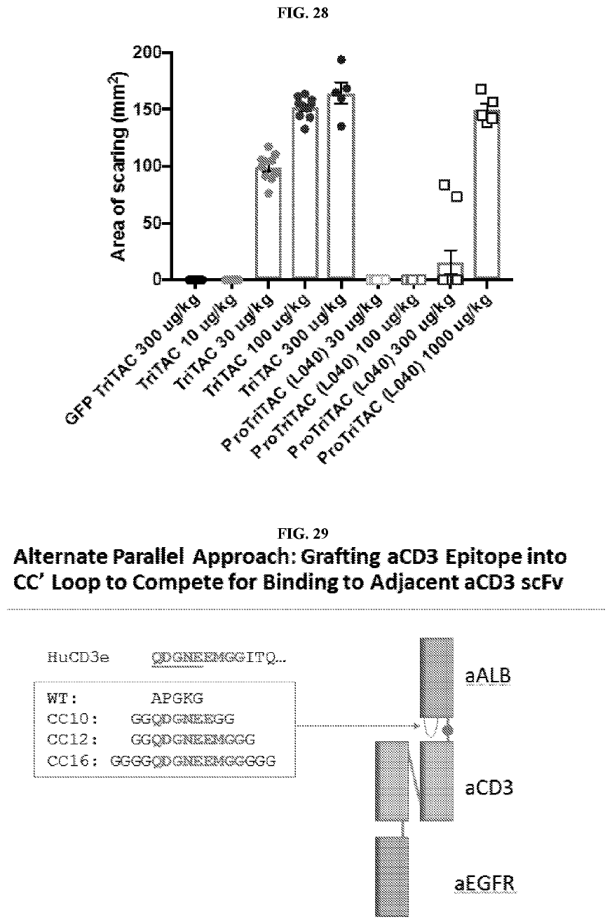

On-target EGFR-related toxicity was determined by measuring the radius of the red scarring skin lesion above the original tumor implantation site with a caliper and applying the equation Area=(radius of lesion)² on day 14. Results provided in FIG. 28 show that an exemplary EGFR ProTriTAC has 30× better tolerability compared to an EGFR TriTAC in the same HCT116 tumor-bearing mice as measured by the onset of red scarring skin lesions above the original tumor implantation site. Therapeutic window is defined as the difference between the minimal dose level required for anti-tumor activity and the highest skin lesion-free dose level.

Results (from FIGS. 27 and 28) show that the exemplary protease-cleavable EGFR ProTriTAC is 3× less potent but 30× more tolerated (i.e., has a 10× improved therapeutic window) than the EGFR TriTAC when efficacy and toxicity are measured on the same tumor-bearing mice, as summarized in below Table. This would make it possible to dose the ProTriTAC at a dose that is about 3× higher than the TriTAC, to get at least the same efficiency and better tolerability.

| | | | Therapeutic |
|---|---|---|---|
| | Min efficacious dose | Max scar-free dose | Window |
| TriTAC | 10 μg/kg | 10 μg/kg | 1 |
| ProTriTAC | 30 μg/kg | 300 μg/kg | 10 |

### Example 13: An Exemplary ProTriTAC Molecule Containing a Dual Binding Moiety with Extended Non-CDR Loop into which a Human CD3ε Epitope is Grafted

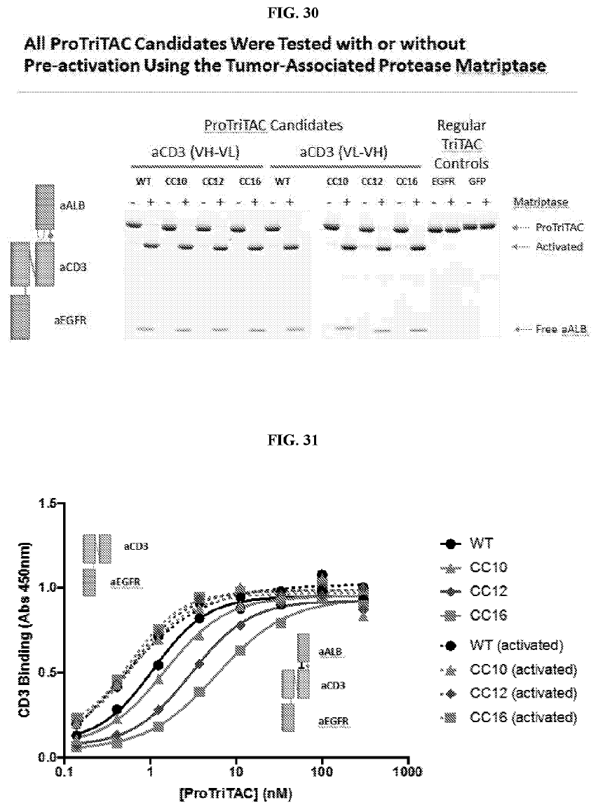

The sequence of a dual binding moiety comprising non-CDR loops (AB, EF, C″D, and CC′) was obtained. A portion of the human CD3ε sequence was grafted into the CC′ loop of the non-CDR loops within the dual binding moiety, along with glycine residues to further extend the CC′ loop. FIG. 29 illustrates three different variants comprising 10, 12, or 16 amino acid extensions to the CC′ loop. In case of the variant CC10, the portion of human CD3a sequence grafted into the CC′ loop, to replace the wild type sequence of APGKG was QDGNEE (SEQ ID NO. 801), and in addition 4 glycine residues were inserted to extend the CC′ loop. In case of the variant CC12, the portion of human CDR sequence grafted into the CC′ loop, to replace the wild type sequence of APGKG was QDGNEEMGG (SEQ ID No. 802), and in addition 3 glycine residues were inserted to extend the CC′ loop. In case of the variant CC12, the portion of human CDR sequence grafted into the CC′ loop, to replace the wild type sequence of APGKG was QDGNEEMGG (SEQ ID No. 803), and in addition 7 glycine residues were inserted to extend the CC′ loop. The dual binding moiety comprising extended non-CDR loops comprising the CD3a sequences, as described above, were cloned into a vector further comprising coding sequences for a protease cleavable linker, a scFv containing a CD3 binding domain, and an EGFR binding domain, to express a ProTriTAC molecule. The ProTriTAC molecule contained an exemplary dual binding moiety of this disclosure, a CD3 binding scFv, and an EGFR binding domain. The ProTriTAC molecule was subsequently exposed to a tumor associated protease, matriptase, to assay activation of the molecule upon cleavage of the protease cleavable linker, which separates the dual binding moiety (depicted as aALB in FIGS. 29 and 30) comprising the cleavable linker from the rest of the molecule, i.e., the scFv containing the CD3 binding domain (depicted as aCD3 in FIGS. 29 and 30) and the EGFR binding domain (aEGFR). FIG. 30 shows the activation of the ProTriTAC molecules, containing the CC10, CC12, or CC16 variants of CC′ non-CDR loop, CD3 binding scFv in a VH-VL (left panel) or a VL-VH (right panel) format, upon treatment with matriptase. A ProTriTAC molecule containing a wild-type CC′ loop in the dual binding moiety was used as a control for the protease activation assay. In addition, a TriTAC molecule that is not in the “pro” form, i.e., a molecule that includes the same domains as the ProTriTAC molecule, except that it has a half-life extension domain, such as albumin, instead of a dual binding moiety, was also treated with matriptase and used as a control. Results indicated that the ProTriTAC molecules were activated upon cleavage, to generate a free albumin binding domain (depicted as Free aALB in FIG. 30) whereas the albumin domain did not separate from the TriTAC molecules. Thus, the ProTriTAC molecules containing a dual binding moiety of this disclosure was able to readily dissociate from half-life extending domain upon cleavage in a tumor microenvironment, unlike the TriTAC versions, and thereby were amenable to rapid clearance from the systemic circulation upon activation.

Further studies were carried out to assay the binding of the dual binding moiety containing the human CD3ε to CD3. It was observed that the activated forms of ProTriTAC molecules that contained the dual binding moiety containing the human CD3ε were about 20 times potent in binding CD3 than their activated forms which did not contain the dual binding moiety. Results are shown in FIG. 31.

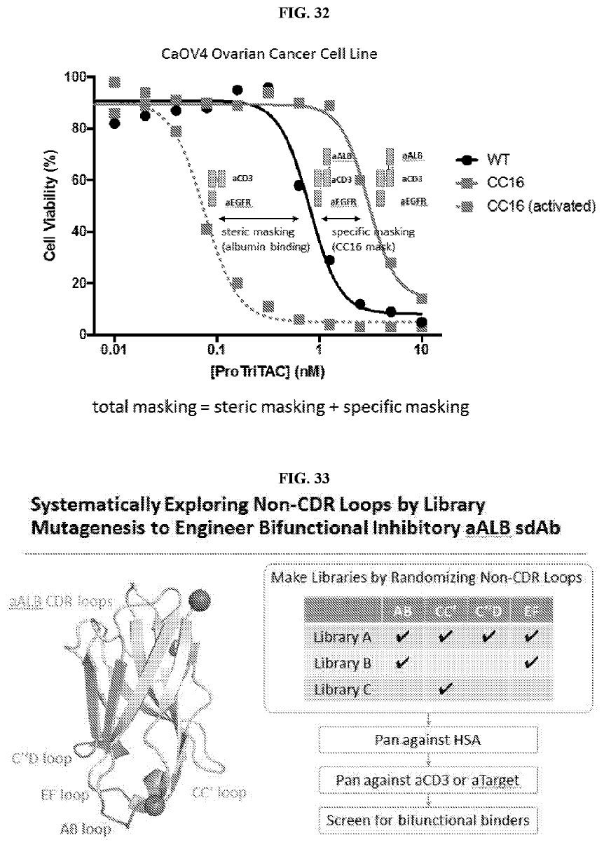

Cell killing potential of a ProTriTAC molecule that contained a dual binding moiety as described herein was also assayed in a study where CaOV4 cell line was treated with the ProTriTAC molecule or its activated form. As shown in FIG. 32, the ProTriTAC molecule containing the CC16 variant of CC′ non-CDR loop was about 20 times more potent in killing cancer cells compared to its activated form, which was separated from the albumin binding domain.

### Example 14: Soft Library Mutagenesis Identified CC′ Loop as Most Amenable to Modification

To identify locations within the non-CDR loops (AB, CC′, C″D, and EF) that were most amenable to modification, for creating a masking capability, libraries were assembled and generated using four groups of overlapping DNA oligos containing randomized degenerate “NNK” codons and with different loop lengths, as indicated in the schematic below:

| AB loop oligos: | |
|---|---|
| (SEQ ID NO: 972) | |
| WT: | LVQPGN(20%) |
| AB0: | XXXXXX (20%) |
| AB1: | XXXXXXX (20%) |
| AB2: | XXXXXXXX (20%) |
| AB3: | XXXXXXXXX (20%) |
| CC′ loop oligos: | |
| WT: | APGKG(20%) |
| CC0: | XXXXX (20%) |
| CC1: | XXXXXX (20%) |
| CC2: | XXXXXXX (20%) |
| CC3: | XXXXXXXX (20%) |
| C″D loop oligos: | |
| (SEQ ID NO: 973) | |
| WT: | DSVKGR(20%) |
| CD0: | XXXXXX (20%) |
| CD1: | XXXXXXX (20%) |
| CD2: | XXXXXXXX (20%) |
| CD3: | XXXXXXXXX (20%) |
| EF loop oligos: | |
| (SEQ ID NO: 974 | |
| WT: | SLRPED(20%) |
| EF0: | XXXXXX (20%) |
| EF1: | XXXXXXX (20%) |
| EF2: | XXXXXXXX (20%) |
| EF3: | XXXXXXXXX (20%) |

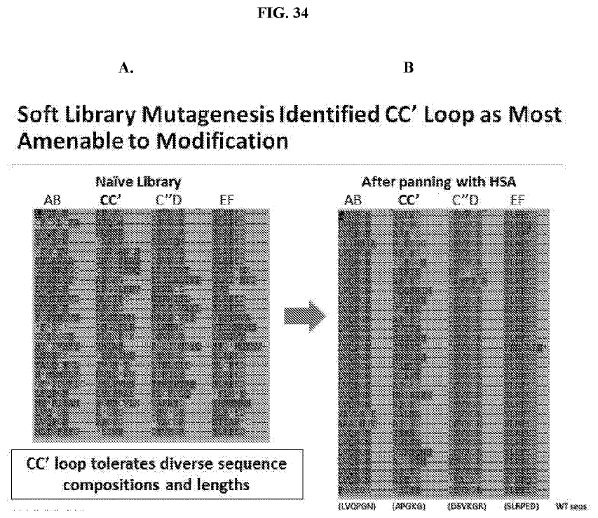

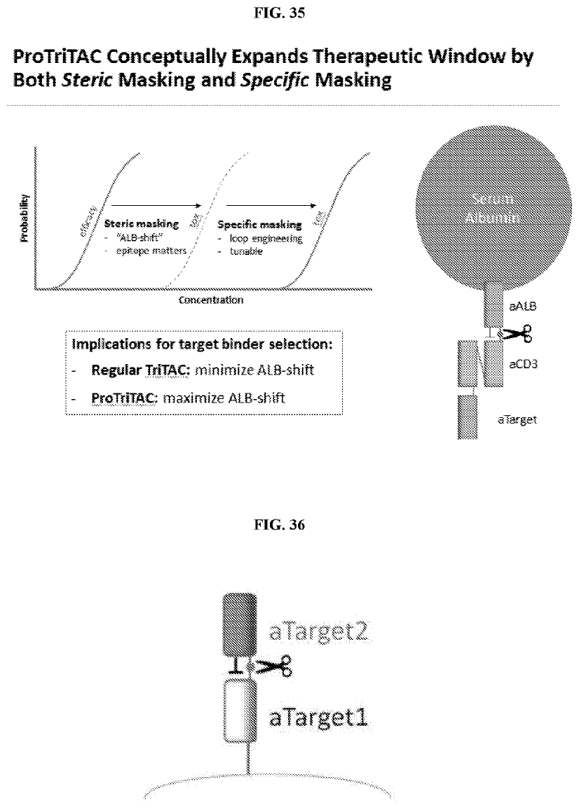

Note: “X” denotes a randomized residue (“NNK” codon) that could be any of the 20 natural amino acids as well as stop codon. The goal was to have approximately 20% of each non-CDR loop be wild-type. These wild-type oligos served as internal benchmarks to gauge the tolerance of each loop to modification (sequence composition and/or length changes). A loop that was less tolerable to change could easily revert to wild-type; in contrast, a loop that was highly amenable to change would maintain the diverse sequence repertoire. To this end, 24 clones were sequenced from the naive library to verify the randomization of non-CDR loops prior to panning with HAS, as shown in FIG. 33. After two rounds of phage panning against HSA, 30 clones were sequenced to examine the non-CDR loop composition. The results showed that 3 out of 4 non-CDR loops (AB, C″D, and EF) mostly utilized the 20% wild-type oligo and reverted back to the wild-type, suggesting that they may be less preferred than the wild-type sequence. However, the CC′ loop kept the diverse sequence repertoire (both sequence and length), suggesting that the CC′ loop may be the loop most tolerable to randomization that we could exploit for specific masking of adjacent domains, as shown in FIG. 34.

### Example 15: Construction of an Exemplary Dual Binding Moiety which Binds to IL-2 and a Target Antigen Binding Domain Whose Target is EGFR

The sequence of an engineered protein scaffold comprising CDR loops capable of binding albumin and non-CDR loops is obtained. Overlapping PCR is used to introduce random mutations in the non-CDR loop regions, thereby generating a library. The resultant sequences are cloned into a phage display vector, thereby generating a phage display library. Escherichia coli cells are transformed with the library and used to construct a phage display library. ELISA is performed using an immobilized target antigen binding domain with specificity for EGFR. A clone with high specificity for EGFR is selected. Affinity maturation is performed by re-randomizing residues in the non-CDR loop regions as before.

Sequence alignment of non-CDR loop regions of the resultant proteins is performed to determine sequence conservation between proteins with high affinity for the target antigen binding domain. Site directed mutagenesis of one or more amino acids within these regions of sequence conservation is performed to generate additional proteins. Binding of the resultant proteins to an immobilized target antigen binding domain whose target is EGFR is measured in an ELISA. A protein with the highest affinity for the target antigen binding domain is selected.

The sequence of this protein is cloned into a vector comprising a sequence for a cleavable linker. The resultant vector is expressed in a heterologous expression system to obtain a dual binding moiety comprising a cleavable linker and non-CDR loops which provide a binding site specific for a target antigen binding domain whose target is EGFR and CDR loops which are specific for IL-2.

### Example 16: Construction of an Exemplary Conditionally Active Chimeric Antigen Receptor

The dual binding moiety of Example 15 is cloned into an expression vector comprising the components of a chimeric antigen receptor, namely anti-PSMAscFv-IgG4-CD28tm-CD28costim-CD3zeta, to obtain a vector coding for a conditionally active chimeric antigen receptor. Primary human peripheral blood derived T-cells are activated and then lentivirally transduced with the conditionally active chimeric antigen receptor vector to obtain the conditionally active chimeric antigen receptor according to the present disclosure.

### Example 17: Construction of an Exemplary Conditionally Active T-Cell Receptor Fusion Protein

The dual binding moiety of Example 15 is cloned into an expression vector comprising the components of a T-cell receptor fusion protein, namely anti-PSMAscFv-IgG4-CD28tm-CD3epsilon, to obtain a vector coding for a conditionally active T-cell receptor fusion protein. Primary human peripheral blood derived T-cells are activated and then lentivirally transduced with the conditionally active T-cell receptor fusion protein vector to obtain the conditionally active T-cell receptor fusion protein according to the present disclosure.

### Example 18: Construction of an Exemplary Conditionally Active T-Cell Receptor

The dual binding moiety of Example 1 is cloned into an expression vector comprising the components of a T-cell receptor to obtain a vector coding for a conditionally active T-cell receptor. Primary human peripheral blood derived T-cells are activated and then lentivirally transduced with the conditionally active T-cell receptor vector to obtain the conditionally active T-cell receptor according to the present disclosure.

### Example 19: T-Cells Comprising a Conditionally Active Chimeric Antigen Receptor Exhibit Reduced Specificity Towards Cell Line which Overexpresses PSMA but is Protease Deficient

Cells overexpressing PSMA and exhibiting low expression of a matrix metalloprotease are separately incubated with T-cells comprising an exemplary conditionally active chimeric antigen receptor according to the present disclosure and T-cells comprising a non-conditionally active control chimeric antigen receptor. Cells expressing normal levels of PSMA and proteases are also incubated with T-cells comprising a conditionally active chimeric antigen receptor according to the present disclosure and T-cells comprising a non-conditionally active control chimeric antigen receptor. Both receptors comprise a target antigen binding domain with specificity toward PSMA.

Results indicate that in the absence of protease secretion, the T-cells comprising a conditionally active chimeric antigen receptor of the present disclosure interact with the protease expressing cells but do not interact with the PSMA expressed on the surface of the protease deficient cells. In contrast, the T-cells comprising a non-conditionally active control chimeric antigen receptor lack the ability to selectively bind the protease expressing cells over the protease deficient ones. Thus, the T-cells comprising an exemplary conditionally active chimeric antigen receptor of the present disclosure are advantageous, for example, in terms of reducing off-tumor toxicity.

### Example 20: T-Cells Comprising a Conditionally Active T-Cell Receptor Fusion Protein Exhibit Reduced Specificity Towards Cell Line which Overexpresses PSMA but is Protease Deficient

Cells overexpressing PSMA and exhibiting low expression of a matrix metalloprotease are separately incubated with T-cells comprising an exemplary conditionally active T-cell receptor fusion protein according to the present disclosure and T-cells comprising a non-conditionally active control T-cell receptor fusion protein. Cells expressing normal levels of PSMA and proteases are also incubated with T-cells comprising a conditionally active T-cell receptor fusion protein according to the present disclosure and T-cells comprising a non-conditionally active control T-cell receptor fusion protein. Both receptors comprise a target antigen binding domain with specificity toward PSMA.

Results indicate that in the absence of protease secretion, the T-cells comprising a conditionally active T-cell receptor fusion protein of the present disclosure interact with the protease expressing cells but do not interact with the PSMA expressed on the surface of the protease deficient cells. In contrast, the T-cells comprising a non-conditionally active control T-cell receptor fusion protein lack the ability to selectively bind the protease expressing cells over the protease deficient ones. Thus, the T-cells comprising an exemplary conditionally active T-cell receptor fusion protein of the present disclosure are advantageous, for example, in terms of reducing off-tumor toxicity.

### Example 21: T-Cells Comprising a Conditionally Active T-Cell Receptor Exhibit Reduced Specificity Towards Cell Line which Overexpresses PSMA but is Protease Deficient

Cells overexpressing PSMA and exhibiting low expression of a matrix metalloprotease are separately incubated with T-cells comprising an exemplary conditionally active T-cell receptor according to the present disclosure and T-cells comprising a non-conditionally active control T-cell receptor. Cells expressing normal levels of PSMA and proteases are also incubated with T-cells comprising a conditionally active T-cell receptor according to the present disclosure and T-cells comprising a non-conditionally active control T-cell receptor. Both receptors comprise a target antigen binding domain with specificity toward PSMA.

Results indicate that in the absence of protease secretion, the T-cells comprising a conditionally active T-cell receptor of the present disclosure interact with the protease expressing cells but do not interact with the PSMA expressed on the surface of the protease deficient cells. In contrast, the T-cells comprising a non-conditionally active control T-cell receptor lack the ability to selectively bind the protease expressing cells over the protease deficient ones. Thus, the T-cells comprising an exemplary conditionally active T-cell receptor of the present disclosure are advantageous, for example, in terms of reducing off-tumor toxicity.

### Example 22: Treatment with an Exemplary Conditionally Active Chimeric Antigen Receptor of the Present Disclosure Inhibits In Vivo Tumor Growth

Murine tumor line CT26 is implanted subcutaneously in Balb/c mice and on day 7 post-implantation the average size of the tumor is measured. Test mice are treated with T-cells comprising an exemplary conditionally active chimeric antigen receptor which has a target antigen binding domain specific for EGFR where in the EGFR-specific domain is bound to a dual binding moiety via its non-CDR loops and the dual binding moiety comprises a cleavable linker and CDR loops specific for IL-2. Control mice are treated with T-cells comprising a chimeric antigen receptor that contains a EGFR-specific domain but does not contain the dual binding moiety or the cleavable linker, and is not conditionally active. Results show that treatment with T-cells comprising an exemplary conditionally active chimeric antigen receptor of the present disclosure inhibits tumor more efficiently than the comparator T-cells comprising a chimeric antigen receptor which does not contain the moiety with non-CDR loops.

### Example 23: Treatment with an Exemplary Conditionally Active T-Cell Receptor Fusion Protein of the Present Disclosure Inhibits In Vivo Tumor Growth

Murine tumor line CT26 is implanted subcutaneously in Balb/c mice and on day 7 post-implantation the average size of the tumor is measured. Test mice are treated with T-cells comprising an exemplary conditionally active T-cell receptor fusion protein which has a target antigen binding domain specific for EGFR where in the EGFR-specific domain is bound to a dual binding moiety via its non-CDR loops and the dual binding moiety comprises a cleavable linker and CDR loops specific for IL-2. Control mice are treated with T-cells comprising a T-cell receptor fusion protein that contains a EGFR-specific domain but does not contain the dual binding moiety or the cleavable linker, and is not conditionally active. Results show that treatment with T-cells comprising an exemplary conditionally active T-cell receptor fusion protein of the present disclosure inhibits tumor more efficiently than the comparator T-cells comprising a T-cell receptor fusion protein which does not contain the moiety with non-CDR loops.

### Example 24: Treatment with an Exemplary Conditionally Active T-Cell Receptor of the Present Disclosure Inhibits In Vivo Tumor Growth

Murine tumor line CT26 is implanted subcutaneously in Balb/c mice and on day 7 post-implantation the average size of the tumor is measured. Test mice are treated with T-cells comprising an exemplary conditionally active T-cell receptor which has a target antigen binding domain specific for EGFR where in the EGFR-specific domain is bound to a dual binding moiety via its non-CDR loops and the dual binding moiety comprises a cleavable linker and CDR loops specific for IL-2. Control mice are treated with T-cells comprising a T-cell receptor that contains a EGFR-specific domain but does not contain the dual binding moiety or the cleavable linker, and is not conditionally active. Results show that treatment with the T-cells comprising an exemplary conditionally active T-cell receptor of the present disclosure inhibits tumor more efficiently than the comparator T-cells comprising a T-cell receptor which does not contain the moiety with non-CDR loops.

### Example 25: Conditionally Active Receptor Constructs

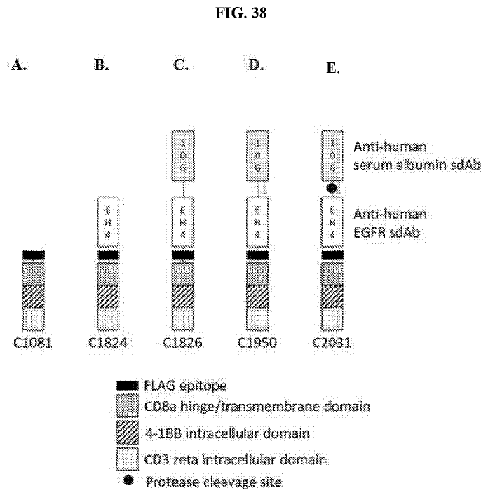

FIGS. 38A-F show examples of receptor constructs which were generated as disclosed herein. Each construct comprises an intracellular domain comprising a CD8 hinge/transmembrane domain (SEQ ID NO: 823), a 4-1BB intracellular domain (SEQ ID NO: 824), and a CD3 zeta intracellular domain (SEQ ID NO: 825). Construct C1081 (SEQ ID NO: 804; FIG. 38A) does not comprise a target antigen binding domain or a dual binding moiety. Construct C1824 (SEQ ID NO: 805; FIG. 38B) comprises the target antigen binding domain EH4 (SEQ ID NO: 821) but does not comprise a dual binding moiety. Construct 1826 (SEQ ID NO: 806; FIG. 38C) comprises target antigen binding domain EH4 and the dual binding moiety 10G with an unmodified non-CDR loop (SEQ ID NO: 817) along with a non-cleavable linker (SEQ ID NO: 826). Construct 1950 (SEQ ID NO: 807; FIG. 38D) comprises target antigen binding domain EH4 and dual binding moiety 10G with a modified non-CDR loop (SEQ ID NO: 818) along with a non-cleavable linker (SEQ ID NO: 826). Construct 2031 (SEQ ID NO: 808; FIG. 38E) comprises target antigen binding domain EH4 and dual binding moiety 10G with a modified non-CDR loop (SEQ ID NO: 818) along with protease cleavable linker 1 (SEQ ID NO: 827).

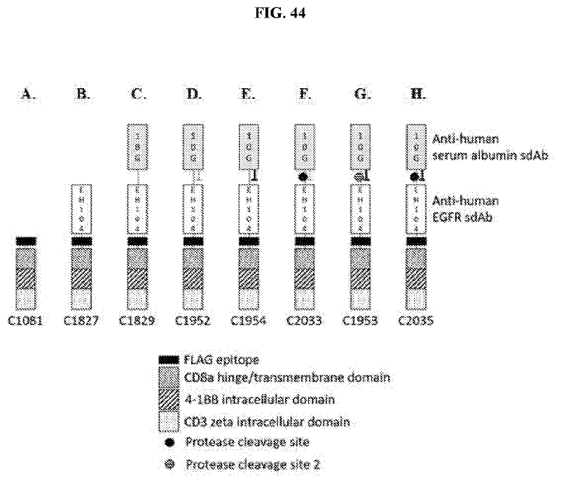

FIGS. 44A-44H show examples of receptor constructs which were generated as disclosed herein. Construct C1081 (SEQ ID NO: 804; FIG. 44A) does not comprise a target antigen binding domain or a dual binding moiety. Construct 1827 (SEQ ID NO: 809; FIG. 44B) comprises the target antigen binding domain EH104 (SEQ ID NO: 822) but does not comprise a dual binding moiety. Construct 1829 (SEQ ID NO: 810; FIG. 44C) comprises target antigen binding domain EH104 and the dual binding moiety 10G with an unmodified non-CDR loop (SEQ ID NO: 817) along with a non-cleavable linker (SEQ ID NO: 826). Construct 1952 (SEQ ID NO: 811; FIG. 44D) comprises target antigen binding domain EH104 and dual binding moiety 10G with a modified non-CDR loop (SEQ ID NO: 819) along with a non-cleavable linker (SEQ ID NO: 826). Construct 1954 (SEQ ID NO: 812; FIG. 44E) comprises target antigen binding domain EH104 and dual binding moiety 10G with a modified non-CDR loop (SEQ ID NO: 59) along with a non-cleavable linker (SEQ ID NO: 820). Construct 2033 (SEQ ID NO: 813; FIG. 44F) comprises target antigen binding domain EH104 and dual binding moiety 10G with a modified non-CDR loop (SEQ ID NO: 819) along with protease cleavable linker 1 (SEQ ID NO: 827). Construct 1953 (SEQ ID NO: 814; FIG. 44G) comprises target antigen binding domain EH104 and dual binding moiety 10G with a modified non-CDR loop (SEQ ID NO: 820) along with protease cleavable linker 2 (SEQ ID NO: 828). Construct 2035 (SEQ ID NO: 815; FIG. 44H) comprises target antigen binding domain EH104 and dual binding moiety 10G with a modified non-CDR loop (SEQ ID NO: 820) along with protease cleavable linker 1 (SEQ ID NO: 827).

### Example 26: Inhibition of CAR-T Cell Killing Activity by Modified Non-CDR Loop Mask

Chimeric antigen receptor-expressing T-cells (CAR T-cells) were generated by infecting isolated CD4/CD8 positive T cells from a healthy donor with lentivirus expressing the indicated constructs. FIG. 38 shows a diagram with the constructs used. CAR T-cells were mixed at a 10:1 ratio with EGFR-expressing cancer cells HCT116, engineered to express luciferase. After 72 hours, luciferase activity was measured as a readout of HCT116 viability. CAR-T cells that lacked a target antigen binding domain (C1081) were used as a negative control. The assay was performed with or without human serum albumin (HSA), as indicated.

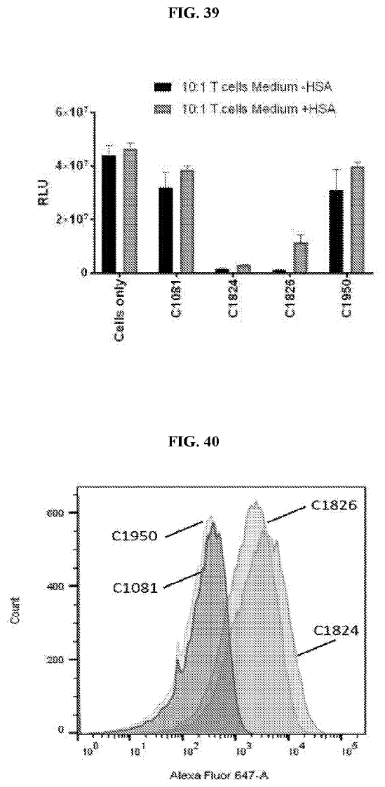

The results of this experiment are shown in FIG. 39. These results indicate that HSA has weak steric blocking activity of CAR T-cell killing activity. These results also demonstrate that the construct containing dual binding moiety 10G with a modified non-CDR loop (C1950) significantly blocks cell killing activity.

### Example 27: Chimeric Antigen Receptor Binding to EGFR

Chimeric antigen receptor-expressing T-cells (CAR T-cells) were generated by infecting isolated CD4/CD8 positive T cells from a healthy donor with lentivirus expressing the indicated constructs. FIG. 38 shows a diagram with the constructs used. Cells were incubated with Fc-tagged EGFR extracellular domain (ECD). Cells were washed to remove unbound Fc-tagged EGFR ECD. Cells were incubated with a secondary antibody conjugated to DyLight 650 that recognizes human Fc. Binding of the secondary antibody to cells was measured by flow cytometry.

The results of this experiment are shown in FIG. 40. Masked EH4 ProCAR (C1950, comprising dual binding moiety 10G with a modified non-CDR loop) demonstrated no detectable binding to its antigen EGFR-Fc while the positive controls (C1824, construct comprising the binding domain EH4 but devoid of dual binding moiety 10G) bound strongly.

The construct C1826, comprising the EGFR binding domain EH4 and the anti-ALB binding moiety 10G with an unmodified non-CDR loop and a non-cleavable linker, i.e., without masking of EH4 binding domain, shows very little steric blocking. This indicates that the anti-ALB binding domain on its own without the mask shows very little steric blocking and that the blocking of EGFR binding, seen in case of C1950, is an effect of the masking.

### Example 28: Protease Activation of Conditionally Active Chimeric Antigen Receptors to EGFR

Chimeric antigen receptor-expressing T-cells (CAR T-cells) were generated by infecting isolated CD4/CD8 positive T cells from a healthy donor with lentivirus expressing the indicated constructs. FIG. 38 shows a diagram with the constructs used. CAR T-cells expressing C2031 were treated with either PBS or recombinant uPA protease, as indicated, for 1 hour at room temperature. All other samples were treated with PBS for 1 hour at room temp. Cells were incubated with Fc-tagged EGFR extracellular domain (ECD) and mouse anti-FLAG antibody. Cells were washed to remove unbound Fc-tagged EGFR ECD and anti-FLAG antibody. Cells were incubated with a secondary antibody conjugated to DyLight 650 that recognizes human Fc and a secondary antibody conjugated to BV421 that recognizes mouse antibodies. Binding of the secondary antibodies to cells was measured by flow cytometry. Samples were analyzed using FLOWJO® software to bin CAR T-cells into low, medium, and high expression groups based on anti-FLAG/BV421 staining. This served to normalize expression between different constructs.

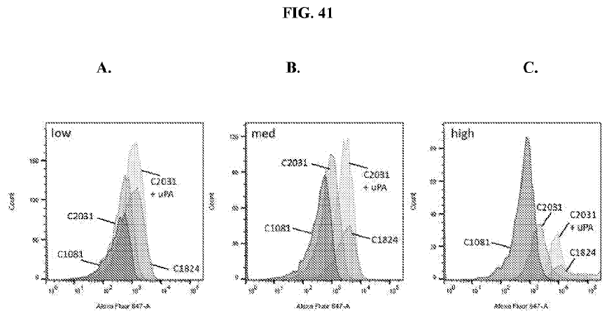

The results of this experiment are shown in FIG. 41. Treatment of the cells expressing the conditionally activated chimeric antigen receptor, C2031, with uPA activated EGFR-binding activity to the same level as the positive control cells expressing C1824.

### Example 29: Protease Cleavage Site-Dependent Activation of CAR T-Cell Activity

Chimeric antigen receptor-expressing T-cells (CAR T-cells) were generated by infecting isolated CD4/CD8 positive T cells from a healthy donor with lentivirus expressing the indicated constructs. FIG. 38 shows a diagram with the constructs used. CAR T-cells were mixed at a 10:1 ratio with EGFR-expressing cancer cells HCT116, engineered to express luciferase. After 72 hours, luciferase activity was measured as a readout of HCT116 viability. CAR-T cells that lacked a target antigen binding domain (C1081) were used as a negative control.

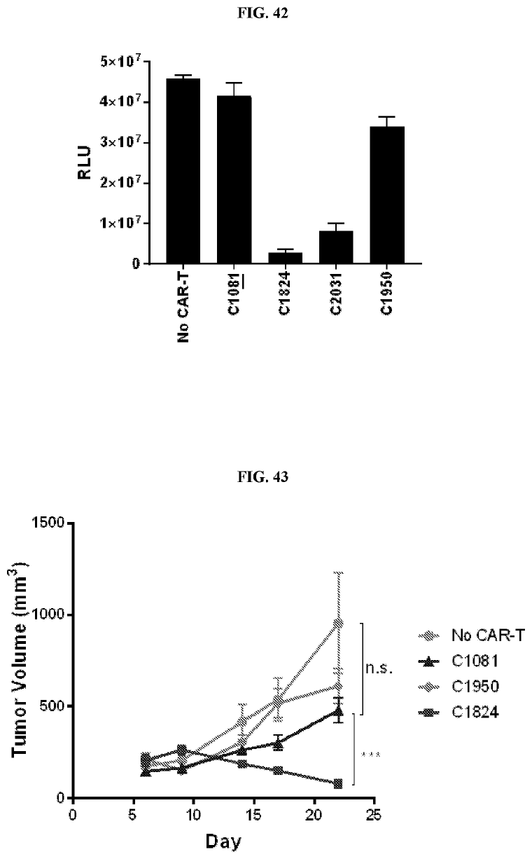

The results of this experiment are shown in FIG. 42. The CAR T-cell expressing a receptor without a protease cleavage site (C1950) showed little cell killing activity. The CAR T-cell expressing a receptor with a protease cleavage site (C2031) showed significant cell killing activity.

### Example 30: In Vivo Masking with Non-Cleavable ProCAR Constructs

NSG mice were subcutaneously implanted with an admixture of HCT116 cancer cells and the indicated CAR-T cells from FIG. 38 at a ratio of 1:1. Tumor volume was measured at the indicated days post-implantation. The results of this experiment are show in FIG. 43. While the CAR-T expressing a receptor with only a target binding domain (C1824) was able to inhibit tumor formation, the CAR-T cell expressing a receptor with a dual binding moiety containing a modified non-CDR loop and a non-cleavable protease linker (C1950) had no activity. Significance was determined by using unpaired t test between groups. *** denotes p<0.0005.

### Example 31: Inhibition of CAR-T Cell Killing Activity by Modified Non-CDR Loop Mask

Chimeric antigen receptor-expressing T-cells (CAR T-cells) were generated by infecting isolated CD4/CD8 positive T cells from a healthy donor with lentivirus expressing the indicated constructs. FIG. 44 shows a diagram with the constructs used. CAR T-cells were mixed at a 10:1 ratio with EGFR-expressing cancer cells HCT116, engineered to express luciferase. After 72 hours, luciferase activity was measured as a readout of HCT116 viability. CAR-T cells that lacked a target antigen binding domain (C1081) were used as a negative control.

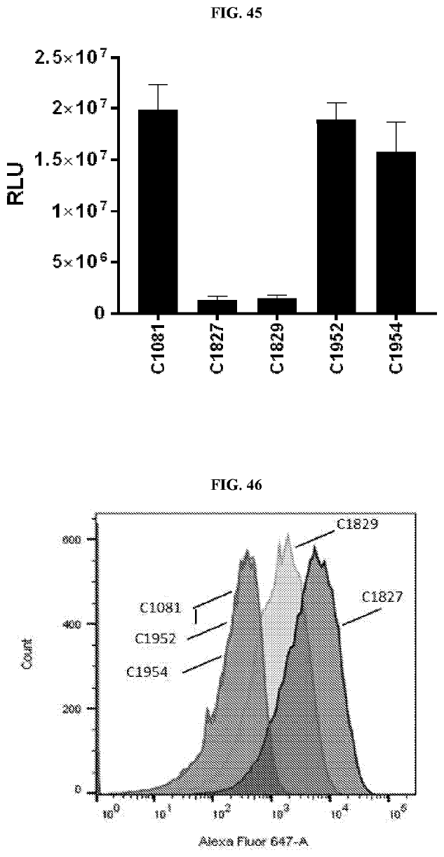

The results of this experiment are shown in FIG. 45. These results demonstrate that the construct containing binding domain 10G with a modified non-CDR loop (C1952 and 1954) significantly blocks cell killing activity. The construct containing binding domain 10G without a modified non-CDR loop (C1829) did not block cell killing activity.

### Example 32: Chimeric Antigen Receptor Binding to EGFR

Chimeric antigen receptor-expressing T-cells (CAR T-cells) were generated by infecting isolated CD4/CD8 positive T cells from a healthy donor with lentivirus expressing the indicated constructs. FIG. 44 shows a diagram with the constructs used. Cells were incubated with Fc-tagged EGFR extracellular domain (ECD). Cells were washed to remove unbound Fc-tagged EGFR ECD. Cells were incubated with a secondary antibody conjugated to DyLight 650 that recognizes human Fc. Binding of the secondary antibody to cells was measured by flow cytometry.

The results of this experiment are shown in FIG. 46. C1954 and C1952, each comprising binding domain 10G with a unique modified non-CDR loop, show no detectable binding to EGFR-Fc, while positive control C1827 (construct comprising the target antigen binding domain EH104 but no dual binding moiety) binds strongly.

The construct C1829, comprising the EGFR binding domain EH104 and the anti-ALB binding moiety 10G with an unmodified non-CDR loop and a non-cleavable linker, i.e., without masking of EH4 binding domain, shows very little steric blocking. This indicates that the anti-ALB binding domain on its own without the mask shows very little steric blocking and that the blocking of EGFR binding, seen in case of C1954 and C1952, is an effect of the masking.

### Example 33: Protease Activation of Conditionally Active Chimeric Antigen Receptors to EGFR

Chimeric antigen receptor-expressing T-cells (CAR T-cells) were generated by infecting isolated CD4/CD8 positive T cells from a healthy donor with lentivirus expressing the indicated constructs. FIG. 44 shows a diagram with the constructs used. CAR T-cells expressing C2033 were treated with either PBS or recombinant uPA protease, as indicated, for 1 hour at room temperature. All other samples were treated with PBS for 1 hour at room temp. Cells were incubated with Fc-tagged EGFR extracellular domain (ECD) and mouse anti-FLAG antibody. Cells were washed to remove unbound Fc-tagged EGFR ECD and anti-FLAG antibody. Cells were incubated with a secondary antibody conjugated to DyLight 650 that recognizes human Fc and a secondary antibody conjugated to BV421 that recognizes mouse antibodies. Binding of the secondary antibodies to cells was measured by flow cytometry. Samples were analyzed using FLOWJO® software to bin CAR T-cells into low, medium, and high expression groups based on anti-FLAG/BV421 staining. This served to normalize expression between different constructs.

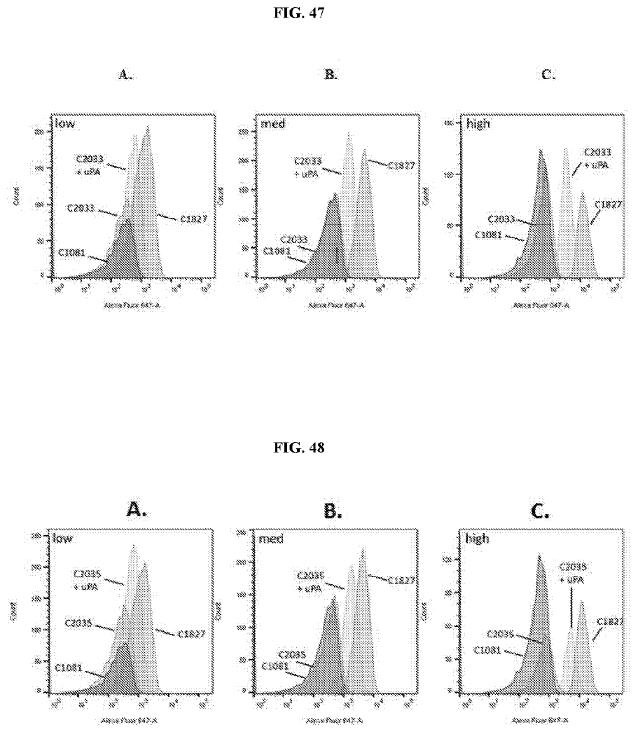

The results of this experiment are shown in FIG. 47A-47C. Treatment of C2033 expressing cells with uPA activated EGFR-binding activity.

### Example 34: Protease Activation of Conditionally Active Chimeric Antigen Receptors to EGFR

Chimeric antigen receptor-expressing T-cells (CAR T-cells) were generated by infecting isolated CD4/CD8 positive T cells from a healthy donor with lentivirus expressing the indicated constructs. FIG. 44 shows a diagram with the constructs used. CAR T-cells expressing C2035 were treated with either PBS or recombinant uPA protease, as indicated, for 1 hour at room temperature. All other samples were treated with PBS for 1 hour at room temp. Cells were incubated with Fc-tagged EGFR extracellular domain (ECD) and mouse anti-FLAG antibody. Cells were washed to remove unbound Fc-tagged EGFR ECD and anti-FLAG antibody. Cells were incubated with a secondary antibody conjugated to DyLight 650 that recognizes human Fc and a secondary antibody conjugated to BV421 that recognizes mouse antibodies. Binding of the secondary antibodies to cells was measured by flow cytometry. Samples were analyzed using FLOWJO® software to bin CAR T-cells into low, medium, and high expression groups based on anti-FLAG/BV421 staining. This served to normalize expression between different constructs.

The results of this experiment are shown in FIGS. 48A-48C. Treatment of C2035 expressing cells with uPA activated EGFR-binding activity.

### Example 35: Protease Cleavage Site-Dependent Activation of CAR T-Cell Activity

Chimeric antigen receptor-expressing T-cells (CAR T-cells) were generated by infecting isolated CD4/CD8 positive T cells from a healthy donor with lentivirus expressing the indicated constructs. FIG. 44 shows a diagram with the constructs used. CAR T-cells were mixed at a 10:1 ratio with EGFR-expressing cancer cells HCT116, engineered to express luciferase. After 72 hours, luciferase activity was measured as a readout of HCT116 viability. CAR-T cells that lacked a target antigen binding domain (C1081) were used as a negative control.

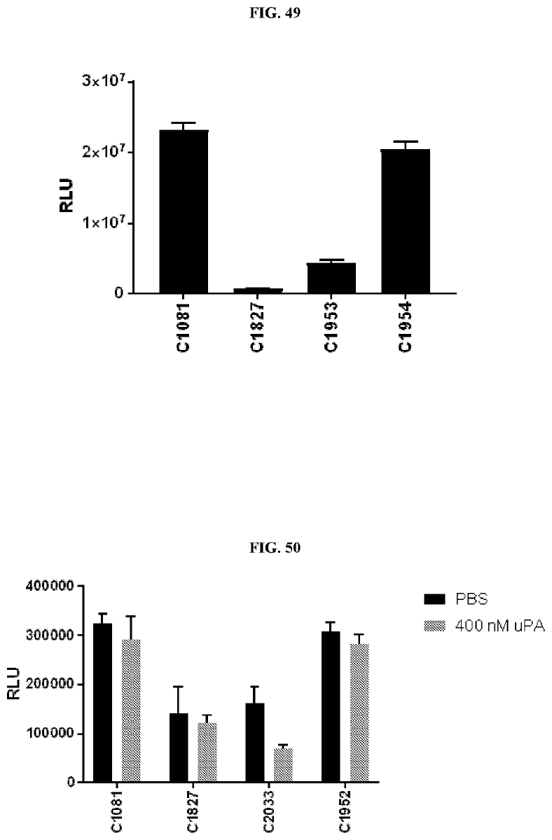

The results of this experiment are shown in FIG. 49. The CAR T-cell expressing a receptor without a protease cleavage site (C1954) showed little cell killing activity. The CAR T-cell expressing a receptor with a protease cleavage site (C1953) showed significant cell killing activity.

### Example 36: Protease Activation of CAR T-Cell Killing Activity

Chimeric antigen receptor-expressing T-cells (CAR T-cells) were generated by infecting isolated CD4/CD8 positive T cells from a healthy donor with lentivirus expressing the indicated constructs. FIG. 44 shows a diagram with the constructs used. CAR-T cells were washed once with PBS and then treated with either PBS or PBS containing 400 nM recombinant uPA protease for 1 hour at room temperature. CAR-T cells were then mixed at a 40:1 ratio with EGFR-expressing HCT116 cells engineered to express luciferase. After 24 hours luciferase activity was measured as a readout of HCT116 viability. CAR-T cells that lacked a target antigen binding domain (C1081) were used as a negative control.

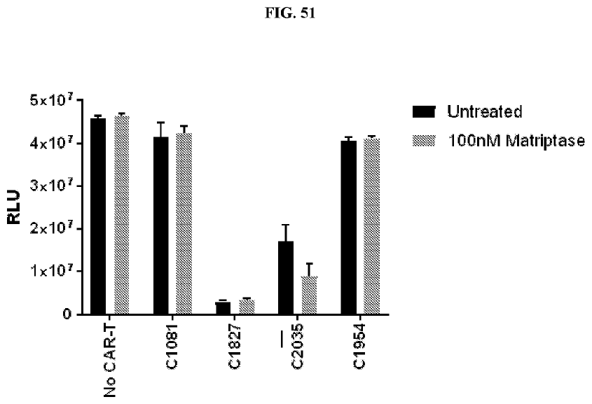

The results of this experiment are shown in FIG. 50. Cells expressing the non-cleavable receptor C1952 showed little cell killing activity. Cells expressing the cleavable receptor C2033 showed moderate cell killing, which was further increased by treatment with recombinant uPA protease.

### Example 37: Protease Activation of CAR T-Cell Killing Activity

Chimeric antigen receptor-expressing T-cells (CAR T-cells) were generated by infecting isolated CD4/CD8 positive T cells from a healthy donor with lentivirus expressing the indicated constructs. FIG. 44 shows a diagram with the constructs used. CAR-T cells were then mixed at a 10:1 ratio with HCT116 cells engineered to express luciferase in T cell culture media with or without 100 nM recombinant Matriptase protease. After 72 hours luciferase activity was measured as a readout of HCT116 viability. CAR-T cells that lacked a target antigen binding domain (C1081) were used as a negative control.

The results of this experiment are shown in FIG. 51. Cells expressing the non-cleavable receptor C1954 showed little cell killing activity. Cells expressing the cleavable receptor C2035 showed moderate cell killing activity, which was further increased by treatment with recombinant Matriptase protease.

### Example 38: Construction and Testing of Exemplary Multivalent Target Binding Proteins Constructs

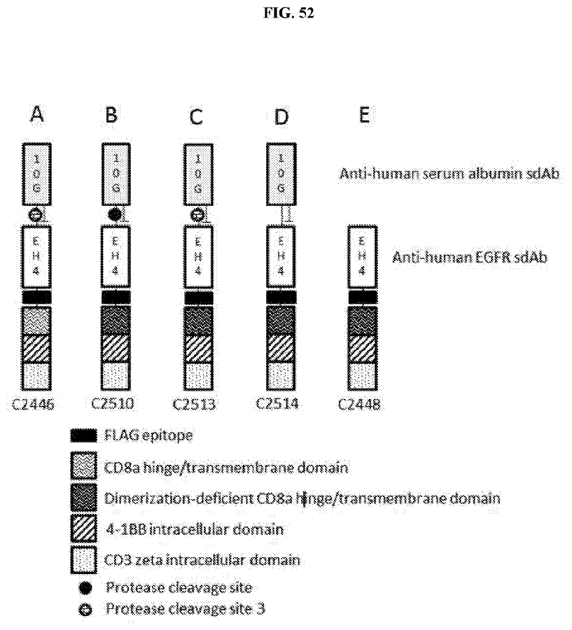

The following constructs were made. Construct C2446 includes an anti-human serine albumin sdAb, a protease cleavage site 3, an anti-human EGFR sdAb, a FLAG epitope, a CD8 hinge/transmembrane domain, a 4-1BB intracellular domain, and a CD3 zeta intracellular domain (FIG. 52A; SEQ ID NO: 832). Construct C2510 includes an anti-human serine albumin sdAb, a protease cleavage site, an anti-human EGFR sdAb, a FLAG epitope, a dimerization-deficient CD8a hinge/transmembrane domain, a 4-1BB intracellular domain, and a CD3 zeta intracellular domain (FIG. 52B; SEQ ID NO: 834). Construct C2513 includes an anti-human serine albumin sdAb, a protease cleavage site 3, an anti-human EGFR sdAb, a FLAG epitope, a dimerization-deficient CD8a hinge/transmembrane domain, a 4-1BB intracellular domain, and a CD3 zeta intracellular domain (FIG. 52C; SEQ ID NO: 835). Construct C2514 includes an anti-human serine albumin sdAb, an anti-human EGFR sdAb, a FLAG epitope, a dimerization-deficient CD8a hinge/transmembrane domain, a 4-1BB intracellular domain, and a CD3 zeta intracellular domain (FIG. 52D; SEQ ID NO: 836). Construct C2448, includes an anti-human EGFR sdAb, a FLAG epitope, a dimerization-deficient CD8a hinge/transmembrane domain, a 4-1BB intracellular domain, and a CD3 zeta intracellular domain (FIG. 52E; SEQ ID NO: 833).

In Vitro Protease Site-Dependent Cell Killing Activity

In a first assay, 300,000 primary human T cells isolated from healthy donors were infected with 1 mL lentiviral supernatant made from the indicated constructs to generate anti-EGFR CAR-T cells. Twenty five thousand CAR-T cells were subsequently co-cultured under standard conditions at a 10:1 ratio (CAR-T:Target cells) with EGFR-expressing cancer cells that stably express luciferase for 72 hours. Luciferase activity is determined as a proxy for cancer cell viability and normalized to the control CAR-T cells that do not contain scFv.

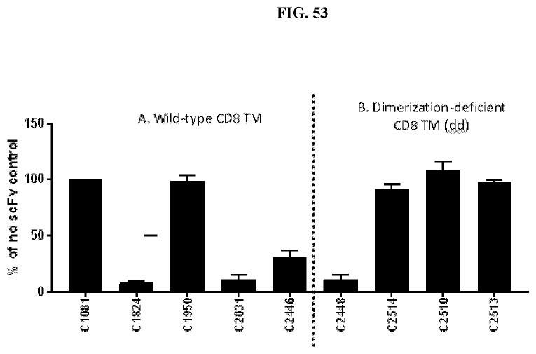

The data demonstrated that the level of protease side-dependent cell killing activity was reduced in ProCAR constructs that lacked the dimerization activity of the CD8a transmembrane domain (FIG. 53B) compared to wild-type (FIG. 53A). The dimerization mutation reduces the amount of cell killing activity in the absence of exogenous protease (e.g., compare C2031 to C2510, or compare C2446 to C2513).

In a second assay, 300,000 primary human T cells isolated from healthy donors were infected with 1 mL lentiviral supernatant made from the indicated constructs to generate anti-EGFR CAR-T cells, which were subsequently co-cultured at ratios of 10:1, 5:1, 2.5:1, and 1:1 (CAR-T:Target cells) with EGF-expressing cancer cells that stably express luciferase for 72 hours. Luciferase activity is determined as a proxy for cancer cell viability and normalized to the control CAR-T cells that do not contain scFv.

The data demonstrates that dimerization-deficient EGFR CAR has similar killing activity compared to wild-type (FIG. 54).

### Example 39: Construction and Testing of Exemplary Multivalent Target Binding Proteins Constructs

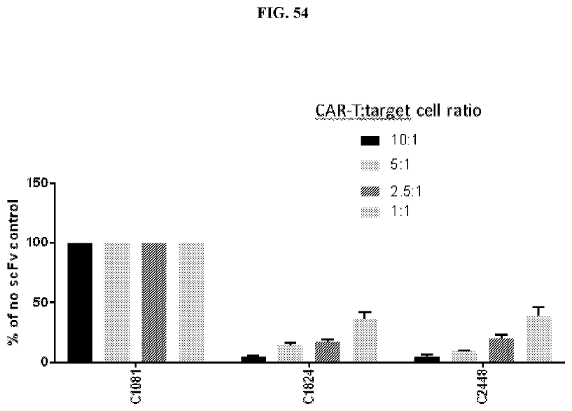

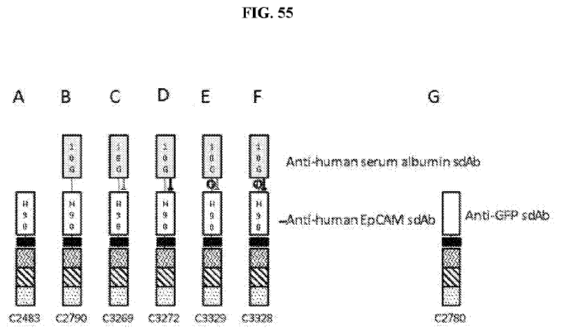

The following ProCAR constructs were made. Construct C2483 includes an anti-human EpCAM sdAb, a FLAG epitope, a CD8 hinge/transmembrane domain, a 4-1BB intracellular domain, and a CD3 zeta intracellular domain (FIG. 55A; SEQ ID NO: 838). Construct C2790 includes an anti-human serum albumin sdAb, an anti-human EpCAM sdAb, a FLAG epitope, a CD8 hinge/transmembrane domain, a 4-1BB intracellular domain, and a CD3 zeta intracellular domain (FIG. 55B; SEQ ID NO: 839). Construct C3269 includes an anti-human serum albumin sdAb, an anti-human EpCAM sdAb, a FLAG epitope, a CD8 hinge/transmembrane domain, a 4-1BB intracellular domain, and a CD3 zeta intracellular domain (FIG. 55C; SEQ ID NO: 840). Construct C3272 includes an anti-human serum albumin sdAb, an anti-human EpCAM sdAb, a FLAG epitope, a CD8 hinge/transmembrane domain, a 4-1BB intracellular domain, and a CD3 zeta intracellular domain (FIG. 55D; SEQ ID NO: 841). Construct C3329 includes an anti-human serum albumin sdAb, a protease cleavage site 3, an anti-human EpCAM sdAb, a FLAG epitope, a CD8 hinge/transmembrane domain, a 4-1BB intracellular domain, and a CD3 zeta intracellular domain (FIG. 55E; SEQ ID NO: 843). Construct C3328 includes an anti-human serum albumin sdAb, a protease cleavage site 3, an anti-human EpCAM sdAb, a FLAG epitope, a CD8 hinge/transmembrane domain, a 4-1BB intracellular domain, and a CD3 zeta intracellular domain (FIG. 55F; SEQ ID NO: 842). Construct C2780 includes an anti-GFP sdAb, a FLAG epitope, a CD8 hinge/transmembrane domain, a 4-1BB intracellular domain, and a CD3 zeta intracellular domain (FIG. 55G; SEQ ID NO: 837).

EpCAM Mask 1 Blocks ProCAR EpCAM-Binding Activity

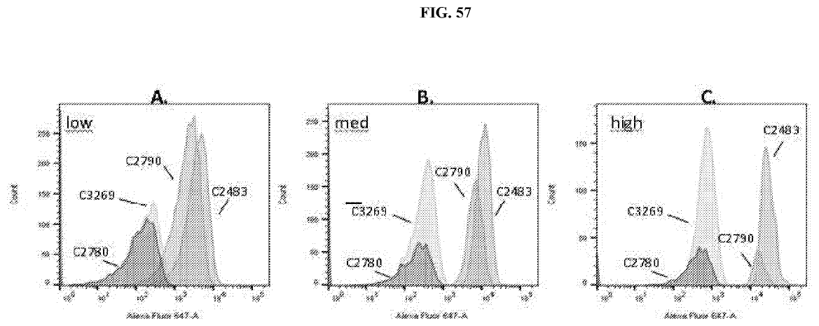

300,000 primary human T cells isolated from healthy donors were infected with 1 mL lentiviral supernatant made from the indicated constructs from FIG. 55 to generate anti-EpCAM CAR-T cells, which were subsequently stained with anti-FLAG antibodies and EGFR-Fc along with the indicated secondary antibodies. The data was analyzed by flow cytometry. FIG. 57 provides histograms of EpCAM-Fc/Alexa Fluor 647 staining of CAR-T cells that have been grouped into low (FIG. 57A), medium (FIG. 57B), or high (FIG. 57C) CAR expression based on anti-FLAG staining.

This data demonstrates the efficacy of EpCAM mask 1 blocking ProCAR EpCAM-binding activity.

EpCAM Mask 2 Blocks ProCAR EpCAM-Binding Activity

300,000 primary human T cells isolated from healthy donors were infected with 1 mL lentiviral supernatant made from the indicated constructs from FIG. 55 to generate anti-EpCAM CAR-T cells, which were subsequently stained with anti-FLAG antibodies and EGFR-Fc along with the indicated secondary antibodies. The data was analyzed by flow cytometry.

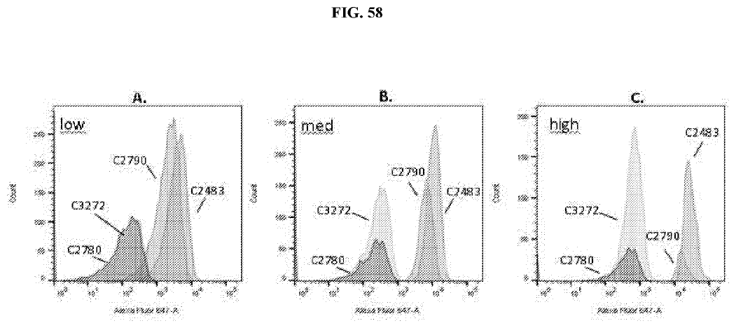

FIG. 58 provides histograms of EpCAM-Fc/Alexa Fluor 647 staining of CAR-T cells from FIG. 58 that have been grouped into low (FIG. 58A), medium (FIG. 58B), or high (FIG. 58C) CAR expression based on anti-FLAG staining that demonstrate the efficacy of EpCAM mask 2 in blocking ProCAR EpCAM-binding activity.

Masking of Anti-EpCAM sdAb H90

300,000 primary human T cells isolated from healthy donors were infected with 1 mL lentiviral supernatant made from the indicated constructs from FIG. 55 to generate anti-EpCAM CAR-T cells, which were subsequently co-cultured with various ratios (CAR-T:Target cells) with EGFR-expressing cancer cells that stably express luciferase. Luciferase activity was read 72 hours later as a proxy for cancer cell viability and normalized to the anti-GFP control CAR-T cells, C2780.

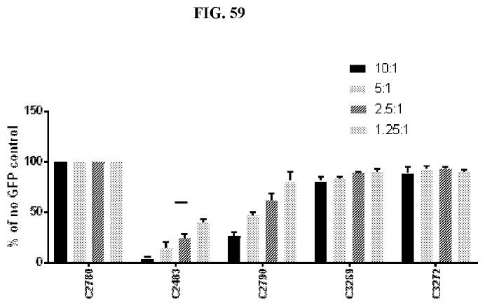

The data provided in FIG. 59 demonstrates masking of the anti-EpCAM sdAb H90. C2843 is the “naked” CAR, i.e., no anti-ALB domain. Addition of the anti-ALB domain (C2790) has a small impact on cell killing activity. Addition of a mask to the CC′ non-CDR loop of the anti-ALB domain has a large impact on cell killing activity due to specific blocking of EpCAM binding.

Steric Blocking by HSA of Anti-EpCAM sdAb H90

300,000 primary human T cells isolated from healthy donors were infected with 1 mL lentiviral supernatant made from the indicated constructs from FIG. 55 to generate anti-EpCAM CAR-T cells, which were subsequently co-cultured at various ratios (CAR-T:Target cells) with EGFR-expressing cancer cells that stably express luciferase in the presence or absence of human serum albumin (HSA). Luciferase activity was read 72 hours later as a proxy for cancer cell viability and normalized to the anti-GFP control CAR-T cells, C2780.

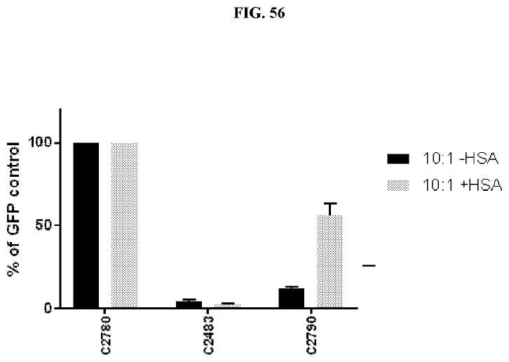

The data provided in FIG. 56 demonstrate steric blocking by HSA of the anti-EpCAM sdAb H90.

### Example 40: EpCAM ProCAR Protease Site-Dependent Cell Killing

300,000 primary human T cells isolated from healthy donors were infected with 1 mL lentiviral supernatant made from the indicated constructs from FIG. 21 to generate anti-EpCAM CAR-T cells, which were subsequently co-cultured at various ratios (CAR-T:Target cells) with EGFR-expressing cancer cells that stably express luciferase. Luciferase activity was read 72 hours later as a proxy for cancer cell viability and normalized to the anti-GFP control CAR-T cells, C2780

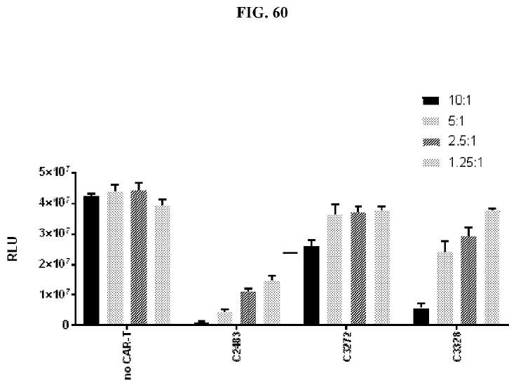

FIG. 60 demonstrates protease site-dependent cell killing activity of CAR-T cells expressing a masked EpCAM ProCAR.

### Example 41: Protease Activation of EpCAM Mask 1 ProCAR Antigen Binding Activity

300,000 primary human T cells isolated from healthy donors were infected with 1 mL lentiviral supernatant made from the indicated constructs from FIG. 21 to generate anti-EpCAM CAR-T cells, which were subsequently washed in PBS, and then treated for 1 hr at room temp with either PBS or PBS containing 400 nM recombinant uPA protease and then stained with anti-FLAG antibodies and EpCAM-Fc along with the anti-mouse BV421 and anti-human Fc Alexa Fluor 647 secondary antibodies and analyzed by flow cytometry.

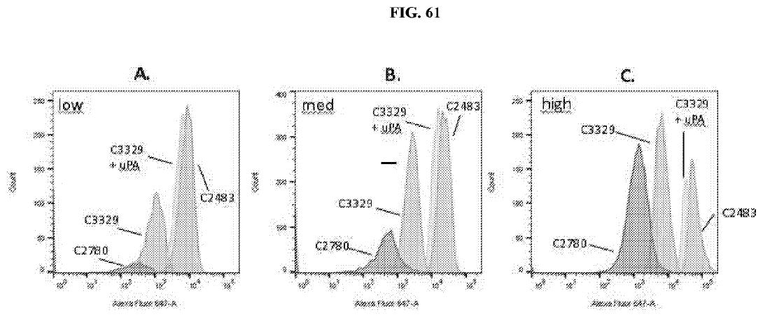

Histograms of EpCAM-Fc staining of CAR-T cells that have been grouped into low (FIG. 61A), medium (FIG. 61B), or high (FIG. 61C) CAR expression based on anti-FLAG staining. These results demonstrate protease activation of EpCAM Mask 1 ProCAR antigen binding activity.

### Example 42: Protease Activation of EpCAM Mask 2 ProCAR Antigen Binding Activity

300,000 primary human T cells isolated from healthy donors were infected with 1 mL lentiviral supernatant made from the indicated constructs from FIG. 55 to generate anti-EpCAM CAR-T cells, which were subsequently washed in PBS, and then treated for 1 hr at room temp with either PBS or PBS containing 400 nM recombinant uPA protease and then stained with anti-FLAG antibodies and EpCAM-Fc along with the anti-mouse BV421 and anti-human Fc Alexa Fluor 647 secondary antibodies and analyzed by flow cytometry.

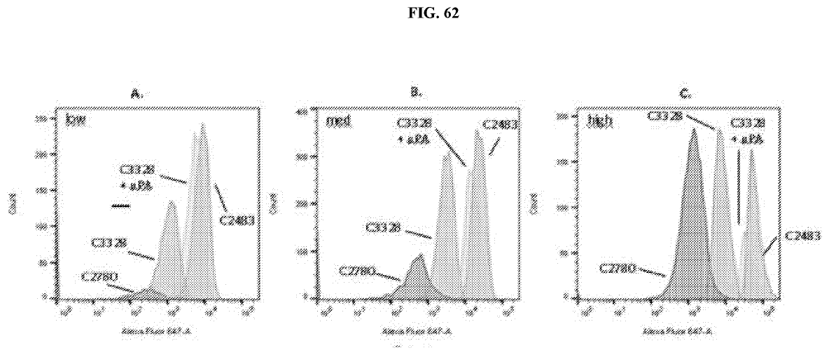

Histograms of EpCAM-Fc staining of CAR-T cells that have been grouped into low (FIG. 62A), medium (FIG. 62B), or high (FIG. 62C) CAR expression based on anti-FLAG staining. These results demonstrate protease activation of EpCAM Mask 2 ProCAR antigen binding activity.

### Example 43: Demonstration of Improved Tolerability in Mouse, Conferred by an Exemplary EpCAM Targeting ProTriTAC Molecule

In this study, the tolerability of an exemplary EpCAM targeting ProTriTAC molecule was assessed. Seven weeks old NSG female tumor free mice were intraperitoneally injected with 2×10⁷ expanded human T cells at the commencement of the study, i.e., at day 0. On day 2, treatment was started by dividing the mice into various groups and administering to them varying concentrations of the exemplary EpCAM targeting ProTriTAC molecule, containing the linker sequence L040, an EpCAMR targeting TriTAC molecule, an EpCAM targeting ProTriTAC molecule containing a non-cleavable linker (EpCAM ProTriTAC (NCLV), and a GFP TriTAC molecule as a control. The molecules were administered once daily for 10 days, at the following dosages: 0.03 mg/kg, 0.1 mg/kg, 0.3 mg/kg, and 1 mg/kg. Starting from day 2, body weight of the animals were recorded daily.

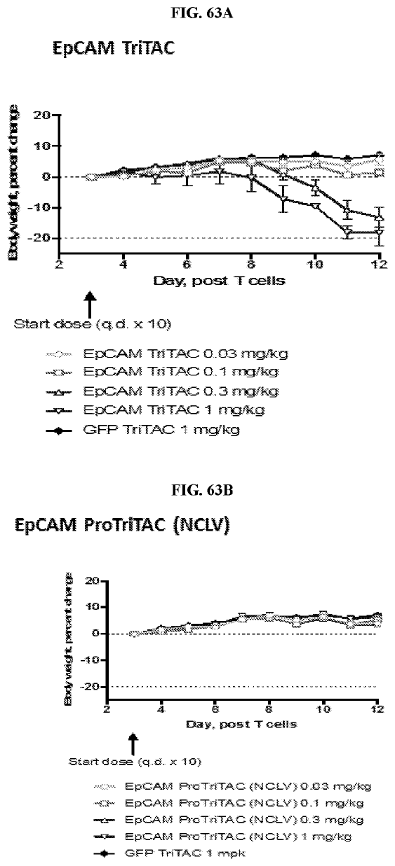

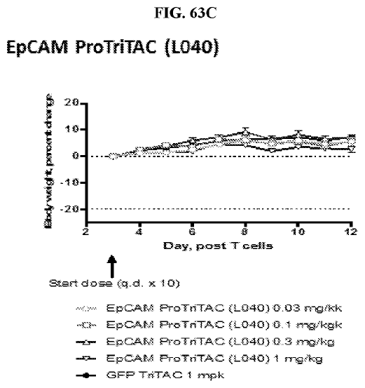

As shown in FIGS. 63A-63C, the EpCAM targeting ProTriTAC molecule containing a non-cleavable linker (ProTriTAC (NCLV)) (SEQ ID No. 993) and a GFP TriTAC (used as a negative control) were very well tolerated in mice even at the highest dose of 1 mg/kg. The EpCAM targeted ProTriTAC molecule containing the linker sequence of L040 (SEQ ID No. 994) was well tolerated at the dosage of 1 mg/kg, whereas the EpCAM targeted TriTAC (SEQ ID No. 992) was well tolerated 0.1 mg/kg. It was thus observed that the EpCAM targeting ProTriTAC containing the L040 linker sequence conferred at least about 10 times improved tolerability, in mouse, compared to the EpCAM targeting TriTAC.

SEQUENCE TABLE

| DESCRIPTION | SEQUENCE |
|---|---|
| PSMA Prodrug C1872 | EVQLVESGGGLVQPGNSLRLSCAASGFTFSKFGMSWVRQGGGGGLDGNEE |
| (SEQ ID NO: 43) | PGGLEWVSSISGSGRDTLYADSVKGRFTISRDNAKTTLYLQMNSLRPEDTAV |
| | YYCTIGGSLSVSSQGTLVTVSSGGGGKPLGLQARVVGGGGT |
| | QTVVTQEPSLTVSPGGTVTLTCASSTGAVTSGNYPNWVQQKPGQAPRGLIG |
| | GTKFLVPGTPARFSGSLLGGKAALTLSGVQPEDEAEYYCTLWYSNRWVFGG |
| | GTKLTVLGGGGSGGGGSGGGGSEVQLVESGGGLVQPGGSLKLSCAASGFTF |
| | NKYAINWVRQAPGKGLEWVARIRSKYNNYATYYADQVKDRFTISRDDSKNT |
| | AYLQMNNLKTEDTAVYYCVRHANFGNSYISYWAYWGQGTLVTVSSGGGGS |
| | GGGSEVQLVESGGGLVQPGGSLTLSCAASRFMISEYHMHWVRQAPGKGLE |
| | WVSTINPAGTTDYAESVKGRFTISRDNAKNTLYLQMNSLKPEDTAVYYCDSY |
| | GYRGQGTQVTVSSHHHHHH |
| PSMA Non-cleavable | EVQLVESGGGLVQPGNSLRLSCAASGFTFSKFGMSWVRQGGGGGLDGNE |
| Prodrug C1873 | EPGGLEWVSSISGSGRDTLYADSVKGRFTISRDNAKTTLYLQMNSLRPEDTA |
| (SEQ ID NO: 44) | VYYCTIGGSLSVSSQGTLVTVSSGGGGSGGGGSGGVVGGGGT |
| | QTVVTQEPSLIVSPGGTVTLTCASSTGAVTSGNYPNWVQQKPGQAPRGLIG |
| | GTKFLVPGTPARFSGSLLGGKAALTLSGVQPEDEAEYYCTLWYSNRWVFGG |
| | GTKLTVLGGGGSGGGGSGGGGSEVQLVESGGGLVQPGGSLKLSCAASGFTF |
| | NKYAINWVRQAPGKGLEWVARIRSKYNNYATYYADQVKDRFTISRDDSKNT |
| | AYLQMNNLKTEDTAVYYCVRHANFGNSYISYWAYWGQGTLVTVSSGGGGS |
| | GGGSEVQLVESGGGLVQPGGSLTLSCAASRFMISEYHMHWVRQAPGKGLE |
| | WVSTINPAGTTDYAESVKGRFTISRDNAKNTLYLQMNSLKPEDTAVYYCDSY |
| | GYRGQGTQVTVSSHHHHHH |
| PSMA Active Drug | VVGGGGTQTVVTQEPSLTVSPGGTVTLTCASSTGAVTSGNYPNWVQQKPG |
| C1875 (SEQ ID NO: 45) | QAPRGLIGGTKFLVPGTPARFSGSLLGGKAALTLSGVQPEDEAEYYCTLWYS |
| | NRWVFGGGTKLTVLGGGGSGGGGSGGGGSEVQLVESGGGLVQPGGSLKL |
| | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSEVQLVESGGGLVQPGGSLTLSCAASRFMISEYHMHWV |
| | RQAPGKGLEWVSTINPAGTTDYAESVKGRFTISRDNAKNTLYLQMNSLKPE |
| | DTAVYYCDSYGYRGQGTQVTVSSHHHHHH |
| EGFR (G8) Prodrug | EVQLVESGGGLVQPGNSLRLSCAASGFTFSKFGMSWVRQGGGGGLDGNE |
| C1486 (SEQ ID NO: 46) | EPGGLEWVSSISGSGRDTLYADSVKGRFTISRDNAKTTLYLQMNSLRPEDTA |
| | VYYCTIGGSLSVSSQGTLVTVSSGGGGKPLGLQARVVGGGGT |
| | QTVVTQEPSLTVSPGGTVTLTCASSTGAVTSGNYPNWVQQKPGQAPRGLIG |
| | GTKFLVPGTPARFSGSLLGGKAALTLSGVQPEDEAEYYCTLWYSNRWVFGG |
| | GTKLTVLGGGGSGGGGSGGGGSEVQLVESGGGLVQPGGSLKLSCAASGFTF |
| | NKYAINWVRQAPGKGLEWVARIRSKYNNYATYYADQVKDRFTISRDDSKNT |
| | AYLQMNNLKTEDTAVYYCVRHANFGNSYISYWAYWGQGTLVTVSSGGGGS |
| | GGGSEVQLVESGGGLVQPGGSLTLSCAASGRTFSSYAMGWFRQAPGKERE |
| | FVVAINWASGSTYYADSVKGRFTISRDNSKNTLYLQMNSLRAEDTAVYYCAA |
| | GYQINSGNYNFKDYEYDYWGQGTLVTVSSHHHHHH |
| EGFR (G8) Non- | EVQLVESGGGLVQPGNSLRLSCAASGFTFSKFGMSWVRQGGGGGLDGNE |
| cleavable Prodrug | EPGGLEWVSSISGSGRDTLYADSVKGRFTISRDNAKTTLYLQMNSLRPEDTA |
| C1756 (SEQ ID NO: 47) | VYYCTIGGSLSVSSQGTLVTVSSGGGGSGGGGSGGVVGGGGT |
| | QTVVTQEPSLTVSPGGTVTLTCASSTGAVTSGNYPNWVQQKPGQAPRGLIG |
| | GTKFLVPGTPARFSGSLLGGKAALTLSGVQPEDEAEYYCTLWYSNRWVFGG |
| | GTKLTVLGGGGSGGGGSGGGGSEVQLVESGGGLVQPGGSLKLSCAASGFTF |
| | NKYAINWVRQAPGKGLEWVARIRSKYNNYATYYADQVKDRFTISRDDSKNT |
| | AYLQMNNLKTEDTAVYYCVRHANFGNSYISYWAYWGQGTLVTVSSGGGGS |
| | GGGSEVQLVESGGGLVQPGGSLTLSCAASGRTFSSYAMGWFRQAPGKERE |
| | FVVAINWASGSTYYADSVKGRFTISRDNSKNTLYLQMNSLRAEDTAVYYCAA |
| | GYQINSGNYNFKDYEYDYWGQGTLVTVSSHHHHHH |
| EGFR (G8) Active Drug | VVGGGGTQTVVTQEPSLTVSPGGTVTLTCASSTGAVTSGNYPNWVQQKPG |
| C1300 (SEQ ID NO: 48) | QAPRGLIGGTKFLVPGTPARFSGSLLGGKAALTLSGVQPEDEAEYYCTLWYS |
| | NRWVFGGGTKLTVLGGGGSGGGGSGGGGSEVQLVESGGGLVQPGGSLKL |
| | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSEVQLVESGGGLVQPGGSLTLSCAASGRTFSSYAMGWF |
| | RQAPGKEREFVVAINWASGSTYYADSVKGRFTISRDNSKNTLYLQMNSLRAE |
| | DTAVYYCAAGYQINSGNYNFKDYEYDYWGQGTLVTVSSHHHHHH |
| GFP TriTAC C646 | QVQLVESGGALVQPGGSLRLSCAASGFPVNRYSMRWYRQAPGKEREWVA |
| (SEQ ID NO: 49) | GMSSAGDRSSYEDSVKGRFTISRDDARNTVYLQMNSLKPEDTAVYYCNVNV |
| | GFEYWGQGTQVTVSSGGGGSGGGSEVQLVESGGGLVQPGNSLRLSCAASG |
| | FTFSKFGMSWVRQAPGKGLEWVSSISGSGRDTLYADSVKGRFTISRDNAKT |
| | TLYLQMNSLRPEDTAVYYCTIGGSLSVSSQGTLVTVSSGGGGSGGGSEVQLV |
| | ESGGGLVQPGGSLKLSCAASGFTFNKYAINWVRQAPGKGLEWVARIRSKYN |
| | NYATYYADQVKDRFTISRDDSKNTAYLQMNNLKTEDTAVYYCVRHANFGNS |
| | YISYWAYWGQGTLVTVSSGGGGSGGGGSGGGGSQTVVTQEPSLTVSPGGT |
| | VTLTCASSTGAVTSGNYPNWVQQKPGQAPRGLIGGTKFLVPGTPARFSGSL |
| | LGGKAALTLSGVQPEDEAEYYCTLWYSNRWVFGGGTKLTVLHHHHHH |
| Exemplary masking | GGGGGLDGNEEPGG |
| sequence (SEQ ID NO: | |
| 50) | |
| Exemplary non- | GGGGSGGGGSGGVVGGGGT |
| cleavable linker | |
| sequence comprising | |
| protease cleavage site | |
| (SEQ ID NO: 51) | |
| PSMA Non-masked Non- | EVQLVESGGGLVQPGNSLRLSCAASGFTFSKFGMSWVRQAPGKGLEWVSSI |
| cleavable Prodrug | SGSGRDTLYADSVKGRFTISRDNAKTTLYLQMNSLRPEDTAVYYCTIGGSLSV |
| C1874 | SSQGTLVTVSSGGGGSGGGGSGGVVGGGGTQTVVTQEPSLTVSPGGTVTL |
| (SEQ ID NO: 52) | TCASSTGAVTSGNYPNWVQQKPGQAPRGLIGGTKFLVPGTPARFSGSLLGG |
| | KAALTLSGVQPEDEAEYYCTLWYSNRWVFGGGTKLTVLGGGGSGGGGSGG |
| | GGSEVQLVESGGGLVQPGGSLKLSCAASGFTFNKYAINWVRQAPGKGLEW |
| | VARIRSKYNNYATYYADQVKDRFTISRDDSKNTAYLQMNNLKTEDTAVYYCV |
| | RHANFGNSYISYWAYWGQGTLVTVSSGGGGSGGGSEVQLVESGGGLVQP |
| | GGSLTLSCAASRFMISEYHMHWVRQAPGKGLEWVSTINPAGTTDYAESVK |
| | GRFTISRDNAKNTLYLQMNSLKPEDTAVYYCDSYGYRGQGTQVIVSSHHHH |
| | HH |
| Exemplary cleavable | GGGGKPLGLQARVVGGGGT |
| linker | |
| (SEQ ID NO: 53) | |
| Exemplary anti-albumin | EVQLVESGGGLVQPGNSLRLSCAASGFTFSKFGMSWVRQGGGGGLDGNE |
| sdAb | EPGGLEWVSSISGSGRDTLYADSVKGRFTISRDNAKTTLYLQMNSLRPEDTA |
| (SEQ ID NO: 54) | VYYCTIGGSLSVSSQGTLVTVSSGGGGKPLGLQARVVGGGGT |
| Exemplary anti-target | EVQLVESGGGLVQPGGSLTLSCAASGRTFSSYAMGWFRQAPGKEREFVVAI |
| (anti-EGFR) sdAb | NWASGSTYYADSVKGRFTISRDNSKNTLYLQMNSLRAEDTAVYYCAAGYQI |
| (SEQ ID NO: 55) | NSGNYNFKDYEYDYWGQGTLVTVSS |
| Exemplary anti-CD3 scFv | QTVVTQEPSLTVSPGGTVTLTCASSTGAVTSGNYPNWVQQKPGQAPRGLIG |
| (SEQ ID NO: 56) | GTKFLVPGTPARFSGSLLGGKAALTLSGVQPEDEAEYYCTLWYSNRWVFGG |
| | GTKLTVLGGGGSGGGGSGGGGSEVQLVESGGGLVQPGGSLKLSCAASGFTF |
| | NKYAINWVRQAPGKGLEWVARIRSKYNNYATYYADQVKDRFTISRDDSKNT |
| | AYLQMNNLKTEDTAVYYCVRHANFGNSYISYWAYWGQGTLVTVSS |
| Exemplary anti-target | EVQLVESGGGLVQPGGSLTLSCAASRFMISEYHMHWVRQAPGKGLEWVST |
| (anti-PSMA) sdAb | INPAGTTDYAESVKGRFTISRDNAKNTLYLQMNSLKPEDTAVYYCDSYGYRG |
| (SEQ ID NO: 57) | QGTQVTVSS |
| Exemplary anti-target | EVQLVESGGGLVQPGGSLRLSCAASRFMISEYHMHWVRQAPGKGLEWVS |
| (anti-PSMA) sdAb | DINPAGTTDYAESVKGRFTISRDNAKNTLYLQMNSLRAEDTAVYYCDSYGYR |
| (SEQ ID NO: 63) | GQGTLVTVSS |
| Exemplary anti-target | EVQLVESGGGLVQPGGSLRLSCAASRFMISEYHMHWVRQAPGKGLEWVST |
| (anti-PSMA) sdAb | INPAGTTDYAESVKGRFTISRDNAKNTLYLQMNSLRAEDTAVYYCDSYGYRG |
| (SEQ ID NO: 64) | QGTLVTVSS |
| Exemplary anti-target | EVQLVESGGGLVQPGGSLRLSCAASRFMISEYSMHWVRQAPGKGLEWVSTI |
| (anti-PSMA) sdAb | NPAKTTDYAESVKGRFTISRDNAKNTLYLQMNSLRAEDTAVYYCDSYGYRG |
| (SEQ ID NO: 65) | QGTLVTVSS |
| Exemplary anti-target | EVQLVESGGGLVQPGGSLRLSCAASRFMISPYSMHWVRQAPGKGLEWVST |
| (anti-PSMA) sdAb | INPAGTTDYAESVKGRFTISRDNAKNTLYLQMNSLRAEDTAVYYCDGYGYRG |
| (SEQ ID NO: 66) | QGTLVTVSS |
| Exemplary anti-target | EVQLVESGGGLVQPGGSLRLSCAASRFMISEYSMHWVRQAPGKGLEWVSTI |
| (anti-PSMA) sdAb | NPAGQTDYAESVKGRFTISRDNAKNTLYLQMNSLRAEDTAVYYCDGYGYRG |
| (SEQ ID NO: 67) | QGTLVTVSS |
| Exemplary anti-target | EVQLVESGGGLVQPGGSLRLSCAASRFMISEYSMHWVRQAPGKGLEWVSTI |
| (anti-PSMA) sdAb | NPAGTTDYAEYVKGRFTISRDNAKNTLYLQMNSLRAEDTAVYYCDGYGYRG |
| (SEQ ID NO: 68) | QGTLVTVSS |
| Exemplary anti-target | EVQLVESGGGLVQPGGSLRLSCAASRFMISEYHMHWVRQAPGKGLEWVS |
| (anti-PSMA) sdAb | DINPAKTTDYAESVKGRFTISRDNAKNTLYLQMNSLRAEDTAVYYCDSYGYR |
| (SEQ ID NO: 69) | GQGTLVTVSS |
| Exemplary anti-target | EVQLVESGGGLVQPGGSLRLSCAASRFMISPYHMHWVRQAPGKGLEWVS |
| (anti-PSMA) sdAb | DINPAGTTDYAESVKGRFTISRDNAKNTLYLQMNSLRAEDTAVYYCDSYGYR |
| (SEQ ID NO: 70) | GQGTLVTVSS |
| Exemplary anti-target | EVQLVESGGGLVQPGGSLRLSCAASRFMISEYHMHWVRQAPGKGLEWVS |
| (anti-PSMA) sdAb | DINPAGQTDYAESVKGRFTISRDNAKNTLYLQMNSLRAEDTAVYYCDSYGYR |
| (SEQ ID NO: 71) | GQGTLVTVSS |
| Exemplary anti-target | EVQLVESGGGLVQPGGSLRLSCAASRFMISEYHMHWVRQAPGKGLEWVS |
| (anti-PSMA) sdAb | DINPAGTTDYAEYVKGRFTISRDNAKNTLYLQMNSLRAEDTAVYYCDSYGYR |
| (SEQ ID NO: 72) | GQGTLVTVSS |
| Exemplary anti-target | EVQLVESGGGLVQPGGSLTLSCAASRFMISEYHMHWVRQAPGKGLEWVSD |
| (anti-PSMA) sdAb | INPAGTTDYAESVKGRFTISRDNAKNTLYLQMNSLKPEDTAVYYCDSYGYRG |
| (SEQ ID NO: 73) | QGTQVTVSS |
| Exemplary anti-CD3 scFv | EVQLVESGGGLVQPGGSLKLSCAASGFTFNKYAINWVRQAPGKGLEWVARI |
| (SEQ ID NO: 74) | RSKYNNYATYYADQVKDRFTISRDDSKNTAYLQMNNLKTEDTAVYYCVRHA |
| | NFGNSYISYWAYWGQGTLVTVSSGGGGSGGGGSGGGGSQTVVTQEPSLT |
| | VSPGGTVTLTCASSTGAVTSGNYPNWVQQKPGQAPRGLIGGTKFLVPGTPA |
| | RFSGSLLGGKAALTLSGVQPEDEAEYYCTLWYSNRWVFGGGTKLTVL |
| Exemplary anti-CD3 scFv | EVQLVESGGGLVQPGGSLKLSCAASGFEFNKYAMNWVRQAPGKGLEWVA |
| (SEQ ID NO: 75) | RIRSKYNKYATYYADSVKDRFTISRDDSKNTAYLQMNNLKTEDTAVYYCVRH |
| | GNFGNSYISYWAYWGQGTLVTVSSGGGGSGGGGSGGGGSQTVVTQEPSL |
| | TVSPGGTVTLTCGSSFGAVTSGNYPNWVQQKPGQAPRGLIGGTKFLAPGTP |
| | ARFSGSLLGGKAALTLSGVQPEDEAEYYCVLWYDNRWVFGGGTKLTVL |
| Exemplary anti-CD3 scFv | EVQLVESGGGLVQPGGSLKLSCAASGFTFNKYAMNWVRQAPGKGLEWVA |
| (SEQ ID NO: 76) | RIRSKYNNYATYYADSVKDRFTISRDDSKNTAYLQMNNLKTEDTAVYYCVRH |
| | GNFGNSHISYWAYWGQGTLVTVSSGGGGSGGGGSGGGGSQTVVTQEPSL |
| | TVSPGGTVTLTCGSSTGYVTSGNYPNWVQQKPGQAPRGLIGGTSFLAPGTP |
| | ARFSGSLLGGKAALTLSGVQPEDEAEYYCVLWYSNRWIFGGGTKLTVL |
| Exemplary anti-CD3 scFv | EVQLVESGGGLVQPGGSLKLSCAASGFMFNKYAMNWVRQAPGKGLEWV |
| (SEQ ID NO: 77) | ARIRSKSNNYATYYADSVKDRFTISRDDSKNTAYLQMNNLKTEDTAVYYCVR |
| | HGNFGNSYISYWATVVGQGTLVTVSSGGGGSGGGGSGGGGSQTVVTQEPS |
| | LTVSPGGTVTLTCGSSFGAVTSGNYPNWVQQKPGQAPRGLIGGTKLLAPGT |
| | PARFSGSLLGGKAALTLSGVQPEDEAEYYCVLWYSNSWVFGGGTKLTVL |
| Exemplary anti-CD3 scFv | EVQLVESGGGLVQPGGSLKLSCAASGFTFNTYAMNWVRQAPGKGLEWVA |
| (SEQ ID NO: 78) | RIRSKYNNYATYYKDSVKDRFTISRDDSKNTAYLQMNNLKTEDTAVYYCVRH |
| | GNFGNSPISYWAYWGQGTLVTVSSGGGGSGGGGSGGGGSQTVVTQEPSL |
| | TVSPGGTVTLTCGSSTGAVVSGNYPNWVQQKPGQAPRGLIGGTEFLAPGTP |
| | ARFSGSLLGGKAALTLSGVQPEDEAEYYCVLWYSNRWVFGGGTKLTVL |
| Exemplary anti-CD3 scFv | EVQLVESGGGLVQPGGSLKLSCAASGFTYNKYAMNWVRQAPGKGLEWVA |
| (SEQ ID NO: 79) | RIRSKYNNYATYYADEVKDRFTISRDDSKNTAYLQMNNLKTEDTAVYYCVRH |
| | GNFGNSPISYWAYWGQGTLVTVSSGGGGSGGGGSGGGGSQTVVTQEPSL |
| | TVSPGGTVTLTCGSSKGAVTSGNYPNWVQQKPGQAPRGLIGGTKELAPGTP |
| | ARFSGSLLGGKAALTLSGVQPEDEAEYYCTLWYSNRWVFGGGTKLTVL |
| Exemplary anti-CD3 scFv | EVQLVESGGGLVQPGGSLKLSCAASGNTFNKYAMNWVRQAPGKGLEWVA |
| (SEQ ID NO: 80) | RIRSKYNNYETYYADSVKDRFTISRDDSKNTAYLQMNNLKTEDTAVYYCVRH |
| | TNFGNSYISYWAYWGQGTLVTVSSGGGGSGGGGSGGGGSQTVVTQEPSLT |
| | VSPGGTVTLTCGSSTGAVTSGYYPNWVQQKPGQAPRGLIGGTYFLAPGTPA |
| | RFSGSLLGGKAALTLSGVQPEDEAEYYCVLWYSNRWVFGGGTKLTVL |
| Exemplary anti-CD3 scFv | EVQLVESGGGLVQPGGSLKLSCAASGFTFNNYAMNWVRQAPGKGLEWVA |
| (SEQ ID NO: 81) | RIRSKYNNYATYYADAVKDRFTISRDDSKNTAYLQMNNLKTEDTAVYYCVRH |
| | GNFGNSQISYWAYWGQGTLVTVSSGGGGSGGGGSGGGGSQTVVTQEPSL |
| | TVSPGGTVTLTCGSSTGAVTDGNYPNWVQQKPGQAPRGLIGGIKFLAPGTP |
| | ARFSGSLLGGKAALTLSGVQPEDEAEYYCVLWYSNRWVFGGGTKLTVL |
| Exemplary anti-CD3 scFv | EVQLVESGGGLVQPGGSLKLSCAASGFTFNKYAVNWVRQAPGKGLEWVAR |
| (SEQ ID NO: 82) | IRSKYNNYATYYADSVKDRFTISRDDSKNTAYLQMNNLKTEDTAVYYCVRHG |
| | NFGNSYISYWAYWGQGTLVTVSSGGGGSGGGGSGGGGSQTVVTQEPSLT |
| | VSPGGTVTLTCGESTGAVTSGNYPNWVQQKPGQAPRGLIGGTKILAPGTPA |
| | RFSGSLLGGKAALTLSGVQPEDEAEYYCVLWYSNRWVFGGGTKLTVL |
| Exemplary anti-CD3 scFv | EVQLVESGGGLVQPGGSLKLSCAASGFTFNKYPMNWVRQAPGKGLEWVA |
| (SEQ ID NO: 83) | RIRSKYNNYATYYADSVKDRFTISRDDSKNTAYLQMNNLKNEDTAVYYCVRH |
| | GNFNNSYISYWAYWGQGTLVTVSSGGGGSGGGGSGGGGSQTVVTQEPSL |
| | TVSPGGTVTLTCGSSTGAVTKGNYPNWVQQKPGQAPRGLIGGTKMLAPGT |
| | PARFSGSLLGGKAALTLSGVQPEDEAEYYCALWYSNRWVFGGGTKLTVL |
| Exemplary anti-CD3 scFv | EVQLVESGGGLVQPGGSLKLSCAASGFTFNGYAMNWVRQAPGKGLEWVA |
| (SEQ ID NO: 84) | RIRSKYNNYATYYADEVKDRFTISRDDSKNTAYLQMNNLKTEDTAVYYCVRH |
| | GNFGNSPISYWAYWGQGTLVTVSSGGGGSGGGGSGGGGSQTVVTQEPSL |
| | TVSPGGTVTLTCGSSTGAVVSGNYPNWVQQKPGQAPRGLIGGTEFLAPGTP |
| | ARFSGSLLGGKAALTLSGVQPEDEAEYYCVLWYSNRWVFGGGTKLTVL |
| Exemplary anti-CD3 scFv | EVQLVESGGGLVQPGGSLKLSCAASGNTFNKYAMNWVRQAPGKGLEWVA |
| (SEQ ID NO: 85) | RIRSKYNNYATYYADSVKDRFTISRDDSKNTAYLQMNNLKTEDTAVYYCVRH |
| | GNFGDSYISYWAYWGQGTLVTVSSGGGGSGGGGSGGGGSQTVVTQEPSL |
| | TVSPGGTVTLTCGSSTGAVTHGNYPNWVQQKPGQAPRGLIGGTKVLAPGT |
| | PARFSGSLLGGKAALTLSGVQPEDEAEYYCVLWYSNRWVFGGGTKLTVL |
| Exemplary anti-CD3 scFv | EVQLVESGGGLVQPGGSLKLSCAASGFTFNNYAMNWVRQAPGKGLEWVA |
| (SEQ ID NO: 86) | RIRSGYNNYATYYADSVKDRFTISRDDSKNTAYLQMNNLKTEDTAVYYCVRH |
| | GNFGNSYISYWAYWGQGTLVTVSSGGGGSGGGGSGGGGSQTVVTQEPSL |
| | TVSPGGTVTLTCGSYTGAVTSGNYPNWVQQKPGQAPRGLIGGTKFNAPGT |
| | PARFSGSLLGGKAALTLSGVQPEDEAEYYCVLWYANRWVFGGGTKLTVL |
| Exemplary anti-CD3 scFv | EVQLVESGGGLVQPGGSLKLSCAASGFEFNKYAMNWVRQAPGKGLEWVA |
| (SEQ ID NO: 87) | RIRSKYNNYETYYADSVKDRFTISRDDSKNTAYLQMNNLKTEDTAVYYCVRH |
| | GNFGNSLISYWAYWGQGTLVTVSSGGGGSGGGGSGGGGSQTVVTQEPSL |
| | TVSPGGTVTLTCGSSSGAVTSGNYPNWVQQKPGQAPRGLIGGTKFGAPGT |
| | PARFSGSLLGGKAALTLSGVQPEDEAEYYCVLWYSNRWVFGGGTKLTVL |
| Exemplary anti-CD3 scFv | EVQLVESGGGLVQPGGSLKLSCAASGFTFNKYAMNWVRQAPGKGLEWVA |
| (SEQ ID NO: 88) | RIRSKYNNYATYYADSVKDRFTISRDDSKNTAYLQMNNLKTEDTAVYYCVRH |
| | GNFGNSYISYWAYWGQGTLVTVSSGGGGSGGGGSGGGGSQTVVTQEPSL |
| | TVSPGGTVTLTCGSSTGAVTSGNYPNWVQQKPGQAPRGLIGGTKFLAPGTP |
| | ARFSGSLLGGKAALTLSGVQPEDEAEYYCVLWYSNRWVFGGGTKLTVL |
| Exemplary anti-CD3 scFv | EVQLVESGGGLVQPGGSLKLSCAASGFTFNKYALNWVRQAPGKGLEWVARI |
| (SEQ ID NO: 89) | RSKYNNYATEYADSVKDRFTISRDDSKNTAYLQMNNLKTEDTAVYYCVRHG |
| | NFGNSPISYWAYWGQGTLVTVSSGGGGSGGGGSGGGGSQTVVTQEPSLT |
| | VSPGGTVTLTCGSSTGAVTSGNYPNWVQQKPGQAPRGLIGGTNFLAPGTP |
| | ERFSGSLLGGKAALTLSGVQPEDEAEYYCVLWYSNRWAFGGGTKLTVL |
| Exemplary anti-CD3 scFv | EVQLVESGGGLVQPGGSLKLSCAASGFTFNEYAMNWVRQAPGKGLEWVA |
| (SEQ ID NO: 90) | RIRSKYNNYATYYADDVKDRFTISRDDSKNTAYLQMNNLKTEDTAVYYCVRH |
| | GNFGNSGISYWAYWGQGTLVTVSSGGGGSGGGGSGGGGSQTVVTQEPSL |
| | TVSPGGTVTLTCGSSTGAVTVGNYPNWVQQKPGQAPRGLIGGTEFLAPGTP |
| | ARFSGSLLGGKAALTLSGVQPEDEAEYYCVLWYSNRWVFGGGTKLTVL |
| Exemplary anti-target | EVQLVESGGGLVQPGRSLTLSCAASTNIFSISPMGWYRQAPGKQRELVAAIH |
| (BCMA) | GFSTLYADSVKGRFTISRDNAKNSIYLQMNSLRPEDTALYYCNKVPWGDYHP |
| (SEQ ID NO: 91) | RNVYWGQGTQVTVSS |
| Exemplary anti-target | EVQLVESGGGLVQPGRSLTLSCAASTDIFSISPMGWYRQAPGKQRELVAAIH |
| (BCMA) | GGSTLYADSVKGRFTISRDNAKNSIYLQMNSLRPEDTALYYCNKVPWGDYH |
| (SEQ ID NO: 92) | PRNVAWGQGTQVTVSS |
| Exemplary anti-target | EVQLVESGGGLVQPGRSLTLSCAASTNDFSISPMGWYRQAPGKQRELVAAI |
| (BCMA) | HGGSTLYADSVKGRFTISRDNAKNSIYLQMNSLRPEDTALYYCNKVPWGDY |
| (SEQ ID NO: 93) | HPRNVAWGQGTQVTVSS |
| Exemplary anti-target | EVQLVESGGGLVQPGRSLTLSCAASTNIFSKSPMGWYRQAPGKQRELVAAI |
| (BCMA) | HGKSTLYADSVKGRFTISRDNAKNSIYLQMNSLRPEDTALYYCNKVPWGDY |
| (SEQ ID NO: 94) | HPRNVVWGQGTQVTVSS |
| Exemplary anti-target | EVQLVESGGGLVQPGRSLTLSCAASTNDESISPMGWYRQAPGKQRELVAAI |
| (BCMA) | HGKSTLYADSVKGRFTISRDNAKNSIYLQMNSLRPEDTALYYCNKVPWGDY |
| (SEQ ID NO: 95) | HPRNVKWGQGTQVTVSS |
| Exemplary anti-target | EVQLVESGGGLVQPGRSLTLSCAASTNQFSISPMGWYRQAPGKQRELVAAI |
| (BCMA) | HGKSTLYADSVKGRFTISRDNAKNSIYLQMNSLRPEDTALYYCNKVPWGDY |
| (SEQ ID NO: 96) | HPRNVVWGQGTQVTVSS |
| Exemplary anti-target | EVQLVESGGGLVQPGRSLTLSCAASTNIFSSSPMGWYRQAPGKQRELVAAI |
| (BCMA) | NGFSTLYADSVKGRFTISRDNAKNSIYLQMNSLRPEDTALYYCNKVPWGDYH |
| (SEQ ID NO: 97) | PRNVHWGQGTQVTVSS |
| Exemplary anti-target | EVQLVESGGGLVQPGRSLTLSCAASTNIFSSSPMGWYRQAPGKQRELVAAI |
| (BCMA) | HGFSTLYADSVKGRFTISRDNAKNSIYLQMNSLRPEDTALYYCNKVPWGDYH |
| (SEQ ID NO: 98) | PRNVYWGQGTQVTVSS |
| Exemplary anti-target | EVQLVESGGGLVQPGRSLTLSCAASTNIFSTSPMGWYRQAPGKQRELVAAI |
| (BCMA) | HGFQTLYADSVKGRFTISRDNAKNSIYLQMNSLRPEDTALYYCNKVPWGDY |
| (SEQ ID NO: 99) | HPRNVVWGQGTQVTVSS |
| Exemplary anti-target | EVQLVESGGGLVQPGRSLTLSCAASTNIFSRSPMGWYRQAPGKQRELVAAI |
| (BCMA) | HGFETLYADSVKGRFTISRDNAKNSIYLQMNSLRPEDTALYYCNKVPWGDYH |
| (SEQ ID NO: 100) | PRNVLWGQGTQVTVSS |
| Exemplary anti-target | EVQLVESGGGLVQPGRSLTLSCAASTNIFSESPMGWYRQAPGKQRELVAAI |
| (BCMA) | HGFTTLYADSVKGRFTISRDNAKNSIYLQMNSLRPEDTALYYCNKVPWGDYH |
| (SEQ ID NO: 101) | PRNVTWGQGTQVTVSS |
| Exemplary anti-target | EVQLVESGGGLVQPGRSLTLSCAASTNIFSDSPMGWYRQAPGKQRELVAAI |
| (BCMA) | HGFSTLYADSVKGRFTISRDNAKNSIYLQMNSLRPEDTALYYCNKVPWGDYH |
| (SEQ ID NO: 102) | PRNVAWGQGTQVTVSS |
| Exemplary anti-target | EVQLVESGGGLVQPGRSLTLSCAASTNIFSNSPMGWYRQAPGKQRELVAAI |
| (BCMA) | HGGSTLYADSVKGRFTISRDNAKNSIYLQMNSLRPEDTALYYCNKVPWGDY |
| (SEQ ID NO: 103) | HPRNVHWGQGTQVTVSS |
| Exemplary anti-target | EVQLVESGGGLVQPGRSLTLSCAASTNIFSNSPMGWYRQAPGKQRELVAAI |
| (BCMA) | HGRSTLYADSVKGRFTISRDNAKNSIYLQMNSLRPEDTALYYCNKVPWGDY |
| (SEQ ID NO: 104) | HPRNVMWGQGTQVTVSS |
| Exemplary anti-target | EVQLVESGGGLVQPGRSLTLSCAASTNIFSNSPMGWYRQAPGKQRELVAAI |
| (BCMA) | HGPSTLYADSVKGRFTISRDNAKNSIYLQMNSLRPEDTALYYCNKVPWGDY |
| (SEQ ID NO: 105) | HPRNVTWGQGTQVTVSS |
| Exemplary anti-target | EVQLVESGGGLVQPGRSLTLSCAASTNIFSNSPMGWYRQAPGKQRELVAAI |
| (BCMA) | HGDSTLYADSVKGRFTISRDNAKNSIYLQMNSLRPEDTALYYCNKVPWGDY |
| (SEQ ID NO: 106) | HPRNVRWGQGTQVTVSS |
| Exemplary anti-target | EVQLVESGGGLVQPGRSLTLSCAASTNIFSRSPMGWYRQAPGKQRELVAAI |
| (BCMA) | HGDSTLYADSVKGRFTISRDNAKNSIYLQMNSLRPEDTALYYCNKVPWGDY |
| (SEQ ID NO: 107) | HPRNVTWGQGTQVTVSS |
| Exemplary anti-target | EVQLVESGGGLVQPGRSLTLSCAASTNIFSKSPMGWYRQAPGKQRELVAAI |
| (BCMA) | HGQSTLYADSVKGRFTISRDNAKNSIYLQMNSLRPEDTALYYCNKVPWGDY |
| (SEQ ID NO: 108) | HPRNVTWGQGTQVTVSS |
| Exemplary anti-target | EVQLVESGGGLVQPGRSLTLSCAASTNIFSSSPMGWYRQAPGKQRELVAAI |
| (BCMA) | HGHSTLYADSVKGRFTISRDNAKNSIYLQMNSLRPEDTALYYCNKVPWGDY |
| (SEQ ID NO: 109) | HPRNVTWGQGTQVTVSS |
| Exemplary anti-target | EVQLVESGGGLVQPGRSLTLSCAASTNIFSSSPMGWYRQAPGKQRELVAAI |
| (BCMA) | HGESTLYADSVKGRFTISRDNAKNSIYLQMNSLRPEDTALYYCNKVPWGDYH |
| (SEQ ID NO: 110) | PRKVYWGQGTQVTVSS |
| Exemplary anti-target | EVQLVESGGGLVQPGRSLTLSCAASTNIFSRSPMGWYRQAPGKQRELVAAI |
| (BCMA) | HGNSTLYADSVKGRFTISRDNAKNSIYLQMNSLRPEDTALYYCNKVPWGIYH |
| (SEQ ID NO: 111) | PRNVYWGQGTQVTVSS |
| Exemplary anti-target | EVQLVESGGGLVQPGRSLTLSCAASTNIFSESPMGWYRQAPGKQRELVAAI |
| (BCMA) | HGNSTLYADSVKGRFTISRDNAKNSIYLQMNSLRPEDTALYYCNKVPWGTY |
| (SEQ ID NO: 112) | HPRNVYWGQGTQVTVSS |
| Exemplary anti-target | EVQLVESGGGLVQPGRSLTLSCAASTNIFSVSPMGWYRQAPGKQRELVAAI |
| (BCMA) | HGNSTLYADSVKGRFTISRDNAKNSIYLQMNSLRPEDTALYYCNKVPWGKY |
| (SEQ ID NO: 113) | HPRNVYWGQGTQVTVSS |
| Exemplary anti-target | EVQLVESGGGLVQPGRSLTLSCAASTNIFSVSPMGWYRQAPGKQRELVAAI |
| (BCMA) | HGNSTLYADSVKGRFTISRDNAKNSIYLQMNSLRPEDTALYYCNKVPWGDY |
| (SEQ ID NO: 114) | HPRNVYWGQGTQVTVSS |
| Exemplary anti-target | EVQLVESGGGLVQPGRSLTLSCAASTNIFSKSPMGWYRQAPGKQRELVAAI |
| (BCMA) | HGNSTLYADSVKGRFTISRDNAKNSIYLQMNSLRPEDTALYYCNKVPWGDY |
| (SEQ ID NO: 115) | HPREVYWGQGTQVTVSS |
| Exemplary anti-target | EVQLVESGGGLVQPGRSLTLSCAASTNIFSDSPMGWYRQAPGKQRELVAAI |
| (BCMA) | HGTSTLYADSVKGRFTISRDNAKNSIYLQMNSLRPEDTALYYCNKVPWGDYH |
| (SEQ ID NO: 116) | PRNVYWGQGTQVTVSS |
| Exemplary anti-target | EVQLVESGGGLVQPGRSLTLSCAASTNIFSRSPMGWYRQAPGKQRELVAAI |
| (BCMA) | HGTSTLYADSVKGRFTISRDNAKNSIYLQMNSLRPEDTALYYCNKVPWGKYH |
| (SEQ ID NO: 117) | PRNVYWGQGTQVTVSS |
| Exemplary anti-target | EVQLVESGGGLVQPGRSLTLSCAASTNIFSHSPMGWYRQAPGKQRELVAAI |
| (BCMA) | HGTSTLYADSVKGRFTISRDNAKNSIYLQMNSLRPEDTALYYCNKVPWGRYH |
| (SEQ ID NO: 118) | PRNVYWGQGTQVTVSS |
| Exemplary anti-target | EVQLVESGGGLVQPGRSLTLSCAASTNIFSTSPMGWYRQAPGKQRELVAAI |
| (BCMA) | HGTSTLYADSVKGRFTISRDNAKNSIYLQMNSLRPEDTALYYCNKVPWGDYH |
| (SEQ ID NO: 119) | PGNVYWGQGTQVTVSS |
| Exemplary anti-target | EVQLVESGGGLVQPGRSLTLSCAASTNIFSTSPMGWYRQAPGKQRELVAAI |
| (BCMA) | HGTSTLYADSVKGRFTISRDNAKNSIYLQMNSLRPEDTALYYCNKVPWGDYH |
| (SEQ ID NO: 120) | PRNVQWGQGTQVTVSS |
| Exemplary anti-target | EVQLVESGGGLVQPGRSLTLSCAASTNIFSLSPMGWYRQAPGKQRELVAAI |
| (BCMA) | HGDSTLYADSVKGRFTISRDNAKNSIYLQMNSLRPEDTALYYCNKVPWGDY |
| (SEQ ID NO: 121) | HPRNVYWGQGTQVTVSS |
| Exemplary anti-target | EVQLVESGGGLVQPGRSLTLSCAASTNIFSISPGGWYRQAPGKQRELVAAIH |
| (BCMA) | GSSTLYADSVKGRFTISRDNAKNSIYLQMNSLRPEDTALYYCNKVPWGDYHP |
| (SEQ ID NO: 122) | RNVYWGQGTQVTVSS |
| Exemplary anti-target | EVQLVESGGGLVQPGRSLTLSCAASTNHFSISPMGWYRQAPGKQRELVAAI |
| (BCMA) | HGSSTLYADSVKGRFTISRDNAKNSIYLQMNSLRPEDTALYYCNKVPWGDYH |
| (SEQ ID NO: 123) | PRVVYWGQGTQVTVSS |
| Exemplary anti-target | EVQLVESGGGLVQPGRSLTLSCAASTNIFSASPMGWYRQAPGKQRELVAAI |
| (BCMA) | HGSSTLYADSVKGRFTISRDNAKNSIYLQMNSLRPEDTALYYCNKVPWGDYH |
| (SEQ ID NO: 124) | PRNVNWGQGTQVTVSS |
| Exemplary anti-target | EVQLVESGGGLVQPGRSLTLSCAASTNIFSASPMGWYRQAPGKQRELVAAI |
| (BCMA) | HGSSTLYADSVKGRFTISRDNAKNSIYLQMNSLRPEDTALYYCNKVPWGRYH |
| (SEQ ID NO: 125) | PRNVYWGQGTQVTVSS |
| Exemplary anti-target | EVQLVESGGGLVQPGRSLTLSCAASTNISSISPMGWYRQAPGKQRELVAAIH |
| (BCMA) | GTSTLYADSVKGRFTISRDNAKNSIYLQMNSLRPEDTALYYCNKVPWGDYHP |
| (SEQ ID NO: 126) | GNVYWGQGTQVTVSS |
| Exemplary anti-target | EVQLVESGGGLVQPGRSLTLSCAASTNIESISPMGWYRQAPGKQRELVAAIH |
| (BCMA) | GTSTLYADSVKGRFTISRDNAKNSIYLQMNSLRPEDTALYYCNKVPWGDYHP |
| (SEQ ID NO: 127) | GNVYWGQGTQVTVSS |
| Exemplary anti-target | EVQLVESGGGLVQPGRSLTLSCAASTNIFSISPYGWYRQAPGKQRELVAAIH |
| (BCMA) | GTSTLYADSVKGRFTISRDNAKNSIYLQMNSLRPEDTALYYCNKVPWGDYHP |
| (SEQ ID NO: 128) | GNVYWGQGTQVTVSS |
| Exemplary anti-target | EVQLVESGGGLVQPGRSLTLSCAASTNIASISPMGWYRQAPGKQRELVAAIH |
| (BCMA) | GTSTLYADSVKGRFTISRDNAKNSIYLQMNSLRPEDTALYYCNKVPWGDYHP |
| (SEQ ID NO: 129) | GNVYWGQGTQVTVSS |
| Exemplary anti-target | EVQLVESGGGLVQPGRSLTLSCAASTNIASISPMGWYRQAPGKQRELVAAIH |
| (BCMA) | GKSTLYADSVKGRFTISRDNAKNSIYLQMNSLRPEDTALYYCNKVPWGDYHP |
| (SEQ ID NO: 130) | GNVYWGQGTQVTVSS |
| Exemplary anti-target | EVQLVESGGGLVQPGRSLTLSCAASTNIASISPMGWYRQAPGKQRELVAAIH |
| (BCMA) | GSSTLYADSVKGRFTISRDNAKNSIYLQMNSLRPEDTALYYCNKVPWGDYHP |
| (SEQ ID NO: 131) | GNVYWGQGTQVTVSS |
| Exemplary anti-target | EVQLVESGGGLVQPGRSLTLSCAASTNITSISPMGWYRQAPGKQRELVAAIH |
| (BCMA) | GDSTLYADSVKGRFTISRDNAKNSIYLQMNSLRPEDTALYYCNKVPWGDYH |
| (SEQ ID NO: 132) | PGNVYWGQGTQVTVSS |
| Exemplary anti-target | EVQLVESGGGLVQPGRSLTLSCAASTNIMSISPMGWYRQAPGKQRELVAAI |
| (BCMA) | HGNSTLYADSVKGRFTISRDNAKNSIYLQMNSLRPEDTALYYCNKVPWGDY |
| (SEQ ID NO: 133) | HPGNVYWGQGTQVTVSS |
| Exemplary anti-target | EVQLVESGGGLVQPGRSLTLSCAASTNITSISPMGWYRQAPGKQRELVAAIH |
| (BCMA) | GNSTLYADSVKGRFTISRDNAKNSIYLQMNSLRPEDTALYYCNKVPWGDYH |
| (SEQ ID NO: 134) | PGNVYWGQGTQVTVSS |
| Exemplary anti-target | EVQLVESGGGLVQPGRSLTLSCAASTNIVSISPMGWYRQAPGKQRELVAAIH |
| (BCMA) | GHSTLYADSVKGRFTISRDNAKNSIYLQMNSLRPEDTALYYCNKVPWGDYH |
| (SEQ ID NO: 135) | PGNVYWGQGTQVTVSS |
| Exemplary anti-target | EVQLVESGGGLVQPGRSLTLSCAASTNIVSISPMGWYRQAPGKQRELVAAIH |
| (BCMA) | GKSTLYADSVKGRFTISRDNAKNSIYLQMNSLRPEDTALYYCNKVPWGDYHP |
| (SEQ ID NO: 136) | GNVYWGQGTQVTVSS |
| Exemplary anti-target | EVQLVESGGGLVQPGRSLTLSCAASTNVVSISPMGWYRQAPGKQRELVAAI |
| (BCMA) | HGKSTLYADSVKGRFTISRDNAKNSIYLQMNSLRPEDTALYYCNKVPWGDY |
| (SEQ ID NO: 137) | HPNNVYWGQGTQVTVSS |
| Exemplary anti-target | EVQLVESGGGLVQPGRSLTLSCAASTNIISISPMGWYRQAPGKQRELVAAIH |
| (BCMA) | GASTLYADSVKGRFTISRDNAKNSIYLQMNSLRPEDTALYYCNKVPWGDYHP |
| (SEQ ID NO: 138) | GNVYWGQGTQVTVSS |
| Exemplary anti-target | EVQLVESGGGLVQPGRSLTLSCAASTNIFSITPMGWYRQAPGKQRELVAAIH |
| (BCMA) | GASTLYADSVKGRFTISRDNAKNSIYLQMNSLRPEDTALYYCNKVPWGDYHP |
| (SEQ ID NO: 139) | GNVYWGQGTQVTVSS |
| Exemplary anti-target | EVQLVESGGGLVQPGRSLTLSCAASTNITSISPMGWYRQAPGKQRELVAAIH |
| (BCMA) | GFETLYADSVKGRFTISRDNAKNSIYLQMNSLRPEDTALYYCNKVPWGDYHP |
| (SEQ ID NO: 140) | GNVYWGQGTQVTVSS |
| Exemplary anti-target | EVQLVESGGGLVQPGRSLTLSCAASTNIQSISPMGWYRQAPGKQRELVAAI |
| (BCMA) | HGFETLYADSVKGRFTISRDNAKNSIYLQMNSLRPEDTALYYCNKVPWGDYH |
| (SEQ ID NO: 141) | PGNVYWGQGTQVTVSS |
| Exemplary anti-target | EVQLVESGGGLVQPGRSLTLSCAASTSDFSISPMGWYRQAPGKQRELVAAI |
| (BCMA) | HGFETLYADSVKGRFTISRDNAKNSIYLQMNSLRPEDTALYYCNKVPWGDYH |
| (SEQ ID NO: 142) | PGNVYWGQGTQVTVSS |
| Exemplary anti-target | EVQLVESGGGLVQPGRSLTLSCAASTNIDSISPMGWYRQAPGKQRELVAAIH |
| (BCMA) | GFQTLYADSVKGRFTISRDNAKNSIYLQMNSLRPEDTALYYCNKVPWGDYH |
| (SEQ ID NO: 143) | PGNVYWGQGTQVTVSS |
| Exemplary anti-target | EVQLVESGGGLVQPGRSLTLSCAASTNIMSISPMGWYRQAPGKQRELVAAI |
| (BCMA) | HGFSTVYADSVKGRFTISRDNAKNSIYLQMNSLRPEDTALYYCNKVPWGDY |
| (SEQ ID NO: 144) | HPGNVYWGQGTQVTVSS |
| Exemplary anti-target | EVQLVESGGGLVQPGRSLTLSCAASTNIESISPMGWYRQAPGKQRELVAAIH |
| (BCMA) | GFSTLYADSVKGRFTISRDNAKNSIYLQMNSLRPEDTALYYCNKVPWGDYHP |
| (SEQ ID NO: 145) | GNVYWGQGTQVTVSS |
| Exemplary anti-target | EVQLVESGGGLVQPGRSLTLSCAASTNIFSSSPMGWYRQAPGKQRELVAAI |
| (BCMA) | HGFKTLYADSVKGRFTISRDNAKNSIYLQMNSLRPEDTARYYCNKVPWGDY |
| (SEQ ID NO: 146) | HPGNVYWGQGTQVTVSS |
| Exemplary anti-target | EVQLVESGGGLVQPGRSLTLSCAASTNIFSSSPMGWYRQAPGKQRELVAAI |
| (BCMA) | HGFSTLYADSVKGRFTISRDNAKNSIYLQMNSLRPEDTALYYCNKVPWGDYH |
| (SEQ ID NO: 147) | PGNVYWGQGTQVTVSS |
| Exemplary anti-target | EVQLVESGGGLVQPGRSLTLSCAASTNIFSNSPMGWYRQAPGKQRELVAAI |
| (BCMA) | HGFSTLYADSVKGRFTISRDNAKNSIYLQMNSLRPEDTALYYCNKVPWGDYH |
| (SEQ ID NO: 148) | PGNVYWGQGTQVTVSS |
| Exemplary anti-target | EVQLVESGGGLVQPGRSLTLSCAASTNIFSTSPMGWYRQAPGKQRELVAAI |
| (BCMA) | HGFSTIYADSVKGRFTISRDNAKNSIYLQMNSLRPEDTALYYCNKVPWGDYH |
| (SEQ ID NO: 149) | PGNVYWGQGTQVTVSS |
| Exemplary anti-target | EVQLVESGGGLVQPGRSLTLSCAASTNIFSTSPMGWYRQAPGKQRELVAAI |
| (BCMA) | HGFSTIYADSVKGRFTISRDNAKNSIYLQMNSLRPEDTALYYCNKVPWGDYH |
| (SEQ ID NO: 150) | PLNVYWGQGTQVTVSS |
| Exemplary anti-target | EVQLVESGGGLVQPGRSLTLSCVASTNIFSTSPMGWYRQAPGKQRELVAAI |
| (BCMA) | HGFSTLYADSVKGRFTISRDNAKNSIYLQMNSLRPEDTALYYCNKVPWGDYH |
| (SEQ ID NO: 151) | PGNVYWGQGTQVTVSS |
| Exemplary anti-target | EVQLVESGGGLVQPGRSLTLSCAASTNIFSDSPMGWYRQAPGKQRELVAAI |
| (BCMA) | HGFSTFYADSVKGRFTISRDNAKNSIYLQMNSLRPEDTALYYCNKVPWGDY |
| (SEQ ID NO: 152) | HPGNVYWGQGTQVTVSS |
| Exemplary anti-target | EVQLVESGGGLVQPGRSLTLSCAASTNIFSQSPMGWYRQAPGKQRELVAAI |
| (BCMA) | HGDSTLYADSVKGRFTISRDNAKNSIYLQMNSLRPEDTALYYCNKVPWGDY |
| (SEQ ID NO: 153) | HPGNVCWGQGTQVTVSS |
| Exemplary anti-target | EVQLVESGGGLVQPGRSLTLSCAASTNIFSQSPMGWYRQAPGKQRELVAAI |
| (BCMA) | HGKSTLYADSVKGRFTISRDNAKNSIYLQMNSLRPEDTALYYCNKVPWGDY |
| (SEQ ID NO: 154) | HPSNVYWGKGTQVTVSS |
| Exemplary anti-target | EVQLVESGGGLVQPGRSLTLSCAASTNIFSRSPMGWYRQAPGKQRELVAAI |
| (BCMA) | HGESTLYADSVKGRFTISRDNAKNSIYLQMNSLRPEDTALYYCNKVPWGRYH |
| (SEQ ID NO: 155) | PGNVYWGQGTQVTVSS |
| Exemplary anti-target | EVQLVESGGGLVQPGRSLTLSCAASTNIFSRSPMGWYRQAPGKQRELVAAI |
| (BCMA) | HGISTLYADSVKGRFTISRDNAKNSIYLQMNSLRPEDTALYYCNKVPWGDYH |
| (SEQ ID NO: 156) | PGNVYWGQGTQVTVSS |
| Exemplary anti-target | EVQLVESGGGLVQPGRSLTLSCAASTNIFSRSPMGWYRQAPGKQRELVAAI |
| (BCMA) | HGSSTLYADSVKGRFTISRDNAKNSIYLQMNSLRPEDTALYYCNKVPWGDYH |
| (SEQ ID NO: 157) | PGNVYWGQGTQVTVSS |
| Exemplary anti-target | EVQLVESGGGLVQPGRSLTLSCAASTNIFSGSPMGWYRQAPGKQRELVAAI |
| (BCMA) | HGNSTLYADSVKGRFTISRDNAKNSIYLQMNSLRPEDTALYYCNKVPWGDY |
| (SEQ ID NO: 158) | HPGNVYWGQGTQVTVSS |
| Exemplary anti-target | EVQLVESGGGLVQPGRSLTLSCAASSNIFSISPMGWYRQAPGKQRELVAAIH |
| (BCMA) | GSSTLYADSVKGRFTISRDNAKNSIYLQMNSLRPEDTALYYCNKVPWGDYHP |
| (SEQ ID NO: 159) | GNVYWGQGTQVTVSS |
| Exemplary anti-target | EVQLVESGGGLVQPGRSLTLSCAASTNIFSIYPMGWYRQAPGKQRELVAAIH |
| (BCMA) | GSSTLYADSVKGRFTISRDNAKNSIYLQMNSLRPEDTALYYCNKVPWGDYHP |
| (SEQ ID NO: 160) | KNVYWGQGTQVTVSS |
| Exemplary anti-target | EVQLVESGGGLVQPGRSLTLSCAASTNIFSKSPMGWYRQAPGKQRELVAAI |
| (BCMA) | HGSSTLYADSVKGRFTISRDNAKNSIYLQMNSLRPEDTALYYCNKVPWGDYH |
| (SEQ ID NO: 161) | PGNVYWGQGTQVTVSS |
| Exemplary anti-target | EVQLVESGGGLVQPGRSLTLSCAASTNIFSKSPMGWYRQAPGKQRELVAAI |
| (BCMA) | HGSSTLYADSVKGRFTISRDNAKNSIYLQMNSLRPEDTALYYCNKVPWGDYH |
| (SEQ ID NO: 162) | PRNVYWGQGTQVTVSS |
| Exemplary anti-target | EVQLVESGGGLVQPGRSLTLSCAASTNEFSISPMGWYRQAPGKQRELVAAI |
| (BCMA) | HGLSTLYADSVKGRFTISRDNAKNSIYLQMNSLRPEDTALYYCNKVPWGAYH |
| (SEQ ID NO: 163) | PRNVYWGQGTQVTVSS |
| Exemplary anti-target | EVQLVESGGGLVQPGRSLTLSCAASTNEFSISPMGWYRQAPGKQRELVAAI |
| (BCMA) | HGESTLYADSVKGRFTISRDNAKNSIYLQMNSLRPEDTALYYCNKVPWGDYH |
| (SEQ ID NO: 164) | PGNVYWGQGTQVTVSS |
| Exemplary anti-target | EVQLVESGGGLVQPGRSLTLSCAASTNIPSISPMGWYRQAPGKQRELVAAIH |
| (BCMA) | GESTLYADSVKGRFTISRDNAKNSIYLQMNSLRPEDTALYYCNKVPWGDYHP |
| (SEQ ID NO: 165) | RNVAWGQGTQVTVSS |
| Exemplary anti-target | EVQLVESGGGLVQPGRSLTLSCAASTNIPSISPMGWYRQAPGKQRELVAAIH |
| (BCMA) | GASTLYADSVKGRFTISRDNAKNSIYLQMNSLRPEDTALYYCNKVPWGDYHP |
| (SEQ ID NO: 166) | RNVAWGQGTQVTVSS |
| Exemplary anti-target | EVQLVESGGGLVQPGRSLTLSCAASTNIPSISPMGWYRQAPGKQRELVAAIH |
| (BCMA) | GESTLYADSVKGRFTISRDNAKNSIYLQMNSLRPEDTALYYCNKVPWGDYHP |
| (SEQ ID NO: 167) | RNVYWGQGTQVTVSS |
| Exemplary anti-target | EVQLVESGGGLVQPGRSLTLSCAASTNIPSISPMGWYRQAPGKQRELVAAIH |
| (BCMA) | GDSTLYADSVKGRFTISRDNAKNSIYLQMNSLRPEDTALYYCNKVPWGDYH |
| (SEQ ID NO: 168) | PRNVYWGQGTQVTVSS |
| Exemplary anti-target | EVQLVESGGGLVQPGRSLTLSCAASTNITSISPMGWYRQAPGKQRELVAAIH |
| (BCMA) | GVSTLYADSVKGRFTISRDNAKNSIYLQMNSLRPEDTALYYCNKVPWGDYHP |
| (SEQ ID NO: 169) | RNVQWGQGTQVTVSS |
| Exemplary anti-target | EVQLVESGGGLVQPGRSLTLSCAASTNIPSISPMGWYRQAPGKQRELVAAIH |
| (BCMA) | GQSTLYADSVKGRFTISRDNAKNSIYLQMNSLRPEDTALYYCNKVPWGDYH |
| (SEQ ID NO: 170) | PRNVQWGQGTQVTVSS |
| Exemplary anti-target | EVQLVESGGGLVQPGRSLTLSCAASTNIVSISPMGWYRQAPGKQRELVAAIH |
| (BCMA) | GDSTLYADSVKGRFTISRDNAKNSIYLQMNSLRPEDTALYYCNKVPWGDYH |
| (SEQ ID NO: 171) | PRNVSWGQGTQVTVSS |
| Exemplary anti-target | EVQLVESGGGLVQPGRSLTLSCAASSNIFSISPMGWYRQAPGKQRELVAAIH |
| (BCMA) | GESTLYADSVKGRFTISRDNAKNSIYLQMNSLRPEDTALYYCNKVPWGDYHP |
| (SEQ ID NO: 172) | RNVTWGQGTQVTVSS |
| Exemplary anti-target | EVQLVESGGGLVQPGRSLTLSCAASTNIDSISPMGWYRQAPGKQRELVAAIH |
| (BCMA) | GESTLYADSVKGRFTISRDNAKNSIYLQMNSLRPEDTALYYCNKVPWGDYHP |
| (SEQ ID NO: 173) | RNVTWGQGTQVTVSS |
| Exemplary anti-target | EVQLVESGGGLVQPGRSLTLSCAASTNIDSISPMGWYRQAPGKQRELVAAIH |
| (BCMA) | GSSTLYADSVKGRFTISRDNAKNSIYLQMNSLRPEDTALYYCNKVPWGDYHP |
| (SEQ ID NO: 174) | RNVTWGQGTQVTVSS |
| Exemplary anti-target | EVQLVESGGGLVQPGRSLTLSCAASTNIRSISPMGWYRQAPGKQRELVAAIH |
| (BCMA) | GSSTLYADSVKGRFTISRDNAKNSIYLQMNSLRPEDTALYYCNKVPWGDYHP |
| (SEQ ID NO: 175) | RNVVWGQGTQVTVSS |
| Exemplary anti-target | EVQLVESGGGLVQPGRSLTLSCAASTNITSISPMGWYRQAPGKQRELVAAIS |
| (BCMA) | GFSTLYADSVKGRFTISRDNAKNSIYLQMNSLRPEDTALYYCNEVPWGDYHP |
| (SEQ ID NO: 176) | RNVYWGQGTQVTVSS |
| Exemplary anti-target | EVQLVESGGGLVQPGRSLTLSCAASTNITSISPMGWYRQAPGKQRELVAAIH |
| (BCMA) | GESTLYADSVKGRFTISRDNAKNSIYLQMNSLRPEDTALYYCNKVPWGDYHP |
| (SEQ ID NO: 177) | RNVYWGQGTQVTVSS |
| Exemplary anti-target | EVQLVESGGGLVQPGRSLTLSCAASTNITSVSPMGWYRQAPGKQRELVAAI |
| (BCMA) | HGPSTLYADSVKGRFTISRDNAKNSIYLQMNSLRPEDTALYYCNKVPWGDY |
| (SEQ ID NO: 178) | HPTNVYWGQGTQVTVSS |
| Exemplary anti-target | EVQLVESGGGLVQPGRSLTLSCAASTNIGSISPMGWYRQAPGKQRELVAAI |
| (BCMA) | HGQSTLYADSVKGRFTISRDNAKNSIYLQMNSLRPEDTALYYCNKVPWGDY |
| (SEQ ID NO: 179) | HPQNVYWGQGTQVTVSS |
| Exemplary anti-target | EVQLVESGGGLVQPGRSLTLSCAASTNIESISPMGWYRQAPGKQRELVAAIH |
| (BCMA) | GKSTLYADSVKGRFTISRDNAKNSIYLQMNSLRPEDTALYYCNKVPWGDYHP |
| (SEQ ID NO: 180) | RRVYWGQGTQVTVSS |
| Exemplary anti-target | EVQLVESGGGLVQPGRSLTLSCAASTNIVSISPMGWYRQAPGKQRELVAAIH |
| (BCMA) | GDSTLYADSVKGRFTISRDNAKNSIYLQMNSLRPEDTALYYCNKVPWGDYH |
| (SEQ ID NO: 189) | PRRVYWGQGTQVTVSS |
| Exemplary anti-target | EVQLVESGGGLVQPGRSLTLSCAASTNIDSISPMGWYRQAPGKQRELVAAIH |
| (BCMA) | GNSTLYADSVKGRFTISRDNAKNSIYLQMNSLRPEDTALYYCNKVPWGDYH |
| (SEQ ID NO: 190) | PRMVYWGQGTQVTVSS |
| Exemplary anti-target | EVQLVESGGGLVQPGRSLTLSCAASTNIFMISPMGWYRQAPGKQRELVAAI |
| (BCMA) | HGDSTLYADSVKGRFTISRDNAKNSIYLQMNSLRPEDTALYYCNKVPWGRY |
| (SEQ ID NO: 191) | HPRNVYWGQGTQVTVSS |
| Exemplary anti-target | EVQLVESGGGLVQPGRSLTLSCAASTNIFRISPMGWYRQAPGKQRELVAAIH |
| (BCMA) | GDSTLYADSVKGRFTISRDNAKNSIYLQMNSLRPEDTALYYCNKVPWGRYHP |
| (SEQ ID NO: 192) | RNVYWGQGTQVTVSS |
| Exemplary anti-target | EVQLVESGGGLVQPGRSLTLSCAASTNIFSISPMGWYRQAPGKQRELVAAIH |
| (BCMA) | GDSTLYADSVKGRFTISRDNAKNSIYLQMNSLRPEDTALYYCNKVPWGEYHP |
| (SEQ ID NO: 193) | RNVYWGQGTQVTVSS |
| Exemplary anti-target | EVQLVESGGGLVQPGRSLTLSCAASTNIFSISPMGWYRQAPGKQRELVAAIH |
| (BCMA) | GDSTLYADSVKGRFTISRDNAKNSIYLQMNSLRPEDTALYYCNKVPWGKYHP |
| (SEQ ID NO: 194) | RNVYWGQGTQVTVSS |
| Exemplary anti-target | EVQLVESGGGLVQPGRSLTLSCAASTNIESISPMGWYRQAPGKQRELVAAIH |
| (BCMA) | GSSTLYADSVKGRFTISRDNAKNSIYLQMNSLRPEDTALYYCNKVPWGRYHP |
| (SEQ ID NO: 195) | RNVYWGQGTQVTVSS |
| Exemplary anti-target | EVQLVESGGGLVQPGRSLTLSCAASTNIESISPMGWYRQAPGKQRELVAAIH |
| (BCMA) | GNSTLYADSVKGRFTISRDNAKNSIYLQMNSLRPEDTALYYCNKVPWGRYHP |
| (SEQ ID NO: 196) | RNVYWGQGTQVTVSS |
| Exemplary anti-target | EVQLVESGGGLVQPGRSLTLSCAASTNISSISPMGWYRQAPGKQRELVAAIH |
| (BCMA) | GFSTLYADSVKGRFTISRDNAKNSIYLQMNSLRPEDTALYYCNKVPWGYYHP |
| (SEQ ID NO: 197) | RNVYWGQGTQVTVSS |
| Exemplary anti-target | EVQLVESGGGLVQPGRSLTLSCAASTNISSISPMGWYRQAPGKQRELVAAIH |
| (BCMA) | GHSTLYADSVKGRFTISRDNAKNSIYLQMNSLRPEDTALYYCNKVPWGRYHP |
| (SEQ ID NO: 198) | RNVYWGQGTQVTVSS |
| Exemplary anti-target | EVQLVESGGGLVQPGRSLTLSCAASTNISSISPMGWYRQAPGKQRELVAAIH |
| (BCMA) | GFSTVYADSVKGRFTISRDNAKNSIYLQMNSLRPEDTALYYCNKVPWGRYHP |
| (SEQ ID NO: 199) | RNVYWGQGTQVTVSS |
| Exemplary anti-target | EVQLVESGGGLVQPGRSLTLSCAASTNIFSIRPMGWYRQAPGKQRELVAAIH |
| (BCMA) | GFSTVYADSVKGRFTISRDNAKNSIYLQMNSLRPEDTALYYCNKVPWGDYH |
| (SEQ ID NO: 200) | PRNVYWGQGTQVTVSS |
| Exemplary anti-target | EVQLVESGGGLVQPGRSLTLSCAASTNIFSIYPMGWYRQAPGKQRELVAAIH |
| (BCMA) | GFSTYYADSVKGRFTISRDNAKNSIYLQMNSLRPEDTALYYCNKVPWGSYHP |
| (SEQ ID NO: 201) | RNVYWGQGTQVTVSS |
| Exemplary anti-target | EVQLVESGGGLVQPGRSLTLSCAASTNIFNISPMGWYRQAPGKQRELVAAI |
| (BCMA) | HGFSTYYADSVKGRFTISRDNAKNSIYLQMNSLRPEDTALYYCNKVPWGRYH |
| (SEQ ID NO: 202) | PRNVYWGQGTQVTVSS |
| Exemplary anti-target | EVQLVESGGGLVQPGRSLTLSCAASTNIFSSSPMGWYRQAPGKQRELVAAI |
| (BCMA) | HGFSTWYADSVKGRFTISRDNAKNSIYLQMNSLRPEDTALYYCNKVPWGRY |
| (SEQ ID NO: 203) | HPRNVYWGQGTQVTVSS |
| Exemplary anti-target | EVQLVESGGGLVQPGRSLTLSCAASTNISSISPMGWYRQAPGKQRELVAAIH |
| (BCMA) | GFDTLYADSVKGRFTISRDNAKNSIYLQMNSLRPEDTALYYCNKVPWGDYH |
| (SEQ ID NO: 204) | PRNVYWGQGTQVTVSS |
| Exemplary anti-target | EVQLVESGGGLVQPGRSLTLSCAASTNIFSINPMGWYRQAPGKQRELVAAI |
| (BCMA) | HGFDTLYADSVKGRFTISRDNAKNSIYLQMNSLRPEDTALYYCNKVPWGDY |
| (SEQ ID NO: 205) | HPRNVSWGQGTQVTVSS |
| Exemplary anti-target | EVQLVESGGGLVQPGRSLTLSCAASTNIFSITPMGWYRQAPGKQRELVAAIH |
| (BCMA) | GRSTLYADSVKGRFTISRDNAKNSIYLQMNSLRPEDTALYYCNKVPWGSYHP |
| (SEQ ID NO: 206) | RNVYWGQGTQVTVSS |
| Exemplary anti-target | EVQLVESGGGLVQPGRSLTLSCAASTNIFSITPMGWYRQAPGKQRELVAAIH |
| (BCMA) | GTSTLYADSVKGRFTISRDNAKNSIYLQMNSLRPEDTALYYCNKVPWGRYHP |
| (SEQ ID NO: 207) | RNVYWGQGTQVTVSS |
| Exemplary anti-target | EVQLVESGGGLVQPGRSLTLSCAASTNIFSITPMGWYRQAPGKQRELVAAIH |
| (BCMA) | GESTLYADSVKGRFTISRDNAKNSIYLQMNSLRPEDTALYYCNKVPWGRYHP |
| (SEQ ID NO: 208) | RNVYWGQGTQVTVSS |
| Exemplary anti-target | EVQLVESGGGLVQPGRSLTLSCAASTNIFSITPMGWYRQAPGKQRELVAAIH |
| (BCMA) | GESTLYADSVKGRFTISRDNAKNSIYLQMNSLRPEDTALYYCNKVPWGDYHP |
| (SEQ ID NO: 209) | RDVYWGQGTQVTVSS |
| Exemplary anti-target | EVQLVESGGGLVQPGRSLTLSCAASTNIFSTSPYGWYRQAPGKQRELVAAIH |
| (BCMA) | GFSTIYADSVKGRFTISRDNAKNSIYLQMNSLRPEDTALYYCNKVPWGDYHP |
| (SEQ ID NO: 210) | GNVYWGQGTQVTVSS |
| Exemplary anti-target | EVQLVESGGGLVQPGRSLTLSCAASTNIFSTSPGGWYRQAPGKQRELVAAIH |
| (BCMA) | GFSTIYADSVKGRFTISRDNAKNSIYLQMNSLRPEDTALYYCNKVPWGDYHP |
| (SEQ ID NO: 211) | GNVYWGQGTQVTVSS |
| Exemplary anti-target | EVQLVESGGGLVQPGRSLTLSCAASTNIFSITPYGWYRQAPGKQRELVAAIH |
| (BCMA) | GASTLYADSVKGRFTISRDNAKNSIYLQMNSLRPEDTALYYCNKVPWGDYHP |
| (SEQ ID NO: 212) | GNVYWGQGTQVTVSS |
| Exemplary anti-target | EVQLVESGGGLVQPGRSLTLSCAASTNIFSITPGGWYRQAPGKQRELVAAIH |
| (BCMA) | GASTLYADSVKGRFTISRDNAKNSIYLQMNSLRPEDTALYYCNKVPWGDYHP |
| (SEQ ID NO: 213) | GNVYWGQGTQVTVSS |
| Exemplary anti-target | QVQLVESGGGLVQPGESLRLSCAASTNIFSISPMGWYRQAPGKQRELVAAI |
| (BCMA) | HGFSTLYADSVKGRFTISRDNAKNTIYLQMNSLKPEDTAVYYCNKVPWGDY |
| (SEQ ID NO: 214) | HPRNVYWGQGTQVTVSS |
| Exemplary anti-target | QVQLVESGGGLVQPGGSLRLSCAASGRTFSVRGMAWYRQAGNNRALVAT |
| (MSLN) | MNPDGFPNYADAVKGRFTISWDIAENTVYLQMNSLNSEDTTVYYCNSGPY |
| (SEQ ID NO: 215) | WGQGTQVTVSS |
| Exemplary anti-target | QVQLVESGGGLVQAGGSLRLSCAASGSIPSIEQMGWYRQAPGKQRELVAAL |
| (MSLN) | TSGGRANYADSVKGRFTISGDNVRNMVYLQMNSLKPEDTAIYYCSAGRFKG |
| (SEQ ID NO: 216) | DYAQRSGMDYWGKGTLVTVSS |
| Exemplary anti-target | QVQLVESGGGLVQAGGSLRLSCAFSGTTYTFDLMSWYRQAPGKQRTVVASI |
| (MSLN) | SSDGRTSYADSVRGRFTISGENGKNTVYLQMNSLKLEDTAVYYCLGQRSGVR |
| (SEQ ID NO: 217) | AFWGQGTQVTVSS |
| Exemplary anti-target | QVQLVESGGGLVQAGGSLRLSCVASGSTSNINNMRWYRQAPGKERELVAV |
| (MSLN) | ITRGGYAIYLDAVKGRFTISRDNANNAIYLEMNSLKPEDTAVYVCNADRVEG |
| (SEQ ID NO: 218) | TSGGPQLRDYFGQGTQVTVSS |
| Exemplary anti-target | QVQLVESGGGLVQAGGSLRLSCAASGSTFGINAMGWYRQAPGKQRELVAV |
| (MSLN) | ISRGGSTNYADSVKGRFTISRDNAENTVSLQMNTLKPEDTAVYFCNARTYTR |
| (SEQ ID NO: 219) | HDYWGQGTQVTVSS |
| Exemplary anti-target | QVRLVESGGGLVQAGGSLRLSCAASISAFRLMSVRWYRQDPSKQREWVATI |
| (MSLN) | DQLGRTNYADSVKGRFAISKDSTRNTVYLQMNMLRPEDTAVYYCNAGGGP |
| (SEQ ID NO: 220) | LGSRWLRGRHWGQGTQVTVSS |
| Exemplary anti-target | QVRLVESGGGLVQAGESLRLSCAASGRPFSINTMGWYRQAPGKQRELVASI |
| (MSLN) | SSSGDFTYTDSVKGRFTISRDNAKNTVYLQMNSLKPEDTAVYYCNARRTYLP |
| (SEQ ID NO: 221) | RRFGSWGQGTQVTVSS |
| Exemplary anti-target | QVQPVESGGGLVQPGGSLRLSCVVSGSDFTEDAMAWYRQASGKERESVAF |
| (MSLN) | VSKDGKRILYLDSVRGRFTISRDIDKKTVYLQMDNLKPEDTGVYYCNSAPGAA |
| (SEQ ID NO: 222) | RNYWGQGTQVTVSS |
| Exemplary anti-target | QVQPVESGGGLVQPGGSLRLSCVVSGSDFTEDAMAWYRQASGKERESVAF |
| (MSLN) | VSKDGKRILYLDSVRGRFTISRDIYKKTVYLQMDNLKPEDTGVYYCNSAPGAA |
| (SEQ ID NO: 223) | RNVWGQGTQVTVSS |
| Exemplary anti-target | EVQLVESGGGLVQPGNSLRLSCAASGFTFSSFGMSWVRQAPGKGLEWVSSI |
| (MSLN) | SGSGSDTLYADSVKGRFTISRDNAKTTLYLQMNSLRPEDTAVYYCTIGGSLSR |
| (SEQ ID NO: 224) | SSQGTLVTVSS |
| Exemplary anti-target | QVQIVESGGGLVQAGGSLRLSCVASGLTYSIVAVGWYRQAPGKEREMVADI |
| (MSLN) | SPVGNTNYADSVKGRFTISKENAKNTVYLQMNSLKPEDTAVYYCHIVRGWL |
| (SEQ ID NO: 225) | DERPGPGPIVYWGQGTQVTVSS |
| Exemplary anti-target | QVQLVESGGGLVQTGGSLRLSCAASGLTFGVYGMEWFRQAPGKQREWVA |
| (MSLN) | SHTSTGYVYYRDSVKGRFTISRDNAKSTVYLQMNSLKPEDTAIYYCKANRGSY |
| (SEQ ID NO: 226) | EYWGQGTQVTVSS |
| Exemplary anti-target | QVQLVESGGGLVQAGGSLRLSCAASTTSSINSMSWYRQAQGKQREPVAVIT |
| (MSLN) | DRGSTSYADSVKGRFTISRDNAKNTVYLQMNSLKPEDTAIYTCHVIADWRGY |
| (SEQ ID NO: 227) | WGQGTQVTVSS |
| Exemplary anti-target | QVQLVESGGGLVQAGGSLRLSCAASGRTLSRYAMGWFRQAPGKERQFVAA |
| (MSLN) | ISRSGGTTRYSDSVKGRFTISRDNAANTFYLQMNNLRPDDTAVYYCNVRRRG |
| (SEQ ID NO: 228) | WGRTLEYWGQGTQVTVSS |
| Exemplary anti-target | QVQLGESGGGLVQAGGSLRLSCAASGSIFSPNAMIWHRQAPGKQREPVASI |
| (MSLN) | NSSGSTNYGDSVKGRFTVSRDIVKNTMYLQMNSLKPEDTAVYYCSYSDFRR |
| (SEQ ID NO: 229) | GTQYWGQGTQVTVSS |
| Exemplary anti-target | QVQLVESGGGLVPSGGSLRLSCAASGATSAITNLGWYRRAPGQVREMVARI |
| (MSLN) | SVREDKEDYEDSVKGRFTISRDNTQNLVYLQMNNLQPHDTAIYYCGAQRW |
| (SEQ ID NO: 230) | GRGPGTTWGQGTQVTVSS |
| Exemplary anti-target | QVQLVESGGGLVQAGGSLRLSCAASGSTFRIRVMRWYRQAPGTERDLVAVI |
| (MSLN) | SGSSTYYADSVKGRFTISRDNAKNTLYLQMNNLKPEDTAVYYCNADDSGIAR |
| (SEQ ID NO: 231) | DYWGQGTQVTVSS |
| Exemplary anti-target | QVQLVESGGGLVQAGESRRLSCAVSGDTSKFKAVGWYRQAPGAQRELLAW |
| (MSLN) | INNSGVGNTAESVKGRFTISRDNAKNTVYLQMNRLTPEDTDVYYCRFYRRFG |
| (SEQ ID NO: 232) | INKNYWGQGTQVTVSS |
| Exemplary anti-target | QVQLVESGGGLVQAGGSLRLSCAASGSTFGNKPMGWYRQAPGKQRELVA |
| (MSLN) | VISSDGGSTRYAALVKGRFTISRDNAKNTVYLQMESLVAEDTAVYYCNALRTY |
| (SEQ ID NO: 233) | YLNDPVVFSWGQGTQVTVSS |
| Exemplary anti-target | QVQLVESGGGLVQAGGSLRLSCAASGSTSSINTMYWYRQAPGKERELVAFIS |
| (MSLN) | SGGSTNVRDSVKGRFSVSRDSAKNIVYLQMNSLTPEDTAVYYCNTYIPLRGTL |
| (SEQ ID NO: 234) | HDYWGQGTQVTVSS |
| Exemplary anti-target | QVQLVESGGGLVQAGGSLRLSCVASGRIDRITTMGWYRQAPGKQRELVAT |
| (MSLN) | ISNRGTSNYANSVKGRFTISRDNAKNTVYLQMNSLKPEDTAVYYCNARKWG |
| (SEQ ID NO: 235) | RNYWGQGTQVTVSS |
| Exemplary anti-target | QVQLVESGGGLVQARGSLRLSCTASGRTIGINDMAWYRQAPGNOBELVATI |
| (MSLN) | TKGGTTDYADSVDGRFTISRDNAKNTVYLQMNSLKPEDTAVYYCNTKRREW |
| (SEQ ID NO: 236) | AKDFEYWGQGTQVTVSS |
| Exemplary anti-target | QVQLVESGGGLVQAGGSLRLSCAASAIGSINSMSWYRQAPGKQREPVAVIT |
| (MSLN) | DRGSTSYADSVKGRFTISRDNAKNTVYLQMNSLKPEDTAIYTCHVIADWRGY |
| (SEQ ID NO: 237) | WGQGTQVTVSS |
| Exemplary anti-target | QVQLVESGGGLVQAGGSLRLSCAASGSTSSINTMYWFRQAPGEERELVATI |
| (MSLN) | NRGGSTNVRDSVKGRFSVSRDSAKNIVYLQMNRLKPEDTAVYYCNTYIPYG |
| (SEQ ID NO: 238) | GTLHDFWGQGTQVTVSS |
| Exemplary anti-target | QVQLVESGGGLVQAGGSLRLSCTTSTTFSINSMSWYRQAPGNQREPVAVIT |
| (MSLN) | NRGTTSYADSVKGRFTISRDNARNTVYLQMDSLKPEDTAIYTCHVIADWRGY |
| (SEQ ID NO: 239) | WGQGTQVTVSS |
| Exemplary anti-target | QVQLVESGGGLVQAGGSLTLSCAASGSTFSIRAMRWYRQAPGTERDLVAVI |
| (MSLN) | YGSSTYYADAVKGRFTISRDNAKNTLYLQMNNLKPEDTAVYYCNADTIGTAR |
| (SEQ ID NO: 240) | DYWGQGTQVTVSS |
| Exemplary anti-target | QVQLVESGGGLVQAGGSLRLSCVASGRTSTIDTMYWHRQAPGNERELVAY |
| (MSLN) | VTSRGTSNVADSVKGRFTISRDNAKNTAYLQMNSLKPEDTAVYYCSVRTTSY |
| (SEQ ID NO: 241) | PVDFWGQGTQVTVSS |
| Exemplary anti-target | QVQLVESGGGLVQAGGSLRLSCAASGSTSSINTMYWYRQAPGKERELVAFIS |
| (MSLN) | SGGSTNVRDSVKGRFSVSRDSAKNIVYLQMNSLKPEDTAVYYCNTYIPYGGT |
| (SEQ ID NO: 242) | LHDFWGQGTQVTVSS |
| Exemplary anti-target | QVQLVESGGGLVQPGGSLRLSCAASGGDWSANFMYWYRQAPGKQRELVA |
| (MSLN) | RISGRGVVDYVESVKGRFTISRDNAKNTVYLQMNSLKPEDTAVYYCAVASY |
| (SEQ ID NO: 243) | WGQGTQVTVSS |
| Exemplary anti-target | EVQLVESGGGLVQPGGSLRLSCAASGGDWSANFMYWYRQAPGKQRELVA |
| (MSLN) | RISGRGVVDYVESVKGRFTISRDNSKNTLYLQMNSLRAEDTAVYYCAVASYW |
| (SEQ ID NO: 244) | GQGTLVTVSS |
| Exemplary anti-target | EVQLVESGGGLVQPGGSLRLSCAASGGDWSANFMYWVRQAPGKGLEWV |
| (MSLN) | SRISGRGVVDYVESVKGRFTISRDNSKNTLYLQMNSLRAEDTAVYYCAVASY |
| (SEQ ID NO: 245) | WGQGTLVTVSS |
| Exemplary anti-target | EVQLVESGGGLVQAGGSLRLSCAASGSTSSINTMYWYRQAPGKERELVAFIS |
| (MSLN) | SGGSTNVRDSVKGRFTISRDNSKNTLYLQMNSLRAEDTAVYYCNTYIPYGGT |
| (SEQ ID NO: 246) | LHDFWGQGTLVTVSS |
| Exemplary anti-target | EVQLVESGGGLVQPGGSLRLSCAASGSTSSINTMYWYRQAPGKERELVAFIS |
| (MSLN) | SGGSTNVRDSVKGRFTISRDNSKNTLYLQMNSLRAEDTAVYYCNTYIPYGGT |
| (SEQ ID NO: 247) | LHDFWGQGTLVTVSS |
| Exemplary anti-target | EVQLVESGGGLVQPGGSLRLSCAASGSTSSINTMYWVRQAPGKGLEWVSFI |
| (MSLN) | SSGGSTNVRDSVKGRFTISRDNSKNTLYLQMNSLRAEDTAVYYCNTYIPYGG |
| (SEQ ID NO: 248) | TLHDFWGQGTLVTVSS |
| Exemplary anti-target | QVQLVESGGGVVQAGGSLRLSCAASGSTFSIRAMRWYRQAPGTERDLVAVI |
| (MSLN) | YGSSTYYADAVKGRFTISRDNSKNTLYLQMNSLRAEDTAVYYCNADTIGTAR |
| (SEQ ID NO: 249) | DYWGQGTLVTVSS |
| Exemplary anti-target | QVQLVESGGGVVQPGGSLRLSCAASGSTFSIRAMRWYRQAPGKERELVAVI |
| (MSLN) | YGSSTYYADAVKGRFTISRDNSKNTLYLQMNSLRAEDTAVYYCNADTIGTAR |
| (SEQ ID NO: 250) | DYWGQGTLVTVSSGG |
| Exemplary anti-target | QVQLVESGGGVVQPGGSLRLSCAASGSTFSIRAMRWVRQAPGKGLEWVSV |
| (MSLN) | IYGSSTYYADAVKGRFTISRDNSKNTLYLQMNSLRAEDTAVYYCNADTIGTAR |
| (SEQ ID NO: 251) | DYWGQGTLVTVSSGG |
| Exemplary anti-target | EVQLVESGGGLVQAGGSLRLSCVASGRTSTIDTMYWHRQAPGNERELVAYV |
| (MSLN) | TSRGTSNVADSVKGRFTISRDNSKNTLYLQMNSLRAEDTAVYYCSVRTTSYP |
| (SEQ ID NO: 252) | VDFWGQGTLVTVSGG |
| Exemplary anti-target | EVQLVESGGGLVQPGGSLRLSCAASGRTSTIDTMYWHRQAPGKERELVAYV |
| (MSLN) | TSRGTSNVADSVKGRFTISRDNSKNTLYLQMNSLRAEDTAVYYCSVRTTSYP |
| (SEQ ID NO: 253) | VDFWGQGTLVTVSS |
| Exemplary anti-target | EVQLVESGGGLVQPGGSLRLSCAASGRTSTIDTMYWVRQAPGKGLEWVSY |
| (MSLN) | VTSRGTSNVADSVKGRFTISRDNSKNTLYLQMNSLRAEDTAVYYCSVRTTSY |
| (SEQ ID NO: 254) | PVDFWGQGTLVTVSS |
| Exemplary anti-target | QVQLVESGGGVVQAGGSLTLSCAASGSTFSIRAMRWYRQAPGTERDLVAVI |
| (MSLN) | YGSSTYYADAVKGRFTISRDNSKNTLYLQMNSLRAEDTAVYYCNADTIGTAR |
| (SEQ ID NO: 255) | DYWGQGTLVTVSS |
| Exemplary anti-target | QVQLVESGGGVVQAGGSLRLSCAASGSTFSIRAMRWYRQAPGTERDLVAVI |
| (MSLN) | YGSSTYYADAVKGRFTISRDNSKNTLYLQMNSLRAEDTAVYYCNADTIGTAR |
| (SEQ ID NO: 256) | DYWGQGTLVTVSS |
| Exemplary anti-target | QVQLVESGGGVVQPGGSLRLSCAASGSTFSIRAMRWYRQAPGKERELVAVI |
| (MSLN) | YGSSTYYADAVKGRFTISRDNSKNTLYLQMNSLRAEDTAVYYCNADTIGTAR |
| (SEQ ID NO: 257) | DYWGQGTLVTVSS |
| Exemplary anti-target | QVQLVESGGGVVQPGGSLRLSCAASGSTFSIRAMRWVRQAPGKGLEWVSV |
| (MSLN) | IYGSSTYYADAVKGRFTISRDNSKNTLYLQMNSLRAEDTAVYYCNADTIGTAR |
| (SEQ ID NO: 258) | DYWGQGTLVTVSS |
| Exemplary anti-target | QVQLQESGGGLVQAGGSLRLSCAASGSIFSIASMGWYRQAPGKQRELVAVI |
| (DLL3) | TSFSSTNYADSVKGRFTISRDNAKNTVYLQMNSLKPEDTGVYYCNARYFERT |
| (SEQ ID NO: 306) | DWGQGTQVTVSS |
| Exemplary anti-target | QVQLQESGGGLVQAGGSLRLSCAAPGSIFSIASMGWYRQAPGKQRELVAVI |
| (DLL3) | TSFSSTNYADSVKGRFTISRDNAKNTVYLQMNSLKPEDTGVYYCNARYFERT |
| (SEQ ID NO: 307) | DWGQGTQVTVSS |
| Exemplary anti-target | QVQLQESGGGLVQAGGSLRLSCAASGSIFSIASMAWYRQAPGKQRELVAAI |
| (DLL3) | TSFSSTNYADSVKGRFTISRDNAKNTVYLQMNSLKPEDTGVYYCNARYFERT |
| (SEQ ID NO: 308) | DWGQGTQVTVSS |
| Exemplary anti-target | QVQLQESGGGLVQAGGSLRLSCAASESIFSINVMAWHRQAPGKQRELVARI |
| (DLL3) | TSGGSTNYADSVKGRFTISRDNAKNTVYLQMNSLKPEDTGVYYCGAYQGLY |
| (SEQ ID NO: 309) | AYWGQGTQVTVSS |
| Exemplary anti-target | QVQLQESGGGLVQAGGSLRLSCVASGSSFSITSMAWYRQAPGKQRDLVAAI |
| (DLL3) | TSFGSTNYADSVKDRFTISRDNAKNTVYLQMNSLKPEDTAVYYCNGRVFDH |
| (SEQ ID NO: 310) | VYWGQGTQVTVSS |
| Exemplary anti-target | QVQLQESGGGLVQAGGSLKLSCAASSSIFSISSMSWYRQAPGKQRELVAAIT |
| (DLL3) | TFDYTNYADSVKGRFTISRDNAKNMMYLQMNSLKPEDTAVYLCNARAFGR |
| (SEQ ID NO: 311) | DYWGQGTQVTVSS |
| Exemplary anti-target | QVQLQESGGGLVQAGGSLKLSCAASSSIFSISSMSWYRQAPGKQRELVAAIT |
| (DLL3) | SFGSTNYADSVKGRFTISRDNAKNMMYLQMNSLKPEDTAVYRCNARTMGR |
| (SEQ ID NO: 312) | DYWGQGTQVTVSS |
| Exemplary anti-target | QVQLQESGGGLVQPGGSLRLSCAASGSTLNIKIMAWHRQAPGKQRELVATL |
| (DLL3) | TSGGNTNYADSVKGRFTISRDNAKNTVYLQMNSLQPEDTAVYYCGLWDGV |
| (SEQ ID NO: 313) | GGAYWGRGTQVTVSS |
| Exemplary anti-target | QVQLQESGGGLVQPGGSLRISCAASGSTLNIKIMAWHRQAPGKQRELVATL |
| (DLL3) | TSGGNTNYADSVKGRFTISRDNAKNTVYLQMNSLQPEDTAVYYCGLWDGV |
| (SEQ ID NO: 314) | GGAYWGRGTQVTVSS |
| Exemplary anti-target | QVQLQESGGGLVQAGGSLRLSCAASGSTFNIKTMAWHRQAPGNQRELVAT |
| (DLL3) | LTSGGNTNYADSVKGRFTISRDNAKNTVYLQMNSLKPEDTAVYYCGLWNG |
| (SEQ ID NO: 315) | VGGAYWGRGTQVTVSS |
| Exemplary anti-target | QVQLQDGGGLVQPGGSLRLSCAASGSTFNIKLMAWHRQAPGNQRELVATL |
| (DLL3) | TSGGNTNYADSVKGRFTISRDNASNIVYLQMNSLKPEDTAVYYCGLWDGVG |
| (SEQ ID NO: 316) | GAYWGRGTQVTVSS |
| Exemplary anti-target | QVQLQESGGGLVQAGGSLRLSCAASGSTFNFKIMAWHRQAPGKQRELVAS |
| (DLL3) | LTSEGLTNYRDSVKGRFTISRDNAKNTVYLQMNNLKPEDTAVYYCGLWDGV |
| (SEQ ID NO: 317) | GGAYWGRGTQVTVSS |
| Exemplary anti-target | QVQLQESGGGLVQPGGSLRLSCAASGFMFSSYSMSWYRQAPGKQRELVAA |
| (DLL3) | ITTWGSTNYADSVKGRFTISRDNAKNTVWLQMNSLEPEDTAVYFCNARSW |
| (SEQ ID NO: 318) | NNYWGQGTQVTVSS |
| Exemplary anti-target | QVQLQESGGGLVQVGGSLRLSCAASGFMFSSYSMSWYRQAPGKQRELVA |
| (DLL3) | AITSYGSTNYADSVKGRFTISRDNAKNTVWLQMNSLKPEDTAVYFCNARSW |
| (SEQ ID NO: 319) | NNYWGQGTQVTVSS |
| Exemplary anti-target | QVQLQESGGGLVQPGGSLRLSCAASGFTFSSHSMSWYRQAPGKQRELVAAI |
| (DLL3) | TTYGSTNYIDSVKGRFTISRDNTKNTVYLQMNSLKPEDTAVYFCNARSWNNY |
| (SEQ ID NO: 320) | WGQGTQVTVSS |
| Exemplary anti-target | QVQLQESGGGLVQAGGSLRLSCVASGSSFSHNTMGWYRQAPGKQRDLVA |
| (DLL3) | RITTFGTTNYADSVKGRFTISRDNAKNNYLQMNSLKPEDTAVYYCNGESFG |
| (SEQ ID NO: 321) | RIWYNWGQGTQVTVSS |
| Exemplary anti-target | QVQLQESGGGLVQAGASLRLTCTASGGRFSYATMGWSRQAPGVIREMVA |
| (DLL3) | RITSSGFSTNYADSVKGRFTISRDNAKNAVYLQMDSLKPEDTAVYYCNAQHF |
| (SEQ ID NO: 322) | GTDSWGQGTQVTVSS |
| Exemplary anti-target | QVQLQESGGGLVQAGASLRLTCTASGSRFSYATMGWSRQAPGKQRELVAR |
| (DLL3) | ITSSGFSTNYADSVKGRFTISRDNAKNAVYLQMDSLKPEDTAVYYCNAQQFG |
| (SEQ ID NO: 323) | TDSWGQGTQVTVSS |
| Exemplary anti-target | QVQLQESGGGLVQAGGSLRLSCAASGSTFTSNVMGWHRQAPGKQRELVA |
| (DLL3) | NMHSGGSTNYADSVKGRFTISRDNAKNIVYLQMNNLKIEDTAVYYCRWYGI |
| (SEQ ID NO: 324) | QRAEGYWGQGTQVTVSS |
| Exemplary anti-target | QVQLQESGGGLVVAGGSLRLSCAASGSSVSFLSMAWYRQAPGKKRELVAGI |
| (DLL3) | SVDGSTNYADSVKGRFTVSRDNAKNTVYLQMNSLCIPEDTAVYYCYAYRWV |
| (SEQ ID NO: 325) | GRDTYWGQGTQVTVSS |
| Exemplary anti-target | QVQLQESGGGLVVAGGSLRLSCAASGSSVSFLSMAWYRQAPGKKRELVAGI |
| (DLL3) | SVDGSTNYADSVKGRFTISRDNAKNTVYLQMNSLQPEDTAVYYCYAYRWV |
| (SEQ ID NO: 326) | GRDTYWGQGTQVTVSS |
| Exemplary anti-target | QVQLQESGGGLVVSGGSLRLSCAASGSSVSFLSMAWYRQAPGKKRELVAGI |
| (DLL3) | SVDGSTNYADSVKGRFTISRDNAKNTVYLQMNSLQPEDTAAYYCYAYRWV |
| (SEQ ID NO: 327) | GRDTYWGQGTQVTVSS |
| Exemplary anti-target | QVQLQESGGGLVVAGGSLRLSCAASGSSVSFLSMAWYRQAPGKKRELVAGI |
| (DLL3) | SVDGSTNYADSVKGRFTISRDNAENTVYLQMNSLQPEDTAVYYCYAYRWEG |
| (SEQ ID NO: 328) | RDTYWGQGTQVTVSS |
| Exemplary anti-target | QVQLQESGGGLVVAGGSLRLSCAASGSSVSFLSMAWYRQAPGKKRELVAGI |
| (DLL3) | SVDGSTNYADSVKGRFTISRDNAENTVYLQMNSLQPEDTAVYYCYAYRWEG |
| (SEQ ID NO: 329) | RNTYWGQGTQVTVSS |
| Exemplary anti-target | QVQLQESGGGLVQPGGSLRLSCAASGSSVSFLSMAWYRQAPGKKRELVAGI |
| (DLL3) | STDGSTNYVDSVKGRFTISRDNAKNTVYLQMNSLQPEDTAVYYCYAYRWVG |
| (SEQ ID NO: 330) | RYTYWGQGTQVTVSS |
| Exemplary anti-target | QVQLQESGGGLVVAGGSLRLSCAASGSSVSFLSMAWYRQAPGKKRELVAGI |
| (DLL3) | STDGTTNYVDSVKDRFTISRDNAKNTVYLQMNSLQPEDTAAYYCYAYRWVG |
| (SEQ ID NO: 331) | RDTYWGQGTQVTVSS |
| Exemplary anti-target | QVQLQESGGGLVVAGGSLRLSCAASGSSVSFLSIAWYRQAPGKKRELVAGIS |
| (DLL3) | TDGTTNYVDSVKDRFTISRDNAKNTVYLQMNSLQPEDTAAYYCYAYRWVG |
| (SEQ ID NO: 332) | RDTYWGQGTQVTVSS |
| Exemplary anti-target | QVQLQESGGGLVQAGGSLTLSCAASGSSVSFLSMAWYRQAPGKKRELVAGI |
| (DLL3) | STDGTTNYVDSVKDRFTISRDNAKNTVYLQMNSLQPEDTAAYYCYAYRWVG |
| (SEQ ID NO: 333) | RDTYWGQGTQVTVSS |
| Exemplary anti-target | QVQLQESGGGLVQAGGSLRLSCAASGSSVSFLSMAWYRQAPGKKRELVAGI |
| (DLL3) | STDGSTNYADSVKGRFTISEGNAKNTVDLQMNSLQPEDTAVYYCYAYRWV |
| (SEQ ID NO: 334) | DRYTYWGQGTQVTVSS |
| Exemplary anti-target | QVQLQESGGGLVVAGGSLRLSCAASGSSVSFLSMAWYRQAPGKKRELVAGI |
| (DLL3) | STDGSTNYADSVKGRFTISEDNAKNIVDLQMNSLQPEDTAVYYCYAYRWID |
| (SEQ ID NO: 335) | RYTYWGQGTQVTVSS |
| Exemplary anti-target | QVQLQESGGGLVQPGGSLRLSCAASGSSVSFLSMAWYRQAPGKKRELVAGI |
| (DLL3) | STDGSTNYADSVKGRFTISEDNAKNIVDLQMNSLQPEDTAVYYCYAYRWV |
| (SEQ ID NO: 336) | DRYTYWGQGTQVTVSS |
| Exemplary anti-target | QVQLQESGGGLVVAGGSLRLSCAASGSSVSFLSMAWYRQAPGKKRELVAGI |
| (DLL3) | SSDGSTNYVDSVKGRFTISRDNAKNIVFLQMNSLQPQDTAVYYCYAYRWVG |
| (SEQ ID NO: 337) | RDTYWGQGTQVTVSS |
| Exemplary anti-target | QVQLQESGGGLVVAGGSLRLSCAASGSSVSFLSMAWYRQAPGKKRELVAGI |
| (DLL3) | SADGSTDYIDSVKGRFTISRDSANNTMYLQMNSLQPEDTAVYYCYAYRWTT |
| (SEQ ID NO: 338) | RYTYWGQGTQVTVSS |
| Exemplary anti-target | QVQLQESGGGLVQPGGSLRLSCAASGFTLDYYAIGWYRQAPGKKRELVAGI |
| (DLL3) | SSDGSTHYVDSVKGRFAISRDNAENTVYLQMNDLQPDDTAVYYCYAYRWV |
| (SEQ ID NO: 339) | GGYTYWGQGTQVTVSS |
| Exemplary anti-target | QVQLQESGGGLVQAGGSLRLSCVASGSTSSINAMGWYRRAPGKQRELVAG |
| (DLL3) | ISSDGSKNYADSVKGRFTISRDNAKNTVYLQMNSLKPEDTAVYYCYYFRTVA |
| (SEQ ID NO: 340) | ASSMQYWGQGTQVTVSS |
| Exemplary anti-target | QVQLQESGGGLVQAGGSLRLSCVASGSTSSINAMGWYRRAPGKQRELVAG |
| (DLL3) | ISSDGSKVYADSVKGRFTISRDNAKNTVYLQMNSLKPEDTAVFYCYYFRTVSG |
| (SEQ ID NO: 341) | SSMRYWGQGTQVTVSS |
| Exemplary anti-target | QVQLQESGGGLVQAGGSLRLSCAASGITSSVYSMGWYRQAPGKQRELVAG |
| (DLL3) | SSSDGSTHYVDSVRGRFTISRDNAKNTVYLQMSSLKPEDTAVYYCYANRGFA |
| (SEQ ID NO: 342) | GAPSYWGQGTQVTVSS |
| Exemplary anti-target | QVQLQESGGGLVQAGGSLRLSCAASGRTSMFNSMGWHRQAPGKQRELV |
| (DLL3) | AIIRSGGSSNYADTVKGRFTISRDNTKNTVYLQMNDLKPEDTAVYYCFYYFQS |
| (SEQ ID NO: 343) | SYWGQGTQVTVSS |
| Exemplary anti-target | QVQLQESGGGLVQAGGSLRLSCAASGRTSMVNSMGWHRQAPGKQRELV |
| (DLL3) | ALITSGGSSNYADTVKGRFTISRDNTKNTVYLQMNDLKPEDTAVYYCFYYFQS |
| (SEQ ID NO: 344) | SYWGQGTQVTVSS |
| Exemplary anti-target | QVQLQESGGGLVQAGGSLRLSCAASGSVSMFNSMGWHRQPPGKQRELVA |
| (DLL3) | IITSGGSSNYADTVKGRFTISRDNTKNTVYLQMNDLKPEDTAVYYCFYYFQSS |
| (SEQ ID NO: 344) | YWGQGTQVTVSS |
| Exemplary anti-target | QVQLQESGGGLVQAGGSLRLSCTASGSIFSIAVMGWYRQVPGKRREWVATI |
| (DLL3) | FDGSYTNYADSVKGRFTISRDNARNKVYLQMNNLKPEDTAVYYCQTHWTQ |
| (SEQ ID NO: 345) | GSVPKESWGQGTQVTVSS |
| Exemplary anti-target | QVQLQESGGGLVQAGGSLRLSCVASSGIFSDMSMVWYRQAPGKQRELVAS |
| (DLL3) | ITTFGSTNYADPVKGRFTISRDNAKNTVYLQMNSLKPEDTAVYYCSGRSYSSD |
| (SEQ ID NO: 346) | YWGRGTQVTVSS |
| Exemplary anti-target | QVQLQESGGGLVQAGGSLRLSCVASGSISSIIVMGWSRQAPGKQRESVATIT |
| (DLL3) | RDGTRNYADSLKGRFTISRDNAKNTSYLQINSLKPEDTAVYSCYARYGDINY |
| (SEQ ID NO: 347) | WGKGTQVTVSS |
| Exemplary anti-target | QVQLQESGGGLVQAGGSLRLSCVASGSISSIIVMGWSRQAPGKQRESLATIS |
| (DLL3) | RGGTRTYADSVKGRFTISRDNAKNTSYLQMNSLKPEDTAVYSCYARYGDINY |
| (SEQ ID NO: 348) | WGKGTQVTVSS |
| Exemplary anti-target | QVQLQESGGGLVQAGGSLRLSCVASGSIFTTNSMGWHRQGPGKQRELVAL |
| (DLL3) | IGSAGSTKYADSVKGRFTISRDNAKNTVSLQMDSLKPEDTAVYYCFYYDSRSY |
| (SEQ ID NO: 349) | WGQGTQVTVSS |
| Exemplary anti-target | QVQLQESGGGMVQPGGSLRLSCAASGSREISTMGWHRQAPGKQRELAARI |
| (DLL3) | TSGGITKYADSVKGRFTISRDNAKKTVYLQMNSLKSEDTAVYYCFAYDNINAY |
| (SEQ ID NO: 350) | WGQGTQVTVSS |
| Exemplary anti-target | QVQLQESGGGWVQAGGSLRLSCAASGSREISTMGWHRQAPGKQRELAAR |
| (DLL3) | ITSGGITKYADSVKGRFTISRDNAKKTVYLQMNSLKSEDTAVYYCFAYDNINA |
| (SEQ ID NO: 351) | YWGQGTQVTVSS |
| Exemplary anti-target | QVQLQESGGGWVQAGGSLRLSCTASGSREISTMGWHRQAPGKQRELAARI |
| (DLL3) | TSGGITKYADSVKGRFTISRDNAKKTVYLQMDSLKSEDTAVYYCFAYDNINAY |
| (SEQ ID NO: 352) | WGQGTQVTVSS |
| Exemplary anti-target | QVQLQESGGGSVQAGRSLGLSCAASGSREISTMGWHRQAPGKQRELAARI |
| (DLL3) | TSGGITKYADSVKGRFTISRDNAKKTVYLQMNSLKSEDTAVYYCFAYDNINAY |
| (SEQ ID NO: 353) | WGQGTQVTVSS |
| Exemplary anti-target | QVQLQESGGGLVQAGGSLRLSCTASGSIFRGAAMYWHRQAPGKQRELVAA |
| (DLL3) | ITTSGNTSYADSVKGRFTISRDNAKNTMYLQIISLKPEDTAVYYCAFWIAGKA |
| (SEQ ID NO: 354) | YWGQGTQVTVSS |
| Exemplary anti-target | QVQLQESGGGLVQPGGSLRLSCAASGSISSFNFMSWHRQAPGKERELAGVI |
| (DLL3) | TRGGATNYADSVKGRFTISRDNVKNTVYLQMNGLKPEDTAVYYCHGRSQLG |
| (SEQ ID NO: 355) | STWGQGTQVTVSS |
| Exemplary anti-target | QVQLQESGGGLVQAGGSLRLSCLASGTIFTASTMGWHRQPPGKQRELVASI |
| (DLL3) | AGDGRTNYAESTEGRFTISRDDAKNTMYLQMNSLKPEDTAVYYCYAYYLDT |
| (SEQ ID NO: 356) | YAYWGQGTQVTVSS |
| Exemplary anti-target | EVQLVESGGGLVQPGGSLTLSCAASGSIFSIASMGWYRQAPGKQRELVAVIT |
| (DLL3) | SFSSTNYADSVKGRFTISRDNAKNSVYLQMNSLRAEDTAVYYCNARYFERTD |
| (SEQ ID NO: 357) | WGQGTLVTVSS |
| Exemplary anti-target | EVQLVESGGGLVQPGGSLTLSCAASGRTSMFNSMGWHRQAPGKQRELVAI |
| (DLL3) | IRSGGSSNYADTVKGRFTISRDNAKNSVYLQMNSLRAEDTAVYYCFYYFQSSY |
| (SEQ ID NO: 358) | WGQGTLVTVSS |
| Exemplary anti-target | EVQLVESGGGLVQPGGSLTLSCAASGSSVSFLSMAWYRQAPGKKRELVAGIS |
| (DLL3) | VDGSTNYADSVKGRFTISRDNAKNSVYLQMNSLRAEDTAVYYCYAYRWVG |
| (SEQ. ID NO: 359) | RDTYWGQGTLVTVSS |
| Exemplary anti-target | EVQLVESGGGLVQPGGSLTLSCTASGSIFRGAAMYWHRQAPGKQRELVAAI |
| (DLL3) | TTSGNTSYADSVKGRFTISRDNAKNSMYLQMNSLRAEDTAVYYCAFWIAGK |
| (SEQ ID NO: 360) | AYWGQGTLVTVSS |
| Exemplary anti-target | EVQLVESGGGLVQPGGSLTLSCAASGSTFNIKTMAWHRQAPGNQRELVATL |
| (DLL3) | TSGGNTNYADSVKGRFTISRDNAKNSVYLQMNSLRAEDTAVYYCGLWNGV |
| (SEQ ID NO: 361) | GGAYWGQGTLVTVSS |
| Exemplary anti-target | EVQLVESGGGLVQPGGSLTLSCAASGSTLNIKIMAWHRQAPGKQRELVATL |
| (DLL3) | TSGGNTNYADSVKGRFTISRDNAKNSVYLQMNSLRAEDTAVYYCGLWDGV |
| (SEQ ID NO: 362) | GGAYWGQGTLVTVSS |
| Exemplary anti-target | EVQLVESGGGLVQPGGSLTLSCAASSSIFSISSMSWYRQAPGKQRELVAAITT |
| (DLL3) | FDYTNYADSVKGRFTISRDNAKNSMYLQMNSLRAEDTAVYYCNARAFGRDY |
| (SEQ ID NO: 363) | WGQGTLVTVSS |
| Exemplary anti-target | EVQLVESGGGLVQPGGSLTLSCVASGSTSSINAMGWYRRAPGKQRELVAGI |
| (DLL3) | SSDGSKNYADSVKGRFTISRDNAKNSVYLQMNSLRAEDTAVYYCYYFRTVAA |
| (SEQ ID NO: 364) | SSMQYWGQGTLVTVSS |
| Exemplary anti-target | EVQLVESGGGLVQPGGSLTLSCAASGFMFSSYSMSWYRQAPGKQRELVAAI |
| (DLL3) | TSYGSTNYADSVKGRFTISRDNAKNSVYLQMNSLRAEDTAVYYCNARSWNN |
| (SEQ ID NO: 365) | YWGQGTLVTVSS |
| Exemplary anti-target | EVQLVESGGGLVQPGGSLTLSCAASGSVSMFNSMGWHRQPPGKQRELVAI |
| (DLL3) | ITSGGSSNYADTVKGRFTISRDNAKNSVYLQMNSLRAEDTAVYYCFYYFQSSY |
| (SEQ ID NO: 366) | WGQGTLVTVSS |
| Exemplary anti-target | EVQLVESGGGLVQPGGSLTLSCTASGGRFSYATMGWSRQAPGKQREMVA |
| (DLL3) | RITSSGFSTNYADSVKGRFTISRDNAKNSVYLQMNSLRAEDTAVYYCNAQHF |
| (SEQ ID NO: 367) | GTDSWGQGTLVTVSS |
| Exemplary anti-target | EVQLVESGGGLVQPGGSLTLSCAASGSISSFNFMSWHRQAPGKERELAGVIT |
| (DLL3) | RGGATNYADSVKGRFTISRDNAKNSVYLQMNSLRAEDTAVYYCHGRSQLGS |
| (SEQ ID NO: 368) | TWGQGTLVTVSS |
| Exemplary anti-target | EVQLVESGGGLVQPGGSLTLSCAASESIFSINVMAWHRQAPGKQRELVARIT |
| (DLL3) | SGGSTNYADSVKGRFTISRDNAKNSVYLQMNSLRAEDTAVYYCGAYQGLYA |
| (SEQ ID NO: 369) | YWGQGTLVTVSS |
| Exemplary anti-target | EVQLVESGGGLVQPGGSLTLSCAASGSREISTMGWHRQAPGKQRELAARIT |
| (DLL3) | SGGITKYADSVKGRFTISRDNAKNSVYLQMNSLRAEDTAVYYCFAYDNINAY |
| (SEQ ID NO: 370) | WGQGTLVTVSS |
| Exemplary anti-target | EVQLVESGGGLVQPGGSLTLSCTASGSIFSIAVMGWYRQVPGKRREWVATI |
| (DLL3) | FDGSYTNYADSVKGRFTISRDNAKNSVYLQMNSLRAEDTAVYYCQTHWTQ |
| (SEQ ID NO: 371) | GSVPKESWGQGTLVTVSS |
| Exemplary anti-target | EVQLVESGGGLVQPGGSLTLSCVASGSTSSINAMGWYRRAPGKQRELVAGI |
| (DLL3) | SSDGSKVYADSVKGRFTISRDNAKNSVYLQMNSLRAEDTAVYYCYYFRTVSG |
| (SEQ ID NO: 372) | SSMRYWGQGTLVTVSS |
| Exemplary anti-target | EVQLVESGGGLVQPGGSLTLSCVASSGIFSDMSMVWYRQAPGKQRELVASI |
| (DLL3) | TTFGSTNYADPVKGRFTISRDNAKNSVYLQMNSLRAEDTAVYYCSGRSYSSD |
| (SEQ ID NO: 373) | YWGQGTLVTVSS |
| Exemplary anti-target | EVQLVESGGGLVQPGGSLTLSCAASGFMFSSYSMSWYRQAPGKQRELVAAI |
| (DLL3) | TTWGSTNYADSVKGRFTISRDNAKNSVYLQMNSLRAEDTAVYYCNARSWN |
| (SEQ ID NO: 374) | NYWGQGTLVTVSS |
| Exemplary anti-target | EVQLVESGGGLVQPGGSLTLSCTASGSRFSYATMGWSRQAPGKQRELVARI |
| (DLL3) | TSSGFSTNYADSVKGRFTISRDNAKNSVYLQMNSLRAEDTAVYYCNAQQFG |
| (SEQ ID NO: 375) | TDSWGQGTLVTVSS |
| Exemplary anti-target | EVQLVESGGGLVQPGGSLTLSCAASGSTFTSNVMGWHRQAPGKQRELVAN |
| (DLL3) | MHSGGSTNYADSVKGRFTISRDNAKNSVYLQMNSLRAEDTAVYYCRWYGI |
| (SEQ ID NO: 376) | QRAEGYWGQGTLVTVSS |
| Exemplary anti-target | EVQLVESGGGLVQPGGSLTLSCAASGSSVSFLSMAWYRQAPGKKRELVAGIS |
| (DLL3) | TDGSTNYVDSVKGRFTISRDNAKNSVYLQMNSLRAEDTAVYYCYAYRWVGR |
| (SEQ ID NO: 377) | YTYWGQGTLVTVSS |
| Exemplary anti-target | EVQLVESGGGLVQPGGSLTLSCVASGSISSIIVMGWSRQAPGKQRESVATIT |
| (DLL3) | RDGTRNYADSLKGRFTISRDNAKNSSYLQMNSLRAEDTAVYYCYARYGDINY |
| (SEQ ID NO: 378) | WGQGTLVTVSS |
| Exemplary anti-target | EVQLVESGGGLVQPGGSLTLSCAASGSSVSFLSMAWYRQAPGKKRELVAGIS |
| (DLL3) | ADGSTDYIDSVKGRFTISRDNAKNSVYLQMNSLRAEDTAVYYCYAYRWTTRY |
| (SEQ ID NO: 379) | TYWGQGTLVTVSS |
| Exemplary anti-target | EVQLVESGGGLVQPGGSLTLSCLASGTIFTASTMGWHRQPPGKQRELVASIA |
| (DLL3) | GDGRTNYAESTEGRFTISRDNAKNSMYLQMNSLRAEDTAVYYCYAYYLDTY |
| (SEQ ID NO: 380) | AYWGQGTLVTVSS |
| Exemplary anti-target | EVQLVESGGGLVQPGGSLTLSCAASGSSVSFLSMAWYRQAPGKKRELVAGIS |
| (DLL3) | VDGSTNYADSVKGRFTISRDNAKNSVYLQMNSLRAEDTAVYYCYAYRWEGR |
| (SEQ ID NO: 381) | NTYWGQGTLVTVSS |
| Exemplary anti-target | EVQLVESGGGLVQPGGSLTLSCVASGSSFSHNTMGWYRQAPGKQRDLVAR |
| (DLL3) | ITTFGTTNYADSVKGRFTISRDNAKNSVYLQMNSLRAEDTAVYYCNGESFGRI |
| (SEQ ID NO: 382) | WYNWGQGTLVTVSS |
| Exemplary anti-target | EVQLVESGGGLVQPGGSLTLSCVASGSISSIIVMGWSRQAPGKQRESLATISR |
| (DLL3) | GGTRTYADSVKGRFTISRDNAKNSSYLQMNSLRAEDTAVYYCYARYGDINY |
| (SEQ ID NO: 383) | WGQGTLVTVSS |
| Exemplary anti-target | EVQLVESGGGLVQPGGSLTLSCVASGSSFSITSMAWYRQAPGKQRDLVAAIT |
| (DLL3) | SFGSTNYADSVKDRFTISRDNAKNSVYLQMNSLRAEDTAVYYCNGRVFDHV |
| (SEQ ID NO: 384) | YWGQGTLVTVSS |
| Exemplary anti-target | EVQLVESGGGLVQPGGSLTLSCAASGRTSMVNSMGWHRQAPGKQRELVA |
| (DLL3) | LITSGGSSNYADTVKGRFTISRDNAKNSVYLQMNSLRAEDTAVYYCFYYFQSS |
| (SEQ ID NO: 385) | YWGQGTLVTVSS |
| Exemplary anti-target | EVQLVESGGGLVQPGGSLTLSCAASGSTFNFKIMAWHRQAPGKQRELVASL |
| (DLL3) | TSEGLTNYRDSVKGRFTISRDNAKNSVYLQMNSLRAEDTAVYYCGLWDGVG |
| (SEQ ID NO: 386) | GAYWGQGTLVTVSS |
| Exemplary anti-target | EVQLVESGGGLVQPGGSLTLSCAASGFTLDYYAIGWYRQAPGKKRELVAGIS |
| (DLL3) | SDGSTHYVDSVKGRFTISRDNAKNSVYLQMNSLRAEDTAVYYCYAYRWVGG |
| (SEQ ID NO: 387) | YTYWGQGTLVTVSS |
| Exemplary anti-target | EVQLVESGGGLVQPGGSLTLSCVASGSIFTTNSMGWHRQGPGKQRELVALI |
| (DLL3) | GSAGSTKYADSVKGRFTISRDNAKNSVYLQMNSLRAEDTAVYYCFYYDSRSY |
| (SEQ ID NO: 388) | WGQGTLVTVSS |
| Exemplary anti-target | EVQLVESGGGLVQPGGSLTLSCAASGITSSVYSMGWYRQAPGKQRELVAGS |
| (DLL3) | SSDGSTHYVDSVRGRFTISRDNAKNSVYLQMNSLRAEDTAVYYCYANRGFA |
| (SEQ ID NO: 389) | GAPSYWGQGTLVTVSS |
| Exemplary anti-target | EVQLVESGGGLVQPGGSLTLSCAASSSIFSISSMSWYRQAPGKQRELVAAITS |
| (DLL3) | FGSTNYADSVKGRFTISRDNAKNSMYLQMNSLRAEDTAVYYCNARTMGRD |
| (SEQ ID NO: 390) | YWGQGTLVTVSS |
| Exemplary anti-target | EVQLVESGGGLVQPGGSLTLSCVASGFTSSINAMGWYRRAPGKQRELVAGI |
| (DLL3) | SSDGSFVYADSVKGRFTISRDNAKNSVYLQMNSLRAEDTAVYYCYYFRHVSG |
| (SEQ ID NO: 391) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYADSVKGRFTISRDNAKNSVYLQMNSLRAEDTAVYYCYYFRTVSG |
| (SEQ ID NO: 392) | SSSRYWGQGTLVTVSS |
| Exemplary anti-target | EVQLVESGGGLVQPGGSLTLSCVASGSTSSINAMGWYRRAPGKQRELVAGI |
| (DLL3) | SSDGSKVYEDSVKGRFTISRDNAKNSVYLQMNSLRAEDTAVYYCYYFRTVSG |
| (SEQ ID NO: 393) | SSMRYWGQGTLVTVSS |
| Exemplary anti-target | EVQLVESGGGLVQPGGSLTLSCVASGSPSSINAMGWYRRAPGKQRELSAGI |
| (DLL3) | SSDGSKVYADSVKGRFTISRDNAKNSVYLQMNSLRAEDTAVYYCYYFRTVRG |
| (SEQ ID NO: 394) | SSMSYWGQGTLVTVSS |
| Exemplary anti-target | EVQLVESGGGLVQPGGSLTLSCVASGSTSSINAMGWYRRAPGKQRELAAGI |
| (DLL3) | SSDGSSVYADSVKGRFTISRDNAKNSVYLQMNSLRAEDTAVYYCYYFRTVSG |
| (SEQ ID NO: 395) | SSKRYWGQGTLVTVSS |
| Exemplary anti-target | EVQLVESGGGLVQPGGSLTLSCVASGSISSINAMGWYRRAPGKQRELVAGIS |
| (DLL3) | SDGSKVYADSVKGRFTISRDNAKNSVYLQMNSLRAEDTAVYYCYYFRMVSG |
| (SEQ ID NO: 396) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKLYADSVKGRFTISRDNAKNSVYLQMNSLRAEDTAVYYCYYFRTVQG |
| (SEQ ID NO: 397) | SSMRYWGQGTLVTVSS |
| Exemplary anti-target | EVQLVESGGGLVQPGGSLTLSCVASGSTSSINAYGWYRRAPGKQRELVAGIS |
| (DLL3) | SDGSKVYADSVKGRFTISRDNAKNSVYLQMNSLRAEDTAVYYCYYFRTVYGS |
| (SEQ ID NO: 398) | SMRYWGQGTLVTVSS |
| Exemplary anti-target | EVQLVESGGGLVQPGGSLTLSCVASGSTSSINAIGWYRRAPGKQRELVAGISS |
| (DLL3) | DGSKVYIDSVKGRFTISRDNAKNSVYLQMNSLRAEDTAVYYCYYFRTVSGSSY |
| (SEQ ID NO: 399) | RYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYSDSVKGRFTISRDNAKNSVYLQMNSLRAEDTAVYYCYYFRTVLG |
| (SEQ ID NO: 400) | SSMRYWGQGTLVTVSS |
| Exemplary anti-target | EVQLVESGGGLVQPGGSLTLSCVASGSTSIINAMGWYRRAPGKQRELAAGIS |
| (DLL3) | SDGSKVIADSVKGRFTISRDNAKNSVYLQMNSLRAEDTAVYYCYYFRRVSGS |
| (SEQ ID NO: 401) | SMRYWGQGTLVTVSS |
| Exemplary anti-target | EVQLVESGGGLVQPGGSLTLSCVASGSTSSINAMGWYRRAPGKQRELVAGI |
| (DLL3) | SSDGSKIYADSVKGRFTISRDNAKNSVYLQMNSLRAEDTAVYYCYYFRTVSGS |
| (SEQ ID NO: 402) | SMRYWGQGTLVTVSS |
| Exemplary anti-target | EVQLVESGGGLVQPGGSLTLSCVASGKTSSINAMAWYRRAPGKQRELVAGI |
| (DLL3) | SSDGSKVYTDSVKGRFTISRDNAKNSVYLQMNSLRAEDTAVYYCYYFRTVSG |
| (SEQ ID NO: 403) | SSARYWGQGTLVTVSS |
| Exemplary anti-target | EVQLVESGGGLVQPGGSLTLSCVASGTTSSINAMGWYRRAPGKQRELVAGI |
| (DLL3) | SSDGSLVYADSVKGRFTISRDNAKNSVYLQMNSLRAEDTAVYYCYYFRIVRGS |
| (SEQ ID NO: 404) | SMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYADSVKGRFTISRDNAKNSVYLQMNSLRAEDTAVYYCYYYRTVSG |
| (SEQ ID NO: 405) | SSMRYWGQGTLVTVSS |
| Exemplary anti-target | EVQLVESGGGLVQPGGSLTLSCVASGSGSSINAMGWYRRAPGKQRELVAGI |
| (DLL3) | SSDGSKVYSDSVKGRFTISRDNAKNSVYLQMNSLRAEDTAVYYCYYFRHVSG |
| (SEQ ID NO: 406) | SSMRYWGQGTLVTVSS |
| Exemplary anti-target | EVQLVESGGGLVQPGGSLTLSCVASGTTSSINAMGWYRRAPGKQRELVAGI |
| (DLL3) | SSDGSKVYVDSVKGRFTISRDNAKNSVYLQMNSLRAEDTAVYYCYYFRFVSG |
| (SEQ ID NO: 407) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYVDSVKGRFTISRDNAKNSVYLQMNSLRAEDTAVYYCYYFRTVSG |
| (SEQ ID NO: 408) | SSMRYWGQGTLVTVSS |
| Exemplary anti-target | EVQLVESGGGLVQPGGSLTLSCVASGSTSRINAMGWYRRAPGKQRELVAGI |
| (DLL3) | SSDGSKVYADSVKGRFTISRDNAKNSVYLQMNSLRAEDTAVYYCYYFRTKSG |
| (SEQ ID NO: 409) | SSMRYWGQGTLVTVSS |
| Exemplary anti-target | EVQLVESGGGLVQPGGSLTLSCVASGSTSRINAMGWYRRAPGKQRELVAGI |
| (DLL3) | SSDGSKVYADSVKGRFTISRDNAKNSVYLQMNSLRAEDTAVYYCYYFRTVYG |
| (SEQ ID NO: 410) | SSMRYWGQGTLVTVSS |
| Exemplary anti-target | EVQLVESGGGLVQPGGSLTLSCVASGSTSSINAMGWYRRAPGKQRELVAGI |
| (DLL3) | SSDGSKVYRDSVKGRFTISRDNAKNSVYLQMNSLRAEDTAVYYCYYFRTVSG |
| (SEQ ID NO: 411) | SSMGYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYSDSVKGRFTISRDNAKNSVYLQMNSLRAEDTAVYYCYYFRTVSG |
| (SEQ ID NO: 412) | SSMRSWGQGTLVTVSS |
| Exemplary anti-target | EVQLVESGGGLVQPGGSLTLSCVASGSTSSINAMAWYRRAPGKQRELVAGI |
| (DLL3) | SSDNSKVYADSVKGRFTISRDNAKNSVYLQMNSLRAEDTAVYYCYYFRTVGG |
| (SEQ ID NO: 413) | SSMRYWGQGTLVTVSS |
| Exemplary anti-target | EVQLVESGGGLVQPGGSLTLSCVASGNTSSINAMAWYRRAPGKQRELVAGI |
| (DLL3) | SSDGSKVYADSVKGRFTISRDNAKNSVYLQMNSLRAEDTAVYYCYYFRTVSG |
| (SEQ ID NO: 414) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYADSVKGRFTISRDNAKNSVYLQMNSLRAEDTAVYYCYYFRTVSG |
| (SEQ ID NO: 415) | SHMRYWGQGTLVTVSS |
| Exemplary anti-target | EVQLVESGGGLVQPGGSLTLSCVASGSTSIINAMGWYRRAPGKQRELVAGIS |
| (DLL3) | SDGSKVYEDSVKGRFTISRDNAKNSVYLQMNSLRAEDTAVYYCYYFRAVSGS |
| (SEQ ID NO: 416) | SMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYADSVKGRFTISRDNAKNSVYLQMNSLRAEDTAVYYCYYFRTVSG |
| (SEQ ID NO: 417) | SSMRYWGQGTLVTVSS |
| Exemplary anti-target | EVQLVESGGGLVQPGGSLTLSCVASGSTSRINAMGWYRRAPGKQRELPAGI |
| (DLL3) | SSDGSKVYAVSVKGRFTISRDNAKNSVYLQMNSLRAEDTAVYYCYYFRTVSG |
| (SEQ ID NO: 418) | SPMRYWGQGTLVTVSS |
| Exemplary anti-target | EVQLVESGGGLVQPGGSLTLSCVASGSTSRINAMGWYRRAPGKQRELVAG |
| (DLL3) | VSSDGSKVYADSVKGRFTISRDNAKNSVYLQMNSLRAEDTAVYYCYYFRTVS |
| (SEQ ID NO: 419) | GSSMSYWGQGTLVTVSS |
| Exemplary anti-target | EVQLVESGGGLVQPGGSLTLSCVASGTTSSINAMGWYRRAPGKQRELVAGI |
| (DLL3) | SSDGSKVYEDSVKGRFTISRDNAKNSVYLQMNSLRAEDTAVYYCYYFRTVSG |
| (SEQ ID NO: 420) | SSMRYWGQGTLVTVSS |
| Exemplary anti-target | EVQLVESGGGLVQPGGSLTLSCVASGITSSINAMGWYRRAPGKQRELVAGIS |
| (DLL3) | SDGSKVYAGSVKGRFTISRDNAKNSVYLQMNSLRAEDTAVYYCYYFRTVRGS |
| (SEQ ID NO: 421) | SMRYWGQGTLVTVSS |
| Exemplary anti-target | EVQLVESGGGLVQPGGSLTLSCVASGSTSDINAMGWYRRAPGKQRELVAGI |
| (DLL3) | SSDKSKVYADSVKGRFTISRDNAKNSVYLQMNSLRAEDTAVYYCYYFRTVRG |
| (SEQ ID NO: 422) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSNGSKVYADSVKGRFTISRDNAKNSVYLQMNSLRAEDTAVYYCYYFRQVSG |
| (SEQ ID NO: 423) | SSMRYWGQGTLVTVSS |
| Exemplary anti-target | EVQLVESGGGLVQPGGSLTLSCVASGSTSSINAIGWYRRAPGKQRELVAGISS |
| (DLL3) | DGSKVLADSVKGRFTISRDNAKNSVYLQMNSLRAEDTAVYYCYYFRIVSGSS |
| (SEQ ID NO: 424) | MGYWGQGTLVTVSS |
| Exemplary anti-target | EVQLVESGGGLVQPGGSLTLSCVASGSTSSKNAMGWYRRAPGKQRELVAGI |
| (DLL3) | SSDGSKVYADSVKGRFTISRDNAKNSVYLQMNSLRAEDTAVYYCYYFRTVSG |
| (SEQ ID NO: 425) | ASMRYWGQGTLVTVSS |
| Exemplary anti-target | EVQLVESGGGLVQPGGSLTLSCVASGSTSSINAMGWYRRAPGKQRELVAGI |
| (DLL3) | SSDNSKVYADSVKGRFTISRDNAKNSVYLQMNSLRAEDTAVYYCYYFRTVHG |
| (SEQ ID NO: 426) | SSMRYWGQGTLVTVSS |
| Exemplary anti-target | EVQLVESGGGLVQPGGSLTLSCVASGLTSSINAMGWYRRAPGKQRELVAGI |
| (DLL3) | SSDGSKVYADSVKGRFTISRDNAKNSVYLQMNSLRAEDTAVYYCYYFRMVS |
| (SEQ ID NO: 427) | GSSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYTDSVKGRFTISRDNAKNSVYLQMNSLRAEDTAVYYCYYFRTISGS |
| (SEQ ID NO: 428) | SMRYWGQGTLVTVSS |
| Exemplary anti-target | EVQLVESGGGLVQPGGSLTLSCVASGSTSSNNAMAWYRRAPGKQRELVAGI |
| (DLL3) | SSDGSKVYTDSVKGRFTISRDNAKNSVYLQMNSLRAEDTAVYYCYYFRTRSG |
| (SEQ ID NO: 429) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSDNSKVYADSVKGRFTISRDNAKNSVYLQMNSLRAEDTAVYYCYYFRTVSG |
| (SEQ ID NO: 430) | HSMRYWGQGTLVTVSS |
| Exemplary anti-target | EVQLVESGGGLVQPGGSLTLSCVASGSTSHINAMGWYRRAPGKQRELVAGI |
| (DLL3) | SSDGSRVYADSVKGRFTISRDNAKNSVYLQMNSLRAEDTAVYYCYYFRTVSG |
| (SEQ ID NO: 431) | GSMRYWGQGTLVTVSS |
| Exemplary anti-target | EVQLVESGGGLVQPGGSLTLSCVASGQTSSINAMGWYRRAPGKQRELVAGI |
| (DLL3) | SSDGSQVYADSVKGRFTISRDNAKNSVYLQMNSLRAEDTAVYYCYYFRTKSG |
| (SEQ ID NO: 432) | SSMRYWGQGTLVTVSS |
| Exemplary anti-target | EVQLVESGGGLVQPGGSLTLSCVASGSTSRINGMGWYRRAPGKQRELPAGI |
| (DLL3) | SSDGSKAYADSVKGRFTISRDNAKNSVYLQMNSLRAEDTAVYYCYYFRTASG |
| (SEQ ID NO: 433) | TSMRYWGQGTLVTVSS |
| Exemplary anti-target | EVQLVESGGGLVQPGGSLTLSCVASGSTSVINAMAWYRRAPGKQRELAAGI |
| (DLL3) | SSDGSKVYAKSAKGRFTISRDNAKNSVYLQMNSLRAEDTAVYYCYYFNTVSG |
| (SEQ ID NO: 434) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYNDSVKGRFTISRDNAKNSVYLQMNSLRAEDTAVYYCYYFRTVRG |
| (SEQ ID NO: 435) | SSQRYWGQGTLVTVSS |
| Exemplary anti-target | EVQLVESGGGLVQPGGSLTLSCVASGKTSSINAMGWYRRAPGKQRELVAGI |
| (DLL3) | SSDGSKVIADSVKGRFTISRDNAKNSVYLQMNSLRAEDTAVYYCYYFRTVLGS |
| (SEQ ID NO: 436) | SMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYTDSVKGRFTISRDNAKNSVYLQMNSLRAEDTAVYYCYYFRTRSG |
| (SEQ ID NO: 437) | SSMRYWGQGTLVTVSS |
| Exemplary anti-target | EVQLVESGGGLVQPGGSLTLSCVASGSVSSINAMGWYRRAPGKQRELVAGI |
| (DLL3) | SSDGSKVYIDSVKGRFTISRDNAKNSVYLQMNSLRAEDTAVYYCYYFRTVSGL |
| (SEQ ID NO: 438) | SMRYWGQGTLVTVSS |
| Exemplary anti-target | EVQLVESGGGLVQPGGSLTLSCVASGNTSSINAMGWYRRAPGKQRELVAGI |
| (DLL3) | SSDGSKVYYDSVKGRFTISRDNAKNSVYLQMNSLRAEDTAVYYCYYFRTVRG |
| (SEQ ID NO: 439) | SSQRYWGQGTLVTVSS |
| Exemplary anti-target | EVQLVESGGGLVQPGGSLTLSCVASGSTSSTNAMGWYRRAPGKQRELVAGI |
| (DLL3) | SSDGSKVYVDSVKGRFTISRDNAKNSVYLQMNSLRAEDTAVYYCYYFRTVSG |
| (SEQ ID NO: 440) | SSMVYWGQGTLVTVSS |
| Exemplary anti-target | EVQLVESGGGLVQPGGSLTLSCVASGSTSSINAMGWYRRAPGKQRELVAGI |
| (DLL3) | SSDGSKVYGDSVKGRFTISRDNAKNSVYLQMNSLRAEDTAVYYCYYFRTVSR |
| (SEQ ID NO: 441) | SSMRYWGQGTLVTVSS |
| Exemplary anti-target | EVQLVESGGGLVQPGGSLTLSCVASGSTSSINAMAWYRRAPGKQRELAAGI |
| (DLL3) | SSDQSKVYADSAKGRFTISRDNAKNSVYLQMNSLRAEDTAVYYCYYFRTVSG |
| (SEQ ID NO: 442) | SSMSYWGQGTLVTVSS |
| Exemplary anti-target | EVQLVESGGGLVQPGGSLTLSCVASGGTSSINAMGWYRRAPGKQRELVAGI |
| (DLL3) | SSDGSKVYSDSVKGRFTISRDNAKNSVYLQMNSLRAEDTAVYYCYYFRTVSG |
| (SEQ ID NO: 443) | SSARYWGQGTLVTVSS |
| Exemplary anti-target | EVQLVESGGGLVQPGGSLTLSCVASGSTRSINAMGWYRRAPGKQRELVAGI |
| (DLL3) | SSDGSKVYADSVKGRFTISRDNAKNSVYLQMNSLRAEDTAVYYCYYFHTVSG |
| (SEQ ID NO: 444) | SSMRYWGQGTLVTVSS |
| Exemplary anti-target | EVQLVESGGGLVQPGGSLTLSCVASGTTSSINAMGWYRRAPGKQRELVAGI |
| (DLL3) | SSDGSKVIADSVKGRFTISRDNAKNSVYLQMNSLRAEDTAVYYCYYFRTVLGS |
| (SEQ ID NO: 445) | SMRYWGQGTLVTVSS |
| Exemplary anti-target | EVQLVESGGGLVQPGGSLTLSCVASGSTSSINAMGWYRRAPGKQRELVAGI |
| (DLL3) | SSDGSKVDADSVKGRFTISRDNAKNSVYLQMNSLRAEDTAVYYCYYFRTVSG |
| (SEQ ID NO: 446) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYKDSVKGRFTISRDNAKNSVYLQMNSLRAEDTAVYYCYYFRNVSG |
| (SEQ ID NO: 447) | SSMRYWGQGTLVTVSS |
| Exemplary anti-target | EVQLVESGGGLVQPGGSLTLSCVASGNTSSINAMGWYRRAPGKQRELVAGI |
| (DLL3) | SSNGSKVYADSVKGRFTISRDNAKNSVYLQMNSLRAEDTAVYYCYYFRTVTG |
| (SEQ ID NO: 448) | SSMRYWGQGTLVTVSS |
| Exemplary anti-target | EVQLVESGGGLVQPGGSLTLSCVASGSTSRINAMGWYRRAPGKQRELVAGI |
| (DLL3) | SSDGSKVYKDSVKGRFTISRDNAKNSVYLQMNSLRAEDTAVYYCYYFRTVSG |
| (SEQ ID NO: 449) | SSMRYWGQGTLVTVSS |
| Exemplary anti-target | EVQLVESGGGLVQPGGSLTLSCVASGSTSSINAIGWYRRAPGKQRELVAGISS |
| (DLL3) | DGSKVYADSVKGRFTISRDNAKNSVYLQMNSLRAEDTAVYYCYYFRTVKGSS |
| (SEQ ID NO: 450) | MRYWGQGTLVTVSS |
| Exemplary anti-target | EVQLVESGGGLVQPGGSLTLSCVASGLTSSINAMGWYRRAPGKQRELVAGI |
| (DLL3) | SSDGSKVYQDSVKGRFTISRDNAKNSVYLQMNSLRAEDTAVYYCYYFRTNSG |
| (SEQ ID NO: 451) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYAESVKGRFTISRDNAKNSVYLQMNSLRAEDTAVYYCYYFRTVSG |
| (SEQ ID NO: 452) | ASMRYWGQGTLVTVSS |
| Exemplary anti-target | EVQLVESGGGLVQPGGSLTLSCVASGSTSSTNAMGWYRRAPGKQRELVAGI |
| (DLL3) | SSDGSKVLADSVKGRFTISRDNAKNSVYLQMNSLRAEDTAVYYCYYFRTVNL |
| (SEQ ID NO: 453) | SSMRYWGQGTLVTVSS |
| Exemplary anti-target | EVQLVESGGGLVQPGGSLTLSCVASGSTSSINAIGWYRRAPGKQRELVAGISS |
| (DLL3) | DGSKYYADSVKGRFTISRDNAKNSVYLQMNSLRAEDTAVYYCYYFRTVTGSS |
| (SEQ ID NO: 454) | MRYWGQGTLVTVSS |
| Exemplary anti-target | EVQLVESGGGLVQPGGSLTLSCVASGSTSSINAIGWYRRAPGKQRELVAGISS |
| (DLL3) | DGSKVYAVSVKGRFTISRDNAKNSVYLQMNSLRAEDTAVYYCYYFRKVSGSS |
| (SEQ ID NO: 455) | ARYWGQGTLVTVSS |
| Exemplary anti-target | EVQLVESGGGLVQPGGSLTLSCVASGSTSSINAMGWYRRAPGKQRELVAGI |
| (DLL3) | SSDGSKVVADSVKGRFTISRDNAKNSVYLQMNSLRAEDTAVYYCYYFRTYSG |
| (SEQ ID NO: 456) | SSMRYWGQGTLVTVSS |
| Exemplary anti-target | EVQLVESGGGLVQPGGSLTLSCVASGSTSSINAMGWYRRAPGKQRELVAGI |
| (DLL3) | SSDGSKVYADSVKGRFTISRDNAKNSVYLQMNSLRAEDTAVYYCYYFRTVSK |
| (SEQ ID NO: 457) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYADSVKGRFTISRDNAKNSVYLQMNSLRAEDTAVYYCYYFKTVSG |
| (SEQ ID NO: 458) | SSMRYWGQGTLVTVSS |
| Exemplary anti-target | EVQLVESGGGLVQPGGSLTLSCVASGSTSSINAMAWYRRAPGKQRELAAGI |
| (DLL3) | SSDNSKVYADSVKGRFTISRDNAKNSVYLQMNSLRAEDTAVYYCYYFRTRSG |
| (SEQ ID NO: 459) | SSMRYWGQGTLVTVSS |
| Exemplary anti-target | EVQLVESGGGLVQPGGSLTLSCVASGSKSSINAMGWYRRAPGKQRELAAGI |
| (DLL3) | SSDGSKVYAQSVKGRFTISRDNAKNSVYLQMNSLRAEDTAVYYCYYFRTSSG |
| (SEQ ID NO: 460) | SSMRYWGQGTLVTVSS |
| Exemplary anti-target | EVQLVESGGGLVQPGGSLTLSCVASGTTSSINAMGWYRRAPGKQRELVAGI |
| (DLL3) | SSDGSKVYVDSVKGRFTISRDNAKNSVYLQMNSLRAEDTAVYYCYYFRFLSG |
| (SEQ ID NO: 461) | SSMRYWGQGTLVTVSS |
| Exemplary anti-target | EVQLVESGGGLVQPGGSLTLSCVASGSTSSINAFGWYRRAPGKQRELVAGIS |
| (DLL3) | SDGSKVYSDSVKGRFTISRDNAKNSVYLQMNSLRAEDTAVYYCYYFRTVSGS |
| (SEQ ID NO: 462) | SMRYWGQGTLVTVSS |
| Exemplary anti-target | EVQLVESGGGLVQPGGSLTLSCVASGSTFSINAMGWYRRAPGKQRELVAGI |
| (DLL3) | SSDGSKVLADSVKGRFTISRDNAKNSVYLQMNSLRAEDTAVYYCYYFRLVSG |
| (SEQ ID NO: 463) | SSMRYWGQGTLVTVSS |
| Exemplary anti-target | EVQLVESGGGLVQPGGSLTLSCVASGSTRSINAMGWYRRAPGKQRELVAGI |
| (DLL3) | SSDGSKVYNDSVKGRFTISRDNAKNSVYLQMNSLRAEDTAVYYCYYFRTVSG |
| (SEQ ID NO: 464) | SSMRFWGQGTLVTVSS |
| Exemplary anti-target | EVQLVESGGGLVQPGGSLTLSCVASGSTSSINAIGWYRRAPGKQRELVAGISS |
| (DLL3) | DGSKVYNDSVKGRFTISRDNAKNSVYLQMNSLRAEDTAVYYCYYFRTQSGSS |
| (SEQ ID NO: 465) | MRYWGQGTLVTVSS |
| Exemplary anti-target | EVQLVESGGGLVQPGGSLTLSCVASGSTSSINAMGWYRRAPGKQRELVAGI |
| (DLL3) | SSDGSKVYVDSVKGRFTISRDNAKNSVYLQMNSLRAEDTAVYYCYYFRTVSG |
| (SEQ ID NO: 466) | SSMPYWGQGTLVTVSS |
| Exemplary anti-target | EVQLVESGGGLVQPGGSLTLSCVASGSTSSINAMGWYRRAPGKQRELVAGI |
| (DLL3) | SSDGSKVVADSVKGRFTISRDNAKNSVYLQMNSLRAEDTAVYYCYYFRTLSG |
| (SEQ ID NO: 467) | SSMRYWGQGTLVTVSS |
| Exemplary anti-target | EVQLVESGGGLVQPGGSLTLSCVASGTTSSINAMGWYRRAPGKQRELVAGI |
| (DLL3) | SSDGSKVYGDSVKGRFTISRDNAKNSVYLQMNSLRAEDTAVYYCYYFRTVSG |
| (SEQ ID NO: 468) | SAMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYTDSVKGRFTISRDNAKNSVYLQMNSLRAEDTAVYYCYYFRTTSG |
| (SEQ ID NO: 469) | SSMRYWGQGTLVTVSS |
| Exemplary anti-target | EVQLVESGGGLVQPGGSLTLSCVASGRISSINAMGWYRRAPGKQRELVAGI |
| (DLL3) | SSDGSKVYNDSVKGRFTISRDNAKNSVYLQMNSLRAEDTAVYYCYYFRTVSG |
| (SEQ ID NO: 470) | TSMRYWGQGTLVTVSS |
| Exemplary anti-target | EVQLVESGGGLVQPGGSLTLSCVASGSTSSRNAMGWYRRAPGKQRELVAGI |
| (DLL3) | SSDGSKVTADSVKGRFTISRDNAKNSVYLQMNSLRAEDTAVYYCYYFRTRSG |
| (SEQ ID NO: 471) | SSMRYWGQGTLVTVSS |
| Exemplary anti-target | EVQLVESGGGLVQPGGSLTLSCVASGSTKSINAMGWYRRAPGKQRELVAGI |
| (DLL3) | SSDGSKVYRDSVKGRFTISRDNAKNSVYLQMNSLRAEDTAVYYCYYFRTSSG |
| (SEQ ID NO: 472) | SSMRYWGQGTLVTVSS |
| Exemplary anti-target | EVQLVESGGGLVQPGGSLTLSCVASGSTSSRNAMGWYRRAPGKQRELVAGI |
| (DLL3) | SSNGSKVYSDSVKGRFTISRDNAKNSVYLQMNSLRAEDTAVYYCYYFRTVSG |
| (SEQ ID NO: 473) | SSMSYWGQGTLVTVSS |
| Exemplary anti-target | EVQLVESGGGLVQPGGSLTLSCVASGSTSSINAIGWYRRAPGKQRELVAGISS |
| (DLL3) | DGSKVYSDSVKGRFTISRDNAKNSVYLQMNSLRAEDTAVYYCYYFRPVSGSS |
| (SEQ ID NO: 474) | MRYWGQGTLVTVSS |
| Exemplary anti-target | EVQLVESGGGLVQPGGSLTLSCVASGSTSLINAMGWYRRAPGKQRELVAGI |
| (DLL3) | SSDGSKVYADSVKGRFTISRDNAKNSVYLQMNSLRAEDTAVYYCYYFRHVSG |
| (SEQ ID NO: 475) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYADSVKGRFTISRDNAKNSVYLQMNSLRAEDTAVYYCYYFRTKSG |
| (SEQ ID NO: 476) | SSMRYWGQGTLVTVSS |
| Exemplary anti-target | EVQLVESGGGLVQPGGSLTLSCVASGSTSSINAMGWYRRAPGKQRELVAGI |
| (DLL3) | SSDGSLVYADSVKGRFTISRDNAKNSVYLQMNSLRAEDTAVYYCYYFTTVSG |
| (SEQ ID NO: 477) | SSMRYWGQGTLVTVSS |
| Exemplary anti-target | EVQLVESGGGLVQPGGSLTLSCVASGTTSSINAMGWYRRAPGKQRELVAGI |
| (DLL3) | SSDGTKVYADSVKGRFTISRDNAKNSVYLQMNSLRAEDTAVYYCYYFHTVSG |
| (SEQ ID NO: 478) | SSMRYWGQGTLVTVSS |
| Exemplary anti-target | EVQLVESGGGLVQPGGSLTLSCVASGSTSSINAFGWYRRAPGKQRELVAGIS |
| (DLL3) | SDGSKVYADSVKGRFTISRDNAKNSVYLQMNSLRAEDTAVYYCYYFRTVRGS |
| (SEQ ID NO: 479) | SMRYWGQGTLVTVSS |
| Exemplary anti-target | EVQLVESGGGLVQPGGSLTLSCVASGSTSSRNAMGWYRRAPGKQRELVAGI |
| (DLL3) | SSDGSKLYLDSVKGRFTISRDNAKNSVYLQMNSLRAEDTAVYYCYYFRTVLGS |
| (SEQ ID NO: 480) | SMRYWGQGTLVTVSS |
| Exemplary anti-target | EVQLVESGGGLVQPGGSLTLSCVASGNTSSINAMGWYRRAPGKQRELVAGI |
| (DLL3) | SSDGSRVYADSVKGRFTISRDNAKNSVYLQMNSLRAEDTAVYYCYYFRTVSG |
| (SEQ ID NO: 481) | SSMRSWGQGTLVTVSS |
| Exemplary anti-target | EVQLVESGGGLVQPGGSLTLSCVASGTTSSINAMGWYRRAPGKQRELVAGI |
| (DLL3) | SSDGSKVYNDSVKGRFTISRDNAKNSVYLQMNSLRAEDTAVYYCYYFRTVRG |
| (SEQ ID NO: 482) | SSMRYWGQGTLVTVSS |
| Exemplary anti-target | EVQLVESGGGLVQPGGSLTLSCVASGSTASINAMGWYRRAPGKQRELVAGI |
| (DLL3) | SSDGSKVYADSVKGRFTISRDNAKNSVYLQMNSLRAEDTAVYYCYYFRYVSG |
| (SEQ ID NO: 483) | SSMRYWGQGTLVTVSS |
| Exemplary anti-target | EVQLVESGGGLVQPGGSLTLSCVASGSTSSINAVGWYRRAPGKQRELVAGIS |
| (DLL3) | SDGSKVYVDSVKGRFTISRDNAKNSVYLQMNSLRAEDTAVYYCYYFRTVYGS |
| (SEQ ID NO: 484) | SMRYWGQGTLVTVSS |
| Exemplary anti-target | EVQLVESGGGLVQPGGSLTLSCVASGSTSSRNAMGWYRRAPGKQRELVAGI |
| (DLL3) | SSDGSKLYADSVKGRFTISRDNAKNSVYLQMNSLRAEDTAVYYCYYFRTVLG |
| (SEQ ID NO: 485) | SSMRYWGQGTLVTVSS |
| Exemplary anti-target | EVQLVESGGGLVQPGGSLTLSCVASGSTNSINAMGWYRRAPGKQRELVAGI |
| (DLL3) | SSDGSKVYKDSVKGRFTISRDNAKNSVYLQMNSLRAEDTAVYYCYYYRTVSG |
| (SEQ ID NO: 486) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYADSVKGRFTISRDNAKNSVYLQMNSLRAEDTAVYYCYYFRSVSG |
| (SEQ ID NO: 487) | SSMRYWGQGTLVTVSS |
| Exemplary anti-target | EVQLVESGGGLVQPGGSLTLSCVASGSTSRINAMGWYRRAPGKQRELVAGI |
| (DLL3) | SSDGSKVYQDSVKGRFTISRDNAKNSVYLQMNSLRAEDTAVYYCYYFRRVSG |
| (SEQ ID NO: 488) | SSMRYWGQGTLVTVSS |
| Exemplary anti-target | EVQLVESGGGLVQPGGSLTLSCVPSGSTSNINAMGWYRRAPGKQRELPAGI |
| (DLL3) | SSDGTKIYADSAKVPFTITRDNAKNSVYLQMNSLRAEDTAVYYCYYFRTVSGT |
| (SEQ ID NO: 489) | SMRYWGQGTLVTVSS |
| Exemplary anti-target | EVQLVESGGGLVQPGGSLTLSCVASGSTSKINAMGWYRRAPGKQRELVAGI |
| (DLL3) | SSDRSKVYADSVKGRFTISRDNAKNSVYLQMNSLRAEDTAVYYCYYFRTVAG |
| (SEQ ID NO: 490) | SSMRYWGQGTLVTVSS |
| Exemplary anti-target | EVQLVESGGGLVQPGGSLTLSCVASGSTSSINALGWYRRAPGKQRELVAGIS |
| (DLL3) | SDGSLVYADSVKGRFTISRDNAKNSVYLQMNSLRAEDTAVYYCYYFRIVSGSS |
| (SEQ ID NO: 491) | MRYWGQGTLVTVSS |
| Exemplary anti-target | EVQLVESGGGLVQPGGSLTLSCVASGKTSSINAMGWYRRAPGKQRELVAGI |
| (DLL3) | SSDGSKVYADSVKGRFTISRDNAKNSVYLQMNSLRAEDTAVYYCYYFRTVSG |
| (SEQ ID NO: 490) | VSMRYWGQGTLVTVSS |
| Exemplary anti-target | EVQLVESGGGLVQPGGSLTLSCVASGSTSSINAVGWYRRAPGKQRELVAGIS |
| (DLL3) | SDGSKVYRDSVKGRFTISRDNAKNSVYLQMNSLRAEDTAVYYCYYFRTVQGS |
| (SEQ ID NO: 491) | SMRYWGQGTLVTVSS |
| Exemplary anti-target | EVQLVESGGGLVQPGGSLTLSCVASGSTSRINAMGWYRRAPGKQRELVAGI |
| (DLL3) | SSDGSKVYADSVKGRFTISRDNAKNSVYLQMNSLRAEDTAVYYCYYFRTASG |
| (SEQ ID NO: 492) | SSMRYWGQGTLVTVSS |
| Exemplary anti-target | EVQLVESGGGLVQPGGSLTLSCVASGSTSSINAMGWYRRAPGKQRELVAGI |
| (DLL3) | SSDGSKVYSDSVKGRFTISRDNAKNSVYLQMNSLRAEDTAVYYCYYFRTVSG |
| (SEQ ID NO: 493) | SSSRYWGQGTLVTVSS |
| Exemplary anti-target | EVQLVESGGGLVQPGGSLTLSCVASGSTSSINAVGWYRRAPGKQRELVAGIS |
| (DLL3) | SDGTKVYRDSVKGRFTISRDNAKNSVYLQMNSLRAEDTAVYYCYYFRTVQGS |
| (SEQ ID NO: 494) | SMRYWGQGTLVTVSS |
| Exemplary anti-target | EVQLVESGGGLVQPGGSLTLSCVASGTTSSINAMGWYRRAPGKQRELAAGI |
| (DLL3) | SSDGSKVYNDSVKGRFTISRDNAKNSVYLQMNSLRAEDTAVYYCYYFRTVRG |
| (SEQ ID NO: 495) | SSMRYWGQGTLVTVSS |
| Exemplary anti-target | EVQLVESGGGLVQPGGSLTLSCVASGSTSSINAMGWYRRAPGKQRELVAGI |
| (DLL3) | SSDGSKVYADSVKGRFTISRDNAKNSVYLQMNSLRAEDTAVYYCYYFRTKSG |
| (SEQ ID NO: 496) | SSMRYWGQGTLVTVSS |
| Exemplary anti-target | EVQLVESGGGLVQPGGSLTLSCVASGTTSSINAMGWYRRAPGKQRELVAGI |
| (DLL3) | SSDGSKVYADSVKGRFTISRDNAKNSVYLQMNSLRAEDTAVYYCYYFRTVW |
| (SEQ ID NO: 497) | GSSMRYWGQGTLVTVSS |
| Exemplary anti-target | EVQLVESGGGLVQPGGSLTLSCVASGKTSSINAMGWYRRAPGKQRELVAGI |
| (DLL3) | SSDGSKVYTDSVKGRFTISRDNAKNSVYLQMNSLRAEDTAVYYCYYFRTRSG |
| (SEQ ID NO: 498) | SSMRYWGQGTLVTVSS |
| Exemplary anti-target | EVQLVESGGGLVQPGGSLTLSCVASGSTSSINAMGWYRRAPFKQGELPAGIS |
| (DLL3) | PDGTKAYADSAKVRFTISRDNAKNSVYLQMNSLRAEDTAVYYCYYFHTVCGT |
| (SEQ ID NO: 499) | SMGYWGQGTLVTVSS |
| Exemplary anti-target | EVQLVESGGGLVQPGGSLTLSCVASGSTSAINAMGWYRRAPGKQRELVAGI |
| (DLL3) | SSDGSKVYVDSVKGRFTISRDNAKNSVYLQMNSLRAEDTAVYYCYYFRTVSG |
| (SEQ ID NO: 500) | SSQRYWGQGTLVTVSS |
| Exemplary anti-target | EVQLVESGGGLVQPGGSLTLSCVASGSPSSINAYGWYRRAPGKQRELVAGIS |
| (DLL3) | SDGSKVYSDSVKGRFTISRDNAKNSVYLQMNSLRAEDTAVYYCYYFRTVSGS |
| (SEQ ID NO: 501) | SMSYWGQGTLVTVSS |
| Exemplary anti-target | EVQLVESGGGLVQPGGSLTLSCVASGSTSSINAMGWYRRAPGKQRELVAGI |
| (DLL3) | SSDGSKVYASSVKGRFTISRDNAKNSVYLQMNSLRAEDTAVYYCYYFRTVRG |
| (SEQ ID NO: 502) | SSMRYWGQGTLVTVSS |
| Exemplary anti-target | EVQLVESGGGLVQPGGSLTLSCVASGSRSSINAMGWYRRAPGKQRELVAGI |
| (DLL3) | SADGSKVYADSVKGRFTISRDNAKNSVYLQMNSLRAEDTAVYYCYYFRTQSG |
| (SEQ ID NO: 503) | SSMRYWGQGTLVTVSS |
| Exemplary anti-target | EVQLVESGGGLVQPGGSLTLSCVASGSVSSINAMGWYRRAPGKQRELVAGI |
| (DLL3) | SSDGSKVYASSAKGRFTISRDNAKNSVYLQMNSLRAEDTAVYYCYYFRTLSGS |
| (SEQ ID NO: 504) | SMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYADSVKGRFTISRDNAKNSVYLQMNSLRAEDTAVYYCYYFHTVSG |
| (SEQ ID NO: 505) | SSMRYWGQGTLVTVSS |
| Exemplary anti-target | EVQLVESGGGLVQPGGSLTLSCVASGSTSSINAIGWYRRAPGKQRELVAGISS |
| (DLL3) | DGSSVYADSVKGRFTISRDNAKNSVYLQMNSLRAEDTAVYYCYYFRRVSGSS |
| (SEQ ID NO: 506) | MRYWGQGTLVTVSS |
| Exemplary anti-target | EVQLVESGGGLVQPGGSLTLSCVASGSTSSINAMGWYRRAPGKQRELVAGI |
| (DLL3) | SSDGSKVYSDSVKGRFTISRDNAKNSVYLQMNSLRAEDTAVYYCYYFRLVSG |
| (SEQ ID NO: 507) | SSMRYWGQGTLVTVSS |
| Exemplary anti-target | EVQLVESGGGLVQPGGSLTLSCVASGSTSSINAVGWYRRAPGKQRELVAGIS |
| (DLL3) | SDGSKVYAGSVKGRFTISRDNAKNSVYLQMNSLRAEDTAVYYCYYFRTVSGS |
| (SEQ ID NO: 508) | YMRYWGQGTLVTVSS |
| Exemplary anti-target | EVQLVESGGGLVQPGGSLTLSCVASGSTSSINAMAWYRRAPGKQRELAAGI |
| (DLL3) | SSDNSKVYADSVKGRFTISRDNAKNSVYLQMNSLRAEDTAVYYCYYFRTVGG |
| (SEQ ID NO: 509) | SSMRYWGQGTLVTVSS |
| Exemplary anti-target | EVQLVESGGGLVQPGGSLTLSCVASGSTSSINAYGWYRRAPGKQRELVAGIS |
| (DLL3) | SDGSAVYADSVKGRFTISRDNAKNSVYLQMNSLRAEDTAVYYCYYFRTHSGS |
| (SEQ ID NO: 510) | SMRYWGQGTLVTVSS |
| Exemplary anti-target | EVQLVESGGGLVQPGGSLTLSCVASGSTSSINAVGWYRRAPGKQRELVAGIS |
| (DLL3) | SDGSSVYADSVKGRFTISRDNAKNSVYLQMNSLRAEDTAVYYCYYFRTVSTS |
| (SEQ ID NO: 511) | SMRYWGQGTLVTVSS |
| Exemplary anti-target | EVQLVESGGGLVQPGGSLTLSCVASGSKSSINAMGWYRRAPGKQRELPAGI |
| (DLL3) | SSNGTKVYADSAKVRFTISRDNAKNSVYLQMNSLRAEDTAVYYCYYFRTVLG |
| (SEQ ID NO: 512) | TSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKLYADSVKGRFTISRDNAKNSVYLQMNSLRAEDTAVYYCYYFRTVSG |
| (SEQ ID NO: 513) | SSMRYWGQGTLVTVSS |
| Exemplary anti-target | EVQLVESGGGLVQPGGSLTLSCVASGSVSSINAMGWYRRAPGKQRELVAGI |
| (DLL3) | SSDGSKVYKDSVKGRFTISRDNAKNSVYLQMNSLRAEDTAVYYCYYFRTVSG |
| (SEQ ID NO: 514) | SSMGYWGQGTLVTVSS |
| Exemplary anti-target | EVQLVESGGGLVQPGGSLTLSCVASGSTSSINAMGWYRRAPGKQRELVAGI |
| (DLL3) | SSDGSLVYADSVKGRFTISRDNAKNSVYLQMNSLRAEDTAVYYCYYFRTVSG |
| (SEQ ID NO: 515) | SSMRAWGQGTLVTVSS |
| Exemplary anti-target | EVQLVESGGGLVQPGGSLTLSCVASGSTSSINAMGWYRRAPGKQRELVAGI |
| (DLL3) | SSDGSLVYADSVKGRFTISRDNAKNSVYLQMNSLRAEDTAVYYCYYFRILSGS |
| (SEQ ID NO: 516) | SMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYADSVKGRFTISRDNAKNSVYLQMNSLRAEDTAVYYCYYFRTVQG |
| (SEQ ID NO: 517) | SSMRYWGQGTLVTVSS |
| Exemplary anti-target | EVQLVESGGGLVQPGGSLTLSCVASGSTSYINAMGWYRRAPGKQRELVAGI |
| (DLL3) | SSDGSKVYADSVKGRFTISRDNAKNSVYLQMNSLRAEDTAVYYCYYFRTVSG |
| (SEQ ID NO: 518) | QSMGYWGQGTLVTVSS |
| Exemplary anti-target | EVQLVESGGGLVQPGGSLTLSCVASGSTSSINAMGWYRRAPGKQRELVAGV |
| (DLL3) | SSDGSKVYADSVKGRFTISRDNAKNSVYLQMNSLRAEDTAVYYCYYFRTVSG |
| (SEQ ID NO: 511) | SSARYWGQGTLVTVSS |
| Exemplary anti-target | EVQLVESGGGLVQPGGSLTLSCVASGSTSSINAMGWYRRAPGKQRELPAGI |
| (DLL3) | SRDGSKVYADSVKGRFTISRDNAKNSVYLQMNSLRAEDTAVYYCYYFRYVSG |
| (SEQ ID NO: 512) | SSMRYWGQGTLVTVSS |
| Exemplary anti-target | EVQLVESGGGLVQPGGSLTLSCVASGSTSSINAMGWYRRAPGKQRELAAGI |
| (DLL3) | SSDGSKLYADSVKGRFTISRDNAKNSVYLQMNSLRAEDTAVYYCYYFRTVSG |
| (SEQ ID NO: 513) | SSMRYWGQGTLVTVSS |
| Exemplary anti-target | EVQLVESGGGLVQPGGSLTLSCVASGSTSRINAMGWYRRAPGKQRELVAGI |
| (DLL3) | SSDGSKVYADSVKGRFTISRDNAKNSVYLQMNSLRAEDTAVYYCYYFRRVSG |
| (SEQ ID NO: 514) | SSMRYWGQGTLVTVSS |
| Exemplary anti-target | EVQLVESGGGLVQPGGSLTLSCVASGSTSSINAMAWYRRAPGKQRELAAGI |
| (DLL3) | SSDGSKVYADSVKGRFTISRDNAKNSVYLQMNSLRAEDTAVYYCYYFHTVSG |
| (SEQ ID NO: 515) | SSMRYWGQGTLVTVSS |
| Exemplary anti-target | EVQLVESGGGLVQPGGSLTLSCVASGSTSSINAMAWYRRAPGKQRELVAGI |
| (DLL3) | SSDGSKVYADSVKGRFTISRDNAKNSVYLQMNSLRAEDTAVYYCYYFRQVSG |
| (SEQ ID NO: 516) | SSMRYWGQGTLVTVSS |
| Exemplary anti-target | EVQLVESGGGLVQPGGSLTLSCVASGSTSSINAMGWYRRAPGKQRELVAGI |
| (DLL3) | SSDTSKVYADSVKGRFTISRDNAKNSVYLQMNSLRAEDTAVYYCYYFRTVSG |
| (SEQ ID NO: 517) | SYMRYWGQGTLVTVSS |
| Exemplary anti-target | EVQLVESGGGLVQPGGSLTLSCVASGSTSTINAMGWYRRAPGKQRELVAGI |
| (DLL3) | SSDGSKVYADSVKGRFTISRDNAKNSVYLQMNSLRAEDTAVYYCYYFRTASG |
| (SEQ ID NO: 518) | SSMRYWGQGTLVTVSS |
| Exemplary anti-target | EVQLVESGGGLVQPGGSLTLSCVASGSTSSINAVGWYRRAPGKQRELVAGIS |
| (DLL3) | SDGSTVYADSVKGRFTISRDNAKNSVYLQMNSLRAEDTAVYYCYYFRTVSGH |
| (SEQ ID NO: 519) | SMRYWGQGTLVTVSS |
| Exemplary anti-target | EVQLVESGGGLVQPGGSLTLSCVASGSTSSINAMGWYRRAPGKQRELAAGI |
| (DLL3) | SKDGSKVYADSAKGRFTISRDNAKNSVYLQMNSLRAEDTAVYYCYYFRTVSG |
| (SEQ ID NO: 520) | SSSRYWGQGTLVTVSS |
| Exemplary anti-target | EVQLVESGGGLVQPGGSLTLSCVASGSPSSINAYGWYRRAPGKQRELVAGIS |
| (DLL3) | SDGSKVYSDSVKGRFTISRDNAKNSVYLQMNSLRAEDTAVYYCYYFRTVSGS |
| (SEQ ID NO: 521) | SYSYWGQGTLVTVSS |
| Exemplary anti-target | EVQLVESGGGLVQPGGSLTLSCVASGSPSSINAYGWYRRAPGKQRELVAGIS |
| (DLL3) | SDGSKVYSDSVKGRFTISRDNAKNSVYLQMNSLRAEDTAVYYCYYFRTVSGS |
| (SEQ ID NO: 522) | SQSYWGQGTLVTVSS |
| Exemplary anti-target | EVQLVESGGGLVQPGGSLTLSCAASGSSVKFLSMAWYRQAPGKKRELVAGI |
| (DLL3) | SADGSTDYIDSVKGRFTISRDNAKNSVYLQMNSLRAEDTAVYYCYAYRWTRR |
| (SEQ ID NO: 523) | YTYWGQGTLVTVSS |
| Exemplary anti-target | EVQLVESGGGLVQPGGSLTLSCAASGSSVSFLSLAWYRQAPGKKRELVAGIS |
| (DLL3) | ADGSTAYIDSVKGRFTISRDNAKNSVYLQMNSLRAEDTAVYYCYAYRWRTRY |
| (SEQ ID NO: 524) | TYWGQGTLVTVSS |
| Exemplary anti-target | EVQLVESGGGLVQPGGSLTLSCAASGSRVSFLSMAWYRQAPGKKRELVAGI |
| (DLL3) | SRDGSTDYIDSVKGRFTISRDNAKNSVYLQMNSLRAEDTAVYYCYAYRWTTR |
| (SEQ ID NO: 525) | RTYWGQGTLVTVSS |
| Exemplary anti-target | EVQLVESGGGLVQPGGSLTLSCAASGSQVSFLSMAWYRQAPGKKRELVAGI |
| (DLL3) | SRDGSTDYIDSVKGRFTISRDNAKNSVYLQMNSLRAEDTAVYYCYAYRWTTR |
| (SEQ ID NO: 526) | YIYWGQGTLVTVSS |
| Exemplary anti-target | EVQLVESGGGLVQPGGSLTLSCAASGSSVSFLSMAWYRQAPGKKRELVAGIS |
| (DLL3) | EAGSTDYIDSVKGRFTISRDNAKNSVYLQMNSLRAEDTAVYYCYAYRWRTRY |
| (SEQ ID NO: 527) | TYWGQGTLVTVSS |
| Exemplary anti-target | EVQLVESGGGLVQPGGSLTLRCAASGSKVSFLSMAWYRQAPGKKRELVAGI |
| (DLL3) | SADGSTDYVDSVKGRFTISRDNAKNSVYLQMNSLRAEDTAVYYCYAYRWTR |
| (SEQ ID NO: 528) | RYTYWGQGTLVTVSS |
| Exemplary anti-target | EVQLVESGGGLVQPGGSLTLSCAASGSSVGFLSMAWYRQAPGKKRELVAGI |
| (DLL3) | SADGSTDYIRSVKGRFTISRDNAKNSVYLQMNSLRAEDTAVYYCYAYRWRTR |
| (SEQ ID NO: 529) | YTYWGQGTLVTVSS |
| Exemplary anti-target | EVQLVESGGGLVQPGGSLTLSCAASGSSVSFLSMAWYRQAPGKKRELVAGIS |
| (DLL3) | ADGSVDYIDSVKGRFTISRDNAKNSVYLQMNSLRAEDTAVYYCYAYRWTTR |
| (SEQ ID NO: 530) | YIYWGQGTLVTVSS |
| Exemplary anti-target | EVQLVESGGGLVQPGGSLTLSCAASGSRVSFLSMAWYRQAPGKKRELVAGI |
| (DLL3) | SADGSTLYIDSVKGRFTISRDNAKNSVYLQMNSLRAEDTAVYYCYAYRWTTR |
| (SEQ ID NO: 531) | RTYWGQGTLVTVSS |
| Exemplary anti-target | EVQLVESGGGLVQPGGSLTLSCAASGSSVSFLSLAWYRQAPGKKRELVAGIS |
| (DLL3) | TDGSTDYIDSVKGRFTISRDNAKNSVYLQMNSLRAEDTAVYYCYAYRWRTRY |
| (SEQ ID NO: 532) | TYWGQGTLVTVSS |
| Exemplary anti-target | EVQLVESGGGLVQPGGSLTLSCAASGSSVSFLSMAWYRQAPGKKRELVAGIS |
| (DLL3) | GDGSTDYIDSVKGRFTISRDNAKNSVYLQMNSLRAEDTAVYYCYAYRWTTR |
| (SEQ ID NO: 533) | YTYWGQGTLVTVSS |
| Exemplary anti-target | EVQLVESGGGLVQPGGSLTLSCAASGSSVQFLSMAWYRQAPGKKRELVAGI |
| (DLL3) | SADGSTDYINSVKGRFTISRDNAKNSVYLQMNSLRAEDTAVYYCYAYRWRT |
| (SEQ ID NO: 534) | RYTYWGQGTLVTVSS |
| Exemplary anti-target | EVQLVESGGGLVQPGGSLTLSCAASGSNVSFLSMAWYRQAPGKKRELVAGI |
| (DLL3) | SARGSTDYIDSVKGRFTISRDNAKNSVYLQMNSLRAEDTAVYYCYAYHWTTR |
| (SEQ ID NO: 535) | YTYWGQGTLVTVSS |
| Exemplary anti-target | EVQLVESGGGLVQPGGSLTLSCVASGSSVKFLSMAWYRQAPGKKRELVAGI |
| (DLL3) | SADGSTTYIDSVKGRFTISRDNAKNSVYLQMNSLRAEDTAVYYCYAYRWTTR |
| (SEQ ID NO: 536) | RTYWGQGTLVTVSS |
| Exemplary anti-target | EVQLVESGGGLVQPGGSLTLSCAASGKSVSFLSMAWYRQAPGKKRELVAGI |
| (DLL3) | SKDGSTDYIDSVKGRFTISRDNAKNSVYLQMNSLRAEDTAVYYCYAYRWTTR |
| (SEQ ID NO: 537) | YTYWGQGTLVTVSS |
| Exemplary anti-target | EVQLVESGGGLVQPGGSLTLSCAASGSRVSFLSMAWYRQAPGKKRELVAGI |
| (DLL3) | SADGSTTYIDSVKGRFTISRDNAKNSVYLQMNSLRAEDTAVYYCYAYRWTTR |
| (SEQ ID NO: 538) | YTYWGQGTLVTVSS |
| Exemplary anti-target | EVQLVESGGGLVQPGGSLTLSCAASGSHVSFLSMAWYRQAPGKKRELVAGI |
| (DLL3) | SANGSTDYIDSVKGRFTISRDNAKNSVYLQMNSLRAEDTAVYYCYAYRWTTR |
| (SEQ ID NO: 539) | YAYWGQGTLVTVSS |
| Exemplary anti-target | EVQLVESGGGLVQPGGSLTLSCAASGSSVSFLSMAWYRQAPGKKRELVAGIS |
| (DLL3) | RDGSTDYIDSVKGRFTISRDNAKNSVYLQMNSLRAEDTAVYYCYAYRWVTR |
| (SEQ ID NO: 540) | YTYWGQGTLVTVSS |
| Exemplary anti-target | EVQLVESGGGLVQPGGSLTLSCAASGSSVSFLSMAWYRQAPGKKRELVAGIS |
| (DLL3) | ADGSADYIDSVKGRFTISRDNAKNSVYLQMNSLRAEDTAVYYCYAYRWVTR |
| (SEQ ID NO: 541) | YTYWGQGTLVTVSS |
| Exemplary anti-target | EVQLVESGGGLVQPGGSLTLSCAASGSSVRFLSMAWYRQAPGKKRELVAGI |
| (DLL3) | SAFIGSTDYIDSVKGRFTISRDNAKNSVYLQMNSLRAEDTAVYYCYAYRWSTR |
| (SEQ ID NO: 542) | YTYWGQGTLVTVSS |
| Exemplary anti-target | EVQLVESGGGLVQPGGSLTLSCAASGSSVRFLSMAWYRQAPGKKRELVAGI |
| (DLL3) | SADGSTIYIDSVKGRFTISRDNAKNSVYLQMNSLRAEDTAVYYCYAYRWRTR |
| (SEQ ID NO: 543) | YTYWGQGTLVTVSS |
| Exemplary anti-target | EVQLVESGGGLVQPGGSLTLSCAASGSSVRFLSMAWYRQAPGKKRELVAGI |
| (DLL3) | SRDGSTVYIDSVKGRFTISRDNAKNSVYLQMNSLRAEDTAVYYCYAYRWTTR |
| (SEQ ID NO: 544) | GTYWGQGTLVTVSS |
| Exemplary anti-target | EVQLVESGGGLVQPGGSLTLSCAASGSHVSFLSMAWYRQAPGKKRELVAGI |
| (DLL3) | SADGPTDYIDSVKGRFTISRDNAKNSVYLQMNSLRAEDTAVYYCYAYRWDT |
| (SEQ ID NO: 545) | RYTYWGQGTLVTVSS |
| Exemplary anti-target | EVQLVESGGGLVQPGGSLTLSCVASGTSVSFLSMAWYRQAPGKKRELVAGI |
| (DLL3) | SADGSTTYIDSVKGRFTISRDNAKNSVYLQMNSLRAEDTAVYYCYAYRWTTR |
| (SEQ ID NO: 546) | RTYWGQGTLVTVSS |
| Exemplary anti-target | EVQLVESGGGLVQPGGSLTLSCAASGTSVSFLSIAWYRQAPGKKRELVAGISA |
| (DLL3) | DGSTDYIASVKGRFTISRDNAKNSVYLQMNSLRAEDTAVYYCYAYRWRTRYT |
| (SEQ ID NO: 547) | YWGQGTLVTVSS |
| Exemplary anti-target | EVQLVESGGGLVQPGGSLTLSCAASGSSVKFLSMAWYRQAPGKKRELVAGI |
| (DLL3) | SLDGSTDYIDSVKGRFTISRDNAKNSVYLQMNSLRAEDTAVYYCYAYRWTGR |
| (SEQ ID NO: 548) | YTYWGQGTLVTVSS |
| Exemplary anti-target | EVQLVESGGGLVQPGGSLTLSCAASGSSVSFLSMAWYRQAPGKKRELVAGIS |
| (DLL3) | ADGSTIYIDSVKGRFTISRDNAKNSVYLQMNSLRAEDTAVYYCYAYRWRTRY |
| (SEQ ID NO: 549) | TYWGQGTLVTVSS |
| Exemplary anti-target | EVQLVESGGGLVQPGGSLTLSCAASGSSVRFLSMAWYRQAPGKKRELVAGI |
| (DLL3) | SAFIGSTDYIDSVKGRFTISRDNAKNSVYLQMNSLRAEDTAVYYCYAYRWRTR |
| (SEQ ID NO: 550) | YTYWGQGTLVTVSS |
| Exemplary anti-target | EVQLVESGGGLVQPGGSLTLSCAASGSSVSFLSMAWYRQAPGKKRELVAGIS |
| (DLL3) | RDGSTDYIDSVKGRFTISRDNAKNSVYLQMNSLRAEDTAVYYCYAYRWITRY |
| (SEQ ID NO: 551) | TYWGQGTLVTVSS |
| Exemplary anti-target | EVQLVESGGGLVQPGGSLTLSCAASGSSVRFLSMAWYRQAPGKKRELVAGI |
| (DLL3) | SRDGSTDYIDSVKGRFTISRDNAKNSVYLQMNSLRAEDTAVYYCYAYRWITR |
| (SEQ ID NO: 552) | YTYWGQGTLVTVSS |
| Exemplary anti-target | EVQLVESGGGLVQPGGSLTLSCAASGSSVVFLSMAWYRQAPGKKRELVAGI |
| (DLL3) | SADGSMDYIDSVKGRFTISRDNAKNSVYLQMNSLRAEDTAVYYCYAYRWRT |
| (SEQ ID NO: 553) | RYTYWGQGTLVTVSS |
| Exemplary anti-target | EVQLVESGGGLVQPGGSLTLSCAASGSSVRFLSMAWYRQAPGKKRELVAGI |
| (DLL3) | SADGSTDYIDSVKGRFTISRDNAKNSVYLQMNSLRAEDTAVYYCYAYRWTRR |
| (SEQ ID NO: 554) | YTYWGQGTLVTVSS |
| Exemplary anti-target | EVQLVESGGGLVQPGGSLTLSCAASGSSVRFLSMAWYRQAPGKKRELVAGI |
| (DLL3) | SADGSTDYIDSVKGRFTISRDNAKNSVYLQMNSLRAEDTAVYYCYAYSWTTR |
| (SEQ ID NO: 555) | YTYWGQGTLVTVSS |
| Exemplary anti-target | EVQLVESGGGLVQPGGSLTLSCAASGSSVRFLSMAWYRQAPGKKRELVAGI |
| (DLL3) | SANGSTDYIDSVKGRFTISRDNAKNSVYLQMNSLRAEDTAVYYCYAYRWTN |
| (SEQ ID NO: 556) | RYTYWGQGTLVTVSS |
| Exemplary anti-target | EVQLVESGGGLVQPGGSLTLSCAASGSSVSRLSMAWYRQAPGKKRELVAGI |
| (DLL3) | SANGSTTYIDSVKGRFTISRDNAKNSVYLQMNSLRAEDTAVYYCYAYRWTTR |
| (SEQ ID NO: 557) | YRYWGQGTLVTVSS |
| Exemplary anti-target | EVQLVESGGGLVQPGGSLTLSCAASGSSKSFLSMAWYRQAPGKKRELVAGIS |
| (DLL3) | ADGSTSYIDSVKGRFTISRDNAKNSVYLQMNSLRAEDTAVYYCYAYRWTTRY |
| (SEQ ID NO: 558) | TYWGQGTLVTVSS |
| Exemplary anti-target | EVQLVESGGGLVQPGGSLTLSCAASGSSVSRLSMAWYRQAPGKKRELVAGI |
| (DLL3) | SADGSRDYIDSVKGRFTISRDNAKNSVYLQMNSLRAEDTAVYYCYAYRWTTR |
| (SEQ ID NO: 559) | YKYWGQGTLVTVSS |
| Exemplary anti-target | EVQLVESGGGLVQPGGSLTLSCAASGSSVKFLSMAWYRQAPGKKRELVAGI |
| (DLL3) | SADGSTMYIDSVKGRFTISRDNAKNSVYLQMNSLRAEDTAVYYCYAYRWHT |
| (SEQ ID NO: 560) | RYTYWGQGTLVTVSS |
| Exemplary anti-target | EVQLVESGGGLVQPGGSLTLSCAASGSGVRFLSMAWYRQAPGKKRELVAGI |
| (DLL3) | SPDGSTDYIDSVKGRFTISRDNAKNSVYLQMNSLRAEDTAVYYCYAYRWTRR |
| (SEQ ID NO: 561) | YTYWGQGTLVTVSS |
| Exemplary anti-target | EVQLVESGGGLVQPGGSLTLSCAASGSSVRFLSMAWYRQAPGKKRELVAGI |
| (DLL3) | SGDGSTDYIDSVKGRFTISRDNAKNSVYLQMNSLRAEDTAVYYCYAYRWMT |
| (SEQ ID NO: 562) | RYTYWGQGTLVTVSS |
| Exemplary anti-target | EVQLVESGGGLVQPGGSLTLSCAASGSSVHFLSMAWYRQAPGKKRELVAGI |
| (DLL3) | SRDGSTDYIDSVKGRFTISRDNAKNSVYLQMNSLRAEDTAVYYCYAYRWTTR |
| (SEQ ID NO: 563) | YRYWGQGTLVTVSS |
| Exemplary anti-target | EVQLVESGGGLVQPGGSLTLSCAASGSSVRFLSMAWYRQAPGKKRELVAGI |
| (DLL3) | SRDGSTDYIDSVKGRFTISRDNAKNSVYLQMNSLRAEDTAVYYCYAYRWSTR |
| (SEQ ID NO: 564) | YTYWGQGTLVTVSS |
| Exemplary anti-target | EVQLVESGGGLVQPGGSLTLSCAASGSKVSFLSMAWYRQAPGKKRELVAGI |
| (DLL3) | SRDGSTDYIDSVKGRFTISRDNAKNSVYLQMNSLRAEDTAVYYCYAYRWTTR |
| (SEQ ID NO: 565) | YTFWGQGTLVTVSS |
| Exemplary anti-target | EVQLVESGGGLVQPGGSLTLSCAASGSSVSFLSMAWYRQAPGKKRELVAGIS |
| (DLL3) | ADGSTDYIDSVKGRFTISRDNAKNSVYLQMNSLRAEDTAVYYCYAYRWRTR |
| (SEQ ID NO: 566) | YTYWGQGTLVTVSS |
| Exemplary anti-target | EVQLVESGGGLVQPGGSLTLSCAASGSKVSFLSMAWYRQAPGKKRELVAGI |
| (DLL3) | STDGSTDYIDSVKGRFTISRDNAKNSVYLQMNSLRAEDTAVYYCYAYRWTTR |
| (SEQ ID NO: 567) | RTYWGQGTLVTVSS |
| Exemplary anti-target | EVQLVESGGGLVQPGGSLTLSCAASGSRVSFLSMAWYRQAPGKKRELVAGI |
| (DLL3) | SADGSTSYIDSVKGRFTISRDNAKNSVYLQMNSLRAEDTAVYYCYAYRWATR |
| (SEQ ID NO: 568) | YTYWGQGTLVTVSS |
| Exemplary anti-target | EVQLVESGGGLVQPGGSLTLSCAASGSRVSFLSMAWYRQAPGKKRELVAGI |
| (DLL3) | SADGSTLYIDSVKGRFTISRDNAKNSVYLQMNSLRAEDTAVYYCYAYRWHTR |
| (SEQ ID NO: 569) | YTYWGQGTLVTVSS |
| Exemplary anti-target | EVQLVESGGGLVQPGGSLTLSCAASGSSVRFLSMAWYRQAPGKKRELVAGI |
| (DLL3) | SRDGSTDYIDSVKGRFTISRDNAKNSVYLQMNSLRAEDTAVYYCYAYRWGT |
| (SEQ ID NO: 570) | RYTYWGQGTLVTVSS |
| Exemplary anti-target | EVQLVESGGGLVQPGGSLTLSCAASYSSVSRLSMAWYRQAPGKKRELVAGIS |
| (DLL3) | ADGSTVYIDSVKGRFTISRDNAKNSVYLQMNSLRAEDTAVYYCYAYRWTTR |
| (SEQ ID NO: 571) | NTYWGQGTLVTVSS |
| Exemplary anti-target | EVQLVESGGGLVQPGGSLTLSCAASGSSVSFLSMAWYRQAPGKKRELVAGIS |
| (DLL3) | TDGSTDYIDSVKGRFTISRDNAKNSVYLQMNSLRAEDTAVYYCYAYRWRTRY |
| (SEQ ID NO: 572) | TYWGQGTLVTVSS |
| Exemplary anti-target | EVQLVESGGGLVQPGGSLTLSCAASGSSVSFLSMAWYRQAPGKKRELVAGIS |
| (DLL3) | ADGSTLYIDSVKGRFTISRDNAKNSVYLQMNSLRAEDTAVYYCYAYRWTTRY |
| (SEQ ID NO: 573) | AYWGQGTLVTVSS |
| Exemplary anti-target | EVQLVESGGGLVQPGGSLTLSCAASGSSVSFLSMAWYRQAPGKKRELVAGIS |
| (DLL3) | ADGRTDYIDSVKGRFTISRDNAKNSVYLQMNSLRAEDTAVYYCYAYRWRTR |
| (SEQ ID NO: 574) | YTYWGQGTLVTVSS |
| Exemplary anti-target | EVQLVESGGGLVQPGGSLTLSCVASGTSVSFLSMAWYRQAPGKKRELVAGI |
| (DLL3) | SADGSTIYIDSVKGRFTISRDNAKNSVYLQMNSLRAEDTAVYYCYAYRWTTR |
| (SEQ ID NO: 575) | RTYWGQGTLVTVSS |
| Exemplary anti-target | EVQLVESGGGLVQPGGSLTLSCAASGSSVKFLSMAWYRQAPGKKRELVAGI |
| (DLL3) | SADGSTLYIDSVKGRFTISRDNAKNSVYLQMNSLRAEDTAVYYCYAYRWTTR |
| (SEQ ID NO: 576) | RTYWGQGTLVTVSS |
| Exemplary anti-target | EVQLVESGGGLVQPGGSLTLSCAASGSSVSFLSMAWYRQAPGKKRELVAGIS |
| (DLL3) | RDGSTDYIDSVKGRFTISRDNAKNSVYLQMNSLRAEDTAVYYCYAYRWTSRY |
| (SEQ ID NO: 577) | TYWGQGTLVTVSS |
| Exemplary anti-target | EVQLVESGGGLVQPGGSLTLSCAASGSRVSFLSMAWYRQAPGKKRELVAGI |
| (DLL3) | SKDGSTDYIDSVKGRFTISRDNAKNSVYLQMNSLRAEDTAVYYCYAYRWTTR |
| (SEQ ID NO: 578) | VTYWGQGTLVTVSS |
| Exemplary anti-target | EVQLVESGGGLVQPGGSLTLSCAASGSSVSVLSMAWYRQAPGKKRELVAGI |
| (DLL3) | SADGSTDYIGSVKGRFTISRDNAKNSVYLQMNSLRAEDTAVYYCYAYRWTTR |
| (SEQ ID NO: 579) | TTYWGQGTLVTVSS |
| Exemplary anti-target | EVQLVESGGGLVQPGGSLTLSCAASGSSVSFLSMAWYRQAPGKKRELVAGIS |
| (DLL3) | VDGSTDYIDSVKGRFTISRDNAKNSVYLQMNSLRAEDTAVYYCYAYRWRTR |
| (SEQ ID NO: 580) | YTYWGQGTLVTVSS |
| Exemplary anti-target | EVQLVESGGGLVQPGGSLTLSCAASGSRVSFLSMAWYRQAPGKKRELVAGI |
| (DLL3) | SADGSTGYIDSVKGRFTISRDNAKNSVYLQMNSLRAEDTAVYYCYAYRWAT |
| (SEQ ID NO: 581) | RYTYWGQGTLVTVSS |
| Exemplary anti-target | EVQLVESGGGLVQPGGSLTLSCVASGSSVKFLSMAWYRQAPGKKRELVAGI |
| (DLL3) | SGDGSTTYIDSVKGRFTISRDNAKNSVYLQMNSLRAEDTAVYYCYAYRWTTR |
| (SEQ ID NO: 582) | RTYWGQGTLVTVSS |
| Exemplary anti-target | EVQLVESGGGLVQPGGSLTLSCAASGSSVRFLSMAWYRQAPGKKRELVAGI |
| (DLL3) | STDGSTDYIDSVKGRFTISRDNAKNSVYLQMNSLRAEDTAVYYCYALRWTTR |
| (SEQ ID NO: 583) | YTYWGQGTLVTVSS |
| Exemplary anti-target | EVQLVESGGGLVQPGGSLTLSCAASGSSVSQLSMAWYRQAPGKKRELVAGI |
| (DLL3) | SADGSTDYFDSVKGRFTISRDNAKNSVYLQMNSLRAEDTAVYYCYAYRWTT |
| (SEQ ID NO: 584) | RGTYWGQGTLVTVSS |
| Exemplary anti-target | EVQLVESGGGLVQPGGSLTLSCAASGSRVSFLSMAWYRQAPGKKRELVAGI |
| (DLL3) | SADGSTSYIDSVKGRFTISRDNAKNSVYLQMNSLRAEDTAVYYCYAYRWRTR |
| (SEQ ID NO: 585) | YTYWGQGTLVTVSS |
| Exemplary anti-target | EVQLVESGGGLVQPGGSLTLSCAASKSSVSFLSMAWYRQAPGKKRELVAGIS |
| (DLL3) | ADGSTDYIDSVKGRFTISRDNAKNSVYLQMNSLRAEDTAVYYCYAYRWTTR |
| (SEQ ID NO: 586) | ATYWGQGTLVTVSS |
| Exemplary anti-target | EVQLVESGGGLVQPGGSLTLSCAASGSSVSFLSMAWYRQAPGKKRELVAGIS |
| (DLL3) | ADGSTAYIDSVKGRFTISRDNAKNSVYLQMNSLRAEDTAVYYCYAYRWTTRR |
| (SEQ ID NO: 587) | TYWGQGTLVTVSS |
| Exemplary anti-target | EVQLVESGGGLVQPGGSLTLSCAASGSSVKFLSMAWYRQAPGKKRELVAGI |
| (DLL3) | SADGSTVYIDSVKGRFTISRDNAKNSVYLQMNSLRAEDTAVYYCYAYRWPTR |
| (SEQ ID NO: 588) | YTYWGQGTLVTVSS |
| Exemplary anti-target | EVQLVESGGGLVQPGGSLTLSCAASGSSVRFLSMAWYRQAPGKKRELVAGI |
| (DLL3) | SQDGSTDYIDSVKGRFTISRDNAKNSVYLQMNSLRAEDTAVYYCYAYRWRT |
| (SEQ ID NO: 589) | RYTYWGQGTLVTVSS |
| Exemplary anti-target | EVQLVESGGGLVQPGGSLTLSCAASGSSVRFLSMAWYRQAPGKKRELVAGI |
| (DLL3) | SNDGSTDYIDSVKGRFTISRDNAKNSVYLQMNSLRAEDTAVYYCYAYRWKT |
| (SEQ ID NO: 590) | RYTYWGQGTLVTVSS |
| Exemplary anti-target | EVQLVESGGGLVQPGGSLTLSCAASGSRVSFLSMAWYRQAPGKKRELVAGI |
| (DLL3) | SARGSTDYIDSVKGRFTISRDNAKNSVYLQMNSLRAEDTAVYYCYAYRWSTR |
| (SEQ ID NO: 591) | YTYWGQGTLVTVSS |
| Exemplary anti-target | EVQLVESGGGLVQPGGSLTLSCAASGSSVSFLSLAWYRQAPGKKRELVAGIS |
| (DLL3) | ADGSTDYIDSVKGRFTISRDNAKNSVYLQMNSLRAEDTAVYYCYAYRWKTR |
| (SEQ ID NO: 592) | RTYWGQGTLVTVSS |
| Exemplary anti-target | EVQLVESGGGLVQPGGSLTLSCAASGSSVRFLSMAWYRQAPGKKRELVAGI |
| (DLL3) | SRDGSTDYIDSVKGRFTISRDNAKNSVYLQMNSLRAEDTAVYYCYAYRWTTR |
| (SEQ ID NO: 593) | RTYWGQGTLVTVSS |
| Exemplary anti-target | EVQLVESGGGLVQPGGSLTLSCAASGSKVSFLSMAWYRQAPGKKRELVAGI |
| (DLL3) | SADGSTLYIDSVKGRFTISRDNAKNSVYLQMNSLRAEDTAVYYCYAYRWTTR |
| (SEQ ID NO: 594) | YRYWGQGTLVTVSS |
| Exemplary anti-target | EVQLVESGGGLVQPGGSLTLSCAASGSSVSFLSMAWYRQAPGKKRELVAGIS |
| (DLL3) | ADGSTNYIDSVKGRFTISRDNAKNSVYLQMNSLRAEDTAVYYCYAYRWRTR |
| (SEQ ID NO: 595) | YTYWGQGTLVTVSS |
| Exemplary anti-target | EVQLVESGGGLVQPGGSLTLSCAASGSSVRFLSMAWYRQAPGKKRELVAGI |
| (DLL3) | SADGSTVYIDSVKGRFTISRDNAKNSVYLQMNSLRAEDTAVYYCYAYRWTTR |
| (SEQ ID NO: 596) | YKYWGQGTLVTVSS |
| Exemplary anti-target | EVQLVESGGGLVQPGGSLTLSCAASGSKVSFLSMAWYRQAPGKKRELVAGI |
| (DLL3) | SADGSTTYIDSVKGRFTISRDNAKNSVYLQMNSLRAEDTAVYYCYAYRWKTR |
| (SEQ ID NO: 597) | YTYWGQGTLVTVSS |
| Exemplary anti-target | EVQLVESGGGLVQPGGSLTLSCAASGSSVKFLSMAWYRQAPGKKRELVAGI |
| (DLL3) | SADGSTDYIGSVKGRFTISRDNAKNSVYLQMNSLRAEDTAVYYCYAYRWTTR |
| (SEQ ID NO: 598) | VTYWGQGTLVTVSS |
| Exemplary anti-target | EVQLVESGGGLVQPGGSLTLSCAASGSSVKFLSMAWYRQAPGKKRELVAGI |
| (DLL3) | SRDGSTDYIDSVKGRFTISRDNAKNSVYLQMNSLRAEDTAVYYCYAYRWTTR |
| (SEQ ID NO: 599) | FTYWGQGTLVIVSS |
| Exemplary anti-target | EVQLVESGGGLVQPGGSLTLSCAASGSRVSFLSMAWYRQAPGKKRELVAGI |
| (DLL3) | SADGSTTYIDSVKGRFTISRDNAKNSVYLQMNSLRAEDTAVYYCYAYRWTTR |
| (SEQ ID NO: 600) | FTYWGQGTLVIVSS |
| Exemplary anti-target | EVQLVESGGGLVQPGGSLTLSCAASGSSVLFLSMAWYRQAPGKKRELVAGV |
| (DLL3) | SSDGSTDYIDSVKGRFTISRDNAKNSVYLQMNSLRAEDTAVYYCYAYRWRTR |
| (SEQ ID NO: 601) | YTYWGQGTLVTVSS |
| Exemplary anti-target | EVQLVESGGGLVQPGGSLTLSCAASGSRVSFLSMAWYRQAPGKKRELVAGI |
| (DLL3) | SADGHTDYIDSVKGRFTISRDNAKNSVYLQMNSLRAEDTAVYYCYAYRWTT |
| (SEQ ID NO: 602) | RYTHWGQGTLVTVSS |
| Exemplary anti-target | EVQLVESGGGLVQPGGSLTLSCAASGSRVSFLSMAWYRQAPGKKRELVAGI |
| (DLL3) | SADGSTDYFDSVKGRFTISRDNAKNSVYLQMNSLRAEDTAVYYCYAYRWTR |
| (SEQ ID NO: 603) | RYTYWGQGTLVTVSS |
| Exemplary anti-target | EVQLVESGGGLVQPGGSLTLSCAASGSSVGFLSMAWYRQAPGKKRELVAGI |
| (DLL3) | SADGSTVYIDSVKGRFTISRDNAKNSVYLQMNSLRAEDTAVYYCYAYRWTTR |
| (SEQ ID NO: 604) | YTYWGQGTLVTVSS |
| Exemplary anti-target | EVQLVESGGGLVQPGGSLTLSCAASGSSVSFMSMAWYRQAPGKKRELVAGI |
| (DLL3) | SADGSTDYIASVKGRFTISRDNAKNSVYLQMNSLRAEDTAVYYCYAYRWTTR |
| (SEQ ID NO: 605) | STYWGQGTLVIVSS |
| Exemplary anti-target | EVQLVESGGGLVQPGGSLTLSCAASGSSVSFLSMAWYRQAPGKKRELVAGIS |
| (DLL3) | ADGSTDYISSVKGRFTISRDNAKNSVYLQMNSLRAEDTAVYYCYAYSWTTRY |
| (SEQ ID NO: 606) | TYWGQGTLVTVSS |
| Exemplary anti-target | EVQLVESGGGLVQPGGSLTLSCAASGSSVTFLSMAWYRQAPGKKRELVAGI |
| (DLL3) | SADGSTVYIDSVKGRFTISRDNAKNSVYLQMNSLRAEDTAVYYCYAYRWTTR |
| (SEQ ID NO: 607) | GTYWGQGTLVTVSS |
| Exemplary anti-target | EVQLVESGGGLVQPGGSLTLSCAASGSSVRFLSMAWYRQAPGKKRELVAGI |
| (DLL3) | SADGSTVYIDSVKGRFTISRDNAKNSVYLQMNSLRAEDTAVYYCYAYRWKTR |
| (SEQ ID NO: 608) | YTYWGQGTLVTVSS |
| Exemplary anti-target | EVQLVESGGGLVQPGGSLTLSCAASGSKVSFLSMAWYRQAPGKKRELVAGI |
| (DLL3) | SADGSTTYIDSVKGRFTISRDNAKNSVYLQMNSLRAEDTAVYYCYAYRWTTR |
| (SEQ ID NO: 609) | FTYWGQGTLVIVSS |
| Exemplary anti-target | EVQLVESGGGLVQPGGSLTLSCAASGSKVSFMSMAWYRQAPGKKRELVAG |
| (DLL3) | ISVDGSTDYIDSVKGRFTISRDNAKNSVYLQMNSLRAEDTAVYYCYAYRWRT |
| (SEQ ID NO: 610) | RYTYWGQGTLVTVSS |
| Exemplary anti-target | EVQLVESGGGLVQPGGSLTLSCAASGSSVSNLSMAWYRQAPGKKRELVAGI |
| (DLL3) | SADGSTAYIDSVKGRFTISRDNAKNSVYLQMNSLRAEDTAVYYCYAYRWTTR |
| (SEQ ID NO: 611) | RTYWGQGTLVTVSS |
| Exemplary anti-target | EVQLVESGGGLVQPGGSLTLSCAASNSSVSKLSMAWYRQAPGKKRELVAGI |
| (DLL3) | SADGSTAYIDSVKGRFTISRDNAKNSVYLQMNSLRAEDTAVYYCYAYRWSTR |
| (SEQ ID NO: 612) | YTYWGQGTLVTVSS |
| Exemplary anti-target | EVQLVESGGGLVQPGGSLTLSCAASGSKVSFLSMAWYRQAPGKKRELVAGI |
| (DLL3) | SADGSKDYIDSVKGRFTISRDNAKNSVYLQMNSLRAEDTAVYYCYAYRWTTR |
| (SEQ ID NO: 613) | LTYWGQGTLVTVSS |
| Exemplary anti-target | EVQLVESGGGLVQPGGSLTLSCVASGSQVSFLSMAWYRQAPGKKRELVAGI |
| (DLL3) | SADGSTDYFDSVKGRFTISRDNAKNSVYLQMNSLRAEDTAVYYCYAYRWTR |
| (SEQ ID NO: 614) | RYTYWGQGTLVTVSS |
| Exemplary anti-target | EVQLVESGGGLVQPGGSLTLSCAASGSKVSFMSMAWYRQAPGKKRELVAG |
| (DLL3) | ISADGSTDYIDSVKGRFTISRDNAKNSVYLQMNSLRAEDTAVYYCYAYRWTT |
| (SEQ ID NO: 615) | RLTYWGQGTLVTVSS |
| Exemplary anti-target | EVQLVESGGGLVQPGGSLTLSCAASGSRVSFLSMAWYRQAPGKKRELVAGI |
| (DLL3) | SADGSTVYIDSVKGRFTISRDNAKNSVYLQMNSLRAEDTAVYYCYAYRWTRR |
| (SEQ ID NO: 616) | YTYWGQGTLVTVSS |
| Exemplary anti-target | EVQLVESGGGLVQPGGSLTLSCAASGSSVRFLSMAWYRQAPGKKRELVAGI |
| (DLL3) | SADGSTVYIDSVKGRFTISRDNAKNSVYLQMNSLRAEDTAVYYCYAYRWTTR |
| (SEQ ID NO: 617) | RTYWGQGTLVTVSS |
| Exemplary anti-target | EVQLVESGGGLVQPGGSLTLSCAASGSKVSFLSMAWYRQAPGKKRELVAGI |
| (DLL3) | SARGSTDYIDSVKGRFTISRDNAKNSVYLQMNSLRAEDTAVYYCYAYQWTTR |
| (SEQ ID NO: 618) | YTYWGQGTLVTVSS |
| Exemplary anti-target | EVQLVESGGGLVQPGGSLTLSCAASGSSVRFLSMAWYRQAPGKKRELVAGI |
| (DLL3) | SATGSTDYIDSVKGRFTISRDNAKNSVYLQMNSLRAEDTAVYYCYAYRWTRR |
| (SEQ ID NO: 619) | YTYWGQGTLVTVSS |
| Exemplary anti-target | EVQLVESGGGLVQPGGSLTLSCAASGSSVSFLSIAWYRQAPGKKRELVAGISK |
| (DLL3) | DGSTDYIDSVKGRFTISRDNAKNSVYLQMNSLRAEDTAVYYCYAYRWTTRM |
| (SEQ ID NO: 620) | TYWGQGTLVTVSS |
| Exemplary anti-target | EVQLVESGGGLVQPGGSLTLSCAASGSSSSFLSMAWYRQAPGKKRELVAGIS |
| (DLL3) | ADGSTVYIDSVKGRFTISRDNAKNSVYLQMNSLRAEDTAVYYCYAYRWTTRR |
| (SEQ ID NO: 621) | TYWGQGTLVTVSS |
| Exemplary anti-target | EVQLVESGGGLVQPGGSLTLSCAASGSSVKFLSMAWYRQAPGKKRELVAGI |
| (DLL3) | SPDGSTDYIDSVKGRFTISRDNAKNSVYLQMNSLRAEDTAVYYCYAYRWTTR |
| (SEQ ID NO: 622) | YRYWGQGTLVTVSS |
| Exemplary anti-target | EVQLVESGGGLVQPGGSLTLSCAASGSSVNFLSMAWYRQAPGKKRELVAGI |
| (DLL3) | SADGSTHYIDSVKGRFTISRDNAKNSVYLQMNSLRAEDTAVYYCYAYRWLTR |
| (SEQ ID NO: 623) | YTYWGQGTLVTVSS |
| Exemplary anti-target | EVQLVESGGGLVQPGGSLTLSCAASGSSVKFLSMAWYRQAPGKKRELVAGI |
| (DLL3) | SADGSTDYILSVKGRFTISRDNAKNSVYLQMNSLRAEDTAVYYCYAYEWTTR |
| (SEQ ID NO: 624) | YTYWGQGTLVTVSS |
| Exemplary anti-target | EVQLVESGGGLVQPGGSLTLSCAASGSSVSFLSMAWYRQAPGKKRELVAGIS |
| (DLL3) | ADGSTDYIHSVKGRFTISRDNAKNSVYLQMNSLRAEDTAVYYCYAYRWRTR |
| (SEQ ID NO: 625) | YTYWGQGTLVTVSS |
| Exemplary anti-target | EVQLVESGGGLVQPGGSLTLSCAASGSSVRFLSMAWYRQAPGKKRELVAGI |
| (DLL3) | SVDGSTDYIDSVKGRFTISRDNAKNSVYLQMNSLRAEDTAVYYCYAYRWRTR |
| (SEQ ID NO: 626) | YTYWGQGTLVTVSS |
| Exemplary anti-target | EVQLVESGGGLVQPGGSLTLSCAASGSSVSFLSVAWYRQAPGKKRELVAGIS |
| (DLL3) | RDGSTDYIDSVKGRFTISRDNAKNSVYLQMNSLRAEDTAVYYCYAYRWRTRY |
| (SEQ ID NO: 627) | TYWGQGTLVTVSS |
| Exemplary anti-target | EVQLVESGGGLVQPGGSLTLSCAASGSQVSFLSMAWYRQAPGKKRELVAGI |
| (DLL3) | SADGSTVYIDSVKGRFTISRDNAKNSVYLQMNSLRAEDTAVYYCYAYRWSTR |
| (SEQ ID NO: 628) | YTYWGQGTLVTVSS |
| Exemplary anti-target | EVQLVESGGGLVQPGGSLTLSCAASGTSVSFLSMAWYRQAPGKKRELVAGI |
| (DLL3) | SADGSTDYIRSVKGRFTISRDNAKNSVYLQMNSLRAEDTAVYYCYAYRWTTR |
| (SEQ ID NO: 629) | LTYWGQGTLVTVSS |
| Exemplary anti-target | EVQLVESGGGLVQPGGSLTLSCAASGSRVSFLSMAWYRQAPGKKRELVAGI |
| (DLL3) | SADGSTMYIDSVKGRFTISRDNAKNSVYLQMNSLRAEDTAVYYCYAYRWTT |
| (SEQ ID NO: 630) | RLTYWGQGTLVTVSS |
| Exemplary anti-target | EVQLVESGGGLVQPGGSLTLSCAASGSSVKFLSMAWYRQAPGKKRELVAGI |
| (DLL3) | STDGSTDYIDSVKGRFTISRDNAKNSVYLQMNSLRAEDTAVYYCYAYKWTTR |
| (SEQ ID NO: 631) | YTYWGQGTLVTVSS |
| Exemplary anti-target | EVQLVESGGGLVQPGGSLTLSCAASGSSVSFLSSAWYRQAPGKKRELVAGIS |
| (DLL3) | ADGSTLYIDSVKGRFTISRDNAKNSVYLQMNSLRAEDTAVYYCYAYRWTTRS |
| (SEQ ID NO: 632) | TYWGQGTLVTVSS |
| Exemplary anti-target | EVQLVESGGGLVQPGGSLTLSCAASGSSVKFLSMAWYRQAPGKKRELVAGI |
| (DLL3) | SADGSTDYIDSVKGRFTISRDNAKNSVYLQMNSLRAEDTAVYYCYAYRWRTR |
| (SEQ ID NO: 633) | YTYWGQGTLVTVSS |
| Exemplary anti-target | EVQLVESGGGLVQPGGSLTLSCAASGSSVSFLSMAWYRQAPGKKRELVAGIS |
| (DLL3) | ATGSTDYIDSVKGRFTISRDNAKNSVYLQMNSLRAEDTAVYYCYAYRWSTRY |
| (SEQ ID NO: 634) | TYWGQGTLVTVSS |
| Exemplary anti-target | EVQLVESGGGLVQPGGSLTLSCAASGSTVSFLSMAWYRQAPGKKRELVAGI |
| (DLL3) | SHDGSTDYIDSVKGRFTISRDNAKNSVYLQMNSLRAEDTAVYYCYAYRWTTR |
| (SEQ ID NO: 635) | YTYWGQGTLVTVSS |
| Exemplary anti-target | EVQLVESGGGLVQPGGSLTLSCAASGSSVQFLSMAWYRQAPGKKRELVAGI |
| (DLL3) | SYDGSTDYIDSVKGRFTISRDNAKNSVYLQMNSLRAEDTAVYYCYAYRWRTR |
| (SEQ ID NO: 636) | YTYWGQGTLVTVSS |
| Exemplary anti-target | EVQLVESGGGLVQPGGSLTLSCAASRSSVSFLSMAWYRQAPGKKRELVAGIS |
| (DLL3) | TDGSTDYIDSVKGRFTISRDNAKNSVYLQMNSLRAEDTAVYYCYAYRWLTRY |
| (SEQ ID NO: 637) | TYWGQGTLVTVSS |
| Exemplary anti-target | EVQLVESGGGLVQPGGSLTLSCAASGSKVSFLSMAWYRQAPGKKRELVAGI |
| (DLL3) | SADGSTAYIDSVKGRFTISRDNAKNSVYLQMNSLRAEDTAVYYCYAYRWRTR |
| (SEQ ID NO: 638) | YTYWGQGTLVTVSS |
| Exemplary anti-target | EVQLVESGGGLVQPGGSLTLSCAASGSRVSFLSMAWYRQAPGKKRELVAGI |
| (DLL3) | SADGSTDYIESVKGRFTISRDNAKNSVYLQMNSLRAEDTAVYYCYAYRWTTR |
| (SEQ ID NO: 639) | YTYWGQGTLVTVSS |
| Exemplary anti-target | EVQLVESGGGLVQPGGSLTLSCAASGSSVSFLSMAWYRQAPGKKRELVAGIS |
| (DLL3) | IDGSTDYIKSVKGRFTISRDNAKNSVYLQMNSLRAEDTAVYYCYAYRWTTRY |
| (SEQ ID NO: 640) | RYWGQGTLVTVSS |
| Exemplary anti-target | EVQLVESGGGLVQPGGSLTLSCAASGSKVSFLSMAWYRQAPGKKRELVAGI |
| (DLL3) | SADGSKDYIDSVKGRFTISRDNAKNSVYLQMNSLRAEDTAVYYCYAYRWTTR |
| (SEQ ID NO: 641) | YTYWGQGTLVTVSS |
| Exemplary anti-target | EVQLVESGGGLVQPGGSLTLSCAASGSSVRFLSMAWYRQAPGKKRELVAGI |
| (DLL3) | SADGSTVYIDSVKGRFTISRDNAKNSVYLQMNSLRAEDTAVYYCYAYRWPTR |
| (SEQ ID NO: 642) | YTYWGQGTLVTVSS |
| Exemplary anti-target | EVQLVESGGGLVQPGGSLTLSCAASGSSVKFLSMAWYRQAPGKKRELVAGI |
| (DLL3) | SRDGSTDYIDSVKGRFTISRDNAKNSVYLQMNSLRAEDTAVYYCYAYRWTTR |
| (SEQ ID NO: 643) | HTYWGQGTLVTVSS |
| Exemplary anti-target | EVQLVESGGGLVQPGGSLTLSCAASGSSVKFLSMAWYRQAPGKKRELVAGI |
| (DLL3) | SADGSTDYIHSVKGRFTISRDNAKNSVYLQMNSLRAEDTAVYYCYAYRWTRR |
| (SEQ ID NO: 644) | YTYWGQGTLVTVSS |
| Exemplary anti-target | EVQLVESGGGLVQPGGSLTLSCAASGSSVSILSMAWYRQAPGKKRELVAGIS |
| (DLL3) | ADGSTIYIDSVKGRFTISRDNAKNSVYLQMNSLRAEDTAVYYCYAYRWHTRY |
| (SEQ ID NO: 645) | TYWGQGTLVTVSS |
| Exemplary anti-target | EVQLVESGGGLVQPGGSLTLSCAASGSSVSFLSVAWYRQAPGKKRELVAGIS |
| (DLL3) | ANGSTDYIDSVKGRFTISRDNAKNSVYLQMNSLRAEDTAVYYCYAYRWTNR |
| (SEQ ID NO: 646) | YTYWGQGTLVTVSS |
| Exemplary anti-target | EVQLVESGGGLVQPGGSLTLSCAASGSSVRFLSMAWYRQAPGKKRELVAGI |
| (DLL3) | STDGSTDYIDSVKGRFTISRDNAKNSVYLQMNSLRAEDTAVYYCYAYRWTTR |
| (SEQ ID NO: 647) | YRYWGQGTLVTVSS |
| Exemplary anti-target | EVQLVESGGGLVQPGGSLTLSCAASGSRVSFLSMAWYRQAPGKKRELVAGI |
| (DLL3) | SYDGSTDYIDSVKGRFTISRDNAKNSVYLQMNSLRAEDTAVYYCYAYRWTTR |
| (SEQ ID NO: 648) | RTYWGQGTLVTVSS |
| Exemplary anti-target | EVQLVESGGGLVQPGGSLTLSCAASGHSVSFLSMAWYRQAPGKKRELVAGI |
| (DLL3) | SADGSTDYIASVKGRFTISRDNAKNSVYLQMNSLRAEDTAVYYCYAYRWSTR |
| (SEQ ID NO: 649) | YTYWGQGTLVTVSS |
| Exemplary anti-target | EVQLVESGGGLVQPGGSLTLSCAASGSSVRFLSMAWYRQAPGKKRELVAGI |
| (DLL3) | SADGSTDYIGSVKGRFTISRDNAKNSVYLQMNSLRAEDTAVYYCYAYRWSTR |
| (SEQ ID NO: 650) | YTYWGQGTLVTVSS |
| Exemplary anti-target | EVQLVESGGGLVQPGGSLTLSCAASGSSVSFLSMAWYRQAPGKKRELVAGIS |
| (DLL3) | ANGSTDYYDSVKGRFTISRDNAKNSVYLQMNSLRAEDTAVYYCYAYRWRTR |
| (SEQ ID NO: 651) | YTYWGQGTLVTVSS |
| Exemplary anti-target | EVQLVESGGGLVQPGGSLTLSCAASGSRVSFLSMAWYRQAPGKKRELVAGI |
| (DLL3) | SADGSTSYIDSVKGRFTISRDNAKNSVYLQMNSLRAEDTAVYYCYAYRWTTR |
| (SEQ ID NO: 652) | YTYWGQGTLVTVSS |
| Exemplary anti-target | EVQLVESGGGLVQPGGSLTLSCAASGSSVKFLSMAWYRQAPGKKRELVAGV |
| (DLL3) | SADGSTDYIDSVKGRFTISRDNAKNSVYLQMNSLRAEDTAVYYCYAYEWTTR |
| (SEQ ID NO: 653) | YTYWGQGTLVTVSS |
| Exemplary anti-target | EVQLVESGGGLVQPGGSLTLSCAASGSSVSRLSMAWYRQAPGKKRELVAGI |
| (DLL3) | SARGSTDYIDSVKGRFTISRDNAKNSVYLQMNSLRAEDTAVYYCYAYRWTTR |
| (SEQ ID NO: 654) | STYWGQGTLVTVSS |
| Exemplary anti-target | EVQLVESGGGLVQPGGSLTLSCAASGRSVSFLSMAWYRQAPGKKRELVAGI |
| (DLL3) | SADGSTIYIDSVKGRFTISRDNAKNSVYLQMNSLRAEDTAVYYCYAYRWTTR |
| (SEQ ID NO: 655) | YTYWGQGTLVTVSS |
| Exemplary anti-target | EVQLVESGGGLVQPGGSLTLSCAASGRSVSFLSMAWYRQAPGKKRELVAGI |
| (DLL3) | SANGSTDYIDSVKGRFTISRDNAKNSVYLQMNSLRAEDTAVYYCYAYRWSTR |
| (SEQ ID NO: 656) | YTYWGQGTLVTVSS |
| Exemplary anti-target | EVQLVESGGGLVQPGGSLTLSCAASGSSVKFLSMAWYRQAPGKKRELVAGI |
| (DLL3) | SADGSTDYVDSVKGRFTISRDNAKNSVYLQMNSLRAEDTAVYYCYAYRWST |
| (SEQ ID NO: 657) | RYTYWGQGTLVTVSS |
| Exemplary anti-target | EVQLVESGGGLVQPGGSLTLSCAASGSSVSKLSMAWYRQAPGKKRELVAGI |
| (DLL3) | SADGSTDYRDSVKGRFTISRDNAKNSVYLQMNSLRAEDTAVYYCYAYRWTY |
| (SEQ ID NO: 658) | RYTYWGQGTLVTVSS |
| Exemplary anti-target | EVQLVESGGGLVQPGGSLTLSCAASGSSVSRLSMAWYRQAPGKKRELVAGI |
| (DLL3) | SVDGSTDYIDSVKGRFTISRDNAKNSVYLQMNSLRAEDTAVYYCYAYRWTTR |
| (SEQ ID NO: 659) | LTYWGQGTLVTVSS |
| Exemplary anti-target | EVQLVESGGGLVQPGGSLTLSCAASGSSVKFLSLAWYRQAPGKKRELVAGIS |
| (DLL3) | ADGSTDYILSVKGRFTISRDNAKNSVYLQMNSLRAEDTAVYYCYAYEWTTRY |
| (SEQ ID NO: 660) | TYWGQGTLVTVSS |
| Exemplary anti-target | EVQLVESGGGLVQPGGSLTLSCAASGSSVSRLSLAWYRQAPGKKRELVAGIS |
| (DLL3) | VDGSTDYIDSVKGRFTISRDNAKNSVYLQMNSLRAEDTAVYYCYAYRWTTRL |
| (SEQ ID NO: 661) | TYWGQGTLVTVSS |
| Exemplary anti-target | EVQLVESGGGLVQPGGSLTLSCVASGTSSSINAMGWYRRAPGKQRELVAGI |
| (DLL3) | SSDGSKVFNESVKGRFTISRDNAKNSVYLQMNSLRAEDTAVYYCYYFRPAAG |
| (SEQ ID NO: 662) | SPMRYWGQGTLVTVSS |
| Exemplary anti-target | EVQLVESGGGLVQPGGSLTLSCVASGTTSSINAIGWYRRAPGKQRELVAGISS |
| (DLL3) | DGSEVYTDSVKGRFTISRDNAKNSVYLQMNSLRAEDTAVYYCYYFRTVDGSP |
| (SEQ ID NO: 663) | LRYWGQGTLVTVSS |
| Exemplary anti-target | EVQLVESGGGLVQPGGSLTLSCVASGSTSSINAMAWYRRAPGKQRELVAGI |
| (DLL3) | SSDDSNVYYESVKGRFTISRDNAKNSVYLQMNSLRAEDTAVYYCYYFRTVSG |
| (SEQ ID NO: 664) | SSKRYWGQGTLVTVSS |
| Exemplary anti-target | EVQLVESGGGLVQPGGSLTLSCVASGQTYRVNAFGWYRRAPGKQRELVAGI |
| (DLL3) | SSDGSKVYADSVKGRFTISRDNAKNSVYLQMNSLRAEDTAVYYCYYFSAGSG |
| (SEQ ID NO: 665) | TEMSYWGQGTLVTVSS |
| Exemplary anti-target | EVQLVESGGGLVQPGGSLTLSCVASGSTSSINAMAWYRRAPGKQRELVAGI |
| (DLL3) | SSDESTLYVDSVKGRFTISRDNAKNSVYLQMNSLRAEDTAVYYCYYFGSLSGS |
| (SEQ ID NO: 666) | STTYWGQGTLVTVSS |
| Exemplary anti-target | EVQLVESGGGLVQPGGSLTLSCVASGSASLTNATGWYRRAPGKQRELVAGI |
| (DLL3) | SSDDSKVYSDSVKGRFTISRDNAKNSVYLQMNSLRAEDTAVYYCYYFGSVSG |
| (SEQ ID NO: 667) | SWTRYWGQGTLVTVSS |
| Exemplary anti-target | EVQLVESGGGLVQPGGSLTLSCVASGYPSLNNAMGWYRRAPGKQRELVAG |
| (DLL3) | ISSDGSQVYGASVKGRFTISRDNAKNSVYLQMNSLRAEDTAVYYCYYFRLVS |
| (SEQ ID NO: 668) | GSSMSYWGQGTLVTVSS |
| Exemplary anti-target | EVQLVESGGGLVQPGGSLTLSCVASGSSSTINAIGWYRRAPGKQRELVAGISS |
| (DLL3) | DGSKVYADSVKGRFTISRDNAKNSVYLQMNSLRAEDTAVYYCYYFRTGSGTS |
| (SEQ ID NO: 669) | KSYWGQGTLVTVSS |
| Exemplary anti-target | EVQLVESGGGLVQPGGSLTLSCVASGSTSYINAMGWYRRAPGKQRELVAGI |
| (DLL3) | SSDGSNMYADSVKGRFTISRDNAKNSVYLQMNSLRAEDTAVYYCYYFSNMS |
| (SEQ ID NO: 670) | GTTRRYWGQGTLVTVSS |
| Exemplary anti-target | EVQLVESGGGLVQPGGSLTLSCVASGSTSSVNALGWYRRAPGKQRELVAGIS |
| (DLL3) | SDGSKVYTDSVKGRFTISRDNAKNSVYLQMNSLRAEDTAVYYCYYFRTVPGS |
| (SEQ ID NO: 671) | AMGYWGQGTLVTVSS |
| Exemplary anti-target | EVQLVESGGGLVQPGGSLTLSCVASGSTSLSNAVGWYRRAPGKQRELVAGIS |
| (DLL3) | SDGSKVSAESVKGRFTISRDNAKNSVYLQMNSLRAEDTAVYYCYYFRAESGS |
| (SEQ ID NO: 672) | SMGYWGQGTLVTVSS |
| Exemplary anti-target | EVQLVESGGGLVQPGGSLTLSCVASGSTSSTNAIGWYRRAPGKQRELVAGIS |
| (DLL3) | SDGSKVYDDSVKGRFTISRDNAKNSVYLQMNSLRAEDTAVYYCYYFRTLYGS |
| (SEQ ID NO: 673) | SRSYWGQGTLVTVSS |
| Exemplary anti-target | EVQLVESGGGLVQPGGSLTLSCVASGLTSTINAMGWYRRAPGKQRELVAGI |
| (DLL3) | SSDGSKVYDDSVKGRFTISRDNAKNSVYLQMNSLRAEDTAVYYCYYFSPFSG |
| (SEQ ID NO: 674) | SDTGYWGQGTLVTVSS |
| Exemplary anti-target | EVQLVESGGGLVQPGGSLTLSCVASGVSPSKNAIGWYRRAPGKQRELVAGIS |
| (DLL3) | SDGSAVYVGSVKGRFTISRDNAKNSVYLQMNSLRAEDTAVYYCYYFSTFSGS |
| (SEQ ID NO: 675) | SISYWGQGTLVTVSS |
| Exemplary anti-target | EVQLVESGGGLVQPGGSLTLSCVASGSTSSINAVGWYRRAPGKQRELVAGIS |
| (DLL3) | SDGSYVYSESVKGRFTISRDNAKNSVYLQMNSLRAEDTAVYYCYYFRTLAGSE |
| (SEQ ID NO: 676) | MRYWGQGTLVTVSS |
| Exemplary anti-target | EVQLVESGGGLVQPGGSLTLSCVASGSTTMNNAMAWYRRAPGKQRELVA |
| (DLL3) | GISSDSSHVYADSVKGRFTISRDNAKNSVYLQMNSLRAEDTAVYYCYYFRTVS |
| (SEQ ID NO: 677) | GSGVRYWGQGTLVTVSS |
| Exemplary anti-target | EVQLVESGGGLVQPGGSLTLSCVASGSTSKINAIGWYRRAPGKQRELVAGIS |
| (DLL3) | SDSSIVYTDSVKGRFTISRDNAKNSVYLQMNSLRAEDTAVYYCYYFRPGAGH |
| (SEQ ID NO: 678) | SNSYWGQGTLVTVSS |
| Exemplary anti-target | EVQLVESGGGLVQPGGSLTLSCVASGQTTALNAMGWYRRAPGKQRELVAG |
| (DLL3) | ISSDGSEVNTDSVKGRFTISRDNAKNSVYLQMNSLRAEDTAVYYCYYFRRAS |
| (SEQ ID NO: 679) | GTAMSYWGQGTLVTVSS |
| Exemplary anti-target | EVQLVESGGGLVQPGGSLTLSCVASGATSSINAIGWYRRAPGKQRELVAGIS |
| (DLL3) | SDGSKLSSDSVKGRFTISRDNAKNSVYLQMNSLRAEDTAVYYCYYFTSASGT |
| (SEQ ID NO: 680) | DLSYWGQGTLVTVSS |
| Exemplary anti-target | EVQLVESGGGLVQPGGSLTLSCVASGSTSTINAMGWYRRAPGKQRELVAGI |
| (DLL3) | SSDNSKVYADSVKGRFTISRDNAKNSVYLQMNSLRAEDTAVYYCYYFRSANG |
| (SEQ ID NO: 681) | SSKRYWGQGTLVTVSS |
| Exemplary anti-target | EVQLVESGGGLVQPGGSLTLSCVASGSTSSINAMGWYRRAPGKQRELVAGI |
| (DLL3) | SSDGSRVYFDSVKGRFTISRDNAKNSVYLQMNSLRAEDTAVYYCYYFKTIAG |
| (SEQ ID NO: 682) | AGMRYWGQGTLVTVSS |
| Exemplary anti-target | EVQLVESGGGLVQPGGSLTLSCVASGSTSLVNAMGWYRRAPGKQRELVAGI |
| (DLL3) | SSDGSLVYAESVKGRFTISRDNAKNSVYLQMNSLRAEDTAVYYCYYFRYGSG |
| (SEQ ID NO: 683) | SSLSYWGQGTLVTVSS |
| Exemplary anti-target | EVQLVESGGGLVQPGGSLTLSCVASGSTSLNNAIGWYRRAPGKQRELVAGIS |
| (DLL3) | SDGSVVYVDSVKGRFTISRDNAKNSVYLQMNSLRAEDTAVYYCYYFRTVPGA |
| (SEQ ID NO: 684) | SMKYWGQGTLVTVSS |
| Exemplary anti-target | EVQLVESGGGLVQPGGSLTLSCVASGSTSPVNAMAWYRRAPGKQRELVAGI |
| (DLL3) | SSDGSKVYVDSVKGRFTISRDNAKNSVYLQMNSLRAEDTAVYYCYYFRTVDG |
| (SEQ ID NO: 685) | SAISYWGQGTLVTVSS |
| Exemplary anti-target | EVQLVESGGGLVQPGGSLTLSCVASGTTSSMNAIGWYRRAPGKQRELVAGI |
| (DLL3) | SSDGSKLYDESVKGRFTISRDNAKNSVYLQMNSLRAEDTAVYYCYYFRNKG |
| (SEQ ID NO: 686) | SGGSYWGQGTLVTVSS |
| Exemplary anti-target | EVQLVESGGGLVQPGGSLTLSCVASGETSSINAMAWYRRAPGKQRELVAGI |
| (DLL3) | SSDYSKLYADSVKGRFTISRDNAKNSVYLQMNSLRAEDTAVYYCYYFRTVSGS |
| (SEQ ID NO: 687) | SRGYWGQGTLVTVSS |
| Exemplary anti-target | EVQLVESGGGLVQPGGSLTLSCVASGSTSTINAIGWYRRAPGKQRELVAGISS |
| (DLL3) | DSSKVYTESVKGRFTISRDNAKNSVYLQMNSLRAEDTAVYYCYYFRPGPGSQ |
| (SEQ ID NO: 688) | MAYWGQGTLVTVSS |
| Exemplary anti-target | EVQLVESGGGLVQPGGSLTLSCVASGSTYSMNAMGWYRRAPGKQRELVA |
| (DLL3) | GISSDGSQVYVDSVKGRFTISRDNAKNSVYLQMNSLRAEDTAVYYCYYFRTV |
| (SEQ ID NO: 689) | AGSASGYWGQGTLVTVSS |
| Exemplary anti-target | EVQLVESGGGLVQPGGSLTLSCVASGSPSSINAYGWYRRAPGKQRELVAGIS |
| (DLL3) | SDGSKVYSDSVKGRFTISRDNAKNSVYLQMNSLRAEDTAVYYCYYFRTVSGS |
| (SEQ ID NO: 690) | SYSYWGQGTLVTVSS |
| Exemplary anti-target | EVQLVESGGGLVQPGGSLTLSCVASGSTSTINAIGWYRRAPGKQRELVAGISS |
| (DLL3) | DGSKVYVDSVKGRFTISRDNAKNSVYLQMNSLRAEDTAVYYCYYFINLKGSS |
| (SEQ ID NO: 691) | MAYWGQGTLVTVSS |
| Exemplary anti-target | EVQLVESGGGLVQPGGSLTLSCVASGSTSSINAIGWYRRAPGKQRELVAGISS |
| (DLL3) | DGSKVYADSVKGRFTISRDNAKNSVYLQMNSLRAEDTAVYYCYYFRMVTGS |
| (SEQ ID NO: 692) | YGGYWGQGTLVTVSS |
| Exemplary anti-target | EVQLVESGGGLVQPGGSLTLSCVASGSISSINAMGWYRRAPGKQRELVAGIS |
| (DLL3) | SDGSSVYADSVKGRFTISRDNAKNSVYLQMNSLRAEDTAVYYCYYFKSSYGL |
| (SEQ ID NO: 693) | PMRYWGQGTLVTVSS |
| Exemplary anti-target | EVQLVESGGGLVQPGGSLTLSCVASGSTQVNNAMAWYRRAPGKQRELVA |
| (DLL3) | GISSDGSQVYYGSVKGRFTISRDNAKNSVYLQMNSLRAEDTAVYYCYYFKTV |
| (SEQ ID NO: 694) | SGQSLRYWGQGTLVTVSS |
| Exemplary anti-target | EVQLVESGGGLVQPGGSLTLSCVASGSTASFNAMAWYRRAPGKQRELVAGI |
| (DLL3) | SSDGSKVYTDSVKGRFTISRDNAKNSVYLQMNSLRAEDTAVYYCYYFRTVTG |
| (SEQ ID NO: 695) | RAARYWGQGTLVTVSS |
| Exemplary anti-target | EVQLVESGGGLVQPGGSLTLSCVASGSPLSINAIGWYRRAPGKQRELVAGISS |
| (DLL3) | DGSKVSADSVKGRFTISRDNAKNSVYLQMNSLRAEDTAVYYCYYFGPAIGAS |
| (SEQ ID NO: 696) | RTYWGQGTLVTVSS |
| Exemplary anti-target | EVQLVESGGGLVQPGGSLTLSCVASGSTTFINAIGWYRRAPGKQRELVAGISS |
| (DLL3) | DGSKVYEDSVKGRFTISRDNAKNSVYLQMNSLRAEDTAVYYCYYFRTVSGAP |
| (SEQ ID NO: 697) | KSYWGQGTLVTVSS |
| Exemplary anti-target | EVQLVESGGGLVQPGGSLTLSCVASGSTSSINAIGWYRRAPGKQRELVAGISS |
| (DLL3) | DRSKVYADSVKGRFTISRDNAKNSVYLQMNSLRAEDTAVYYCYYFHTVSGSS |
| (SEQ ID NO: 698) | MSYWGQGTLVTVSS |
| Exemplary anti-target | EVQLVESGGGLVQPGGSLTLSCVASGETDTINAVGWYRRAPGKQRELVAGI |
| (DLL3) | SSDGSKVYAESVKGRFTISRDNAKNSVYLQMNSLRAEDTAVYYCYYFRRLEG |
| (SEQ ID NO: 699) | YSNRYWGQGTLVTVSS |
| Exemplary anti-target | EVQLVESGGGLVQPGGSLTLSCVASGSTSPINAIGWYRRAPGKQRELVAGIS |
| (DLL3) | SDGSVVTTESVKGRFTISRDNAKNSVYLQMNSLRAEDTAVYYCYYFRTGSGS |
| (SEQ ID NO: 700) | SMGYWGQGTLVTVSS |
| Exemplary anti-target | EVQLVESGGGLVQPGGSLTLSCVASGSITSSNAMGWYRRAPGKQRELVAGI |
| (DLL3) | SSDGSHVHQESVKGRFTISRDNAKNSVYLQMNSLRAEDTAVYYCYYFTTVTG |
| (SEQ ID NO: 701) | SSMSYWGQGTLVTVSS |
| Exemplary anti-target | EVQLVESGGGLVQPGGSLTLSCAASRYSVSNLSMAWYRQAPGKKRELVAGI |
| (DLL3) | SADGSTVYVESVKGRFTISRDNAKNSVYLQMNSLRAEDTAVYYCYAYYWTE |
| (SEQ ID NO: 702) | RRPYWGQGTLVTVSS |
| Exemplary anti-target | EVQLVESGGGLVQPGDSLTLSCAASMSTV5VLSMAWYRQAPGKKRELVAGI |
| (DLL3) | SSDGSTVYIDSVKGRFTISRDNAKNSVYLQMNSLRAEDTAIYYCYAYSWDDA |
| (SEQ ID NO: 703) | HPYWGQGTLVTVSS |
| Exemplary anti-target | EVQLVESGGGLVQPGGSLTLSCAASDSYVSLLSMAWYRQAPGKKRELVAGIS |
| (DLL3) | VDGSTHYVASVKGRFTISRDNAKNSVYLQMNSLRAEDTAVYYCYAYRWMT |
| (SEQ ID NO: 704) | RLTYWGQGTLVTVSS |
| Exemplary anti-target | EVQLVESGGGLVQPGGSLTLSCAASDSAVSVLSIAWYRQAPGKKRELVAGIS |
| (DLL3) | TDGSKHYIDSVKGRFTISRDNAKNSVYLQMNSLRAEDTAVYYCYAYDWADA |
| (SEQ ID NO: 705) | QPYWGQGTLVTVSS |
| Exemplary anti-target | EVQLVESGGGLVQPGGSLTLSCAASHSSVTSLSLAWYRQAPGKKRELVAGIS |
| (DLL3) | YDGSKYYAESVKGRFTISRDNAKNSVYLQMNSLRAEDTAVYYCYAYSWTDRL |
| (SEQ ID NO: 706) | PYWGQGTLVTVSS |
| Exemplary anti-target | EVQLVESGGGLVQPGGSLTLSCAASDSVVKFLSMAWYRQAPGKKRELVAGI |
| (DLL3) | SANGSRTYMESVKGRFTISRDNAKNSVYLQMNSLRAEDTAVYYCYAYRWAT |
| (SEQ ID NO: 707) | RLPYWGQGTLVTVSS |
| Exemplary anti-target | EVQLVESGGGLVQPGGSLTLSCAASDPSVWNLSMAWYRQAPGKKRELVAG |
| (DLL3) | ISPDGSTDYVDSVKGRFTISRDNAKNSVYLQMNSLRAEDTAVYYCYAYKWS |
| (SEQ ID NO: 708) | NRLPYWGQGTLVTVSS |
| Exemplary anti-target | EVQLVESGGGLVQPGGSLTLSCAASGTSVMLLSLAWYRQAPGKKRELVAGIS |
| (DLL3) | PNGSAVYTESVKGRFTISRDNAKNSVYLQMNSLRAEDTAVYYCYAYGWKTR |
| (SEQ ID NO: 709) | QPYWGQGTLVTVSS |
| Exemplary anti-target | EVQLVESGGGLVQPGGSLTLSCAASSSPVSNLSLAWYRQAPGKKRELVAGIS |
| (DLL3) | PDGSTAYMESVKGRFTISRDNAKNSVYLQMNSLRAEDTAVYYCYAYRWPN |
| (SEQ ID NO: 710) | RRGYWGQGTLVTVSS |
| Exemplary anti-target | EVQLVESGGGLVQPGGSLTLSCAASWRSVLLLSVAWYRQAPGKKRELVAGIS |
| (DLL3) | NDGSTDYIDSVKGRFTISRDNAKNSVYLQMNSLRAEDTAVYYCYAYDWTTR |
| (SEQ ID NO: 711) | QRYWGQGTLVTVSS |
| Exemplary anti-target | EVQLVESGGGLVQPGGSLTLSCAASSSSVQYLSMAWYRQAPGKKRELVAGI |
| (DLL3) | STDGSAVYFDSVKGRFTISRDNAKNSVYLQMNSLRAEDTAVYYCYAYNWSY |
| (SEQ ID NO: 712) | AQPYWGQGTLVTVSS |
| Exemplary anti-target | EVQLVESGGGLVQPGGSLTLSCAASGTSVSLLSLAWYRQAPGKKRELVAGIS |
| (DLL3) | TGGSTHYIESVKGRFTISRDNAKNSVYLQMNSLRAEDTAVYYCYAYNWTDSL |
| (SEQ ID NO: 713) | QYWGQGTLVTVSS |
| Exemplary anti-target | EVQLVESGGGLVQPGGSLTLSCAASLSSVSNLSIAWYRQAPGKKRELVAG1ST |
| (DLL3) | DGSTVYIDSVKGRFTISRDNAKNSVYLQMNSLRAEDTAVYYCYAYSWTTSLP |
| (SEQ ID NO: 714) | YWGQGTLVTVSS |
| Exemplary anti-target | EVQLVESGGGLVQPGGSLTLSCAASMYSVSFLSMAWYRQAPGKKRELVAGI |
| (DLL3) | SNEGSTYYMDSVKGRFTISRDNAKNSVYLQMNSLRAEDTAVYYCYAYKWRS |
| (SEQ ID NO: 715) | RSTYWGQGTLVTVSS |
| Exemplary anti-target | EVQLVESGGGLVQPGGSLTLSCAASKSSVSHLSLAWYRQAPGKKRELVAGIS |
| (DLL3) | ADGSHVYTNSVKGRFTISRDNAKNSVYLQMNSLRAEDTAVYYCYAYSQTTR |
| (SEQ ID NO: 716) | DPYWGQGTLVTVSS |
| Exemplary anti-target | EVQLVESGGGLVQPGGSLTLSCAASYTSVLDLSIAWYRQAPGKKRELVAGIS |
| (DLL3) | DDGSRYYTDSVKGRFTISRDNAKNSVYLQMNSLRAEDTAVYYCYAYRWTAR |
| (SEQ ID NO: 717) | DTYWGQGTLVTVSS |
| Exemplary anti-target | EVQLVESGGGLVQPGGSLTLSCAASMSDVSFLSMAWYRQAPGKKRELVAGI |
| (DLL3) | SAEGSTLYMESVKGRFTISRDNAKNSVYLQMNSLRAEDTAVYYCYAYRWTS |
| (SEQ ID NO: 718) | RLSYWGQGTLVTVSS |
| Exemplary anti-target | EVQLVESGGGLVQPGGSLTLSCAASESSVSFLSSAWYRQAPGKKRELVAGIST |
| (DLL3) | DGSTVYIDSVKGRFTISRDNAKNSVYLQMNSLRAEDTAVYYCYAYSWTTRSR |
| (SEQ ID NO: 719) | YWGQGTLVTVSS |
| Exemplary anti-target | EVQLVESGGGLVQPGGSLTLSCAASGDSVSLLSMAWYRQAPGKKRELVAGI |
| (DLL3) | SANGSTSYIDSVKGRFTISRDNAKNSVYLQMNSLRAEDTAVYYCYAYNWTSR |
| (SEQ ID NO: 720) | YRYWGQGTLVTVSS |
| Exemplary anti-target | EVQLVESGGGLVQPGGSLTLSCAASGSDVWYLSLAWYRQAPGKKRELVAGI |
| (DLL3) | SDDGSRHYIESVKGRFTISRDNAKNSVYLQMNSLRAEDTAVYYCYAYSWKTR |
| (SEQ ID NO: 721) | FPYWGQGTLVTVSS |
| Exemplary anti-target | EVQLVESGGGLVQPGGSLTLSCAASKSAVAFLSIAWYRQAPGKKRELVAGIS |
| (DLL3) | PDGSTVYIESVKGRFTISRDNAKNSVYLQMNSLRAEDTAVYYCYAYSWTTRY |
| (SEQ ID NO: 722) | PYWGQGTLVTVSS |
| Exemplary anti-target | EVQLVESGGGLVQPGGSLTLSCAASFSAVAYLSMAWYRQAPGKKRELVAGI |
| (DLL3) | SDDGSTVYVDSVKGRFTISRDNAKNSVYLQMNSLRAEDTAVYYCYAYEWTN |
| (SEQ ID NO: 723) | ALPYWGQGTLVTVSS |
| Exemplary anti-target | EVQLVESGGGLVQPGGSLTLSCAASVYSVYDLSTAWYRQAPGKKRELVAGIS |
| (DLL3) | DDGSTVYFDSVKGRFTISRDNAKNSVYLQMNSLRAEDTAVYYCYAYSWITRS |
| (SEQ ID NO: 724) | PYWGQGTLVTVSS |
| Exemplary anti-target | EVQLVESGGGLVQPGGSLTLSCAASGDSVSFLSMAWYRQAPGKKRELVAGI |
| (DLL3) | SDEGSTVYIGSVKGRFTISRDNAKNSVYLQMNSLRAEDTAVYYCYAYSVVTTR |
| (SEQ ID NO: 725) | RQYWGQGTLVTVSS |
| Exemplary anti-target | EVQLVESGGGLVQPGGSLTLSCAASSSSVSLLSLAWYRQAPGKKRELVAGISD |
| (DLL3) | DGSIVYMDSVKGRFTISRDNAKNSVYLQMNSLRAEDTAVYYCYAYSWITRSP |
| (SEQ ID NO: 726) | YWGQGTLVTVSS |
| Exemplary anti-target | EVQLVESGGGLVQPGGSLTLSCAASADSVSFLSIAWYRQAPGKKRELVAGIS |
| (DLL3) | DDGSKHYFDSVKGRFTISRDNAKNSVYLQMNSLRAEDTAVYYCYAYRWEES |
| (SEQ ID NO: 727) | RQYWGQGTLVTVSS |
| Exemplary anti-target | EVQLVESGGGLVQPGGSLTLSCAASASSVTLLSIAWYRQAPGKKRELVAGIST |
| (DLL3) | DGSTDYLHSVKGRFTISRDNAKNSVYLQMNSLRAEDTAVYYCYAYTWTTRLP |
| (SEQ ID NO: 728) | YWGQGTLVTVTS |
| Exemplary anti-target | EVQLVESGGGLVQPGGSLTLSCAASADSVSFLSIAWYRQAPGKKRELVAGIS |
| (DLL3) | DDGSKHYFDSVKGRFTISRDNAKNSVYLQMNSLRAEDTAVYYCYAYRWEES |
| (SEQ ID NO: 729) | RQYWGQGTLVTVSS |
| Exemplary anti-target | EVQLVESGGGLVQPGGSLTLSCAASGTSVWLLSMAWYRQAPGKKRELVAGI |
| (DLL3) | SYDGSTVYVESVKGRFTISRDNAKNSVYLQMNSLRAEDTAVYYCYAYSWTTR |
| (SEQ ID NO: 730) | QPYWGQGTLVTVSS |
| Exemplary anti-target | EVQLVESGGGLVQPGGSLTLSCAASGSSVSILSIAWYRQAPGKKRELVAGISD |
| (DLL3) | DGSTVYIDSVKGRFTISRDNAKNSVYLQMNSLRAEDTAVYYCYAYVWGTRLP |
| (SEQ ID NO: 731) | YWGQGTLVTVSS |
| Exemplary anti-target | EVQLVESGGGLVQPGGSLTLSCAASGTAVSNLSIAWYRQAPGKKRELVAGIS |
| (DLL3) | DDGSTVYVDSVKGRFTISRDNAKNSVYLQMNSLRAEDTAVYYCYAYEWTNA |
| (SEQ ID NO: 732) | LPYWGQGTLVTVSS |
| Exemplary anti-target | EVQLVESGGGLVQPGGSLTLSCAASGSAVSMLSLAWYRQAPGKKRELVAGI |
| (DLL3) | SDDGSQVYIDSVKGRFTISRDNAKNSVYLQMNSLRAEDTAVYYCYAYRWED |
| (SEQ ID NO: 733) | ALTYWGQGTLVTVSS |
| Exemplary anti-target | EVQLVESGGGLVQPGGSLTLSCAASGMTVFFLSMAWYRQAPGKKRELVAGI |
| (DLL3) | SVDGSTVYSDSVKGRFTISRDNAKNSVYLQMNSLRAEDTAVYYCYAYSWTTR |
| (SEQ ID NO: 734) | YPYWGQGTLVTVSS |
| Exemplary anti-target | EVQLVESGGGLVQPGGSLTLSCAASQYSVTFLSVAWYRQAPGKKRELVAGIS |
| (DLL3) | DDGSNVYIDSVKGRFTISRDNAKNSVYLQMNSLRAEDTAVYYCYAYSWIDSL |
| (SEQ ID NO: 735) | RYWGQGTLVTVSS |
| Exemplary anti-target | EVQLVESGGGLVQPGGSLTLSCAASGETVSFLSLAWYRQAPGKKRELVAGIS |
| (DLL3) | TDGSTVYFVSVKGRFTISRDNAKNSVYLQMNSLRAEDTAVYYCYAYSWTTPR |
| (SEQ ID NO: 736) | AYWGQGTLVTVSS |
| Exemplary anti-target | QVQLQESGGGLVQAGGSLRLSCAASGSIAYIYTMDWYRQAPGKQRELVATS |
| (EGFR) | TRDGNVDYAESVKGRFTISRDNAKNTVYLQMNSLKPEDTAVYYCNADLRTA |
| (SEQ ID NO: 737) | VDLIRANYWGQGTQVTVSS |
| Exemplary anti-target | QVQLQESGGGLVQAGDSLRLSCVVSGRTDSWYVMGWFRQAPGKDREFVA |
| (EGFR) | GVSWSYGNTYYADSVKGRFTASRDNAKNTAYLQMNSLNAEDTAVYYCAAR |
| (SEQ ID NO: 738) | VSREVIPTRWDLYNYWGQGTQVTVSS |
| Exemplary anti-target | QVQLQESGGGSVQPGGSLRVSCVVSRTIISINAMTWYHQAPGKRRELVAIIT |
| (EGFR) | SGGETNYADSVKGRFTISRDNAKNTAYLQMNNLKPEDTGVYYCNVVPPLGS |
| (SEQ ID NO: 739) | WGQGTQVTVSS |
| Exemplary anti-target | QVQLQESGGGRVQAGGSLRLSCSASARTLRLYAVGWFRQAPGKEREFVAGI |
| (EGFR) | GRSERTYYTDSVKGRFTLSRDNAKNTVFLEMNDLEPEDTAVYFCALTFQTTD |
| (SEQ ID NO: 740) | MVDVPTTQHEYDYWGRGTQVTVSS |
| Exemplary anti-target | QVQLQESGGGLVQAGGSLRLSCAASGSIAYIYTMDWYRQAPGKQRELVATS |
| (EGFR) | THDGNTDYADSVKGRFTISRDNVKNTVYLQMNSLKPEDTAVYYCNADLRTA |
| (SEQ ID NO: 741) | VDLIRANYWGQGTQVTVSS |
| Exemplary anti-target | QVQLQESGGGLVQPGGSLRLSCAASGSIAYIYTMDWYRQAPGKQRELVATS |
| (EGFR) | TRDGNTDYADSVKGRFTISRDNAKDTVYLQMNSLKPEDTAVYYCNADLRTA |
| (SEQ ID NO: 742) | VDLIRANYWGQGTQVTVSS |
| Exemplary anti-target | QVQLQESGGGLVQAGGSLTLSCAASGRYQMAWFRQAPEKEREFVGTISSG |
| (EGFR) | DSTWYTNSVKGRFAISRDSARNTVYLQMNDLKPEDTAIYYCAAALYYRDSRR |
| (SEQ ID NO: 743) | AADYPYWGQGTQVTVSS |
| Exemplary anti-target | QVQLQESGGGRVQAGESLRLSCSTSTRTLKLYAVGWFRQAPGKERDFVAGI |
| (EGFR) | GRSERIYYIDSVKGRFTLSRDNAKNTVFLEMNDLEPEDTAVYFCAATFQTSDN |
| (SEQ ID NO: 744) | VGVPTVQHEYDYWGQGTQVTVSS |
| Exemplary anti-target | QVQLQESGGGLVQAGGSLRLSCAASGSIAYIYTMDWYRQAPGKQRELVATS |
| (EGFR) | THDGNTDYADSVKGRFTISRDNAKNTVTLQMNSLKPEDTAVYYCNADLRTA |
| (SEQ ID NO: 745) | VDLIRANYWGQGTQVTVSS |
| Exemplary anti-target | QVQLQESGGGLVQAGGSLRLSCAASGSIAYIYTMDWYRQAPGKQRELVATF |
| (EGFR) | TRDGNTDYADSVKGRFTISRDNAKNTVYLQMNSLKPEDTAVYYCNTDLRTA |
| (SEQ ID NO: 746) | VDLIRANYWGQGTQVTVSS |
| Exemplary anti-target | QVQLQESGGGLVQAGGSLRLSCAASGSIAYIYTMDWYRQAPGKQRELVATS |
| (EGFR) | THDGNTDYADSLKGRFTISRDNAKNTVYLQMNSLKPEDTAVYYCNADLRTA |
| (SEQ ID NO: 747) | VDLIRANYWGQGTQVTVSS |
| Exemplary anti-target | QVQLQESGGGSVQAGGSLRLSCAASGRYQMAWFRQAPEKEREFVGTISSG |
| (EGFR) | DSTWYTNSLKGRFAISRDSARDTVYLQMNDLKPEDTAVYYCAAALYYRDSRR |
| (SEQ ID NO: 748) | AADYPYWGQGTQVTVSS |
| Exemplary anti-target | QVQLQESGGGLVQTGGSLRLSCAVSGSIVTINAMTWYRQAPGKRRELVAIIT |
| (EGFR) | SGGETNYADSVKGRFTISRDNAKNTAHLQMNSLNPEDTGVYYCNVVPPLGS |
| (SEQ ID NO: 749) | WGQGTQVTVSS |
| Exemplary anti-target | QVQLQESGGGLVQTGGSLRLSCAVSRSIVSIKSMTWYRQAPGKRRELVALIT |
| (EGFR) | SGGETNYSDSVKGRFTISRDNAKNTVYLQMNSLKPEDTGVYYCNVVPPLGS |
| (SEQ ID NO: 750) | WGQGTQVTVSS |
| Exemplary anti-target | QVQLQESGGGLVETGGSLRLSCAGSGSTFRHHAMAWFRQTPGKEREFVSAI |
| (EGFR) | NDHGDRTKYLDSVRGRFTISRDNTDNMVYLQMTDLRPEDTANYSCAAGPL |
| (SEQ ID NO: 751) | VDYLETTPLVYTYWGHGTQVTVSS |
| Exemplary anti-target | QVQLQESGGGLVQAGGSLRLSCAASGSIAYIYTMDWYRQAPGKQRELVATS |
| (EGFR) | THDGNTDYADSVKGRFTISRDNAKNTVYLQMNSLKPEDTAVYYCNADLRTA |
| (SEQ ID NO: 752) | VDLIRANYWGQGTQVTVSS |
| Exemplary anti-target | QVQLQESGGGSVQAGGSLTLSCAASGSIAYIYTMDWYRQAPGKQRELVATS |
| (EGFR) | THDGNTDYADSVKGRFTISRDNAKNTVYLQMNSLKPEDTAVYYCNADLRTA |
| (SEQ ID NO: 753) | VDLIRANYWGQGTQVTVSS |
| Exemplary anti-target | QVQLQESGGGLVQPGGSLRLSCTASVSIFSVNAVDWYRQSPGKERELVAIM |
| (EGFR) | TSDGSTNYGDSVKGRFTISRDNAKNTVYLQMNNLKPEDTAVYYCNTVPPRY |
| (SEQ ID NO: 754) | WGQGTQVTVSS |
| Exemplary anti-target | QVQLQESGGGLVQPGGSLRLSCAASGSIAYIYTMDWYRQAPGKQRELVATS |
| (EGFR) | TRDGNIDYADSVKGRFTISRDSAKNTVYLQMSSLKPEDTAVYYCNADLRTAV |
| (SEQ ID NO: 755) | DLIRANYWGQGTQVTVSS |
| Exemplary anti-target | QVQLQESGGGLVQAGGSLTLSCAASGRYQLAWFRQAPEKVREFVGTISSGD |
| (EGFR) | STWYTNSVKGRFAISRDSARNTVYLQMNDLKPEDTAVYYCAAALYYRDSRR |
| (SEQ ID NO: 756) | AADYPYWGQGTQVTVSS |
| Exemplary anti-target | QVQLQESGGGSVQAGGSLRLSCAASGRYHMAWFRQAPEKEREFVGTISSG |
| (EGFR) | DSTWYTNSVKGRFAISRDSARNTAYLQMNDLKPEDTAVYYCAAALYYGDSR |
| (SEQ ID NO: 757) | RAADYPYWGQGTQVTVSS |
| Exemplary anti-target | QVQLQESGGGSVQAGGSLRLSCAASGSIAYIYTMDWYRQAPGKQRELVATS |
| (EGFR) | THDGNTDYTDSVKGRFTISRDNAKNTVYLQMNSLKPEDTAVYYCNADLRTA |
| (SEQ ID NO: 758) | VDLIRANYWGQGTQVTVSS |
| Exemplary anti-target | QVQLQESGGGLVQAGGSLRLSCAASGSIAYIYTMDWYRQTPGKQRELVATS |
| (EGFR) | TRDANTDYAGSVKGRFTISRDNAKDTVYLQMNSLKPEDTAVYYCHADLRTA |
| (SEQ ID NO: 759) | VDLIRANYWGQGTQVTVSS |
| Exemplary anti-target | QVQLQESGGGLVQAGGSLRLSCAASGRYNMAWFRQAPEKEREFVGTITSA |
| (EGFR) | DSTWYTNSVKGRFAITQDSARNTVYLQMNDLKPEDTAVYYCAAALYYGDSR |
| (SEQ ID NO: 760) | RAADYPYWGQGTQVTVSS |
| Exemplary anti-target | QVQLQESGGGLVQPGGSLRLSCAASGRYQMAWFRQAPEKEREFVGTISSG |
| (EGFR) | DSTWYTNSVKGRFAISRDSARTTVYLQMNDLKPEDTAVYYCAAALYYRDSW |
| (SEQ ID NO: 761) | RAADYPYWGQGTQVTVSS |
| Exemplary anti-target | QVQLQESGGGLVQPGESLRLSCAATGRYHLAWFRQAPEKEREFVGTITSAD |
| (EGFR) | STWYTNSVKGRFAITRDSARNTVYLQMNDLKPEDTAVYYCAAALYYGDSRR |
| (SEQ ID NO: 762) | AADYPYWGQGTQVTVSS |
| Exemplary anti-target | QVQLQESGGGLVQAGGSLKLSCADSGRSFSNYIMGWFRQAPGKEREFVAG |
| (EGFR) | LGWSPGNTYYADSVKGRFTISRDNAKNMVYLQMNSLNPEDTAVYYCAARR |
| (SEQ ID NO: 763) | GDVIYTTPWNYVYWGQGTQVTVSS |
| Exemplary anti-target | QVQLQESGGGLVQAGGSLRLSCAASGSIAYIYTMDWYRQAPGKQRELVATS |
| (EGFR) | THDGNTDYADSVKGRFTISRDNAKNTVYLQMNSLKPEDTAVYYCNADLRTP |
| (SEQ ID NO: 764) | VDLIRANYWGQGTQVTVSS |
| Exemplary anti-target | QVQLQESGGGSVQAGGSLRLSCAAPGRYQMAWFRQAPEKEREFVGTISSG |
| (EGFR) | DSTWYTNSVKGRFAISRDSARNTVYLQMNDLKSEDTAVYYCAAALYYRDSR |
| (SEQ ID NO: 765) | RAIDYPYWGQGTQVTVSS |
| Exemplary anti-target | QVQLQESGGGSVQAGGSLRLSCAASGLTFSSYAMAWFRQAPGKQRELVAR |
| (EGFR) | ITSGGTTDYADSVKGRFTISRDNAKNTMYLQMNSLKPEDTAVYYCAADLTYR |
| (SEQ ID NO: 766) | NLLLKLPHYWGQGTQVTVSS |
| Exemplary anti-target | QVQLQESGGGLVQAGGSLRLSCAASGNIAYIYTMDWYRQAPGKQRELVAT |
| (EGFR) | STHDGSTDYADSVKGRFTISRDNAKNTVYLQMNSLKPEDTAVYYCNADLRT |
| (SEQ ID NO: 767) | PVDLIRANYWGQGTQVTVSS |
| Exemplary anti-target | QVQLQESGGGLVQPGGSLRLSCAASGSIAYIYTMDWYRQAPGKQRELVATS |
| (EGFR) | TWDGNTDYADSVKGRFTISRDNAKNTVYLQMNSLKPEDTAVYYCNADLRT |
| (SEQ ID NO: 768) | AVDLIRANYWGQGTQVTVSS |
| Exemplary anti-target | QVQLQESGGGLVQAGGSLRLSCAASGSIAYIYTMDWYRQAPGKQRELVATS |
| (EGFR) | THDGNTDYADSVKGRFTISRDNAKNTVYLQMSSLKPDDTAVYYCNADLRTA |
| (SEQ ID NO: 769) | VDLIRANYWGQGTQVTVSS |
| Exemplary anti-target | QVQLQESGGGLVQAGESLSLSCAASGNDFVITDMHWYRQAPGKQREWVA |
| (EGFR) | TITRFATTNYADSVKGRFTISRDNAKNTWYLQMNSLKPDDTAVYYCKAIGLR |
| (SEQ ID NO: 770) | GVPDVNRQFEVWGQGTQVTVSS |
| Exemplary anti-target | QVQLQESGGGLVQAGGSLRLSCAASGAIAYIYGMGWYRQAPGNQRELVAA |
| (EGFR) | ISSGGSTDYADSVKGRFTISRDNAKNTVYLQMSSLKPEDTAVYYCNADVRTS |
| (SEQ ID NO: 771) | RNLVRSDYWGQGTQVTVSS |
| Exemplary anti-target | QVQLQESGGGLVQAGGSLRLSCAASGRYHTAWFRQAPEKEREFVGTISSGD |
| (EGFR) | STWYTNSVKGRFAISRDSARNTVYLQMNDLKPEDTAVYYCAAALYYGDSRR |
| (SEQ ID NO: 772) | AGDYPYWGQGTQVTVSS |
| Exemplary anti-target | QVQLQESGGGLVQAGGSLRLSCAASGNIAYIYTMNWYRQAPGKQRELVAT |
| (EGFR) | STHAGNTDYADSVKGRFTISRDNAKNTVYLQMNSLKPEDTAVYYCNVDLRT |
| (SEQ ID NO: 773) | AVDLIRANYWGQGTQVTVSS |
| Exemplary anti-target | QVQLQESGGGLVQPGGSLRLSCAASGNlAYIYTMGWYRQAPGKQRELVAT |
| (EGFR) | STHDGNSDYADSVKGRFTISRDNAKNTVYLQMNTLKPDDTAVYYCNADLRT |
| (SEQ ID NO: 774) | PVDRIRGNFWGQGTQVTVSS |
| Exemplary anti-target | EVQLLESGGGLVQPGGSLTLSCAASGSIAYIYTMDWYRQAPGKQRELVATST |
| (EGFR) | RDGNVDYADSVKGRFTISRDNSKNTLYLQMNSLRAEDTAVYYCNADLRTAV |
| (SEQ ID NO: 775) | DLIRANYWGLGTQVTVSS |
| Exemplary anti-target | EVQLLESGGGLVQPGGSLTLSCAASGRTDSWYVMGWFRQAPGKDREFVA |
| (EGFR) | GVSWSYGNTYYADSVKGRFTISRDNSKNTLYLQMNSLRAEDTAVYYCAARV |
| (SEQ ID NO: 776) | SREVIPTRWDLYNYWGLGTQVTVSS |
| Exemplary anti-target | EVQLLESGGGLVQPGGSLTLSCAASARTLRLYAVGWFRQAPGKEREFVAGIG |
| (EGFR) | RSERTYYTDSVKGRFTISRDNSKNTLYLQMNSLRAEDTAVYYCALTFQTTDM |
| (SEQ ID NO: 777) | VDVPTTQHEYDYWGLGTQVTVSS |
| Exemplary anti-target | EVQLLESGGGLVQPGGSLTLSCAASGSIVTINAMTWYRQAPGKRRELVAIITS |
| (EGFR) | GGETNYADSVKGRFTISRDNSKNTLYLQMNSLRAEDTAVYYCNVVPPLGSW |
| (SEQ ID NO: 778) | GLGTQVTVSS |
| Exemplary anti-target | EVQLLESGGGLVQPGGSLTLSCAASGRYHMAWFRQAPGKEREFVGTISSGD |
| (EGFR) | STWYTNSVKGRFTISRDNSKNTLYLQMNSLRAEDTAVYYCAAALYYGDSRRA |
| (SEQ ID NO: 779) | ADYPYWGLGTQVTVSS |
| Exemplary anti-target | EVQLLESGGGLVQPGGSLTLSCAASGSTFRHHAMAWFRQTPGKEREFVSAI |
| (EGFR) | NDHGDRTKYLDSVKGRFTISRDNSKNTLYLQMNSLRAEDTAVYYCAAGPLV |
| (SEQ ID NO: 780) | DYLETTPLVYTYWGLGTQVTVSS |
| Exemplary anti-target | EVQLLESGGGLVQPGGSLTLSCAASGRSFSNYIMGWFRQAPGKEREFVAGL |
| (EGFR) | GWSPGNTYYADSVKGRFTISRDNSKNTLYLQMNSLRAEDTAVYYCAARRGD |
| (SEQ ID NO: 781) | VIYTTPWNYVYWGLGTQVTVSS |
| Exemplary anti-target | EVQLLESGGGLVQPGGSLTLSCAASGLTFSSYAMAWFRQAPGKQRELVARIT |
| (EGFR) | SGGTTDYADSVKGRFTISRDNSKNTLYLQMNSLRAEDTAVYYCAADLTYRNL |
| (SEQ ID NO: 782) | LLKLPHYWGLGTQVTVSS |
| Exemplary anti-target | EVQLLESGGGLVQPGGSLTLSCAASVSIFSVNAVDWYRQSPGKERELVAIMT |
| (EGFR) | SDGSTNYDDSVKGRFTISRDNSKNTLYLQMNSLRAEDTAVYYCNTVPPRYW |
| (SEQ ID NO: 783) | GLGTQVTVSS |
| Exemplary anti-target | EVQLLESGGGLVQPGGSLTLSCAASGNDFVITDMHWYRQAPGKQREWVA |
| (EGFR) | TITRFATTNYADSVKGRFTISRDNSKNTLYLQMNSLRAEDTAVYYCKAIGLRG |
| (SEQ ID NO: 784) | VPDVNRQFEVWGLGTQVTVSS |
| Exemplary anti-target | EVQLLESGGGLVQPGGSLTLSCAASGAIAYIYGMGWYRQAPGKQRELVAAIS |
| (EGFR) | SGGSTDYADSVKGRFTISRDNSKNTLYLQMNSLRAEDTAVYYCNADVRTSR |
| (SEQ ID NO: 785) | NLVRSDYWGLGTQVTVSS |
| Exemplary EGFR | EVQLVESGGGLVQPGNSLRLSCAASGFTFSKFGMSWVRQ [symbol] |
| ProTriTAC containing | [symbol] LEWVSSISGSGRDTLYADSVKGRFTISRDNAKTTLYLQMNSLRPEDTA |
| linker sequence L001 | VYYCTIGGSLSVSSQGTLVTVSSGGGGKPLGLQARVVGGGGT |
| (identified by residues in | QTVVTQEPSLTVSPGGTVTLTCASSTGAVTSGNYPNWVQQKPGQAPRGLIG |
| bold and italics) and | GTKFLVPGTPARFSGSLLGGKAALTLSGVQPEDEAEYYCTLWYSNRWVFGG |
| masking sequence | GTKLTVLGGGGSGGGGSGGGGSEVQLVESGGGLVQPGGSLKLSCAASGFTF |
| M027(identified by | NKYAINWVRQAPGKGLEWVARIRSKYNNYATYYADQVKDRFTISRDDSKNT |
| residues that are bold | AYLQMNNLKTEDTAVYYCVRHANFGNSYISYWAYWGQGTLVTVSSGGGGS |
| and underlined) | GGGSEVQLLESGGGLVQPGGSLTLSCAASGRTDSWYVMGWFRQAPGKDR |
| (SEQ ID No. 786) | EFVAGVSWSYGNTYYADSVKGRFTISRDNSKNTLYLQMNSLRAEDTAVYYC |
| | AARVSREVIPTRWDLYNYWGLGTQVTVSSHHHHHH |
| Exemplary EGFR | EVQLVESGGGLVQPGNSLRLSCAASGFTFSKFGMSWVRQ [symbol] |
| ProTriTAC containing | [symbol] LEWVSSISGSGRDTLYADSVKGRFTISRDNAKTTLYLQMNSLRPEDTA |
| linker sequence L040 | VYYCTIGGSLSVSSQGTLVTVSSGGGGPQASTGRSGGGGGGT |
| (identified by residues in | QTVVTQEPSLTVSPGGTVTLTCASSTGAVTSGNYPNWVQQKPGQAPRGLIG |
| bold and italics) and | GTKFLVPGTPARFSGSLLGGKAALTLSGVQPEDEAEYYCTLWYSNRWVFGG |
| masking sequence | GTKLTVLGGGGSGGGGSGGGGSEVQLVESGGGLVQPGGSLKLSCAASGFTF |
| M027(identified by | NKYAINWVRQAPGKGLEWVARIRSKYNNYATYYADQVKDRFTISRDDSKNT |
| residues that are bold | AYLQMNNLKTEDTAVYYCVRHANFGNSYISYWAYWGQGTLVTVSSGGGGS |
| and underlined) | GGGSEVQLLESGGGLVQPGGSLTLSCAASGRTDSWYVMGWFRQAPGKDR |
| (SEQ ID No. 787) | EFVAGVSWSYGNTYYADSVKGRFTISRDNSKNTLYLQMNSLRAEDTAVYYC |
| | AARVSREVIPTRWDLYNYWGLGTQVTVSSHHHHHH |
| Exemplary EGFR | EVQLVESGGGLVQPGNSLRLSCAASGFTFSKFGMSWVRQ [symbol] |
| ProTriTAC containing | [symbol] LEWVSSISGSGRDTLYADSVKGRFTISRDNAKTTLYLQMNSLRPEDTA |
| linker sequence L041 | VYYCTIGGSLSVSSQGTLVTVSSGGGGPQGSTGRAAGGGGGT |
| (identified by residues in | QTVVTQEPSLTVSPGGTVTLTCASSTGAVTSGNYPNWVQQKPGQAPRGLIG |
| bold and italics) and | GTKFLVPGTPARFSGSLLGGKAALTLSGVQPEDEAEYYCTLWYSNRWVFGG |
| masking sequence | GTKLTVLGGGGSGGGGSGGGGSEVQLVESGGGLVQPGGSLKLSCAASGFTF |
| M027(identified by | NKYAINWVRQAPGKGLEWVARIRSKYNNYATYYADQVKDRFTISRDDSKNT |
| residues that are bold | AYLQMNNLKTEDTAVYYCVRHANFGNSYISYWAYWGQGTLVTVSS |
| and underlined) | GGGGSGGGSEVQLLESGGGLVQPGGSLTLSCAASGRTDSWYVMGWFRQA |
| (SEQ ID No. 788) | PGKDREFVAGVSWSYGNTYYADSVKGRFTISRDNSKNTLYLQMNSLRAEDT |
| | AVYYCAARVSREVIPTRWDLYNYWGLGTQVTVSSHHHHHH |
| Exemplary EGFR | EVQLVESGGGLVQPGNSLRLSCAASGFTFSKFGMSWVRQ [symbol] |
| ProTriTAC containing | [symbol] LEWVSSISGSGRDTLYADSVKGRFTISRDNAKTTLYLQMNSLRPEDTA |
| linker sequence L042 | VYYCTIGGSLSVSSQGTLVTVSSGGGGPPASSGRAGGGGGT |
| (identified by residues in | QTVVTQEPSLTVSPGGTVTLTCASSTGAVTSGNYPNWVQQKPGQAPRGLIG |
| bold and italics) and | GTKFLVPGTPARFSGSLLGGKAALTLSGVQPEDEAEYYCTLWYSNRWVFGG |
| masking sequence | GTKLTVLGGGGSGGGGSGGGGSEVQLVESGGGLVQPGGSLKLSCAASGFTF |
| M027(identified by | NKYAINWVRQAPGKGLEWVARIRSKYNNYATYYADQVKDRFTISRDDSKNT |
| residues that are bold | AYLQMNNLKTEDTAVYYCVRHANFGNSYISYWAYWGQGTLVTVSS |
| and underlined) | GGGGSGGGSEVQLLESGGGLVQPGGSLTLSCAASGRTDSWYVMGWFRQA |
| (SEQ ID No. 789) | PGKDREEVAGVSWSYGNTYYADSVKGRFTISRDNSKNTLYLQMNSLRAEDT |
| | AVYYCAARVSREVIPTRWDLYNYWGLGTQVTVSSHHHHHH |
| Exemplary EGFR | EVQLVESGGGLVQPGNSLRLSCAASGFTFSKFGMSWVRQ [symbol] |
| ProTriTAC containing | [symbol] LEWVSSISGSGRDTLYADSVKGRFTISRDNAKTTLYLQMNSLRPEDTA |
| linker sequence L045 | VYYCTIGGSLSVSSQGTLVTVSSGGGGPIPVQGRAHGGGGTQTVVTQEPSL |
| (identified by residues in | TVSPGGTVTLTCASSTGAVTSGNYPNWVQQKPGQAPRGLIGGTKFLVPGTP |
| bold and italics) and | ARFSGSLLGGKAALTLSGVQPEDEAEYYCTLWYSNRWVFGGGTKLTVLGGG |
| masking sequence | GSGGGGSGGGGSEVQLVESGGGLVQPGGSLKLSCAASGFTFNKYAINWVR |
| M027(identified by | QAPGKGLEWVARIRSKYNNYATYYADQVKDRFTISRDDSKNTAYLQMNNLK |
| residues that are bold | TEDTAVYYCVRHANFGNSYISYWAYWGQGTLVTVSSGGGGSGGGS |
| and underlined) | EVQLLESGGGLVQPGGSLTLSCAASGRTDSWYVMGWFRQAPGKDREFVA |
| (SEQ ID No. 790) | GVSWSYGNTYYADSVKGRFTISRDNSKNTLYLQMNSLRAEDTAVYYCAARV |
| | SREVIPTRWDLYNYWGLGTQVTVSSHHHHHH |
| Exemplary EGFR | EVQLVESGGGLVQPGNSLRLSCAASGFTFSKFGMSWVRQGGGGGLDGNEE |
| ProTriTAC containing a | PGGLEWVSSISGSGRDTLYADSVKGRFTISRDNAKTTLYLQMNSLRPEDTAV |
| non-cleavable linker | YYCTIGGSLSVSSQGTLVTVSSGGGGSGGGGSGGVVGGGGTQTVVTQEPSL |
| sequence(identified by | TVSPGGTVTLTCASSTGAVTSGNYPNWVQQKPGQAPRGLIGGTKFLVPGTP |
| residues in bold and | ARFSGSLLGGKAALTLSGVQPEDEAEYYCTLWYSNRWVFGGGTKLTVLGGG |
| italics) and masking | GSGGGGSGGGGSEVQLVESGGGLVQPGGSLKLSCAASGFTFNKYAINWVR |
| sequence | QAPGKGLEWVARIRSKYNNYATYYADQVKDRFTISRDDSKNTAYLQMNNLK |
| M027(identified by | TEDTAVYYCVRHANFGNSYISYWAYWGQGTLVTVSS |
| residues that are bold | GGGGSGGGSEVQLLESGGGLVQPGGSLTLSCAASGRTDSWYVMGWFRQA |
| and underlined) | PGKDREFVAGVSWSYGNTYYADSVKGRFTISRDNSKNTLYLQMNSLRAEDT |
| (SEQ ID No. 791) | AVYYCAARVSREVIPTRWDLYNYWGLGTQVTVSSHHHHHH |
| Exemplary GFP TriTAC | QVQLVESGGALVQPGGSLRLSCAASGFPVNRYSMRWYRQAPGKEREWVA |
| Sequence | GMSSAGDRSSYEDSVKGRFTISRDDARNTVYLQMNSLKPEDTAVYYCNVNV |
| (SEQ ID No. 792) | GFEYWGQGTQVTVSSGGGGSGGGSEVQLVESGGGLVQPGNSLRLSCAASG |
| | FTFSKFGMSWVRQAPGKGLEWVSSISGSGRDTLYADSVKGRFTISRDNAKT |
| | TLYLQMNSLRPEDTAVYYCTIGGSLSVSSQGTLVTVSSGGGGSGGGSEVQLV |
| | ESGGGLVQPGGSLKLSCAASGFTFNKYAINWVRQAPGKGLEWVARIRSKYN |
| | NYATYYADQVKDRFTISRDDSKNTAYLQMNNLKTEDTAVYYCVRHANFGNS |
| | YISYWAYWGQGTLVTVSSGGGGSGGGGSGGGGSQTVVTQEPSLIVSPGGT |
| | VTLTCASSTGAVTSGNYPNWVQQKPGQAPRGLIGGTKFLVPGTPARFSGSL |
| | LGGKAALTLSGVQPEDEAEYYCTLWYSNRWVFGGGTKLTVLHHHHHH |
| Exemplary EGFR TriTAC | EVQLLESGGGLVQPGGSLTLSCAASGRTDSWYVMGWFRQAPGKDREFVA |
| Sequence | GVSWSYGNTYYADSVKGRFTISRDNSKNTLYLQMNSLRAEDTAVYYCAARV |
| (SEQ ID No. 793) | SREVIPTRWDLYNYWGLGTQVTVSSGGGGSGGGSEVQLVESGGGLVQPGN |
| | SLRLSCAASGFTFSKFGMSWVRQAPGKGLEWVSSISGSGRDTLYADSVKGR |
| | FTISRDNAKTTLYLQMNSLRPEDTAVYYCTIGGSLSVSSQGTLVTVSSGGGGS |
| | GGGSEVQLVESGGGLVQPGGSLKLSCAASGFTFNKYAINWVRQAPGKGLE |
| | WVARIRSKYNNYATYYADQVKDRFTISRDDSKNTAYLQMNNLKTEDTAVYY |
| | CVRHANFGNSYISYWAYWGQGTLVTVSSGGGGSGGGGSGGGGSQTVVTQ |
| | EPSLTVSPGGTVTLTCASSTGAVTSGNYPNWVQQKPGQAPRGLIGGTKFLV |
| | PGTPARFSGSLLGGKAALTLSGVQPEDEAEYYCTLWYSNRWVFGGGTKLTVL |
| | HHHHHH |
| Exemplary anti-CD3 scFv | QTVVTQEPSLIVSPGGTVTLTCASSTGAVISGNYPNWVQQKPGQAPRGLIG |
| (SEQ ID NO: 794) | GTKFLVPGTPARFSGSLLGGKAALTLSGVQPEDEAEYYCTLWYSNRWVFGG |
| | GTKLTVLGGGGSGGGGSGGGGSEVQLVESGGGLVQPGGSLKLSCAASGFTF |
| | NKYAINWVRQAPGKGLEWVARIRSKYNNYATYYADQVKDRFTISRDDSKNT |
| | AYLQMNNLKTEDTAVYYCVRHANFGNSYISYWAYWGQGTLVTVSS |
| Exemplary proCAR | MALPVTALLLPLALLLHAARPTSDYKDDDDKTTTPAPRPPTPAPTIASQPLSLR |
| construct C1081 (SEQ ID | PEACRPAAGGAVHTRGLDFACDIYIWAPLAGTCGVLLLSLVITLYKRGRKKLLY |
| No. 804) | IFKQPFMRPVQTTQEEDGCSCRFPEEEEGGCELRVKFSRSADAPAYQQGQN |
| | QLYNELNLGRREEYDVLDKRRGRDPEMGGKPRRKNPQEGLYNELQKDKMA |
| | EAYSEIGMKGERRRGKGHDGLYQGLSTATKDTYDALHMQALPPR |
| Exemplary proCAR | MALPVTALLLPLALLLHAARPEVQLLESGGGLVQPGGSLTLSCAASGRSFSNY |
| construct C1824 (SEQ ID | IMGWFRQAPGKEREFVAGLGWSPGNTYYADSVKGRFTISRDNSKNTLYLQ |
| No. 805) | MNSLRAEDTAVYYCAARRGDVIYTTPWNYVYWGLGTQVTVSSTSDYKDDD |
| | DKTTTPAPRPPTPAPTIASQPLSLRPEACRPAAGGAVHTRGLDFACDIYIWAP |
| | LAGTCGVLLLSLVITLYKRGRKKLLYIFKQPFMRPVQTTQEEDGCSCRFPEEEE |
| | GGCELRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGLST |
| | ATKDTYDALHMQALPPR |
| Exemplary proCAR | MALPVTALLLPLALLLHAARPEVQLVESGGGLVQPGNSLRLSCAASGFTFSKF |
| construct C1826 (SEQ ID | GMSWVRQAPGKGLEWVSSISGSGRDTLYADSVKGRFTISRDNAKTTLYLQ |
| No. 806) | MNSLRPEDTAVYYCTIGGSLSVSSQGTLVTVSSGSGGSGGGGSGGGGGSGE |
| | VQLLESGGGLVQPGGSLTLSCAASGRSFSNYIMGWFRQAPGKEREFVAGLG |
| | WSPGNTYYADSVKGRFTISRDNSKNTLYLQMNSLRAEDTAVYYCAARRGDV |
| | IYTTPWNYVYWGLGTQVTVSSTSDYKDDDDKTTTPAPRPPTPAPTIASQPLS |
| | LRPEACRPAAGGAVHTRGLDFACDIYIWAPLAGTCGVLLLSLVITLYKRGRKK |
| | LLYIFKQPFMRPVQTTQEEDGCSCRFPEEEEGGCELRVKFSRSADAPAYQQG |
| | QNQLYNELNLGRREEYDVLDKRRGRDPEMGGKPRRKNPQEGLYNELQKDK |
| | MAEAYSEIGMKGERRRGKGHDGLYQGLSTATKDTYDALHMQALPPR |
| Exemplary proCAR | MALPVTALLLPLALLLHAARPEVQLVESGGGLVQPGNSLRLSCAASGFTFSKF |
| construct C1950 (SEQ ID | GMSWVRQANPWVPPREGLEWVSSISGSGRDTLYADSVKGRFTISRDNAKT |
| No. 807) | TLYLQMNSLRPEDTAVYYCTIGGSLSVSSQGTLVTVSSGSGGSGGGGSGGG |
| | GGSGEVQLLESGGGLVQPGGSLTLSCAASGRSFSNYIMGWFRQAPGKEREF |
| | VAGLGWSPGNTYYADSVKGRFTISRDNSKNTLYLQMNSLRAEDTAVYYCAA |
| | RRGDVIYTTPWNYVYWGLGTQVTVSSTSDYKDDIDDKTTTPAPRPPTPAPTI |
| | ASQPLSLRPEACRPAAGGAVHTRGLDFACDIYIWAPLAGTCGVLLLSLVITLYK |
| | RGRKKLLYIFKQPFMRPVQTTQEEDGCSCRFPEEEEGGCELRVKFSRSADAP |
| | AYQQGQNQLYNELNLGRREEYDVLDKRRGRDPEMGGKPRRKNPQEGLYN |
| | ELQKDKMAEAYSEIGMKGERRRGKGHDGLYQGLSTATKDTYDALHMQALP |
| | PR |
| Exemplary proCAR | MALPVTALLLPLALLLHAARPEVQLVESGGGLVQPGNSLRLSCAASGFTFSKF |
| construct C2031 (SEQ ID | GMSWVRQANPWVPPREGLEWVSSISGSGRDTLYADSVKGRFTISRDNAKT |
| No. 808) | TLYLQMNSLRPEDTAVYYCTIGGSLSVSSQGTLVTVSSGGGGLSGRSDNHGG |
| | GGTEVQLLESGGGLVQPGGSLTLSCAASGRSFSNYIMGWFRQAPGKEREFV |
| | AGLGWSPGNTYYADSVKGRFTISRDNSKNTLYLQMNSLRAEDTAVYYCAAR |
| | RGDVIYTTPWNYVYWGLGTQVTVSSTSDYKDDDDKTTTPAPRPPTPAPTIAS |
| | QPLSLRPEACRPAAGGAVHTRGLDFACDIYIWAPLAGTCGVLLLSLVITLYKR |
| | GRKKLLYIFKQPFMRPVQTTQEEDGCSCRFPEEEEGGCELRVKFSRSADAPA |
| | YQQGQNQLYNELNLGRREEYDVLDKRRGRDPEMGGKPRRKNPQEGLYNEL |
| | QKDKMAEAYSEIGMKGERRRGKGHDGLYQGLSTATKDTYDALHMQALPPR |
| Exemplary proCAR | MALPVTALLLPLALLLHAARPEVQLLESGGGLVQPGGSLTLSCAASGRTDSW |
| construct C1827 (SEQ ID | YVMGWFRQAPGKDREFVAGVSWSYGNTYYADSVKGRFTISRDNSKNTLYL |
| No. 809) | QMNSLRAEDTAVYYCAARVSREVIPTRWDLYNYWGLGTQVTVSSTSDYKD |
| | DDDKTTTPAPRPPTPAPTIASQPLSLRPEACRPAAGGAVHTRGLDFACDIYI |
| | WAPLAGTCGVLLLSLVITLYKRGRKKLLYIFKQPFMRPVQTTQEEDGCSCRFP |
| | EEEEGGCELRVKFSRSADAPAYQQGQNQLYNELNLGRREEYDVLDKRRGRD |
| | PEMGGKPRRKNPQEGLYNELQKDKMAEAYSEIGMKGERRRGKGHDGLYQ |
| | GLSTATKDTYDALHMQALPPR |
| Exemplary proCAR | MALPVTALLLPLALLLHAARPEVQLVESGGGLVQPGNSLRLSCAASGFTFSKF |
| construct C1829 (SEQ ID | GMSWVRQAPGKGLEWVSSISGSGRDTLYADSVKGRFTISRDNAKTTLYLQ |
| No. 810) | MNSLRPEDTAVYYCTIGGSLSVSSQGTLVTVSSGSGGSGGGGSGGGGGSGE |
| | VQLLESGGGLVQPGGSLTLSCAASGRTDSWYVMGWFRQAPGKDREFVAG |
| | VSWSYGNTYYADSVKGRFTISRDNSKNTLYLQMNSLRAEDTAVYYCAARVS |
| | REVIPTRWDLYNYWGLGTQVTVSSTSDYKDDDDKTTTPAPRPPTPAPTIASQ |
| | PLSLRPEACRPAAGGAVHTRGLDFACDIYIWAPLAGTCGVLLLSLVITLYKRGR |
| | KKLLYIFKQPFMRPVQTTQEEDGCSCRFPEEEEGGCELRVKFSRSADAPAYQ |
| | QGQNQLYNELNLGRREEYDVLDKRRGRDPEMGGKPRRKNPQEGLYNELQK |
| | DKMAEAYSEIGMKGERRRGKGHDGLYQGLSTATKDTYDALHMQALPPR |
| Exemplary proCAR | MALPVTALLLPLALLLHAARPEVQLVESGGGLVQPGNSLRLSCAASGFTFSKF |
| construct C1952 (SEQ ID | GMSWVRQAHEPLTCRGLEWVSSISGSGRDTLYADSVKGRFTISRDNAKTTL |
| No. 811) | YLQMNSLRPEDTAVYYCTIGGSLSVSSQGTLVTVSSGSGGSGGGGSGGGGG |
| | SGEVQLLESGGGLVQPGGSLTLSCAASGRTDSWYVMGWFRQAPGKDREFV |
| | AGVSWSYGNTYYADSVKGRFTISRDNSKNTLYLQMNSLRAEDTAVYYCAAR |
| | VSREVIPTRWDLYNYWGLGTQVTVSSTSDYKDDDDKTTTPAPRPPTPAPTIA |
| | SQPLSLRPEACRPAAGGAVHTRGLDFACDIYIWAPLAGTCGVLLLSLVITLYKR |
| | GRKKLLYIFKQPFMRPVQTTQEEDGCSCRFPEEEEGGCELRVKFSRSADAPA |
| | YQQGQNQLYNELNLGRREEYDVLDKRRGRDPEMGGKPRRKNPQEGLYNEL |
| | QKDKMAEAYSEIGMKGERRRGKGHDGLYQGLSTATKDTYDALHMQALPPR |
| Exemplary proCAR | MALPVTALLLPLALLLHAARPEVQLVESGGGLVQPGNSLRLSCAASGFTFSKF |
| construct C1954 (SEQ ID | GMSWVRQASHPMSCRGLEWVSSISGSGRDTLYADSVKGRFTISRDNAKTTL |
| No. 812) | YLQMNSLRPEDTAVYYCTIGGSLSVSSQGTLVTVSSGSGGSGGGGSGGGGG |
| | SGEVQLLESGGGLVQPGGSLTLSCAASGRTDSWYVMGWFRQAPGKDREFV |
| | AGVSWSYGNTYYADSVKGRFTISRDNSKNTLYLQMNSLRAEDTAVYYCAAR |
| | VSREVIPTRWDLYNYWGLGTQVTVSSTSDYKDDDDKTTTPAPRPPTPAPTIA |
| | SQPLSLRPEACRPAAGGAVHTRGLDFACDIYIWAPLAGTCGVLLLSLVITLYKR |
| | GRKKLLYIFKQPFMRPVQTTQEEDGCSCRFPEEEEGGCELRVKFSRSADAPA |
| | YQQGQNQLYNELNLGRREEYDVLDKRRGRDPEMGGKPRRKNPQEGLYNEL |
| | QKDKMAEAYSEIGMKGERRRGKGHDGLYQGLSTATKDTYDALHMQALPPR |
| Exemplary proCAR | MALPVTALLLPLALLLHAARPEVQLVESGGGLVQPGNSLRLSCAASGFTFSKF |
| construct C2033 (SEQ ID | GMSWVRQAHEPLTCRGLEWVSSISGSGRDTLYADSVKGRFTISRDNAKTTL |
| No. 813) | YLQMNSLRPEDTAVYYCTIGGSLSVSSQGTLVTVSSGGGGLSGRSDNHGGG |
| | GTEVQLLESGGGLVQPGGSLTLSCAASGRTDSWYVMGWFRQAPGKDREFV |
| | AGVSWSYGNTYYADSVKGRFTISRDNSKNTLYLQMNSLRAEDTAVYYCAAR |
| | VSREVIPTRWDLYNYWGLGTQVTVSSTSDYKDDDDKTTTPAPRPPTPAPTIA |
| | SQPLSLRPEACRPAAGGAVHTRGLDFACDIYIWAPLAGTCGVLLLSLVITLYKR |
| | GRKKLLYIFKQPFMRPVQTTQEEDGCSCRFPEEEEGGCELRVKFSRSADAPA |
| | YQQGQNQLYNELNLGRREEYDVLDKRRGRDPEMGGKPRRKNPQEGLYNEL |
| | QKDKMAEAYSEIGMKGERRRGKGHDGLYQGLSTATKDTYDALHMQALPPR |
| Exemplary proCAR | MALPVTALLLPLALLLHAARPEVQLVESGGGLVQPGNSLRLSCAASGFTFSKF |
| construct C1953 (SEQ ID | GMSWVRQASHPMSCRGLEWVSSISGSGRDTLYADSVKGRFTISRDNAKTTL |
| No. 814) | YLQMNSLRPEDTAVYYCTIGGSLSVSSQGTLVTVSSGSGGPLGLQARVVGGS |
| | GEVQLLESGGGLVQPGGSLTLSCAASGRTDSWYVMGWFRQAPGKDREFV |
| | AGVSWSYGNTYYADSVKGRFTISRDNSKNTLYLQMNSLRAEDTAVYYCAAR |
| | VSREVIPTRWDLYNYWGLGTQVTVSSTSDYKDDDDKTTTPAPRPPTPAPTIA |
| | SQPLSLRPEACRPAAGGAVHTRGLDFACDIYIWAPLAGTCGVLLLSLVITLYKR |
| | GRKKLLYIFKQPFMRPVQTTQEEDGCSCRFPEEEEGGCELRVKFSRSADAPA |
| | YQQGQNQLYNELNLGRREEYDVLDKRRGRDPEMGGKPRRKNPQEGLYNEL |
| | QKDKMAEAYSEIGMKGERRRGKGHDGLYQGLSTATKDTYDALHMQALPPR |
| Exemplary proCAR | MALPVTALLLPLALLLHAARPEVQLVESGGGLVQPGNSLRLSCAASGFTFSKF |
| construct C2035 (SEQ ID | GMSWVRQASHPMSCRGLEWVSSISGSGRDTLYADSVKGRFTISRDNAKTTL |
| No. 815) | YLQMNSLRPEDTAVYYCTIGGSLSVSSQGTLVTVSSGGGGLSGRSDNHGGG |
| | GTEVQLLESGGGLVQPGGSLTLSCAASGRTDSWYVMGWFRQAPGKDREFV |
| | AGVSWSYGNTYYADSVKGRFTISRDNSKNTLYLQMNSLRAEDTAVYYCAAR |
| | VSREVIPTRWDLYNYWGLGTQVTVSSTSDYKDDDDKTTTPAPRPPTPAPTIA |
| | SQPLSLRPEACRPAAGGAVHTRGLDFACDIYIWAPLAGTCGVLLLSLVITLYKR |
| | GRKKLLYIFKQPFMRPVQTTQEEDGCSCRFPEEEEGGCELRVKFSRSADAPA |
| | YQQGQNQLYNELNLGRREEYDVLDKRRGRDPEMGGKPRRKNPQEGLYNEL |
| | QKDKMAEAYSEIGMKGERRRGKGHDGLYQGLSTATKDTYDALHMQALPPR |
| CD8a signal peptide | MALPVTALLLPLALLLHAARP |
| (SEQ ID No. 816) | |
| 10G sdAb (SEQ ID No. | EVQLVESGGGLVQPGNSLRLSCAASGFTFSKFGMSWVRQAPGKGLEWVSSI |
| 817) | SGSGRDTLYADSVKGRFTISRDNAKTTLYLQMNSLRPEDTAVYYCTIGGSLSV |
| | SSQGTLVTVSS |
| 10G sdAb EH4 Mask 1 | EVQLVESGGGLVQPGNSLRLSCAASGFTFSKFGMSWVRQANPWVPPREGL |
| (SEQ ID No. 818) | EWVSSISGSGRDTLYADSVKGRFTISRDNAKTTLYLQMNSLRPEDTAVYYCTI |
| | GGSLSVSSQGTLVTVSS |
| 10G sdAb EH104 Mask 1 | EVQLVESGGGLVQPGNSLRLSCAASGFTFSKFGMSWVRQAHEPLTCRGLE |
| (SEQ ID No. 819) | WVSSISGSGRDTLYADSVKGRFTISRDNAKTTLYLQMNSLRPEDTAVYYCTIG |
| | GSLSVSSQGTLVTVSS |
| 10G sdAb EH104 Mask 2 | EVQLVESGGGLVQPGNSLRLSCAASGFTFSKFGMSWVRQASHPMSCRGLE |
| (SEQ ID No. 820) | WVSSISGSGRDTLYADSVKGRFTISRDNAKTTLYLQMNSLRPEDTAVYYCTIG |
| | GSLSVSSQGTLVTVSS |
| EH4 sdAb (SEQ ID No. | EVQLLESGGGLVQPGGSLTLSCAASGRSFSNYIMGWFRQAPGKEREFVAGL |
| 821) | GWSPGNTYYADSVKGRFTISRDNSKNTLYLQMNSLRAEDTAVYYCAARRGD |
| | VIYTTPWNYVYWGLGTQVTVSS |
| EH104 sdAb (SEQ ID No. | EVQLLESGGGLVQPGGSLTLSCAASGRTDSWYVMGWFRQAPGKDREFVA |
| 822) | GVSWSYGNTYYADSVKGRFTISRDNSKNTLYLQMNSLRAEDTAVYYCAARV |
| | SREVIPTRWDLYNYWGLGTQVTVSS |
| CD8a hinge/TM (SEQ ID | TTTPAPRPPTPAPTIASQPLSLRPEACRPAAGGAVHTRGLDFACDIYIWAPLA |
| No. 823) | GTCGVLLLSLVITLY |
| 4-1BB intracellular | KRGRKKLLYIFKQPFMRPVQTTQEEDGCSCRFPEEEEGGCEL |
| domain (SEQ ID No. 824) | |
| CD3z intracellular | RVKFSRSADAPAYQQGQNQLYNELNLGRREEYDVLDKRRGRDPEMGGKPR |
| domain (SEQ ID No. 825) | RKNPQEGLYNELQKDKMAEAYSEIGMKGERRRGKGHDGLYQGLSTATKDT |
| | YDALHMQALPPR |
| non-cleavable linker | GSGGSGGGGSGGGGGSG |
| (SEQ ID No. 826) | |
| protease cleavable | GGGGLSGRSDNHGGGGT |
| linker 1 (SEQ ID No. 827) | |
| protease cleavable | GSGGPLGLQARVVGGSG |
| linker 2 (SEQ ID No. 828) | |
| EH4 Mask 1 (SEQ ID No. | ANPWVPPREG |
| 829) | |
| EH 104 Mask 1 (SEQ | IDAHEPLTCRG |
| No. 830) | |
| EH104 Mask 2 (SEQ ID | ASHPMSCRG |
| No. 831) | |
| Exemplary proCAR | MALPVTALLLPLALLLHAARPEVQLVESGGGLVQPGNSLRLSCAASGFTFSKF |
| construct C2446 (SEQ ID | GMSWVRQANPWVPPREGLEWVSSISGSGRDTLYADSVKGRFTISRDNAKT |
| No. 832) | TLYLQMNSLRPEDTAVYYCTIGGSLSVSSQGTLVTVSSGGGGPPASSGRAGG |
| | GGGGTEVQLLESGGGLVQPGGSLTLSCAASGRSFSNYIMGWFRQAPGKERE |
| | FVAGLGWSPGNTYYADSVKGRFTISRDNSKNTLYLQMNSLRAEDTAVYYCA |
| | ARRGDVIYTTPWNYVYWGLGTQVTVSSTSDYKDDDDKTTTPAPRPPTPAPT |
| | IASQPLSLRPEACRPAAGGAVHTRGLDFACDIYIWAPLAGTCGVLLLSLVITLY |
| | KRGRKKLLYIFKQPFMRPVQTTQEEDGCSCRFPEEEEGGCELRVKFSRSADA |
| | PAYQQGQNQLYNELNLGRREEYDVLDKRRGRDPEMGGKPRRKNPQEGLY |
| | NELQKDKMAEAYSEIGMKGERRRGKGHDGLYQGLSTATKDTYDALHMQAL |
| | PPR |
| Exemplary proCAR | MALPVTALLLPLALLLHAARPEVQLLESGGGLVQPGGSLTLSCAASGRSFSNY |
| construct C2448 (SEQ ID | IMGWFRQAPGKEREFVAGLGWSPGNTYYADSVKGRFTISRDNSKNTLYLQ |
| No. 833) | MNSLRAEDTAVYYCAARRGDVIYTTPWNYVYWGLGTQVTVSSTSDYKDDD |
| | DKTTTPAPRPPTPAPTIASQPLSLRPEACRPAAGGAVHTRGLDFASDIYIWAP |
| | LAGTCGVLLLSLVITLYKRGRKKLLYIFKQPFMRPVQTTQEEDGCSCRFPEEEE |
| | GGCELRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGLST |
| | ATKDTYDALHMQALPPR |
| Exemplary proCAR | MALPVTALLLPLALLLHAARPEVQLVESGGGLVQPGNSLRLSCAASGFTFSKF |
| construct C2510 (SEQ ID | GMSWVRQANPWVPPREGLEWVSSISGSGRDTLYADSVKGRFTISRDNAKT |
| No. 834) | TLYLQMNSLRPEDTAVYYCTIGGSLSVSSQGTLVTVSSGGGGLSGRSDNHGG |
| | GGTEVQLLESGGGLVQPGGSLTLSCAASGRSFSNYIMGWFRQAPGKEREFV |
| | AGLGWSPGNTYYADSVKGRFTISRDNSKNTLYLQMNSLRAEDTAVYYCAAR |
| | RGDVIYTTPWNYVYWGLGTQVTVSSTSDYKDDDDKTTTPAPRPPTPAPTIAS |
| | QPLSLRPEACRPAAGGAVHTRGLDFASDIYIWAPLAGTCGVLLLSLVITLYKR |
| | GRKKLLYIFKQPFMRPVQTTQEEDGCSCRFPEEEEGGCELRVKFSRSADAPA |
| | YQQGQNQLYNELNLGRREEYDVLDKRRGRDPEMGGKPRRKNPQEGLYNEL |
| | QKDKMAEAYSEIGMKGERRRGKGHDGLYQGLSTATKDTYDALHMQALPPR |
| Exemplary proCAR | MALPVTALLLPLALLLHAARPEVQLVESGGGLVQPGNSLRLSCAASGFTFSKF |
| construct C2513 (SEQ ID | GMSWVRQANPWVPPREGLEWVSSISGSGRDTLYADSVKGRFTISRDNAKT |
| No. 835) | TLYLQMNSLRPEDTAVYYCTIGGSLSVSSQGTLVTVSSGGGGPPASSGRAGG |
| | GGGGTEVQLLESGGGLVQPGGSLTLSCAASGRSFSNYIMGWFRQAPGKERE |
| | FVAGLGWSPGNTYYADSVKGRFTISRDNSKNTLYLQMNSLRAEDTAVYYCA |
| | ARRGDVIYTTPWNYVYWGLGTQVTVSSTSDYKDDDDKTTTPAPRPPTPAPT |
| | IASQPLSLRPEACRPAAGGAVHTRGLDFASDIYIWAPLAGTCGVLLLSLVITLY |
| | KRGRKKLLYIFKQPFMRPVQTTQEEDGCSCRFPEEEEGGCELRVKFSRSADA |
| | PAYQQGQNQLYNELNLGRREEYDVLDKRRGRDPEMGGKPRRKNPQEGLY |
| | NELQKDKMAEAYSEIGMKGERRRGKGHDGLYQGLSTATKDTYDALHMQAL |
| | PPR |
| Exemplary proCAR | MALPVTALLLPLALLLHAARPEVQLVESGGGLVQPGNSLRLSCAASGFTFSKF |
| construct C2514 (SEQ ID | GMSWVRQANPWVPPREGLEWVSSISGSGRDTLYADSVKGRFTISRDNAKT |
| No. 836) | TLYLQMNSLRPEDTAVYYCTIGGSLSVSSQGTLVTVSSGSGGSGGGGSGGG |
| | GGSGEVQLLESGGGLVQPGGSLTLSCAASGRSFSNYIMGWFRQAPGKEREF |
| | VAGLGWSPGNTYYADSVKGRFTISRDNSKNTLYLQMNSLRAEDTAVYYCAA |
| | RRGDVIYTTPWNYVYWGLGTQVTVSSTSDYKDDDDKTTTPAPRPPTPAPTI |
| | ASQPLSLRPEACRPAAGGAVHTRGLDFASDIYIWAPLAGTCGVLLLSLVITLYK |
| | RGRKKLLYIFKQPFMRPVQTTQEEDGCSCRFPEEEEGGCELRVKFSRSADAP |
| | AYQQGQNQLYNELNLGRREEYDVLDKRRGRDPEMGGKPRRKNPQEGLYN |
| | ELQKDKMAEAYSEIGMKGERRRGKGHDGLYQGLSTATKDTYDALHMQALP |
| | PR |
| Exemplary proCAR | MALPVTALLLPLALLLHAARPQVQLVESGGALVQPGGSLRLSCAASGFPVNR |
| construct C2780 (SEQ ID | YSMRWYRQAPGKEREWVAGMSSAGDRSSYEDSVKGRFTISRDDARNTVYL |
| No. 837) | QMNSLKPEDTAVYYCNVNVGFEYWGQGTQVTVSSTSDYKDDDDKTTTPAP |
| | RPPTPAPTIASQPLSLRPEACRPAAGGAVHTRGLDFACDIYIWAPLAGTCGVL |
| | LLSLVITLYKRGRKKLLYIFKQPFMRPVQTTQEEDGCSCRFPEEEEGGCELRVK |
| | FSRSADAPAYQQGQNQLYNELNLGRREEYDVLDKRRGRDPEMGGKPRRKN |
| | PQEGLYNELQKDKMAEAYSEIGMKGERRRGKGHDGLYQGLSTATKDTYDA |
| | LHMQALPPR |
| Exemplary proCAR | MALPVTALLLPLALLLHAARPEVQLLESGGGLVQPGGSLTLSCAASGFIFRAA |
| construct C2483 (SEQ ID | SMAWYRQSPGNERELVASISSGAFTNYADSVKARFTISRDNSKNTLYLQMN |
| No. 838) | SLRAEDTAVYYCGATFLRSDGHHTINGQGTLVTVSSTSDYKDDDDKITTPAP |
| | RPPTPAPTIASQPLSLRPEACRPAAGGAVHTRGLDFACDIYIWAPLAGTCGVL |
| | LLSLVITLYKRGRKKLLYIFKQPFMRPVQTTQEEDGCSCRFPEEEEGGCELRVK |
| | FSRSADAPAYQQGQNQLYNELNLGRREEYDVLDKRRGRDPEMGGKPRRKN |
| | PQEGLYNELQKDKMAEAYSEIGMKGERRRGKGHDGLYQGLSTATKDTYDA |
| | LHMQALPPR |
| Exemplary proCAR | MALPVTALLLPLALLLHAARPEVQLVESGGGLVQPGNSLRLSCAASGFTFSKF |
| construct C2790 (SEQ ID | GMSWVRQAPGKGLEWVSSISGSGRDTLYADSVKGRFTISRDNAKTTLYLQ |
| No. 839) | MNSLRPEDTAVYYCTIGGSLSVSSQGTLVTVSSGSGGSGGGGSGGGGGSGE |
| | VQLLESGGGLVQPGGSLTLSCAASGFIFRAASMAWYRQSPGNERELVASISS |
| | GAFTNYADSVKARFTISRDNSKNTLYLQMNSLRAEDTAVYYCGATFLRSDGH |
| | HTINGQGTLVTVSSTSDYKDDDDKTTTPAPRPPTPAPTIASQPLSLRPEACRP |
| | AAGGAVHTRGLDFACDIYIWAPLAGTCGVLLLSLVITLYKRGRKKLLYIFKQPF |
| | MRPVQTTQEEDGCSCRFPEEEEGGCELRVKFSRSADAPAYQQGQNQLYNE |
| | LNLGRREEYDVLDKRRGRDPEMGGKPRRKNPQEGLYNELQKDKMAEAYSEI |
| | GMKGERRRGKGHDGLYQGLSTATKDTYDALHMQALPPR |
| Exemplary proCAR | MALPVTALLLPLALLLHAARPEVQLVESGGGLVQPGNSLRLSCAASGFTFSKF |
| construct C3269 (SEQ ID | GMSWVRQRQSTFMAMDMPILEWVSSISGSGRDTLYADSVKGRFTISRDNA |
| No. 840) | KTTLYLQMNSLRPEDTAVYYCTIGGSLSVSSQGTLVTVSSGSGGSGGGGSGG |
| | GGGSGEVQLLESGGGLVQPGGSLTLSCAASGFIFRAASMAWYRQSPGNERE |
| | LVASISSGAFTNYADSVKARFTISRDNSKNTLYLQMNSLRAEDTAVYYCGATF |
| | LRSDGHHTINGQGTLVTVSSTSDYKDDDDKTTTPAPRPPTPAPTIASQPLSLR |
| | PEACRPAAGGAVHTRGLDFACDIYIWAPLAGTCGVLLLSLVITLYKRGRKKLLY |
| | IFKQPFMRPVQTTQEEDGCSCRFPEEEEGGCELRVKFSRSADAPAYQQGQN |
| | QLYNELNLGRREEYDVLDKRRGRDPEMGGKPRRKNPQEGLYNELQKDKMA |
| | EAYSEIGMKGERRRGKGHDGLYQGLSTATKDTYDALHMQALPPR |
| Exemplary proCAR | MALPVTALLLPLALLLHAARPEVQLVESGGGLVQPGNSLRLSCAASGFTFSKF |
| construct C3272 (SEQ ID | GMSWVRQQAYWWGNSRDLEWVSSISGSGRDTLYADSVKGRFTISRDNAK |
| No. 841) | TTLYLQMNSLRPEDTAVYYCTIGGSLSVSSQGTLVTVSSGSGGSGGGGSGGG |
| | GGSGEVQLLESGGGLVQPGGSLTLSCAASGFIFRAASMAWYRQSPGNEREL |
| | VASISSGAFTNYADSVKARFTISRDNSKNTLYLQMNSLRAEDTAVYYCGATFL |
| | RSDGHHTINGQGTLVTVSSTSDYKDDDDKTTTPAPRPPTPAPTIASQPLSLRP |
| | EACRPAAGGAVHTRGLDFACDIYIWAPLAGTCGVLLLSLVITLYKRGRKKLLYI |
| | FKQPFMRPVQTTQEEDGCSCRFPEEEEGGCELRVKFSRSADAPAYQQGQN |
| | QLYNELNLGRREEYDVLDKRRGRDPEMGGKPRRKNPQEGLYNELQKDKMA |
| | EAYSEIGMKGERRRGKGHDGLYQGLSTATKDTYDALHMQALPPR |
| Exemplary proCAR | MALPVTALLLPLALLLHAARPEVQLVESGGGLVQPGNSLRLSCAASGFTFSKF |
| construct C3328 (SEQ ID | GMSWVRQQAYWWGNSRDLEWVSSISGSGRDTLYADSVKGRFTISRDNAK |
| No. 842) | TTLYLQMNSLRPEDTAVYYCTIGGSLSVSSQGTLVTVSSGGGGPQGSTGRAA |
| | GGGGGTEVQLLESGGGLVQPGGSLTLSCAASGFIFRAASMAWYRQSPGNE |
| | RELVASISSGAFTNYADSVKARFTISRDNSKNTLYLQMNSLRAEDTAVYYCGA |
| | TFLRSDGHHTINGQGTLVTVSSTSDYKDDDDKTTTPAPRPPTPAPTIASQPLS |
| | LRPEACRPAAGGAVHTRGLDFACDIYIWAPLAGTCGVLLLSLVITLYKRGRKK |
| | LLYIFKQPFMRPVQTTQEEDGCSCRFPEEEEGGCELRVKFSRSADAPAYQQG |
| | QNQLYNELNLGRREEYDVLDKRRGRDPEMGGKPRRKNPQEGLYNELQKDK |
| | MAEAYSEIGMKGERRRGKGHDGLYQGLSTATKDTYDALHMQALPPR |
| Exemplary proCAR | MALPVTALLLPLALLLHAARPEVQLVESGGGLVQPGNSLRLSCAASGFTFSKF |
| construct C3329 (SEQ ID | GMSWVRQRQSTFMAMDMPILEWVSSISGSGRDTLYADSVKGRFTISRDNA |
| No. 843) | KTTLYLQMNSLRPEDTAVYYCTIGGSLSVSSQGTLVTVSSGGGGPQGSTGRA |
| | AGGGGGTEVQLLESGGGLVQPGGSLTLSCAASGFIFRAASMAWYRQSPGN |
| | ERELVASISSGAFTNYADSVKARFTISRDNSKNTLYLQMNSLRAEDTAVYYCG |
| | ATFLRSDGHHTINGQGTLVTVSSTSDYKDDDDKITTPAPRPPTPAPTIASQPL |
| | SLRPEACRPAAGGAVHTRGLDFACDIYIWAPLAGTCGVLLLSLVITLYKRGRK |
| | KLLYIFKQPFMRPVQTTQEEDGCSCRFPEEEEGGCELRVKFSRSADAPAYQQ |
| | GQNQLYNELNLGRREEYDVLDKRRGRDPEMGGKPRRKNPQEGLYNELQKD |
| | KMAEAYSEIGMKGERRRGKGHDGLYQGLSTATKDTYDALHMQALPPR |
| dimerization deficient | TTTPAPRPPTPAPTIASQPLSLRPEACRPAAGGAVHTRGLDFASDIYIWAPLA |
| CD8a hinge/TM (SEQ ID | GTCGVLLLSLVITLY |
| No. 844) | |
| protease cleavable | GGGGPPASSGRAGGGGGGT |
| linker 3 (SEQ ID No. 845) | |
| 10G sdAb H90 Mask 1 | EVQLVESGGGLVQPGNSLRLSCAASGFTFSKFGMSWVRQRQSTFMAMDM |
| (SEQ ID No. 846) | PILEWVSSISGSGRDTLYADSVKGRFTISRDNAKTTLYLQMNSLRPEDTAVYY |
| | CTIGGSLSVSSQGTLVTVSS |
| 10G sdAb H90 Mask 2 | EVQLVESGGGLVQPGNSLRLSCAASGFTFSKFGMSWVRQQAYWWGNSRD |
| (SEQ ID No. 847) | LEWVSSISGSGRDTLYADSVKGRFTISRDNAKTTLYLQMNSLRPEDTAVYYCT |
| | IGGSLSVSSQGTLVTVSS |
| H90 sdAb (SEQ ID No. | EVQLLESGGGLVQPGGSLTLSCAASGFIFRAASMAWYRQSPGNERELVASIS |
| 848) | SGAFTNYADSVKARFTISRDNSKNTLYLQMNSLRAEDTAVYYCGATFLRSDG |
| | HHTINGQGTLVTVSS |
| GFP sdAb (SEQ ID No. | QVQLVESGGALVQPGGSLRLSCAASGFPVNRYSMRWYRQAPGKEREWVA |
| 849) | GMSSAGDRSSYEDSVKGRFTISRDDARNTVYLQMNSLKPEDTAVYYCNVNV |
| | GFEYWGQGTQVTVSS |
| H90 Mask 1 (SEQ ID No. | RQSTFMAMDMPI |
| 850) | |
| H90 Mask 2 (SEQ ID No. | QAYWWGNSRD |
| 851) | |
| metalloproteinase | Gly-Pro-Leu-Gly-Ile-Ala-Gly-Gln |
| cleavage site (SEQ ID | |
| No. 852) | |
| metalloproteinase | Ala-Val-Arg-Trp-Leu-Leu-Thr-Ala |
| cleavage site (SEQ ID | |
| No. 853) | |
| furin cleavage site (SEQ | Arg-Arg-Arg-Arg-Arg-Arg |
| ID No. 854) | |
| Exemplary | IYIWAPLAGTCGVLLLSLVITLYC (SEQ ID NO: 94) |
| transmembrane (TM) | |
| sequence (SEQ ID No. | |
| 855) | |
| CD8 beta derived TM | GLLVAGVLVLLVSLGVAIHLCC |
| (SEQ ID No. 856) | |
| CD4 derived TM(SEQ ID | ALIVLGGVAGLLLFIGLGIFFCVRC |
| No. 857) | |
| CD3 zeta derived TM | LCYLLDGILFIYGVILTALFLRV |
| (SEQ ID No. 858) | |
| CD28 derived TM(SEQ | WVLVVVGGVLACYSLLVTVAFIIFWV |
| ID No. 859) | |
| CD134 (0X40) derived | AAILGLGLVLGLLGPLAILLALYLL |
| TM: (SEQ ID No. 860) | |
| CD7 derived TM(SEQ ID | ALPAALAVISFLLGLGLGVACVLA |
| No. 861) | |
| Hinge region (SEQ ID | CPEPKSCDTPPPCPR |
| No. 862) | |
| Hinge region (SEQ ID | ELKTPLGDTTHT |
| No. 863) | |
| Hinge region (SEQ ID | KCCVDCP |
| No. 864) | |
| Hinge region (SEQ ID | KYGPPCP |
| No. 865) | |
| Human IgG1 hinge | EPKSCDKTHTCPPCP |
| region (SEQ ID No. 866) | |
| Human IgG2 hinge | ERKCCVECPPCP |
| region (SEQ ID No. 867) | |
| Human IgG3 hinge | ELKTPLGDTTHTCPRCP |
| region (SEQ ID No. 868) | |
| Human IgG4 hinge | SPNMVPHAHHAQ |
| region (SEQ ID No. 869) | |
| His229 of human IgG1 | EPKSCDKTYTCPPCP |
| hinge region substituted | |
| with Tyr (SEQ ID No. | |
| 870) | |
| CD8 hinge region (SEQ | TTTPAPRPPTPAPTIASQPLSLRPEACRPAAGGAVHTRGLDFACD |
| ID No. 871) | |
| Hinge region (SEQ ID | KSCDKTHTCP |
| No. 872) | |
| Exemplary anti-target | QVQLQESGGGLVQAGGSLRLSCAASGFIFRAASMAWYRQSPGNERELVASI |
| (EpCAM) | SSGAFTNYADSVKARFTISRDNAKNTVYLQMNSLKPEDTAVYFCGATFLRSD |
| (SEQ ID NO: 873) EPL90 | GHHTINGQGTQVTVSS |
| Exemplary anti-target | QVQLQESGGGLVQAGGSLRLSCAASGFIFRAASMGWFRQSPGNERELVAT |
| (EpCAM) | VSSGDFTNYADSVKGRFTISRDNAKNTVYLQMNSLKPEDTAVYFCGATFVRS |
| (SEQ ID NO: 874) | DGHHTIYGQGTQVTVSS |
| EPL118 | |
| Exemplary anti-target | QVQLQESGGGLVQAGGSLRLSCAASGFIFRAASMDWYRQFPGNERESIATI |
| (EpCAM) | SSGGFTNYADSVKGRFTISRDNAKNTVYLQMNSLKPEDTAVYFCGATFLRSD |
| (SEQ ID NO: 875) | GHHTINGQGTQVIVSS |
| EPL138 | |
| Exemplary anti-target | QVQLQESGGGLVQAGGSLRLSCAASGFIFRAASMGWFRQSPGNERELVAT |
| (EpCAM) | VSSGGFTNYADSVKGRFTISRDNAKNTVYLQMNSLKPEDTAVYFCGATFVRS |
| (SEQ ID NO: 876) | DGHHTIYGQGTQVTVSS |
| EPL145 | |
| Exemplary anti-target | QVQLQESGGGLVQAGGSLRLSCAASGFIFRAASMDWYRQSPGTQPELVATI |
| (EpCAM) | SSTGFTNYANSVKGRFTISRDNAKNTVYLQMNSLKPEDTAVYFCGATFLRSD |
| (SEQ ID NO: 877) | GQHSIYGQGTQVTVSS |
| EPL164 | |
| Exemplary anti-target | QVQLQESGGGLVHTGGSLRLSCAASGDTFLRYAMGWFRQAPGKEREFVAA |
| (EpCAM) | ITWNGGNTDYAGSLKGRFTISRDNTKNTVYLQMNSLKPEDTAVYYCAADLT |
| (SEQ ID NO: 878) EPL31 | FGLASSHYQYDYWGQGTQVTVSS |
| Exemplary anti-target | QVQLQESGGGLVQAGGSLRLSCAASGDTFLRYAMGWFRQAPGKEREFVAA |
| (EpCAM) | ITWNGGNTDYAGSLKGRFTISRDNTKNTVYLQMNSLKPEDTAVYYCAADLT |
| (SEQ ID NO: 879) EPL55 | FGLASSHYQYDYWGQGTQVTVSS |
| Exemplary anti-target | QVQLQESGGGLVHTGGSLRLSCAFSGDTFLRYAMGWFRQAPGKEREFVAAI |
| (EpCAM) | TWNGGNTDYADSLKGRFTISRDNTKNTVYLQMNSLRPEDTAVYYCAADLTF |
| (SEQ ID NO: 880) EPL57 | GLASSHYQYDYWGQGTQVTVSS |
| Exemplary anti-target | QVQLQESGGGLVQPGGSLRLSCAASGDTFLRYAMGWFRQAPGKEREFVAA |
| (EpCAM) | ITWNGGNTDYAGSLKGRFTISRDNTKNTVYLQMNSLKPEDTAVYYCAADLT |
| (SEQ ID NO: 881) | FGLASSHYQYDYWGQGTQVTVSS |
| EPL136 | |
| Exemplary anti-target | QVQLQESGGGSVLAGGSLRLSCAASGFTFSSYYMSWVRQAPGKGLEWVSG |
| (EpCAM) | IHYTGDWTNYADSVKGRFTISRDNAKNELYLEMNNLKPEDTAVYYCARGSD |
| (SEQ ID NO: 882) EPL15 | KGQGTQVTVSS |
| Exemplary anti-target | QVQLQESGGGSVQAGGSLRLSCAASGFTFSSYYMSWVRQAPGKGLEWVS |
| (EpCAM) | GIHYTGDWTNYADSVKGRFTISRDNAKNELYLEMNNLKPEDTAVYYCARGS |
| (SEQ ID NO: 883) EPL34 | DKGQGTQVTVSS |
| Exemplary anti-target | QVQLQESGGGLVQPGGSLRLSCAASGFTFSDWAMSWVRQAPGKGLEWVS |
| (EpCAM) | GIHYGDHTTHYADFVKGRFTISRDDAKNTLYLQMNSLKPEDTAVYYCARGST |
| (SEQ ID NO: 884) EPL86 | KGQGTQVTVSS |
| Exemplary anti-target | QVQLQESGGGLVQPGGSLRLSCAASGFTFSDWAMSWVRQAPGKGLEWVS |
| (EpCAM) | SIHYGDHTTHYADFVKGRFTISRDDAKNTLYLQMNSLKPEDTAVYYCEKGTT |
| (SEQ ID NO: 885) | RGQGTQVTVSS |
| EPL153 | |
| Exemplary anti-target | QVQLQESGGGLVQAGGSLKLSCAASGNVFRAATMAWYRQAPEKQREMV |
| (EpCAM) | ATIASGGTTNYADFVKGRFTISRDNAKNTVYLQMNTLKPEDTAVYYCNAGYL |
| (SEQ ID NO: 886) EPL20 | TSLGPKNYWGQGTQVTVSS |
| Exemplary anti-target | QVQLQESGGGLVQPGGSLRLSCAASGNVFRAATMAWYRQAPEKXREMVA |
| (EpCAM) | TIASGGTTNYADFVKGRFTISRDNAKNTVYLQMNTLKPEDTAVYYCNAGYLT |
| (SEQ ID NO: 887) EPL70 | SLGPKNYWGQGTQVTVSS |
| Exemplary anti-target | QVQLQESGGGLVQAGGSLRLSCAASGNVFRAATMAWYRQVPEKQREMV |
| (EpCAM) | ATIASGGTTNYADEVKGRFTISRDNAKNTVYLQMNTLKPEDTAVYYCNALYL |
| (SEQ ID NO: 888) | TSLGPKSYWGQGTQVTVSS |
| EPL125 | |
| Exemplary anti-target | QVQLQESGGGLVQPGGSLRLSCAASGFAFGNHWMYWYRQAPGRGRELV |
| (EpCAM) | ASISSGGSTNYVDSVKGRFTISRDNARNTVYLQMYSLKPEDTAVYYCGTSDN |
| (SEQ ID NO: 889) EPL13 | WGQGTQVTVSS |
| Exemplary anti-target | QVQLQESGGGLVQAGGSLRLSCAASGFAFGNHWMYWYRQAPGRGRELV |
| (EpCAM) | ASISSGGSTNYVDSVKGRFTISRDNARNTVYLQMYSLRPEDTAVYYCGTSDN |
| (SEQ ID NO: 890) | WGQGTQVTVSS |
| EPL129 | |
| Exemplary anti-target | QVQLQESGGGLVQAGGSLRLSCAASGFAFGNHWMYWYRQAPGRGRELV |
| (EpCAM) | ASISSGGSTNYVDSVKGRFTISRDNARNTVYLQMYSLKPEDTAVYYCGTSDN |
| (SEQ ID NO: 891) | WGQGTQVTVSS |
| EPL159 | |
| Exemplary anti-target | QVQLQESGGGLVQAGGSLRLSCAASGFIFRAASISWYRQSPGNERELVATIN |
| (EpCAM) | SGGFTNYADSVLGRFTISRDNAKNTGYLQMNSLKPEDTAVYFCAATFLRSDG |
| (SEQ ID NO: 892) | QPPIWGQGTQVTVSS |
| EPL120 | |
| Exemplary anti-target | QVQLQESGGGLVQAGGSLRLSCAASGFIFRAASMGWYRQSPGNERELVATI |
| (EpCAM) | NSGGETNYADSVKGRFTISRDNAKNTGYLQMNSLKPEDTAVYFCAATFLRSD |
| (SEQ ID NO: 893) | GQPPIWGQGTQVTVSS |
| EPL126 | |
| Exemplary anti-target | QVQLQESGGGLVQAGGSLRLSCAASEYILSMYRMAWYRQAPGKVRELVAD |
| (EpCAM) | MSSGGTTNYADFVKGRFTISRDNDRNTVYLQMNRLQPEDTAAYYCNVAGR |
| (SEQ ID NO: 894) EPL60 | TGPPSYDAFNNWGQGTQVTVSS |
| Exemplary anti-target | QVQLQESGGGLVQPGASLRVSCAASEYILSMYRMAWYRQAPGKVRELVAD |
| (EpCAM) | MSSGGTTNYADFVKGRFTISRDNDRNTVYLQMNRLQPEDTAAYYCNVAGR |
| (SEQ ID NO: 895) | TGPPSYDAFNNWGQGTQVTVSS |
| EPL156 | |
| Exemplary anti-target | QVQLQESGGGLVQPGGSLRLSCAASESISSFIAVGWYRQAPGKERELVAGIN |
| (EpCAM) | RSGFTYYTDSVKGRFSISRDNAKNTVLLQMTSLKPEDTAVYYCNAGGLYFSN |
| (SEQ ID NO: 896) EPL2 | AYTQGDYWGQGTQVTVSS |
| Exemplary anti-target | QVQLQESGGGLVQTGGSLRLSCAASESISSFIAVGWYRQAPGKERELVAGIN |
| (EpCAM) | RSGFTYYTDSVKGRFSISRDNAKNTVLLQMTSLKPEDTAVYYCNAGGLYFSN |
| (SEQ ID NO: 897) EPL43 | AYTQGDYWGQGTQVTVSS |
| Exemplary anti-target | QVQLQESGGGLVQAGGSLRLSCAASGSVFRANVMGWYRQAPGKQHELVA |
| (EpCAM) | RIDPGGTTTYADPVKGRFTISRDNAKKTVYLQMNSLKPDDTAVYYCNAIILLS |
| (SEQ ID NO: 898) EPL10 | GGPKDYWGQGTQVTVSS |
| Exemplary anti-target | QVQLQESGGGLVQAGGSLRLSCAPSGRTSSIFGMGWFRQAPGKEREFVASI |
| (EpCAM) | NWSGGSTSYADSVKGRFTISRDNAKNEMYLQMNSLKFEDTAVYVCAAAVL |
| (SEQ ID NO: 899) EPL49 | TNKPSWNFWGQGTQVTVSS |
| Exemplary anti-target | QVQLQESGGGLVQAGGSLRLSCAASGPIFSDTIRTMGWYRQAAGKQRELV |
| (EpCAM) | ATIASFPSRTNYVDSVKGRFTISRDIAKNTVYLQMDSLKPEDTAVYYCNVDLA |
| (SEQ ID NO: 900) EPL58 | SIPTKTYWGQGTQVTVSS |
| Exemplary anti-target | QVQLQESGGGLVQAGGSLRLSCAASGSIFGINAMGWYRQAPGKQRESVAF |
| (EpCAM) | ITIGGNTNYLDSVKGRFTISRDNAKNTVYLQMNGLKPEDTAVYYCNTNPPLIL |
| (SEQ ID NO: 901) EPL74 | TAGGLYWGQGTQVTVSS |
| Exemplary anti-target | QVQLQESGGGLVQPGGSLRLSCATSANRFNINVMGWYRQAPGQQRELVA |
| (EpCAM) | TINIGGSTDYADSVKGRFTISRDNAKNTVYLQLSDLKPEDTAVYYCNVKLRVS |
| (SEQ ID NO: 902) EPL78 | GPTGPNVYWGQGTQVTVSS |
| Exemplary anti-target | QVQLQESGGGLVQAGGSLKLSCTASGTILSTMAWYRQAPGKQRELVATISR |
| (EpCAM) | GGTTNYSDSVKGRFAISRDSTKNTVYLQMNSLKPEDTAVYYCNTPLTDYGM |
| (SEQ ID NO: 903) EPL82 | GYNWGQGTQVTVSS |
| Exemplary anti-target | QVQLQESGGGLVQAGGSLRLSCAVSGSIFSLNTLAWYRQAPGRQRDLIARIT |
| (EpCAM) | GGGTTVYADSVKGRFTISRDNAKNTVYLQMNSLKPEDTAVYYCNLMVRHPS |
| (SEQ ID NO: 904) EPL83 | GSTYEYWGQGTQVTVSS |
| Exemplary anti-target | QVQLQESGGGLVQAGGSLRLSCAASGIIFRGTTMGWFRQAPGKQRESVASI |
| (EpCAM) | SPLGTTSYSGSVEGRFTVSRDNAKNTLFLQMNSLKSEDTAVYYCNAIQVTNV |
| (SEQ ID NO: 905) EPL97 | GPRVYWGQGTQVTVSS |
| Exemplary anti-target | QVQLQESGGGLVQPGGSLRLSCASSGFTLDDYTIGWFRQAPGKEREGVSCIS |
| (EpCAM) | RRDDSTYYADSVKGRFTISRDNAKNTVDLQMISLRPEDTAVYYCAATPRSYTL |
| (SEQ ID NO: 906) | RCLGKFDFQGQGTQVTVSS |
| EPL109 | |
| Exemplary anti-target | QVQLQESGGGLVQAGGSLRLSCAASGNIVRMTNMAWYRQAPGKQREFVA |
| (EpCAM) | TISAGGSTTYVDSVKDRFTISRDNTKNTVYLQMNYLKPEDTAVYYCATGSILT |
| (SEQ ID NO: 907) | NRGAIPGSWGHGTQVTVSS |
| EPL117 | |
| Exemplary anti-target | QVQLQESGGGLVQAGGSLRLSCAAPGFAFNDHAILWFRQAPGKEREGVSEI |
| (EpCAM) | CRDGTTYYTDSVKGRFTISSDNAKNTVYLQMNSVKTDDTAVYYCAVDRRRY |
| (SEQ ID NO: 908) | YCSGNRAFSSDYYYVVGQGTQVTVSS |
| EPL127 | |
| Exemplary anti-target | QVQLQESGGGLVQAGGSLRLSCVHSGSIFRASTMAWYRQAPGKQRELVAQ |
| (EpCAM) | IMSGGGTNYAGSVKGRFTISRDNANNTVYLQMNSLKPEDTAVYYCNAAQIT |
| (SEQ ID NO: 909) | SWGPKVYWGQGTQVTVSS |
| EPL152 | |
| Exemplary anti-target | QVQLQESGGGLVQPGGSLRLSCAASGRINSINTMGWYRQAPGNQRELVAE |
| (EpCAM) | ITRGGTTNYADSVQGRYAISRDNAKNLVYLQMNSLKPEDTDVYYCNAQTFP |
| (SEQ ID NO: 910) | TFSRPTGLDYWGQGTQVTVSS |
| EPL189 | |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 911) | NRWVFGGGTKLTVLGGGGSGGGGSGGGGSEVQLVESGGGLVQPGGSLKL |
| EPL10 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAASGSVFRANVMGW |
| | YRQAPGKQHELVARIDPGGTTTYADPVKGRFTISRDNAKKTVYLQMNSLKP |
| | DDTAVYYCNAIILLSGGPKDYWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 912) | NRWVFGGGTKLTVLGGGGSGGGGSGGGGSEVQLVESGGGLVQPGGSLKL |
| EPL109 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQPGGSLRLSCASSGFTLDDYTIGWFR |
| | QAPGKEREGVSCISRRDDSTYYADSVKGRFTISRDNAKNTVDLQMISLRPED |
| | TAVYYCAATPRSYTLRCLGKFDFQGQGTQVTVSSHHHHHH* |
| Exemplary anti- | VVGGGGTQTANTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 913) | NRWVFGGGTKLTVLGGGGSGGGGSGGGGSEVQLVESGGGLVQPGGSLKL |
| EPL117 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAASGNIVRMTNMA |
| | WYRQAPGKQREFVATISAGGSTTYVDSVKDRFTISRDNTKNTVYLQMNYLK |
| | PEDTAVYYCATGSILTNRGAIPGSWGHGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 914) | NRWVFGGGTKLTVLGGGGSGGGGSGGGGSEVQLVESGGGLVQPGGSLKL |
| EPL120 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAASGFIFRAASISWYR |
| | QSPGNERELVATINSGGFTNYADSVLGRFTISRDNAKNTGYLQMNSLKPEDT |
| | AVYFCAATFLRSDGQPPIWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 915) | NRWVFGGGTKLTVLGGGGSGGGGSGGGGSEVQLVESGGGLVQPGGSLKL |
| EPL125 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAASGNVFRAATMAW |
| | YRQVPEKQREMVATIASGGTTNYADFVKGRFTISRDNAKNTVYLQMNTLKP |
| | EDTAVYYCNALYLTSLGPKSYWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 916) | NRWVFGGGTKLTVLGGGGSGGGGSGGGGSEVQLVESGGGLVQPGGSLKL |
| EPL127 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAAPGFAFNDHAILWF |
| | RQAPGKEREGVSEICRDGTTYYTDSVKGRFTISSDNAKNTVYLQMNSVKTDD |
| | TAVYYCAVDRRRYYCSGNRAFSSDYYYWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 917) | NRWVFGGGTKLTVLGGGGSGGGGSGGGGSEVQLVESGGGLVQPGGSLKL |
| EPL13 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQPGGSLRLSCAASGFAFGNHWMY |
| | WYRQAPGRGRELVASISSGGSTNYVDSVKGRFTISRDNARNTVYLQMYSLK |
| | PEDTAVYYCGTSDNWGQGTQVIVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 918) | NRWVFGGGTKLTVLGGGGSGGGGSGGGGSEVQLVESGGGLVQPGGSLKL |
| EPL136 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQPGGSLRLSCAASGDTFLRYAMGW |
| | FRQAPGKEREFVAAITWNGGNTDYAGSLKGRFTISRDNTKNTVYLQMNSLK |
| | PEDTAVYYCAADLTFGLASSHYQYDYWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 919) | NRWVFGGGTKLTVLGGGGSGGGGSGGGGSEVQLVESGGGLVQPGGSLKL |
| EPL138 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAASGFIFRAASMDWY |
| | RQFPGNERESIATISSGGFTNYADSVKGRFTISRDNAKNTVYLQMNSLKPEDT |
| | AVYFCGATFLRSDGHHTINGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 920) | NRWVFGGGTKLTVLGGGGSGGGGSGGGGSEVQLVESGGGLVQPGGSLKL |
| EPL145 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAASGFIFRAASMGWF |
| | RQSPGNERELVATVSSGGFTNYADSVKGRFTISRDNAKNTVYLQMNSLKPE |
| | DTAVYFCGATFVRSDGHHTIYGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 921) | NRWVFGGGTKLTVLGGGGSGGGGSGGGGSEVQLVESGGGLVQPGGSLKL |
| EPL152 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCVHSGSIFRASTMAWY |
| | RQAPGKQRELVAQIMSGGGTNYAGSVKGRFTISRDNANNTVYLQMNSLKP |
| | EDTAVYYCNAAQITSWGPKVYWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 922) | NRWVFGGGTKLTVLGGGGSGGGGSGGGGSEVQLVESGGGLVQPGGSLKL |
| EPL153 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQPGGSLRLSCAASGFTFSDWAMSW |
| | VRQAPGKGLEWVSSIHYGDHTTHYADFVKGRFTISRDDAKNTLYLQMNSLK |
| | PEDTAVYYCEKGTTRGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 923) | NRWVFGGGTKLTVLGGGGSGGGGSGGGGSEVQLVESGGGLVQPGGSLKL |
| EPL156 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQPGASLRVSCAASEYILSMYRMAWY |
| | RQAPGKVRELVADMSSGGTTNYADFVKGRFTISRDNDRNTVYLQMNRLQP |
| | EDTAAYYCNVAGRTGPPSYDAFNNWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 924) | NRWVFGGGTKLTVLGGGGSGGGGSGGGGSEVQLVESGGGLVQPGGSLKL |
| EPL164 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAASGFIFRAASMDWY |
| | RQSPGTQPELVATISSTGFTNYANSVKGRFTISRDNAKNTVYLQMNSLKPED |
| | TAVYFCGATFLRSDGQHSIYGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 924) | NRWVFGGGTKLTVLGGGGSGGGGSGGGGSEVQLVESGGGLVQPGGSLKL |
| EPL189 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQPGGSLRLSCAASGRINSINTMGWY |
| | RQAPGNORELVAEITRGGTTNYADSVQGRYAISRDNAKNLVYLQMNSLKPE |
| | DTDVYYCNAQTFPTFSRPTGLDYWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 925) | NRWVFGGGTKLTVLGGGGSGGGGSGGGGSEVQLVESGGGLVQPGGSLKL |
| EPL2 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQPGGSLRLSCAASESISSFIAVGWYR |
| | QAPGKERELVAGINRSGFTYYTDSVKGRFSISRDNAKNTVLLQMTSLKPEDT |
| | AVYYCNAGGLYFSNAYTQGDYWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 926) | NRWVFGGGTKLTVLGGGGSGGGGSGGGGSEVQLVESGGGLVQPGGSLKL |
| EPL20 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLKLSCAASGNVFRAATMAW |
| | YRQAPEKQREMVATIASGGTTNYADEVKGRFTISRDNAKNTVYLQMNTLKP |
| | EDTAVYYCNAGYLTSLGPKNYWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ. ID No. 927) | NRWVFGGGTKLTVLGGGGSGGGGSGGGGSEVQLVESGGGLVQPGGSLKL |
| EPL34 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGSVQAGGSLRLSCAASGFTFSSYYMSWV |
| | RQAPGKGLEWVSGIHYTGDWTNYADSVKGRFTISRDNAKNELYLEMNNLK |
| | PEDTAVYYCARGSDKGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 928) | NRWVFGGGTKLTVLGGGGSGGGGSGGGGSEVQLVESGGGLVQPGGSLKL |
| EPL49 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAPSGRTSSIFGMGWF |
| | RQAPGKEREFVASINWSGGSTSYADSVKGRFTISRDNAKNEMYLQMNSLKF |
| | EDTAVYVCAAAVLTNKPSWNFWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 929) | NRWVFGGGTKLTVLGGGGSGGGGSGGGGSEVQLVESGGGLVQPGGSLKL |
| EPL58 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAASGPIFSDTIRTMG |
| | WYRQAAGKQRELVATIASFPSRTNYVDSVKGRFTISRDIAKNTVYLQMDSLK |
| | PEDTAVYYCNVDLASIPTKTYWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ. ID No. 930) | NRWVFGGGTKLTVLGGGGSGGGGSGGGGSEVQLVESGGGLVQPGGSLKL |
| EPL74 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAASGSIFGINAMGWY |
| | RQAPGKQRESVAFITIGGNTNYLDSVKGRFTISRDNAKNTVYLQMNGLKPED |
| | TAVYYCNTNPPLILTAGGLYWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 931) | NRWVFGGGTKLTVLGGGGSGGGGSGGGGSEVQLVESGGGLVQPGGSLKL |
| EPL78 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQPGGSLRLSCATSANRFNINVMGW |
| | YRQAPGQQRELVATINIGGSTDYADSVKGRFTISRDNAKNTVYLQLSDLKPE |
| | DTAVYYCNVKLRVSGPIGPNVYWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 932) | NRWVFGGGTKLTVLGGGGSGGGGSGGGGSEVQLVESGGGLVQPGGSLKL |
| EPL82 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLKLSCTASGTILSTMAWYRQ |
| | APGKQRELVATISRGGTTNYSDSVKGRFAISRDSTKNTVYLQMNSLKPEDTA |
| | VYYCNTPLTDYGMGYNWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 933) | NRWVFGGGTKLTVLGGGGSGGGGSGGGGSEVQLVESGGGLVQPGGSLKL |
| EPL83 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAVSGSIFSLNTLAWYR |
| | QAPGRQRDLIARITGGGTTVYADSVKGRFTISRDNAKNTVYLQMNSLKPEDT |
| | AVYYCNLMVRHPSGSTYEYWGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 934) | NRWVFGGGTKLTVLGGGGSGGGGSGGGGSEVQLVESGGGLVQPGGSLKL |
| EPL86 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQPGGSLRLSCAASGFTFSDWAMSW |
| | VRQAPGKGLEWVSGIHYGDHTTHYADFVKGRFTISRDDAKNTLYLQMNSLK |
| | PEDTAVYYCARGSTKGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 935) | NRWVFGGGTKLTVLGGGGSGGGGSGGGGSEVQLVESGGGLVQPGGSLKL |
| EPL90 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAASGFIFRAASMAWY |
| | RQSPGNERELVASISSGAFTNYADSVKARFTISRDNAKNTVYLQMNSLKPED |
| | TAVYFCGATFLRSDGHHTINGQGTQVTVSSHHHHHH* |
| Exemplary anti- | VVGGGGTQTVVTQEPSLTVSPGGTVTLTCASSTGAVTSGNYPNWVQQKPG |
| EpCAM/Anti-CD3 fusion | QAPRGLIGGTKFLVPGTPARFSGSLLGGKAALTLSGVQPEDEAEYYCTLWYS |
| protein (SEQ ID No. 936) | NRWVFGGGTKLTVLGGGGSGGGGSGGGGSEVQLVESGGGLVQPGGSLKL |
| EPL97 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSQVQLQESGGGLVQAGGSLRLSCAASGIIFRGTTMGWF |
| | RQAPGKQRESVASISPLGTTSYSGSVEGRFTVSRDNAKNTLFLQMNSLKSED |
| | TAVYYCNAIQVTNVGPRVYWGQGTQVTVSSHHHHHH* |
| Exemplary anti- | GGGSQVQLQESGGGLVQAGGSLRLSCAASGSVFRANVMGWYRQAPGKQ |
| CD3/Anti-EpCAM fusion | HELVARIDPGGTTTYADPVKGRFTISRDNAKKTVYLQMNSLKPDDTAVYYCN |
| protein (SEQ ID No. 937) | AIILLSGGPKDYWGQGTQVTVSSGGGGSGGGSEVQLVESGGGLVQPGGSLK |
| EPL10 | LSCAASGFTFNKYAINWVRQAPGKGLEWVARIRSKYNNYATYYADQVKDRF |
| | TISRDDSKNTAYLQMNNLKTEDTAVYYCVRHANFGNSYISYWAYWGQGTL |
| | VTVSSGGGGSGGGGSGGGGSQTVVTQEPSLTVSPGGTVTLTCASSTGAVTS |
| | GNYPNWVQQKPGQAPRGLIGGTKFLVPGTPARFSGSLLGGKAALTLSGVQP |
| | EDEAEYYCTLWYSNRWVFGGGTKLTVLHHHHHH* |
| Exemplary anti- | GGGSQVQLQESGGGLVQPGGSLRLSCASSGFTLDDYTIGWFRQAPGKEREG |
| CD3/Anti-EpCAM fusion | VSCISRRDDSTYYADSVKGRFTISRDNAKNTVDLQMISLRPEDTAVYYCAATP |
| protein (SEQ ID No. 938) | RSYTLRCLGKFDFQGQGTQVTVSSGGGGSGGGSEVQLVESGGGLVQPGGS |
| EPL109 | LKLSCAASGFTFNKYAINWVRQAPGKGLEWVARIRSKYNNYATYYADQVKD |
| | RFTISRDDSKNTAYLQMNNLKTEDTAVYYCVRHANFGNSYISYWAYWGQG |
| | TLVTVSSGGGGSGGGGSGGGGSQTVVTQEPSLTVSPGGTVTLTCASSTGAV |
| | TSGNYPNWVQQKPGQAPRGLIGGTKFLVPGTPARFSGSLLGGKAALTLSGV |
| | QPEDEAEYYCTLWYSNRWVFGGGTKLTVLHHHHHH* |
| Exemplary anti- | GGGSQVQLQESGGGLVQAGGSLRLSCAASGNIVRMTNMAWYRQAPGKQ |
| CD3/Anti-EpCAM fusion | REFVATISAGGSTTYVDSVKDRFTISRDNTKNTVYLQMNYLKPEDTAVYYCAT |
| protein (SEQ ID No. 939) | GSILTNRGAIPGSWGHGTQVTVSSGGGGSGGGSEVQLVESGGGLVQPGGS |
| EPL117 | LKLSCAASGFTFNKYAINWVRQAPGKGLEWVARIRSKYNNYATYYADQVKD |
| | RFTISRDDSKNTAYLQMNNLKTEDTAVYYCVRHANFGNSYISYWAYWGQG |
| | TLVTVSSGGGGSGGGGSGGGGSQTVVTQEPSLTVSPGGTVTLTCASSTGAV |
| | TSGNYPNWVQQKPGQAPRGLIGGTKFLVPGTPARFSGSLLGGKAALTLSGV |
| | QPEDEAEYYCTLWYSNRWVFGGGTKLTVLHHHHHH* |
| Exemplary anti- | GGGSQVQLQESGGGLVQAGGSLRLSCAASGFIFRAASISWYRQSPGNEREL |
| CD3/Anti-EpCAM fusion | VATINSGGFTNYADSVLGRFTISRDNAKNTGYLQMNSLKPEDTAVYFCAATF |
| protein (SEQ ID No. 940) | LRSDGQPPIWGQGTQVTVSSGGGGSGGGSEVQLVESGGGLVQPGGSLKLS |
| EPL120 | CAASGFTFNKYAINWVRQAPGKGLEWVARIRSKYNNYATYYADQVKDRFTI |
| | SRDDSKNTAYLQMNNLKTEDTAVYYCVRHANFGNSYISYWAYWGQGTLVT |
| | VSSGGGGSGGGGSGGGGSQTVVTQEPSLTVSPGGTVTLTCASSTGAVTSGN |
| | YPNWVQQKPGQAPRGLIGGTKFLVPGTPARFSGSLLGGKAALTLSGVQPED |
| | EAEYYCTLWYSNRWVFGGGTKLTVLHHHHHH* |
| Exemplary anti- | GGGSQVQLQESGGGLVQAGGSLRLSCAASGNVFRAATMAWYRQVPEKQR |
| CD3/Anti-EpCAM fusion | EMVATIASGGTTNYADFVKGRFTISRDNAKNTVYLQMNTLKPEDTAVYYCN |
| protein (SEQ ID No. 941) | ALYLTSLGPKSYWGQGTQVTVSSGGGGSGGGSEVQLVESGGGLVQPGGSL |
| EPL125 | KLSCAASGFTFNKYAINWVRQAPGKGLEWVARIRSKYNNYATYYADQVKDR |
| | FTISRDDSKNTAYLQMNNLKTEDTAVYYCVRHANFGNSYISYWAYWGQGTL |
| | VTVSSGGGGSGGGGSGGGGSQTVVTQEPSLTVSPGGTVTLTCASSTGAVTS |
| | GNYPNWVQQKPGQAPRGLIGGTKFLVPGTPARFSGSLLGGKAALTLSGVQP |
| | EDEAEYYCTLWYSNRWVFGGGTKLTVLHHHHHH* |
| Exemplary anti- | GGGSQVQLQESGGGLVQAGGSLRLSCAAPGFAFNDHAILWFRQAPGKERE |
| CD3/Anti-EpCAM fusion | GVSEICRDGTTYYTDSVKGRFTISSDNAKNTVYLQMNSVKTDDTAVYYCAVD |
| protein (SEQ ID No. 942) | RRRYYCSGNRAFSSDYYYWGQGTQVTVSSGGGGSGGGSEVQLVESGGGLV |
| EPL127 | QPGGSLKLSCAASGFTFNKYAINWVRQAPGKGLEWVARIRSKYNNYATYYA |
| | DQVKDRFTISRDDSKNTAYLQMNNLKTEDTAVYYCVRHANFGNSYISYWAY |
| | WGQGTLVTVSSGGGGSGGGGSGGGGSQTVVTQEPSLTVSPGGTVTLTCAS |
| | STGAVTSGNYPNWVQQKPGQAPRGLIGGTKFLVPGTPARFSGSLLGGKAAL |
| | TLSGVQPEDEAEYYCTLWYSNRWVFGGGTKLTVLHHHHHH* |
| Exemplary anti- | GGGSQVQLQESGGGLVQPGGSLRLSCAASGFAFGNHWMYWYRQAPGRG |
| CD3/Anti-EpCAM fusion | RELVASISSGGSTNYVDSVKGRFTISRDNARNTVYLQMYSLKPEDTAVYYCGT |
| protein (SEQ ID No. 943) | SDNWGQGTQVTVSSGGGGSGGGSEVQLVESGGGLVQPGGSLKLSCAASG |
| EPL13 | FTFNKYAINWVRQAPGKGLEWVARIRSKYNNYATYYADQVKDRFTISRDDS |
| | KNTAYLQMNNLKTEDTAVYYCVRHANFGNSYISYWAYWGQGTLVTVSSGG |
| | GGSGGGGSGGGGSQTVVTQEPSLTVSPGGTVTLTCASSTGAVTSGNYPNW |
| | VQQKPGQAPRGLIGGTKFLVPGTPARFSGSLLGGKAALTLSGVQPEDEAEYY |
| | CTLWYSNRWVFGGGTKLTVLHHHHHId* |
| Exemplary anti- | GGGSQVQLQESGGGLVQPGGSLRLSCAASGDTFLRYAMGWFRQAPGKER |
| CD3/Anti-EpCAM fusion | EFVAAITWNGGNTDYAGSLKGRFTISRDNTKNTVYLQMNSLKPEDTAVYYC |
| protein (SEQ ID No. 944) | AADLTFGLASSHYQYDYWGQGTQVTVSSGGGGSGGGSEVQLVESGGGLV |
| EPL136 | QPGGSLKLSCAASGFTFNKYAINWVRQAPGKGLEWVARIRSKYNNYATYYA |
| | DQVKDRFTISRDDSKNTAYLQMNNLKTEDTAVYYCVRHANFGNSYISYWAY |
| | WGQGTLVTVSSGGGGSGGGGSGGGGSQTVVTQEPSLTVSPGGTVTLTCAS |
| | STGAVTSGNYPNWVQQKPGQAPRGLIGGTKFLVPGTPARFSGSLLGGKAAL |
| | TLSGVQPEDEAEYYCTLWYSNRWVFGGGTKLTVLHHHHHH* |
| Exemplary anti- | GGGSQVQLQESGGGLVQAGGSLRLSCAASGFIFRAASMDWYRQFPGNERE |
| CD3/Anti-EpCAM fusion | SIATISSGGFTNYADSVKGRFTISRDNAKNTVYLQMNSLKPEDTAVYFCGATF |
| protein (SEQ ID No. 945) | LRSDGHHTINGQGTQVTVSSGGGGSGGGSEVQLVESGGGLVQPGGSLKLS |
| EPL138 | CAASGFTFNKYAINWVRQAPGKGLEWVARIRSKYNNYATYYADQVKDRFTI |
| | SRDDSKNTAYLQMNNLKTEDTAVYYCVRHANFGNSYISYWAYWGQGTLVT |
| | VSSGGGGSGGGGSGGGGSQTVVTQEPSLTVSPGGTVTLTCASSTGAVTSGN |
| | YPNWVQQKPGQAPRGLIGGTKFLVPGTPARFSGSLLGGKAALTLSGVQPED |
| | EAEYYCTLWYSNRWVFGGGTKLTVLHHHHHH* |
| Exemplary anti- | GGGSQVQLQESGGGLVQAGGSLRLSCAASGFIFRAASMGWFRQSPGNERE |
| CD3/Anti-EpCAM fusion | LVATVSSGGFTNYADSVKGRFTISRDNAKNTVYLQMNSLKPEDTAVYFCGAT |
| protein (SEQ ID No. 946) | FVRSDGHHTIYGQGTQVTVSSGGGGSGGGSEVQLVESGGGLVQPGGSLKLS |
| Exemplary anti- | CAASGFTFNKYAINWVRQAPGKGLEWVARIRSKYNNYATYYADQVKDRFTI |
| CD3/Anti-EpCAM fusion | SRDDSKNTAYLQMNNLKTEDTAVYYCVRHANFGNSYISYWAYWGQGTLVT |
| protein (SEQ ID No. 947) | VSSGGGGSGGGGSGGGGSQTVVTQEPSLTVSPGGTVTLTCASSTGAVTSGN |
| EPL145 | YPNWVQQKPGQAPRGLIGGTKFLVPGTPARFSGSLLGGKAALTLSGVQPED |
| | EAEYYCTLWYSNRWVFGGGTKLTVLHHHHHH* |
| Exemplary anti- | GGGSQVQLQESGGGLVQAGGSLRLSCVHSGSIFRASTMAWYRQAPGKQRE |
| CD3/Anti-EpCAM fusion | LVAQIMSGGGTNYAGSVKGRFTISRDNANNTVYLQMNSLKPEDTAVYYCN |
| protein (SEQ ID No. 948) | AAQITSWGPKVYWGQGTQVTVSSGGGGSGGGSEVQLVESGGGLVQPGGS |
| EPL152 | LKLSCAASGFTFNKYAINWVRQAPGKGLEWVARIRSKYNNYATYYADQVKD |
| | RFTISRDDSKNTAYLQMNNLKTEDTAVYYCVRHANFGNSYISYWAYWGQG |
| | TLVTVSSGGGGSGGGGSGGGGSQTVVTQEPSLTVSPGGTVTLTCASSTGAV |
| | TSGNYPNWVQQKPGQAPRGLIGGTKFLVPGTPARFSGSLLGGKAALTLSGV |
| | QPEDEAEYYCTLWYSNRWVFGGGTKLTVLHHHHHH* |
| Exemplary anti- | GGGSQVQLQESGGGLVQPGGSLRLSCAASGFTFSDWAMSWVRQAPGKGL |
| CD3/Anti-EpCAM fusion | EWVSSIHYGDHTTHYADFVKGRFTISRDDAKNTLYLQMNSLKPEDTAVYYCE |
| protein (SEQ ID No. 949) | KGTTRGQGTQVTVSSGGGGSGGGSEVQLVESGGGLVQPGGSLKLSCAASG |
| EPL153 | FTFNKYAINWVRQAPGKGLEWVARIRSKYNNYATYYADQVKDRFTISRDDS |
| | KNTAYLQMNNLKTEDTAVYYCVRHANFGNSYISYWAYWGQGTLVTVSSGG |
| | GGSGGGGSGGGGSQTVVTQEPSLTVSPGGTVTLTCASSTGAVTSGNYPNW |
| | VQQKPGQAPRGLIGGTKFLVPGTPARFSGSLLGGKAALTLSGVQPEDEAEYY |
| | CTLWYSNRWVFGGGTKLTVLHHHHHH* |
| Exemplary anti- | GGGSQVQLQESGGGLVQPGASLRVSCAASEYILSMYRMAWYRQAPGKVRE |
| CD3/Anti-EpCAM fusion | LVADMSSGGTTNYADFVKGRFTISRDNDRNTVYLQMNRLQPEDTAAYYCN |
| protein (SEQ ID No. 950) | VAGRTGPPSYDAFNNWGQGTQVTVSSGGGGSGGGSEVQLVESGGGLVQP |
| EPL156 | GGSLKLSCAASGFTFNKYAINWVRQAPGKGLEWVARIRSKYNNYATYYADQ |
| | VKDRFTISRDDSKNTAYLQMNNLKTEDTAVYYCVRHANFGNSYISYWAYW |
| | GQGTLVTVSSGGGGSGGGGSGGGGSQTVVTQEPSLTVSPGGTVTLTCASST |
| | GAVTSGNYPNWVQQKPGQAPRGLIGGTKFLVPGTPARFSGSLLGGKAALTL |
| | SGVQPEDEAEYYCTLWYSNRWVFGGGTKLTVLHHHHHH* |
| Exemplary anti- | GGGSQVQLQESGGGLVQAGGSLRLSCAASGFIFRAASMDWYRQSPGTQPE |
| CD3/Anti-EpCAM fusion | LVATISSTGFTNYANSVKGRFTISRDNAKNTVYLQMNSLKPEDTAVYFCGATF |
| protein (SEQ ID No. 951) | LRSDGQHSIYGQGTQVTVSSGGGGSGGGSEVQLVESGGGLVQPGGSLKLSC |
| EPL164 | AASGFTFNKYAINWVRQAPGKGLEWVARIRSKYNNYATYYADQVKDRFTIS |
| | RDDSKNTAYLQMNNLKTEDTAVYYCVRHANFGNSYISYWAYWGQGTLVTV |
| | SSGGGGSGGGGSGGGGSQTVVTQEPSLTVSPGGTVTLTCASSTGAVTSGNY |
| | PNWVQQKPGQAPRGLIGGTKFLVPGTPARFSGSLLGGKAALTLSGVQPEDE |
| | AEYYCTLWYSNRWVFGGGTKLTVLHHHHHH* |
| Exemplary anti- | GGGSQVQLQESGGGLVQPGGSLRLSCAASGRINSINTMGWYRQAPGNQR |
| CD3/Anti-EpCAM fusion | ELVAEITRGGTTNYADSVQGRYAISRDNAKNLVYLQMNSLKPEDTDVYYCN |
| protein (SEQ ID No. 952) | AQTFPTFSRPTGLDYWGQGTQVTVSSGGGGSGGGSEVQLVESGGGLVQPG |
| EPL189 | GSLKLSCAASGFTFNKYAINWVRQAPGKGLEWVARIRSKYNNYATYYADQV |
| | KDRFTISRDDSKNTAYLQMNNLKTEDTAVYYCVRHANFGNSYISYWAYWG |
| | QGTLVTVSSGGGGSGGGGSGGGGSQTVVTQEPSLTVSPGGTVTLTCASSTG |
| | AVTSGNYPNWVQQKPGQAPRGLIGGTKFLVPGTPARFSGSLLGGKAALTLS |
| | GVQPEDEAEYYCTLWYSNRWVFGGGTKLTVLHHHHHH* |
| Exemplary anti- | GGGSQVQLQESGGGLVQPGGSLRLSCAASESISSFIAVGWYRQAPGKERELV |
| CD3/Anti-EpCAM fusion | AGINRSGFTYYTDSVKGRFSISRDNAKNTVLLQMTSLKPEDTAVYYCNAGGL |
| protein (SEQ ID No. 953) | YFSNAYTQGDYWGQGTQVTVSSGGGGSGGGSEVQLVESGGGLVQPGGSL |
| EPL2 | KLSCAASGFTFNKYAINWVRQAPGKGLEWVARIRSKYNNYATYYADQVKDR |
| | FTISRDDSKNTAYLQMNNLKTEDTAVYYCVRHANFGNSYISYWAYWGQGTL |
| | VTVSSGGGGSGGGGSGGGGSQTVVTQEPSLTVSPGGTVTLTCASSTGAVTS |
| | GNYPNWVQQKPGQAPRGLIGGTKFLVPGTPARFSGSLLGGKAALTLSGVQP |
| | EDEAEYYCTLWYSNRWVFGGGTKLTVLHHHHHH* |
| Exemplary anti- | GGGSQVQLQESGGGLVQAGGSLKLSCAASGNVFRAATMAWYRQAPEKQR |
| CD3/Anti-EpCAM fusion | EMVATIASGGTTNYADFVKGRFTISRDNAKNTVYLQMNTLKPEDTAVYYCN |
| protein (SEQ ID No. 954) | AGYLTSLGPKNYWGQGTQVTVSSGGGGSGGGSEVQLVESGGGLVQPGGSL |
| EPL20 | KLSCAASGFTFNKYAINWVRQAPGKGLEWVARIRSKYNNYATYYADQVKDR |
| | FTISRDDSKNTAYLQMNNLKTEDTAVYYCVRHANFGNSYISYWAYWGQGTL |
| | VTVSSGGGGSGGGGSGGGGSQTVVTQEPSLTVSPGGTVTLTCASSTGAVTS |
| | GNYPNWVQQKPGQAPRGLIGGTKFLVPGTPARFSGSLLGGKAALTLSGVQP |
| | EDEAEYYCTLWYSNRWVFGGGTKLTVLHHHHHH* |
| Exemplary anti- | GGGSQVQLQESGGGSVQAGGSLRLSCAASGFTFSSYYMSWVRQAPGKGLE |
| CD3/Anti-EpCAM fusion | WVSGIHYTGDWTNYADSVKGRFTISRDNAKNELYLEMNNLKPEDTAVYYCA |
| protein (SEQ ID No. 955) | RGSDKGQGTQVTVSSGGGGSGGGSEVQLVESGGGLVQPGGSLKLSCAASG |
| EPL34 | FTFNKYAINWVRQAPGKGLEWVARIRSKYNNYATYYADQVKDRFTISRDDS |
| | KNTAYLQMNNLKTEDTAVYYCVRHANFGNSYISYWAYWGQGTLVTVSSGG |
| | GGSGGGGSGGGGSQTVVTQEPSLTVSPGGTVTLTCASSTGAVTSGNYPNW |
| | VQQKPGQAPRGLIGGTKFLVPGTPARFSGSLLGGKAALTLSGVQPEDEAEYY |
| | CTLWYSNRWVFGGGTKLTVLHHHHHH* |
| Exemplary anti- | GGGSQVQLQESGGGLVQAGGSLRLSCAPSGRTSSIFGMGWFRQAPGKERE |
| CD3/Anti-EpCAM fusion | FVASINWSGGSTSYADSVKGRFTISRDNAKNEMYLQMNSLKFEDTAVYVCA |
| protein (SEQ ID No. 956) | AAVLTNKPSWNFWGQGTQVTVSSGGGGSGGGSEVQLVESGGGLVQPGG |
| EPL49 | SLKLSCAASGFTFNKYAINWVRQAPGKGLEWVARIRSKYNNYATYYADQVK |
| | DRFTISRDDSKNTAYLQMNNLKTEDTAVYYCVRHANFGNSYISYWAYWGQ |
| | GTLVTVSSGGGGSGGGGSGGGGSQTVVTQEPSLTVSPGGTVTLTCASSTGA |
| | VTSGNYPNWVQQKPGQAPRGLIGGTKFLVPGTPARFSGSLLGGKAALTLSG |
| | VQPEDEAEYYCTLWYSNRWVFGGGTKLTVLHHHHHH* |
| Exemplary anti- | GGGSQVQLQESGGGLVQAGGSLRLSCAASGPIFSDTIRTMGWYRQAAGKQ |
| CD3/Anti-EpCAM fusion | RELVATIASFPSRTNYVDSVKGRFTISRDIAKNTVYLQMDSLKPEDTAVYYCN |
| protein (SEQ ID No. 957) | VDLASIPTKTYWGQGTQVTVSSGGGGSGGGSEVQLVESGGGLVQPGGSLK |
| EPL58 | LSCAASGFTFNKYAINWVRQAPGKGLEWVARIRSKYNNYATYYADQVKDRF |
| | TISRDDSKNTAYLQMNNLKTEDTAVYYCVRHANFGNSYISYWAYWGQGTL |
| | VTVSSGGGGSGGGGSGGGGSQTVVTQEPSLTVSPGGTVTLTCASSTGAVTS |
| | GNYPNWVQQKPGQAPRGLIGGTKFLVPGTPARFSGSLLGGKAALTLSGVQP |
| | EDEAEYYCTLWYSNRWVFGGGTKLTVLHHHHHH* |
| Exemplary anti- | GGGSQVQLQESGGGLVQAGGSLRLSCAASGSIFGINAMGWYRQAPGKQR |
| CD3/Anti-EpCAM fusion | ESVAFITIGGNTNYLDSVKGRFTISRDNAKNTVYLQMNGLKPEDTAVYYCNT |
| protein (SEQ ID No. 958) | NPPLILTAGGLYWGQGTQVTVSSGGGGSGGGSEVQLVESGGGLVQPGGSL |
| EPL74 | KLSCAASGFTFNKYAINWVRQAPGKGLEWVARIRSKYNNYATYYADQVKDR |
| | FTISRDDSKNTAYLQMNNLKTEDTAVYYCVRHANFGNSYISYWAYWGQGTL |
| | VTVSSGGGGSGGGGSGGGGSQTVVTQEPSLTVSPGGTVTLTCASSTGAVTS |
| | GNYPNWVQQKPGQAPRGLIGGTKFLVPGTPARFSGSLLGGKAALTLSGVQP |
| | EDEAEYYCTLWYSNRWVFGGGTKLTVLHHHHHH* |
| Exemplary anti- | GGGSQVQLQESGGGLVQPGGSLRLSCATSANRFNINVMGWYRQAPGQQR |
| CD3/Anti-EpCAM fusion | ELVATINIGGSTDYADSVKGRFTISRDNAKNTVYLQLSDLKPEDTAVYYCNVK |
| protein (SEQ ID No. 959) | LRVSGPTGPNVYWGQGTQVTVSSGGGGSGGGSEVQLVESGGGLVQPGGS |
| EPL78 | LKLSCAASGFTFNKYAINWVRQAPGKGLEWVARIRSKYNNYATYYADQVKD |
| | RFTISRDDSKNTAYLQMNNLKTEDTAVYYCVRHANFGNSYISYWAYWGQG |
| | TLVTVSSGGGGSGGGGSGGGGSQTVVTQEPSLTVSPGGTVTLTCASSTGAV |
| | TSGNYPNWVQQKPGQAPRGLIGGTKFLVPGTPARFSGSLLGGKAALTLSGV |
| | QPEDEAEYYCTLWYSNRWVFGGGTKLTVLHHHHHH* |
| Exemplary anti- | GGGSQVQLQESGGGLVQAGGSLKLSCTASGTILSTMAWYRQAPGKQRELV |
| CD3/Anti-EpCAM fusion | ATISRGGTTNYSDSVKGRFAISRDSTKNTVYLQMNSLKPEDTAVYYCNTPLTD |
| protein (SEQ ID No. 960) | YGMGYNWGQGTQVTVSSGGGGSGGGSEVQLVESGGGLVQPGGSLKLSCA |
| EPL82 | ASGFTFNKYAINWVRQAPGKGLEWVARIRSKYNNYATYYADQVKDRFTISR |
| | DDSKNTAYLQMNNLKTEDTAVYYCVRHANFGNSYISYWAYWGQGTLVTVS |
| | SGGGGSGGGGSGGGGSQTVVTQEPSLTVSPGGTVTLTCASSTGAVTSGNYP |
| | NWVQQKPGQAPRGLIGGTKFLVPGTPARFSGSLLGGKAALTLSGVQPEDEA |
| | EYYCTLWYSNRWVFGGGTKLTVLHHHHHH* |
| Exemplary anti- | GGGSQVQLQESGGGLVQAGGSLRLSCAVSGSIFSLNTLAWYRQAPGRQRD |
| CD3/Anti-EpCAM fusion | LIARITGGGTTVYADSVKGRFTISRDNAKNTVYLQMNSLKPEDTAVYYCNLM |
| protein (SEQ ID No. 961) | VRHPSGSTYEYWGQGTQVTVSSGGGGSGGGSEVQLVESGGGLVQPGGSLK |
| EPL83 | LSCAASGFTFNKYAINWVRQAPGKGLEWVARIRSKYNNYATYYADQVKDRF |
| | TISRDDSKNTAYLQMNNLKTEDTAVYYCVRHANFGNSYISYWAYWGQGTL |
| | VTVSSGGGGSGGGGSGGGGSQTVVTQEPSLTVSPGGTVTLTCASSTGAVTS |
| | GNYPNWVQQKPGQAPRGLIGGTKFLVPGTPARFSGSLLGGKAALTLSGVQP |
| | EDEAEYYCTLWYSNRWVFGGGTKLTVLHHHHHH* |
| Exemplary anti- | GGGSQVQLQESGGGLVQPGGSLRLSCAASGFTFSDWAMSWVRQAPGKGL |
| CD3/Anti-EpCAM fusion | EWVSGIHYGDHTTHYADFVKGRFTISRDDAKNTLYLQMNSLKPEDTAVYYC |
| protein (SEQ ID No. 962) | ARGSTKGQGTQVTVSSGGGGSGGGSEVQLVESGGGLVQPGGSLKLSCAAS |
| EPL86 | GFTFNKYAINWVRQAPGKGLEWVARIRSKYNNYATYYADQVKDRFTISRDD |
| | SKNTAYLQMNNLKTEDTAVYYCVRHANFGNSYISYWAYWGQGTLVTVSSG |
| | GGGSGGGGSGGGGSQTVVTQEPSLTVSPGGTVTLTCASSTGAVTSGNYPN |
| | WVQQKPGQAPRGLIGGTKFLVPGTPARFSGSLLGGKAALTLSGVQPEDEAE |
| | YYCTLWYSNRWVFGGGTKLTVLHHHHHH* |
| Exemplary anti- | GGGSQVQLQESGGGLVQAGGSLRLSCAASGFIFRAASMAWYRQSPGNERE |
| CD3/Anti-EpCAM fusion | LVASISSGAFTNYADSVKARFTISRDNAKNTVYLQMNSLKPEDTAVYFCGATF |
| protein (SEQ ID No. 963) | LRSDGHHTINGQGTQVTVSSGGGGSGGGSEVQLVESGGGLVQPGGSLKLS |
| EPL90 | CAASGFTFNKYAINWVRQAPGKGLEWVARIRSKYNNYATYYADQVKDRFTI |
| | SRDDSKNTAYLQMNNLKTEDTAVYYCVRHANFGNSYISYWAYWGQGTLVT |
| | VSSGGGGSGGGGSGGGGSQTVVTQEPSLTVSPGGTVTLTCASSTGAVTSGN |
| | YPNWVQQKPGQAPRGLIGGTKFLVPGTPARFSGSLLGGKAALTLSGVQPED |
| | EAEYYCTLWYSNRWVFGGGTKLTVLHHHHHH* |
| Exemplary anti- | GGGSQVQLQESGGGLVQAGGSLRLSCAASGIIFRGTTMGWFRQAPGKQRE |
| CD3/Anti-EpCAM fusion | SVASISPLGTTSYSGSVEGRFTVSRDNAKNTLFLQMNSLKSEDTAVYYCNAIQ |
| protein (SEQ ID No. 964) | VTNVGPRVYWGQGTQVTVSSGGGGSGGGSEVQLVESGGGLVQPGGSLKL |
| EPL97 | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGGSGGGGSQTVVTQEPSLTVSPGGTVTLTCASSTGAVTSG |
| | NYPNWVQQKPGQAPRGLIGGTKFLVPGTPARFSGSLLGGKAALTLSGVQPE |
| | DEAEYYCTLWYSNRWVFGGGTKLTVLHHHHHH* |
| Exemplary humanized | EVQLVESGGGLVQPGGSLTLSCAASGFAFGNHWMYWYRQAPGRGRELVA |
| EpCAM binder H13 (SEQ | SISSGGSTNYVDSVKGRFTISRDNAKNTLYLQMNSLRAEDTAVYYCGTSDNW |
| ID No. 965) | GQGTLVTVSS |
| Exemplary humanized | EVQLLESGGGLVQPGGSLTLSCAASGFIFRAASMDWYRQFPGNERESIATISS |
| EpCAM binder H138 | GGFTNYADSVKGRFTISRDNSKNTLYLQMNSLRAEDTAVYYCGATFLRSDGH |
| (SEQ ID No. 966) | HTINGQGTLVTVSS |
| Exemplary humanized | EVQLLESGGGLVQPGGSLTLSCAASGFIFRAASMAWYRQSPGNERELVASIS |
| EpCAM binder H90 (SEQ | SGAFTNYADSVKARFTISRDNSKNTLYLQMNSLRAEDTAVYYCGATFLRSDG |
| ID No. 967) | HHTINGQGTLVTVSS |
| Humanized Anti- | VVGGGGTQTVVTQEPSLTVSPGGTVTLTCASSTGAVTSGNYPNWVQQKPG |
| CD3/Anti-EpCAM | QAPRGLIGGTKFLVPGTPARFSGSLLGGKAALTLSGVQPEDEAEYYCTLWYS |
| sequence name H13 | NRWVFGGGTKLTVLGGGGSGGGGSGGGGSEVQLVESGGGLVQPGGSLKL |
| (SEQ ID No. 968) | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSEVQLVESGGGLVQPGGSLTLSCAASGFAFGNHWMYW |
| | YRQAPGRGRELVASISSGGSTNYVDSVKGRFTISRDNAKNTLYLQMNSLRAE |
| | DTAVYYCGTSDNWGQGTLVTVSSHHHHHH* |
| Humanized Anti- | VVGGGGTQTVVTQEPSLTVSPGGTVTLTCASSTGAVTSGNYPNWVQQKPG |
| CD3/Anti-EpCAM | QAPRGLIGGTKFLVPGTPARFSGSLLGGKAALTLSGVQPEDEAEYYCTLWYS |
| sequence name H138 | NRWVFGGGTKLTVLGGGGSGGGGSGGGGSEVQLVESGGGLVQPGGSLKL |
| (SEQ ID No. 969) | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSEVQLLESGGGLVQPGGSLTLSCAASGFIFRAASMDWYR |
| | QFPGNERESIATISSGGFTNYADSVKGRFTISRDNSKNTLYLQMNSLRAEDTA |
| | VYYCGATFLRSDGHHTINGQGTLVTVSSHHHHHH* |
| Humanized Anti- | VVGGGGTQTVVTQEPSLTVSPGGTVTLTCASSTGAVTSGNYPNWVQQKPG |
| CD3/Anti-EpCAM | QAPRGLIGGTKFLVPGTPARFSGSLLGGKAALTLSGVQPEDEAEYYCTLWYS |
| sequence name H90 | NRWVFGGGTKLTVLGGGGSGGGGSGGGGSEVQLVESGGGLVQPGGSLKL |
| (SEQ ID No. 970) | SCAASGFTFNKYAINWVRQAPGKGLEWVARIRSKYNNYATYYADQVKDRFT |
| | ISRDDSKNTAYLQMNNLKTEDTAVYYCVRHANFGNSYISYWAYWGQGTLV |
| | TVSSGGGGSGGGSEVQLLESGGGLVQPGGSLTLSCAASGFIFRAASMAWYR |
| | QSPGNERELVASISSGAFTNYADSVKARFTISRDNSKNTLYLQMNSLRAEDTA |
| | VYYCGATFLRSDGHHTINGQGTLVTVSSHHHHHH* |
| HuCD3e | QDGNEEMGGITQ |
| (SEQ ID No. 989) | |
| AB loop oligo WT: | LVQPGN |
| SEQ ID No. 990 | |
| C″D loop oligo WT: | DSVKGR |
| SEQ ID No.990 | |
| EF loop oligo WT: | SLRPED |
| SEQ ID No. 991 | |
| EpCAM H90 TriTAC | EVQLVESGGGLVQPGNSLRLSCAASGFTFSKFGMSWVRQAPGKGLEWVSSISGSGR |
| C2854 | DTLYADSVKGRFTISRDNAKTTLYLQMNSLRPEDTAVYYCTIGGSLSVSSQGTLVT |
| (SEQ ID No. 992) | VSSGGGGSGGGSQTVVTQEPSLTVSPGGTVTLTCASSTGAVTSGNYPNWVQQKPGQ |
| | APRGLIGGTKFLVPGTPARFSGSLLGGKAALTLSGVQPEDEAEYYCTLWYSNRWVF |
| | GGGTKLTVLGGGGSGGGGSGGGGSEVQLVESGGGLVQPGGSLKLSCAASGFTFNKY |
| | AINWVRQAPGKGLEWVARIRSKYNNYATYYADQVKDRFTISRDDSKNTAYLQMNNL |
| | KTEDTAVYYCVRHANFGNSYISYWAYWGQGTLVTVSSGGGGSGGGSEVQLLESGGG |
| | LVQPGGSLTLSCAASGFIFRAASMAWYRQSPGNERELVASISSGAFTNYADSVKAR |
| | FTISRDNSKNTLYLQMNSLRAEDTAVYYCGATFLRSDGHHTINGQGTLVTVSSHHH |
| | HHH |
| EpCAM H90 ProTriTAC | QTVVTQEPSLTVSPGGTVTLTCASSTGAVTSGNYPNWVQQKPGQAPRGLIGGTKFL |
| (L040) C2704 | VPGTPARFSGSLLGGKAALTLSGVQPEDEAEYYCTLWYSNRWVFGGGTKLTVLGGG |
| (SEQ ID No. 993) | GSGGGGSGGGGSEVQLVESGGGLVQPGGSLKLSCAASGFTFNKYAINWVRQAPGKG |
| | LEWVARIRSKYNNYATYYADQVKDRFTISRDDSKNTAYLQMNNLKTEDTAVYYCVR |
| | HANFGNSYISYWAYWGQGTLVTVSSGGGGSGGGSEVQLLESGGGLVQPGGSLTLSC |
| | AASGFIFRAASMAWYRQSPGNERELVASISSGAFTNYADSVKARFTISRDNSKNTL |
| | YLQMNSLRAEDTAVYYCGATFLRSDGHHTINGQGTLVTVSSHHHHHH |
| EpCAM H90 ProTriTAC | EVQLVESGGGLVQPGNSLRLSCAASGFTFSKFGMSWVRQGGGGGLDGNEEPGGLEW |
| (NCLV) C2302 | VSSISGSGRDTLYADSVKGRFTISRDNAKTTLYLQMNSLRPEDTAVYYCTIGGSLS |
| (SEQ ID No. 994) | VSSQGTLVTVSSGGGGSGGGGSGGVVGGGGTQTVVTQEPSLTVSPGGTVTLTCASS |
| | TGAVTSGNYPNWVQQKPGQAPRGLIGGTKFLVPGTPARFSGSLLGGKAALTLSGVQ |
| | PEDEAEYYCTLWYSNRWVFGGGTKLTVLGGGGSGGGGSGGGGSEVQLVESGGGLVQ |
| | PGGSLKLSCAASGFTFNKYAINWVRQAPGKGLEWVARIRSKYNNYATYYADQVKDR |
| | FTISRDDSKNTAYLQMNNLKTEDTAVYYCVRHANFGNSYISYWAYWGQGTLVTVSS |
| | GGGGSGGGSEVQLLESGGGLVQPGGSLTLSCAASGFTFRAASMAWYRQSPGNEREL |
| | VASISSGAFTNYADSVKARFTISRDNSKNTLYLQMNSLRAEDTAVYYCGATFLRSD |
| | GHHTINGQGTLVTVSSHHHHHH |

1. A dual binding moiety comprising a non-CDR loop and a cleavable linker, wherein the dual binding moiety is capable of an interaction with a domain by specific binding or by steric occlusion,
wherein upon the interaction between the dual binding moiety and the domain, the dual binding moiety is capable of masking the domain from binding its target, and
wherein upon cleavage of the cleavable linker, the dual binding moiety is capable of unmasking the domain.

2. The dual binding moiety of claim 1, wherein the dual binding moiety is further capable of interacting with a half-life extending protein.

3. (canceled)

4. The dual binding moiety of claim 1, wherein the non-CDR loop provides a binding site specific for the domain.

5. The dual binding moiety of claim 4, wherein the non-CDR loop comprises AB, C″D, EF, and CC′ loops and wherein the binding site specific for the domain is within the AB, C″D, EF, or CC′ loop.

6. (canceled)

7. (canceled)

8. The dual binding moiety of claim 5, wherein a CD3 binding epitope is grafted into at least one of the AB, C″D, EF, and CC′ loops.

9. (canceled)

10. (canceled)

11. The dual binding moiety of claim 1, wherein the dual binding moiety comprises a natural peptide, a synthetic peptide, an engineered scaffold, or an engineered bulk serum protein.

12. The dual binding moiety of claim 11, wherein the engineered scaffold comprises at least one of: an sdAb, an scFv, an Fab, a VHH, a IgNAR, a VH, a VL, a fibronectin type III domain, an immunoglobulin-like scaffold, a bacterial albumin-binding domain, an adnectin, a monobody, an affibody, an affilin, an affimer, an affitin, an alphabody, an anticalin, an avimer, a centyrin, a DARPin, a cystine knot peptide, a lipocalin, a three-helix bundle scaffold, a protein G-related albumin-binding module, a DNA or RNA aptamer scaffold, or any combinations thereof.

13. The dual binding moiety of claim 1, wherein the non-CDR-loop is from at least one of: a variable domain of an immunoglobulin molecule, a constant domain of an immunoglobulin molecule, a C1-set domain of an immunoglobulin molecule, a C2-set domain of an immunoglobulin molecule, an I-domain of an immunoglobulin molecule, or any combinations thereof.

14. (canceled)

15. The dual binding moiety of claim 1, wherein the dual binding moiety comprises albumin.

16. The dual binding moiety of claim 1, wherein the dual binding moiety further comprises a complementarity determining region (CDR).

17. The dual binding moiety of claim 1, wherein the dual binding moiety comprises a binding site specific for a bulk serum protein.

18. The dual binding moiety of claim 17, wherein the bulk serum protein is at least one of: albumin, transferrin, IgG1, IgG2, IgG4, IgG3, IgA monomer, Factor XIII, Fibrinogen, IgE, and pentameric IgM, or any combinations thereof.

19.-21. (canceled)

22. The dual binding moiety of claim 1, wherein the dual binding moiety comprises a binding site specific for a CD3ε domain, and wherein the binding site for the CD3ε domain comprises at least one of the following motifs: QDGNE, QDGNEE, DGNE, and DGNEE.

23. The dual binding moiety of claim 1, wherein the cleavable linker comprises a cleavage site.

24. The dual binding moiety of claim 23, wherein the cleavage site is a protease cleavage site.

25. The dual binding moiety of claim 1, wherein the domain is a target antigen binding domain, and wherein the dual binding moiety is capable of masking the target antigen binding domain from binding to a target.

26. The dual binding moiety of claim 25, wherein the target comprises a tumor antigen.

27. The dual binding moiety of claim 26, wherein the tumor antigen comprises EpCAM, EGFR, HER-2, HER-3, c-Met, FoIR, PSMA, CD38, BCMA, CEA. 5T4, AFP, B7-H3, Cadherin-6, CAIX, CD117, CD123, CD138, CD166, CD19, CD20, CD205, CD22, CD30, CD33, CD40, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, P-cadherin, NY-ESO-1, PRLR, PSCA, PTK7, ROR1, SLC44A4, SLTRKS, SLTRK6, STEAP1, TIM1, Trop2, or WT1.

28.-31. (canceled)

32. The dual binding moiety of claim 24, wherein the protease cleavage site is recognized by a serine protease, a cysteine protease, an aspartate protease, a threonine protease, a glutamic acid protease, a metalloproteinase, a gelatinase, or a asparagine peptide lyase.

33. The dual binding moiety of claim 24, wherein the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP7, a MMP8, a MMP9, a MMP10, a MMP11, a MMP12, a MMP13, a MMP14, an ADAMS, an ADAM10, an ADAM12, an urokinase plasminogen activator (uPA), an enterokinase, a prostate-specific target (PSA, hK3), an interleukin-1β converting enzyme, a thrombin, a FAP (FAP-α), a dipeptidyl peptidase, a type II transmembrane serine protease (TTSP), a neutrophil elastase, a cathepsin G, a proteinase 3, a neutrophil serine protease 4, a mast cell chymase, or a mast cell tryptase.

34-72. (canceled)

73. The dual binding moiety of claim 1, wherein the dual binding moiety comprises a sequence selected from the group consisting of SEQ ID Nos: 50 and 259-301.
